(19) United States
(12) Patent Application Publication
(10) Pub. No.: US 2009/0023798 A1
(43) Pub. Date: Jan. 22, 2009

(54) THIOHYDROXAMATES AS INHIBITORS OF HISTONE DEACETYLASE

(75) Inventors: Darren Magda, Cupertino, CA (US); Zhong Wang, Sunnyvale, CA (US)

Correspondence Address: WILSON SONSINI GOODRICH & ROSATI, 650 PAGE MILL ROAD, PALO ALTO, CA, 94304-1050, US

(73) Assignee: PHARMACYCLICS, INC., Sunnyvale, CA (US)

(21) Appl. No.: 12/140,034

(22) Filed: Jun. 16, 2008

Related U.S. Application Data
(60) Provisional application No. 60/944,425, filed on Jun. 15, 2007.

(51) Int. Cl. A61K 31/404; C07D 307/78; A61K 31/343; A61P 35/00; C07D 209/04

(52) U.S. Cl. 514/419; 549/467; 514/469; 548/491

(57) ABSTRACT

Described herein are compounds and pharmaceutical compositions containing such compounds, which inhibit the activity of histone deacetylase. Also described herein are methods of using such HDAC inhibitors, alone and in combination with other compounds, for treating diseases or conditions that would benefit from inhibition of HDAC activity.

### RELATED APPLICATIONS

This application claims the benefit of U.S. Provisional Patent Application No. 60/944,425, filed Jun. 15, 2007, the contents of which are herein incorporated by reference in their entirety.

### FIELD OF THE INVENTION

Described herein are compounds, methods of making such compounds, pharmaceutical compositions and medicaments that include such compounds, and methods of using such compounds to inhibit the activity of histone deacetylase.

### BACKGROUND OF THE INVENTION

Histone deacetylases (HDACs) catalyze the removal of acetyl groups from histones, proteins that organize and modulate the structure of chromatin in nucleosomes. HDAC-mediated deacetylation of chromatin-bound histones regulates the expression of a variety of genes throughout the genome. Importantly, HDACs have been linked to cancer, as well as other health conditions. To date, eleven major HDAC isoforms have been described (HDACs 1-11). HDACs are categorized into two classes. Class I HDACs include HDAC1, HDAC2, HDAC3, HDAC8 and HDAC11. Class II HDACs include HDAC4, HDAC5, HDAC6, HDAC7, HDAC9 and HDAC10. Small molecule HDAC inhibitors that are isoform-selective are useful as therapeutic agents with reduced toxicity and as tools for probing the biology of the HDAC isoforms.

### SUMMARY OF THE INVENTION

Compounds disclosed herein are HDAC inhibitor compound. Compounds disclosed herein include compounds of Formula (I), Formula (Ia), Formula (Ib), Formula (Ic), Formula (II), Formula (IIa), Formula (IIIa), Formula (IIIb), Formula (IVa), Formula (IVb).

In one aspect, disclosed herein are compounds of Formula (I):

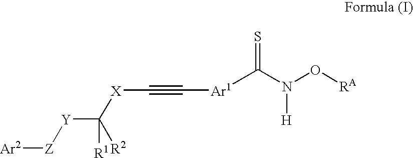

wherein:
R^{A} is hydrogen, alkyl, or alkylcarbonyl;
Ar¹ is arylene or heteroarylene wherein said Ar¹ is optionally substituted with one or two substituents independently selected from alkyl, halo, alkoxy, haloalkoxy, or haloalkyl;
X and Y are independently selected from bond or alkylene wherein alkylene is optionally substituted with halo, haloalkyl, hydroxy, alkoxy, haloalkoxy, amino, alkylamino, or dialkylamino;
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form cycloalkylene or heterocycloalkylene;
Z is —CONR³—, —NR³CO—, SO₂NR³—, —NR³SO₂—, —NR³CONR⁴—, —NR³SO₂NR⁴—, —OCONR³—, or NR³COO— where R³ and R⁴ are independently selected from hydrogen, alkyl, hydroxyalkyl, haloalkyl, haloalkoxy, alkoxyalkyl, aralkyl, or heteroaralkyl; and
Ar² is aryl, aralkyl, aralkenyl, heteroaryl, heteroaralkyl, heteroaralkenyl, heterocycloalkyl, or heterocycloalkylalkyl; and
   individual stereoisomers, individual geometric isomers, or mixtures thereof; or
a pharmaceutically acceptable salt thereof provided that the thiohydroxamic acid and the acetylenic groups are not adjacent to each other on Ar¹.

For any and all of the embodiments of Formula (I), substituents can be selected from among from a subset of the listed alternatives. For example, in some embodiments, Z is —CONR³—, —NR³CO—, SO₂NR³—, or —NR³SO₂—.

In one aspect, Z is —CONH—, SO₂NH—, or —NHCONH—. In another aspect, Z is —CONH—.

In one aspect, R^{A} is hydrogen; and Ar¹ is arylene optionally substituted with one or two substituents independently selected from alkyl, halo, or haloalkyl.

In one aspect, Ar¹ is phenylene and Ar²-Z-Y—(CR¹R²)—X-≡- is attached to Ar¹ in the para position relative to the C(═S)NHOR^{A} group.

In one aspect, R^{A} is hydrogen; Ar¹ is heteroarylene optionally substituted with one or two substituents independently selected from among alkyl, halo, and haloalkyl.

In one aspect, X and Y are a bond; and R¹ and R² are independently hydrogen or alkyl.

In one aspect, X and Y are independently a bond or alkylene provided that both X and Y are not a bond.

In some embodiments, X and Y are a bond; and R¹ and R² together with the carbon atom to which they are attached form a cycloalkylene.

In some embodiments, X and Y are a bond; and R¹ and R² together with the carbon atom to which they are attached form a heterocycloalkylene.

In some embodiments, X and Y are independently a bond or alkylene provided that both are not a bond; and R¹ and R² together with the carbon atom to which they are attached form cycloalkylene.

In some embodiments, X and Y are independently a bond or alkylene provided that both are not bond; and R¹ and R² together with the carbon atom to which they are attached form heterocycloalkylene.

In some embodiments, Ar² is aryl. In some other embodiments, Ar² is phenyl optionally substituted with one, two, or three substituents independently selected from among alkyl, alkoxy, alkoxyalkyl, halo, haloalkyl, haloalkoxy, alkylamino, dialkylamino, hydroxy, hydroxyalkyl, hydroxyalkyloxy, aminoalkyl, aminoalkoxy, alkoxyalkyloxy, optionally substituted phenyloxyalkyl, optionally substituted heteroaralkyloxy, optionally substituted heteroaryloxyalkyl, optionally substituted heterocycloalkylalkyloxy, and optionally substituted heterocycloalkylalkyl. In yet other embodiments, Ar² is phenyl optionally substituted with one, two, or three substituents independently selected from among methyl, ethyl, methoxy, chloro, trifluoromethyl, fluoro, 2-methoxyethoxy, 2-(morpholin-4-yl)ethoxy, pyridin-3-ylmethoxy, 2-hydroxyethoxy, 2-(N,N-dimethylamino)ethoxy, methoxymethyl, 2-morpholino-4-ylethyl, morpholino-4-ylmethyl, N,N-dimethylaminomethyl, iso-propoxymethyl, and phenoxymethyl.

In some embodiments, Ar² is heteroaryl. In other embodiments, Ar² is thienyl, pyridinyl, quinolinyl, thiazolyl, benzthiazolyl, benzoxazolyl, furanyl, benzimidazolyl, benzothienyl, pyrrolyl, indolyl, isoindolyl, or isoquinolinyl, optionally substituted with one or two substituents independently selected from among alkyl, alkoxy, alkoxyalkyl, halo, haloalkyl, haloalkoxy, alkylamino, dialkylamino, hydroxy, hydroxyalkyl, hydroxyalkyloxy, aminoalkyl, aminoalkoxy, alkoxyalkyloxy, optionally substituted phenyl, optionally substituted heteroaryl, optionally substituted phenyloxyalkyl, optionally substituted heteroaralkyloxy, optionally substituted heteroaryloxyalkyl, optionally substituted heterocycloalkylalkyloxy, or optionally substituted heterocycloalkylalkyl. In some embodiments, Ar² is optionally substituted with one or two substituents independently selected from methyl, ethyl, methoxy, chloro, trifluoromethyl, fluoro, 2-methoxyethoxy, 2-(morpholin-4-yl)ethoxy, pyridin-3-ylmethoxy, 2-hydroxyethoxy, 2-(N,N-dimethylamino)ethoxy, methoxymethyl, 2-morpholino-4-ylethyl, morpholino-4-ylmethyl, N,N-dimethylamino-methyl, 2-N,N-dimethylaminoethyl, aminomethyl, iso-propoxymethyl, phenyl, or phenoxymethyl. In other embodiments, Ar² is thienyl, pyridinyl, quinolinyl, thiazolyl, benzthiazolyl, benzoxazolyl, furanyl, benzimidazolyl, benzothienyl, pyrrolyl, indolyl, isoindolyl, or isoquinolinyl optionally substituted with one or two substituents independently selected from alkyl, alkoxy, alkoxyalkyl, halo, haloalkyl, haloalkoxy, alkylamino, dialkylamino, hydroxy, hydroxyalkyl, hydroxyalkyloxy, aminoalkyl, aminoalkoxy, alkoxyalkyloxy, optionally substituted phenyl, optionally substituted heteroaryl, optionally substituted phenyloxyalkyl, optionally substituted heteroaralkyloxy, optionally substituted heteroaryloxyalkyl, optionally substituted heterocycloalkylalkyloxy, or optionally substituted heterocycloalkylalkyl.

In some embodiments, Ar² is thiazolyl, quinolinyl, oxazolyl, benzothienyl, indolyl, benzofuranyl, benzthiazolyl, or benzimidazolyl optionally substituted with a substitutent selected from halo, haloalkyl, alkoxy, haloalkoxyalkyl, aminoalkoxy, aminoalkyl, alkoxyalkyloxy, alkoxyalkyloxyalkyl, optionally substituted heterocycloalkyloxy, optionally substituted heterocycloalkylalkyloxy, or phenyl optionally substituted with -(alkylene)NRR′ where R and R′ are independently hydrogen or alkyl.

In some embodiments, disclosed herein are compounds of Formula (Ic):

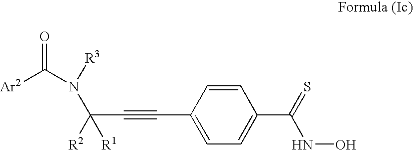

wherein:
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form a cycloalkylene or heterocycloalkylene;
R³ is hydrogen, alkyl, haloalkyl, alkoxyalkyl, aralkyl, or heteroaralkyl; and
Ar² is aryl, aralkyl, aralkenyl, heteroaryl, heteroaralkyl, heteroaralkenyl, heterocycloalkyl, or heterocycloalkylalkyl; and
individual stereoisomers, individual geometric isomers, or mixtures thereof; or
a pharmaceutically acceptable salt thereof.

For any and all of the embodiments, substituents can be selected from among from a subset of the listed alternatives. For example, in some embodiments, R³ is hydrogen or alkyl.

In some embodiments, R¹ is hydrogen or alkyl; R² is hydrogen or alkyl; or R¹ and R² together with the carbon to which they are attached form a cycloalkylene or heterocycloalkylene.

In some embodiments, Ar² is aryl, aralkyl, aralkenyl, heteroaryl, heteroaralkyl, or heteroaralkenyl.

In some embodiments, Ar² is selected from among phenyl, phenyl-CH═CH—, oxazolyl, thiazolyl, benzthiazolyl, benzimidazolyl, benzothienyl, quinolinyl, indolyl, and benzofuranyl, and if Ar² is substituted, then each substituent is independently selected from among hydrogen, phenyl; 4-H₂NCH₂-phenyl; F; Cl; CF₃—; CF₃O—; CF₃CH₂OCH₂; MeO; (CH₃)₂NCH₂—; (CH₃)₂N(CH₂)₂—;(CH₃)₂N(CH₂)₂O—; CH₃—O—(CH₂)₂OCH₂—; tetrahydropyran-4-yl-O—; 2-pyrrolidin-1-ylethyloxy; CH₃—O—(CH₂)₂O—; 4-(CH₃)₂N(CH₂)₂O—; (1-CF₃CH₂-piperidin-4-yloxy); (1-cyclopropylpiperidin-4-yloxy); tetrahydropyran-4-ylmethoxy; and (2-morpholin-4-ylethoxy).

In one aspect, disclosed herein is a pharmaceutical composition comprising a therapeutically effective amount of a compound of Formula (I) and a pharmaceutically acceptable excipient.

In one aspect, is a method for treating a disease in an animal mediated by HDAC which method comprises administering to the animal a pharmaceutical composition comprising a therapeutically effective amount of a compound of Formula (I) and a pharmaceutically acceptable excipient. In one aspect, the disease is cancer and the animal is a human.

In one aspect, disclosed herein are compounds of Formula (II):

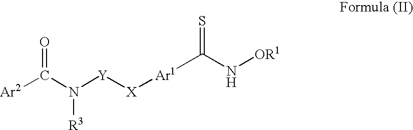

wherein:
R^{A} is hydrogen or alkyl;
X is a bond, O—, —NR¹—, or S(O)ₙ;
n is 0, 1, or 2;
R¹ is hydrogen or alkyl;
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

For any and all of the embodiments of Formula (II), substituents can be selected from among from a subset of the listed alternatives. For example, in some embodiments, R^{A} is hydrogen or alkyl; X is O—, —NR¹—, or S(O)ₙ; n is 0, 1 or 2; Y is alkylene optionally substituted with cycloalkyl, optionally substituted phenyl, alkylthio, alkysulfonyl, optionally substituted phenylalkylthio, optionally substituted phenylalkylsulfonyl, or hydroxy; Ar¹ is phenylene or heteroarylene wherein said Ar¹ is optionally substituted with one or two groups independently selected from alkyl, halo, hydroxy, alkoxy, haloalkoxy, or haloalkyl; R³ is hydrogen, alkyl, hydroxyalkyl, or optionally substituted phenyl; and Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted.

In some embodiments, X is O—; and R^{A} is hydrogen.

In some embodiments, X is S(O)ₙ; and R^{A} is hydrogen.

In some embodiments, wherein Y is alkylene. In some embodiments, Y is alkylene substituted with cycloalkyl, optionally substituted phenyl, alkylthio, alkylsulfinyl, alkysulfonyl, optionally substituted phenylalkylthio, optionally substituted phenylalkylsulfonyl, hydroxyl, or optionally substituted phenoxy.

In some embodiments, Ar¹ is phenylene, where C(═S)NHOR^{A} and Ar²—C(═O)NR³—Y—X— are para to each other on the phenylene ring; X is O—; R^{A} is hydrogen.

In some embodiments, Ar¹ is heteroarylene.

In some embodiments, R³ is hydrogen; methyl; ethyl; propyl; butyl; i-propyl; 2-hydroxy-ethyl; or phenyl. In some embodiments, R³ is hydrogen.

In some embodiments, Y is —CH₂—CH₂—; —CH₂—CH(CH₃)—; —CH(CH₃)—CH₂—; —CH(CH₂CH₃)—CH₂—; —CH(i-propyl)-CH₂—; —CH(i-butyl)-CH₂—; —CH(n-butyl)-CH₂—; —CH(cyclohexyl-methyl)-CH₂—; —CH(benzyl)-CH₂—; —CH(2-phenylethyl)-CH₂—; —CH(benzylS-methyl)-CH₂—; —CH(benzylSO₂-methyl)-CH₂—; —CH(2-MeS-ethyl)-CH₂—; —CH(hydroxy-methyl)-CH₂—; —CH(2-MeSO₂-ethyl)-CH₂—; —CH(4-Cl-benzyl)-CH₂—; —CH(benzyl-5-methyl)-CH₂—; —CH(phenyl)-CH₂—; —CH(benzylSO₂-methyl)-CH₂—; —CH₂—CH₂—CH₂—; —CH(CH₃Smethyl)-CH₂—; —CH(CH₃SO₂-methyl)-CH₂—; or —CH₂CH-(phenoxymethyl)-.

In some embodiments, Ar² is aryl, aralkyl, aralkenyl, heteroaryl, heteroaralkyl, or heteroaralkenyl, where Ar² is substituted or unsubstituted.

In some embodiments, Ar² is aryl(C₂-C₃)alkenyl, where Ar² is substituted or unsubstituted. In some embodiments, Ar² is trans-phenyl-CH═CH—, trans-(4-methoxy-phenyl)-CH═CH—, trans-(3,4-methylenedioxyphenyl)-CH═CH—, trans-(3-hydroxyphenyl)-CH═CH—, trans-(4-hydroxyphenyl)-CH═CH—, trans-(2-methoxyphenyl)-CH═CH—, trans-(3-methoxyphenyl)-CH═CH—, trans-(3-tolyl)-CH═CH—, trans-(4-tolyl)-CH═CH—, trans-(4-dimethylaminophenyl)-CH═CH—, trans-(2-tolyl)-CH═CH—, or trans-(2-hydroxyphenyl)-CH═CH—.

In some embodiments, Ar² is heteroaryl(C₂-C₃)alkenyl, where Ar² is substituted or unsubstituted. In some embodiments, Ar² is trans pyridin-3-yl-CH═CH—, trans-(5-hydroxybenzofuran-2-yl)-C(CH₃)═CH—, trans-(5-(1-cyclopropylpiperidin-4-yloxy)benzofuran-2-yl)-C(CH₃)═CH—, trans-(5-methoxybenzofuran-2-yl)-C(CH₃)═CH—, trans-(benzofuran-2-yl)-CH═CH—, trans-(5-bromothien-2-yl)-CH═CH—, trans-(furan-3-yl)-CH═CH—, trans-(thien-3-yl)-CH═CH—, trans-(thien-2-yl)-CH═CH—, trans-(benzofuran-2-yl)-C(CH₃)═CH—, cis-(benzofuran-2-yl)-C(CH₃)═CH—, trans-(indol-3-yl)-CH═CH—, trans-(7-methoxybenzofuran-2-yl)-CH═CH—, trans-(5-methoxybenzofuran-2-yl)-C(CH₃)═CH—, or trans-(furan-2-yl)-CH═CH.

In some embodiments, Ar² is aryl, where Ar² is substituted or unsubstituted. In some embodiments, Ar² is aryl and the substitutents on the aryl ring are independently selected from among hydrogen, optionally substituted phenyl, alkyl, alkoxy, halo, optionally substituted heteroaryl, optionally substituted cycloalkenyloxy, optionally substituted heteroaralkyloxy, optionally substituted heterocycloalkyl, optionally substituted phenylcarbonylamino, and methylenedioxy. In some embodiments, Ar² is phenyl, 4-biphenyl, 3-biphenyl, 4-tert-butylphenyl, 4-pyrrol-1-ylphenyl, 4-(pyridin-3-yl)phenyl, 4-(pyridin-2-yl)phenyl, 4-(benzoylamino)phenyl, 2,4-difluorophenyl, 3,4-methylenedioxyphenyl, 3,4-dimethoxyphenyl, 3,5-dimethoxyphenyl, 3,4-difluorophenyl, 2,5-dimethylphenyl, 2,3-dichlorophenyl, 2,3-dimethylphenyl, 4-chloro-2-methoxyphenyl, 3-ethoxyphenyl, 4-methoxy-2-methylphenyl, 3-fluoro-4-methoxyphenyl, 2-(thien-2-ylmethoxy)phenyl, 3-(thien-2-ylmethoxy)-phenyl, 2-biphenyl, naphth-1-yl, 2-pyrrol-1-yl-phenyl, 4-fluoronaphth-1-yl, 3-methoxy-naphth-2-yl, 2-methoxy-naphth-1-yl, naphth-2-yl, 4-(2-pyridin-4-ylthiazol-5-yl)phenyl, 4-[2-(4-methylpiperazin-1-yl)thiazol-5-yl]-phenyl, 4-(2-pyridin-4-ylaminothiazol-5-yl)phenyl, 4-(4-methylpiperazin-1-yl)phenyl, 4-(4-hydroxypiperidin-1-yl)phenyl, 4-[2-(4-methylpiperazin-1-ylmethyl)thiazol-5-yl]phenyl, 1-methoxynaphth-2-yl, 3′-(2-hydroxyethyl)biphen-4-yl, 3′-(2-hydroxyethyl)biphen-3-yl, 2′-(2-hydroxyethyl)biphen-4-yl, 2′-(2-hydroxyethyl)biphen-3-yl or 4-[4-(2-morpholin-4-yl-ethyl)thiazol-2-yl]phenyl.

In some embodiments, Ar² is heteroaryl, where Ar² is substituted or unsubstituted. In some embodiments, Ar² is heteroaryl optionally substituted with one or two substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR^{c}-alkyleneCONR^{c}R^{d} (where R^{c} is hydroxyl and R^{d} and R^{e} are independently hydrogen or alkyl), -alkylene-N(heteroalkyl)₂, or carboxyalkylaminoalkyl.

In some embodiments, Ar² is thien-2-yl, pyridin-3-yl, quinolin-6-yl, benzothiazol-2-yl, benzoxazol-2-yl, benzofuran-2-yl, benzofuran-5-yl, benzothien-2-yl, furan-2-yl, 1H-benzimidazol-2-yl, 1H-pyrrol-2-yl, thiazol-2-yl, 1H-indol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, quinolin-1-yl, pyridin-2-yl, quinolin-2-yl, furan-3-yl, or thien-3-yl, wherein Ar² is optionally substituted with one or two substituents selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R^{a} is hydroxyalkyl or optionally substituted phenyl), -alkylene-NR^{c}-alkyleneCONR^{c}R^{d} (where R^{c} is hydroxyl and R^{d} and R^{e} are independently hydrogen or alkyl), -alkylene-N(heteroalkyl)₂, and carboxyalkylaminoalkyl.

In some embodiments, Ar² is benzofuran-2-yl and is monosubstituted at the 3-position of the benzofuran-2-yl ring wherein the substituent is N,N-dimethylaminomethyl, N,N-diethylaminomethyl, 2-fluorophenoxymethyl, 3-fluorophenoxymethyl, 4-fluorophenoxy-methyl, hydroxyl-4-yloxymethyl, 2,4,6-trifluorophenoxy-methyl, 2-oxopyridin-1-ylmethyl, 2,2,2-trifluoroethoxy-methyl, 4-imidazol-1-ylphenoxy-methyl, 4-[1.2.4-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenylethyl), N-methyl-N-benzylaminomethyl, N-methyl-N-2-phenylethylaminomethyl, 3-hydroxypropyl-thiomethyl, 3-hydroxypropylsulfinyl-methyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In one embodiment, compounds of Formula (II) have a structure of Formula (IIa):

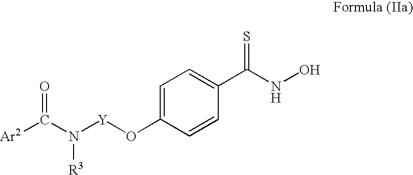

wherein:
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

In some embodiments, R³ is hydrogen; methyl; ethyl; propyl; butyl; i-propyl; 2-hydroxy-ethyl; phenyl; or benzyl.

In some embodiments, Y is —CH₂—CH₂-; —CH₂—CH(CH₃)—; —CH(CH₃)—CH₂—; —CH(CH₂CH₃)—CH₂—; —CH(i-propyl)-CH₂—; —CH(i-butyl)-CH₂—; —CH(n-butyl)-CH₂—; —CH(cyclohexyl-methyl)-CH₂—; —CH(benzyl)-CH₂—; —CH(2-phenylethyl)-CH₂—; —CH(benzylS-methyl)-CH₂—; —CH(benzylSO₂-methyl)-CH₂—; —CH(2-MeS-ethyl)-CH₂—; —CH(hydroxy-methyl)-CH₂—; —CH(2-MeSO₂-ethyl)-CH₂—; —CH(4-Cl-benzyl)-CH₂—; —CH(benzyl-5-methyl)-CH₂—; —CH(phenyl)-CH₂—; —CH(benzylSO₂-methyl)-CH₂—; —CH₂—CH₂—CH₂—; —CH(CH₃Smethyl)-CH₂—; —CH(CH₃SO₂-methyl)-CH₂—; or —CH₂CH-(phenoxymethyl)-.

In some embodiments, Ar² is aryl, aralkyl, aralkenyl, heteroaryl, heteroaralkyl, or heteroaralkenyl, where Ar² is substituted or unsubstituted.

In some embodiments, Ar² is aryl(C₂-C₃)alkenyl, where Ar² is substituted or unsubstituted. In some embodiments, Ar² is trans-phenyl-CH═CH—, trans-(4-methoxy-phenyl)-CH═CH—, trans-(3,4-methylenedioxyphenyl)-CH═CH—, trans-(3-hydroxyphenyl)-CH═CH—, trans-(4-hydroxyphenyl)-CH═CH—, trans-(2-methoxyphenyl)-CH═CH—, trans-(3-methoxyphenyl)-CH═CH—, trans-(3-tolyl)-CH═CH—, trans-(4-tolyl)-CH═CH—, trans-(4-dimethylaminophenyl)-CH═CH—, trans-(2-tolyl)-CH═CH—, or trans-(2-hydroxyphenyl)-CH═CH—.

In some embodiments, Ar² is heteroaryl(C₂-C₃)alkenyl, where Ar² is substituted or unsubstituted. In some embodiments, Ar² is trans pyridin-3-yl-CH═CH—, trans-(5-hydroxybenzofuran-2-yl)-C(CH₃)═CH—, trans-(5-(1-cyclopropylpiperidin-4-yloxy)benzofuran-2-yl)-C(CH₃)═CH—, trans-(5-methoxybenzofuran-2-yl)-C(CH₃)═CH—, trans-(benzofuran-2-yl)-CH═CH—, trans-(5-bromothien-2-yl)-CH═CH—, trans-(furan-3-yl)-CH═CH—, trans-(thien-3-yl)-CH═CH—, trans-(thien-2-yl)-CH═CH—, trans-(benzofuran-2-yl)-C(CH₃)═CH—, cis-(benzofuran-2-yl)-C(CH₃)═CH—, trans-(indol-3-yl)-CH═CH—, trans-(7-methoxybenzofuran-2-yl)-CH═CH—, trans-(5-methoxybenzofuran-2-yl)-C(CH₃)═CH—, or trans-(furan-2-yl)-CH═CH.

In some embodiments, Ar² is aryl, where Ar² is substituted or unsubstituted. In some embodiments, Ar² is aryl and the substitutents on the aryl ring are independently selected from among hydrogen, optionally substituted phenyl, alkyl, alkoxy, halo, optionally substituted heteroaryl, optionally substituted cycloalkenyloxy, optionally substituted heteroaralkyloxy, optionally substituted heterocycloalkyl, optionally substituted phenylcarbonylamino, and methylenedioxy. In some embodiments, Ar² is phenyl, 4-biphenyl, 3-biphenyl, 4-tert-butylphenyl, 4-pyrrol-1-ylphenyl, 4-(pyridin-3-yl)phenyl, 4-(pyridin-2-yl)phenyl, 4-(benzoylamino)phenyl, 2,4-difluorophenyl, 3,4-methylenedioxyphenyl, 3,4-dimethoxyphenyl, 3,5-dimethoxyphenyl, 3,4-difluorophenyl, 2,5-dimethylphenyl, 2,3-dichlorophenyl, 2,3-dimethylphenyl, 4-chloro-2-methoxyphenyl, 3-ethoxyphenyl, 4-methoxy-2-methylphenyl, 3-fluoro-4-methoxyphenyl, 2-(thien-2-ylmethoxy)phenyl, 3-(thien-2-ylmethoxy)-phenyl, 2-biphenyl, naphth-1-yl, 2-pyrrol-1-yl-phenyl, 4-fluoronaphth-1-yl, 3-methoxy-naphth-2-yl, 2-methoxy-naphth-1-yl, naphth-2-yl, 4-(2-pyridin-4-ylthiazol-5-yl)phenyl, 4-[2-(4-methylpiperazin-1-yl)thiazol-5-yl]-phenyl, 4-(2-pyridin-4-ylaminothiazol-5-yl)phenyl, 4-(4-methylpiperazin-1-yl)phenyl, 4-(4-hydroxypiperidin-1-yl)phenyl, 4-[2-(4-methylpiperazin-1-ylmethyl)thiazol-5-yl]phenyl, 1-methoxynaphth-2-yl, 3′-(2-hydroxyethyl)biphen-4-yl, 3′-(2-hydroxyethyl)biphen-3-yl, 2′-(2-hydroxyethyl)biphen-4-yl, 2′-(2-hydroxyethyl)biphen-3-yl or 4-[4-(2-morpholin-4-yl-ethyl)thiazol-2-yl]phenyl.

In some embodiments, Ar² is heteroaryl, where Ar² is substituted or unsubstituted. In some embodiments, Ar² is heteroaryl optionally substituted with one or two substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR^{c}-alkyleneCONR^{c}R^{d} (where R^{c} is hydroxyl and R^{d} and R^{e} are independently hydrogen or alkyl), -alkylene-N(heteroalkyl)₂, or carboxyalkylaminoalkyl.

In some embodiments, Ar² is thien-2-yl, pyridin-3-yl, quinolin-6-yl, benzothiazol-2-yl, benzoxazol-2-yl, benzofuran-2-yl, benzofuran-5-yl, benzothien-2-yl, furan-2-yl, 1H-benzimidazol-2-yl, 1H-pyrrol-2-yl, thiazol-2-yl, 1H-indol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, quinolin-1-yl, pyridin-2-yl, quinolin-2-yl, furan-3-yl, or thien-3-yl, wherein Ar² is optionally substituted with one or two substituents selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R^{a} is hydroxyalkyl or optionally substituted phenyl), -alkylene-NR^{c}-alkyleneCONR^{c}R^{d} (where R^{c} is hydroxyl and R^{d} and R^{e} are independently hydrogen or alkyl), -alkylene-N(heteroalkyl)₂, and carboxyalkylaminoalkyl.

In one aspect, disclosed herein is a pharmaceutical composition comprising a therapeutically effective amount of a compound of Formula (II) or pharmaceutically acceptable salts thereof and a pharmaceutically acceptable excipient. In one aspect is the use of a compound of Formula (II) in the preparation of a medicament for treating a disease in an animal mediated by HDAC.

In one aspect, disclosed is a method for treating a disease in an animal mediated by HDAC which method comprises administering to the animal a pharmaceutical composition comprising a therapeutically effective amount of a compound of Formula (II) and a pharmaceutically acceptable excipient.

In one aspect, the disease is cancer and the animal is a human.

In one aspect, disclosed is a method for treating cancer which method comprises administering to the animal a pharmaceutical composition comprising a therapeutically effective amount of a compound of Formula (II) and a pharmaceutically acceptable excipient in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

In one aspect, provided is a method for treating cancer which method comprises administering to the animal a pharmaceutical composition comprising a therapeutically effective amount of a compound of Formula (I) or Formula (II) and a pharmaceutically acceptable excipient in combination with radiation therapy and one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

In one aspect, the method of treating cancer with a compound of Formula (I) or Formula (II) further includes one or more compound(s) independently selected from among abarelix (Plenaxis®); aldesleukin (Prokine®); Aldesleukin (Proleukin®); Alemtuzumab (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacizumab (Avastin®); bexarotene (Targretin®); bleomycin (Blenoxane®); bortezomib (Velcade®); busulfan (Busulfex®); busulfan (Myleran®); calusterone (Methosarb®); capecitabine (Xeloda®); carboplatin Paraplatin®); carmustine (BCNU, BiCNU); carmustine (Gliadel®); celecoxib (Celebrex®); cetuximab (Erbitux®); chlorambucil (Leukeran®); cisplatin (Platinol®); cladribine (Leustatin®); clofarabine (Clolar®); cyclophosphamide (Cytoxan®); cytarabine (Cytosar-U®); cytarabine liposomal (DepoCyt); dacarbazine (DTIC-Dome); dactinomycin(actinomycin D, Cosmegen®); Darbepoetin alfa (Aranesp®); dasatinib (Sprycel®); daunorubicin liposomal (DanuoXome); daunorubicin (daunomycin, Daunorubicin®); daunorubicin(daunomycin, Cerubidine®); decitabine (Dacogen®); denileukin (Ontak®); dexrazoxane (Zinecard®); docetaxel (Taxotere®); doxorubicin (Adriamycin®); doxorubicin liposomal (Doxil®); dromostanolone propionate; epirubicin (Ellence®); Epirubicin; Epoetin alfa (EPOGEN®); erlotinib (Tarceva®); estramustine (Emcyt®); etoposide phosphate (Etopophos®); etoposide (VP-16; Vepesid®); exemestane (AROMASIN®); Filgrastim (Neupogen®); floxuridine (FUDR); fludarabine (Fludara®); fluorouracil (5-FU, Adrucil); fulvestrant (Faslodex®); gefitinib (Iressa®); gemcitabine (Gemzar®); gemtuzumab ozogamicin (Mylotarg®); goserelin acetate (Zoladex®); histrelin acetate (Histrelin®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Leucovorin®); Leuprolide Acetate (Eligard®); levamisole (Ergamisol®); lomustine, CCNU CeeBU®); meclorethamine (nitrogen mustard, Mustargen®); megestrol acetate (Megace®); melphalan (Alkeran®); mercaptopurine (6-MP, Purinethol®); methotrexate (Rheumatrex®, Trexall®); methoxsalen (Uvadex®); mitomycin C (Mutamycin®); mitomycin C (Mitozytrex®); mitotane (Lysodren®); mitoxantrone (Novantrone®); nandrolone phenpropionate (Durabolin-50); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pegademase (Adagen®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); rituximab (Rituxan®); sargramostim (Leukine®); Sargramostim (Prokine®); sorafenib (Nexavar®); streptozocin (Zanosar®); sunitinib maleate (Sutent®); tamoxifen (Nolvadex®); temozolomide (Temodar®); teniposide (VM-26, Vumon®); testolactone (Teslac®); thalidomide (Thalomid®); thioguanine (6-TG, Thioguanine®); thiotepa (Thioplex®); topotecan (Hycamtin®); toremifene (Fareston®); tositumomab (Bexxar®); tositumomab/I-131 tositumomab (Bexxar®); trastuzumab (Herceptin®); tretinoin (ATRA, Vesanoid®); Uracil Mustard; valrubicin (Valstar®); vinblastine (Velban®); vincristine (Oncovin®); vinorelbine (Navelbine®); vorinostat (Zolinza®); zoledronate (Zometa®); and zoledronic acid (Zometa®).

In one aspect, provided is the use of a compound of Formula (II) in the preparation of a medicament for treating cancer in an animal in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, DNA methyltransferase inhibitor, or an angiogenesis inhibitor.

In one aspect, provided is the use of a compound of Formula (II) in the preparation of a medicament for treating cancer in an animal in combination with radiation therapy and optionally in the presence of one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, DNA methyltransferase inhibitor, or an angiogenesis inhibitor.

In one aspect, provided is the use of a compound of Formula (II) in the preparation of a medicament for treating hepatitis C in an animal.

In one aspect provided herein are indole-6-thiohydroxamic acid compounds and indole-5-thiohydroxamic acid compounds, pharmaceutically acceptable salts, pharmaceutically acceptable N-oxides, pharmaceutically active metabolites, pharmaceutically acceptable prodrugs, and pharmaceutically acceptable solvates thereof, which selectively inhibit HDAC8 activity and may be used to treat patients where inhibition of HDAC8 activity would provide benefit. Compounds described herein are selective HDAC8 inhibitors.

Described herein are compounds, compositions and methods for selectively inhibiting HDAC8 activity. In one embodiment, described herein are indole-6-thiohydroxamic acid compounds and indole-5-thiohydroxamic acid compounds that are selective HDAC8 inhibitors.

In one embodiment, described herein is a 1,3-disubstituted-1H-indole-6-thiohydroxamic acid compound, wherein the substituent at the 1-position is X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; C(═O)—, and C(═O)—C₁-C₆alkylene;
R² is a substituted or unsubstituted group selected from among aryl, heteroaryl, cycloalkyl, and heterocycloalkyl;
   where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
      R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
      R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
   X⁶ is a C₁-C₆alkylene, C₁-C₆fluoroalkylene, C₂-C₆alkenylene, C₂-C₆heteroalkylene;
   R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷
   X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
   R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
      R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
      R^{a} and R⁷ together with the N atom to which they are attached form a 5-, 6-, or 7-membered heterocycloalkyl;
         or an active metabolite, pharmaceutically acceptable solvate, pharmaceutically acceptable salt, pharmaceutically acceptable N-oxide, or pharmaceutically acceptable prodrug thereof.

For any and all of the embodiments, substituents can be selected from among from a subset of the listed alternatives. For example, in some embodiments, X² is a substituted or unsubstituted group selected from among C₁-C₆alkylene, C₂-C₆alkenylene, C₁-C₆fluoroalkylene, C₂-C₆fluoroalkenylene, and C₁-C₆heteroalkylene. In other embodiments, X² is a substituted or unsubstituted group selected from among C₁-C₆alkylene, and C₂-C₆alkenylene.

In some embodiments, R² is an optionally substituted group selected from among phenyl, naphthyl, monocyclic heteroaryl, bicyclic heteroaryl, C₃-C₈ cycloalkyl, monocyclic heterocycloalkyl, and bicyclic heterocycloalkyl. In other embodiments, R² is an optionally substituted group selected from among phenyl, naphthyl, (monocyclic heteroaryl containing 0-2 N atoms, 0-10 atoms, and 0-1 S atoms), (bicyclic heteroaryl containing 0-2 N atoms, 0-10 atoms, and 0-1 S atoms), C₃-C₈ cycloalkyl, monocyclic heterocycloalkyl containing 0-2 N atoms, and bicyclic heterocycloalkyl 0-2 N atoms; where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹, —S(═O)—R¹, —S(═O)₂—R¹, —NR¹⁰C(═O)—R¹, —C(═O)N(R¹⁰)₂, —S(═O)₂N(R¹⁰)₂, —NR¹⁰S(═O)₂—R¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, phenyl, and heteroaryl; R¹¹ is a substituted or unsubstituted group selected from among C₁-C₆alkyl, C₁-C₆fluoroalkyl, C₃-C₈cycloalkyl, C₂-C₈heterocycloalkyl, phenyl, and heteroaryl.

In some embodiments, R² is an optionally substituted group selected from among phenyl, naphthyl, (monocyclic heteroaryl containing 0-2 N atoms, 0-10 atoms, and 0-1 S atoms), (bicyclic heteroaryl containing 0-2 N atoms, 0-10 atoms, and 0-1 S atoms), C₃-C₈ cycloalkyl; where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹, —S(═O)—R¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, and phenyl; R¹¹ is a substituted or unsubstituted group selected from among C₁-C₆alkyl, C₁-C₆fluoroalkyl, and phenyl.

In some embodiments, R³ is hydrogen, halogen, substituted or unsubstituted C₁-C₆alkyl, substituted or unsubstituted C₂-C₆alkenyl, substituted or unsubstituted C₁-C₆heteroalkyl, substituted or unsubstituted phenyl, or —X⁶—R⁶; X⁶ is a C₁-C₆alkylene, C₁-C₆fluoroalkylene, C₂-C₆alkenylene, or C₂-C₆heteroalkylene; R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷; X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—; R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R¹ is selected from among hydrogen, C₁-C₆alkyl, C₂-C₆alkenyl, hydroxy, C₁-C₆alkoxy, C₁-C₆fluoroalkoxy, C₁-C₆heteroalkyl; or R^{a} and R⁷ together with the N atom to which they are attached form a 5-, 6-, or 7-membered heterocycloalkyl.

In some embodiments, R³ is hydrogen, halogen, substituted or unsubstituted C₁-C₆alkyl, or —X⁶—R⁶.

In some embodiments, X⁶ is C₁-C₆alkylene; R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl containing 0-2 N atoms, phenyl, heteroaryl containing 0-2 N atoms, or X⁷—R⁷; X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—; R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or R^{a} and R⁷ together with the N atom to which they are attached form a 5-, 6-, or 7-membered heterocycloalkyl.

In some embodiments, R⁶ is X⁷—R⁷.

In some embodiments, X⁷ is a bond, —O—, —S—, —S(═O)—, —S(═O)₂—, —NR^{a}—, or —C(═O)—.

In some embodiments, R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, phenyl, phenylC₁-C₄alkyl, heteroaryl, heteroarylC₁-C₄alkyl; R¹ is selected from among hydrogen, C₁-C₆alkyl, hydroxy, C₁-C₆alkoxy, C₁-C₆fluoroalkoxy, C₁-C₆heteroalkyl; or R^{a} and R⁷ together with the N atom to which they are attached form a 5-, or 6-membered heterocycloalkyl.

In some embodiments, X⁷ is a bond, —O—, or —NR^{a}—. In some embodiments, X⁷ is a bond, or —NR^{a}—.

In some embodiments, R^{a} is selected from among hydrogen, C₁-C₆alkyl, hydroxy, C₁-C₆alkoxy, C₁-C₆heteroalkyl. In other embodiments, R^{a} is selected from among hydrogen, C₁-C₆alkyl, and C₁-C₆heteroalkyl.

In some embodiments, R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, phenyl, phenylC₁-C₄alkyl, heteroaryl, heteroarylC₁-C₄alkyl; R^{a} is selected from among hydrogen, C₁-C₆alkyl, hydroxy, C₁-C₆alkoxy, C₁-C₆heteroalkyl; or R^{a} and R⁷ together with the N atom to which they are attached form a 5-, or 6-membered heterocycloalkyl.

Any combination of the groups described above for the various variables is contemplated herein.

Compounds described herein are selective histone deacetylase 8 (HDAC8) inhibitors. In one embodiment, the selective HDAC8 inhibitor has an IC₅₀ for histone deacetylase 8 activity that is at least 10 fold lower than the IC₅₀ of histone deacetylase 1, histone deacetylase 2, histone deacetylase 3, histone deacetylase 6, histone deacetylase 10, or histone deacetylase 11.

In one embodiment, provided herein is a 1,3-disubstituted-1H-indole-5-thiohydroxamic acid compound, wherein the substituent at the 1-position is R⁴ and the substituent at the 3-position is X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or X⁸—R⁸;
   X⁸ is a C₂-C₆alkylene, C₂-C₆fluoroalkylene, C₂-C₆alkenylene, or C₂-C₆heteroalkylene;
   R^{a} is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹;
   X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
   R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
      R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
      R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, C(═O)—, and C(═O)—C₁-C₆alkylene;
R⁵ is a substituted or unsubstituted group selected from among aryl, heteroaryl, C₃-C₈cycloalkyl, and heterocycloalkyl;
   where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
      R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
      R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
         or an active metabolite, pharmaceutically acceptable solvate, pharmaceutically acceptable salt, pharmaceutically acceptable N-oxide, or pharmaceutically acceptable prodrug thereof.

For any and all of the embodiments, substituents can be selected from among from a subset of the listed alternatives. For example, in some embodiments, R⁴ is hydrogen, substituted or unsubstituted C₁-C₆alkyl, substituted or unsubstituted C₂-C₆alkenyl, substituted or unsubstituted C₁-C₆alkoxy, substituted or unsubstituted C₁-C₆heteroalkyl, substituted or unsubstituted phenyl, or X⁸—R⁸; X⁸ is a C₂-C₆alkylene, C₂-C₆fluoroalkylene, C₂-C₆alkenylene, or C₂-C₆heteroalkylene; R^{a} is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹; X⁹ is a bond, —O—, —S—, —NR^{a}—, —C(═O)—; R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, and C₁-C₆heteroalkyl; or R^{a} and R⁹ together with the N atom to which they are attached form a 5-, or 6-membered heterocycloalkyl.

In other embodiments, R⁴ is hydrogen, substituted or unsubstituted C₁-C₆alkyl, substituted or unsubstituted C₁-C₆heteroalkyl, substituted or unsubstituted phenyl, or X¹—R⁸; X⁸ is a C₂-C₆alkylene; R^{a} is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹; X⁹ is a bond, —O—, —S—, —NR^{a}, —C(═O)—; R⁹ is hydrogen, C₁-C₆alkyl, C₁-C₆heteroalkyl, C₁-C₆haloalkyl, C₂-C₈heterocycloalkyl, heterocycloalkylalkyl, phenyl, phenylalkyl, heteroaryl, heteroarylalkyl; R^{a} is selected from among hydrogen, C₁-C₆alkyl, hydroxy, C₁-C₆alkoxy; or R^{a} and R⁹ together with the N atom to which they are attached form a 5-, or 6-membered heterocycloalkyl.

In some embodiments, X⁵ is a bond, or a substituted or unsubstituted group selected from among C₁-C₆alkylene, C₂-C₆alkenylene, C₁-C₆fluoroalkylene, and C₁-C₆heteroalkylene. In other embodiments, X⁵ is a bond, or a substituted or unsubstituted C₁-C₆alkylene.

In some embodiment, R⁵ is a substituted or unsubstituted group selected from among aryl, heteroaryl, C₃-C₈cycloalkyl, and heterocycloalkyl; where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl; R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl; provided that R⁵ is not optionally substituted pyrrole or optionally substituted 2,5-dioxo-pyrrole.

In some embodiments, R⁵ is a substituted or unsubstituted group selected from among phenyl, naphthyl, (heteroaryl containing 0-2 N atoms, 0-10 atoms, 0-1 S atoms), C₃-C₈cycloalkyl, and heterocycloalkyl containing 0-2 N atoms, provided that R⁵ is not optionally substituted pyrrole or optionally substituted 2,5-dioxo-pyrrole.

In some embodiments, R⁵ is a substituted or unsubstituted group selected from among phenyl, naphthyl, (heteroaryl containing 0-2 N atoms, 0-10 atoms, 0-1 S atoms), C₃-C₈cycloalkyl, and heterocycloalkyl containing 0-2 N atoms.

In some embodiments, if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted heterocycloalkyl, substituted or unsubstituted phenyl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, phenyl, and heteroaryl; R¹¹ is a substituted or unsubstituted group selected from among C₁-C₆alkyl, C₁-C₆fluoroalkyl, phenyl, and heteroaryl.

In some embodiments, R⁵ is a substituted or unsubstituted group selected from among phenyl, naphthyl, (monocyclic heteroaryl containing 0-2 N atoms, 0-10 atoms, 0-1 S atoms), and C₂-C₈heterocycloalkyl containing 0-2 N atoms.

In some embodiment, R⁵ is not optionally substituted pyrrole or optionally substituted 2,5-dioxo-pyrrole.

In some embodiments, R⁵ is a substituted or unsubstituted group selected from among phenyl, naphthyl, (monocyclic heteroaryl containing 0-2 N atoms, 0-10 atoms, 0-1 S atoms), and C₂-C₈heterocycloalkyl containing 0-2 N atoms, provided that R⁵ is not optionally substituted pyrrole or optionally substituted 2,5-dioxo-pyrrole.

Any combination of the groups described above for the various variables is contemplated herein.

Compounds described herein are selective histone deacetylase 8 (HDAC8) inhibitors. In one embodiment, the selective HDAC8 inhibitor has an IC₅₀ for histone deacetylase 8 activity that is at least 10 fold lower than the IC₅₀ of the selective HDAC8 inhibitor for histone deacetylase 1, histone deacetylase 2, histone deacetylase 3, histone deacetylase 6, histone deacetylase 10, or histone deacetylase 11. In some embodiments, compounds described have an IC₅₀ for histone deacetylase 8 that is at least 20 fold lower than the IC₅₀ for histone deacetylase 1, histone deacetylase 2, histone deacetylase 3, histone deacetylase 6, and histone deacetylase 10.

In one aspect, provided herein is a pharmaceutical composition, which includes an effective amount of a compound provided herein, and a pharmaceutically acceptable excipient.

In another aspect, provided herein are pharmaceutical compositions that include a compound, pharmaceutically acceptable salt, pharmaceutically acceptable N-oxide, pharmaceutically active metabolite, pharmaceutically acceptable prodrug, or pharmaceutically acceptable solvate of any compound described herein. In a further aspect, provided herein are compositions further including a pharmaceutically acceptable diluent, excipient or binder. In a further aspect, provided are compositions further including a second pharmaceutically active ingredient.

In certain embodiments, provided herein is a pharmaceutical composition containing: i) a physiologically acceptable carrier, diluent, and/or excipient; and ii) one or more compounds described herein.

In any of the aforementioned aspects are further embodiments that include single administrations of the effective amount of the compound, including further embodiments in which (i) the compound is administered once; (ii) the compound is administered to the mammal multiple times over the span of one day; (iii) continually; or (iv) continuously.

In any of the aforementioned aspects are further embodiments that include multiple administrations of the effective amount of the compound, including further embodiments in which (i) the compound is administered in a single dose; (ii) the time between multiple administrations is every 6 hours; (iii) the compound is administered to the mammal every 8 hours;. In further or alternative embodiments, the method comprises a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In one aspect, provided herein is a method of treating T-cell lymphoma or leukemia in a subject in need thereof, comprising administering to the subject a pharmaceutical composition containing a therapeutically effective amount of a compound described herein.

In one aspect, the T-cell lymphoma is peripheral T cell lymphoma. In another aspect, the T-cell lymphoma or leukemia is T cell lymphoblastic leukemia/lymphoma. In yet another aspect, the T-cell lymphoma is cutaneous T cell lymphoma. In another aspect, the T-cell lymphoma is adult T cell lymphoma.

In one aspect, the methods described herein include administering to the subject a second therapeutic agent other than a selective inhibitor of histone deacetylase 8. In one aspect, the second therapeutic agent is selected from among abarelix (Plenaxis®); aldesleukin (Prokine®); Aldesleukin (Proleukin®); Alemtuzumab (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacizumab (Avastin®); bexarotene (Targretin®); bleomycin (Blenoxane®); bortezomib (Velcade®); busulfan (Busulfex®); busulfan (Myleran®); calusterone (Methosarb®); capecitabine (Xeloda®); carboplatin Paraplatin®); carmustine (BCNU, BiCNU); carmustine (Gliadel®); celecoxib (Celebrex®); cetuximab (Erbitux®); chlorambucil (Leukeran®); cisplatin (Platinol®); cladribine (Leustatin®); clofarabine (Clolar®); cyclophosphamide (Cytoxan®); cytarabine (Cytosar-U®); cytarabine liposomal (DepoCyt); dacarbazine (DTIC-Dome); dactinomycin(actinomycin D, Cosmegen®); Darbepoetin alfa (Aranesp®); dasatinib (Sprycel®); daunorubicin liposomal (DanuoXome); daunorubicin (daunomycin, Daunorubicin®); daunorubicin(daunomycin, Cerubidine®); decitabine (Dacogen®); denileukin (Ontak®); dexrazoxane (Zinecard®); docetaxel (Taxotere®); doxorubicin (Adriamycin®); doxorubicin liposomal (Doxil®); dromostanolone propionate; epirubicin (Ellence®); Epirubicin; Epoetin alfa (EPOGEN®); erlotinib (Tarceva®); estramustine (Emcyt®); etoposide phosphate (Etopophos®); etoposide (VP-16; Vepesid®); exemestane (AROMASIN®); Filgrastim (Neupogen®); floxuridine (FUDR); fludarabine (Fludara®); fluorouracil (5-FU, Adrucil); fulvestrant (Faslodex®); gefitinib (Iressa®); gemcitabine (Gemzar®); gemtuzumab ozogamicin (Mylotarg®); goserelin acetate (Zoladex®); histrelin acetate (Histrelin®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Leucovorin®); Leuprolide Acetate (Eligard®); levamisole (Ergamisol®); lomustine, CCNU CeeBU®); meclorethamine(nitrogen mustard, Mustargen®); megestrol acetate (Megace®); melphalan (Alkeran®); mercaptopurine (6-MP, Purinethol®); methotrexate (Rheumatrex®, Trexall®); methoxsalen (Uvadex®); mitomycin C (Mutamycin®); mitomycin C (Mitozytrex®); mitotane (Lysodren®); mitoxantrone (Novantrone®); nandrolone phenpropionate (Durabolin-50); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pegademase (Adagen®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); rituximab (Rituxan®); sargramostim (Leukine®); Sargramostim (Prokine®); sorafenib (Nexavar®); streptozocin (Zanosar®); sunitinib maleate (Sutent®); tamoxifen (Nolvadex®); temozolomide (Temodar®); teniposide (VM-26, Vumon®); testolactone (Teslac®); thalidomide (Thalomid®); thioguanine (6-TG, Thioguanine®); thiotepa (Thioplex®); topotecan (Hycamtin®); toremifene (Fareston®); tositumomab (Bexxar®); tositumomab/1-131 tositumomab (Bexxar®); trastuzumab (Herceptin®); tretinoin (ATRA, Vesanoid®); Uracil Mustard; valrubicin (Valstar®); vinblastine (Velban®); vincristine (Oncovin®); vinorelbine (Navelbine®); vorinostat (Zolinza®); zoledronate (Zometa®); and zoledronic acid (Zometa®).

In one aspect, provided herein is a method of treating a disease or condition mediated by interleukin-1 beta (IL-1b), comprising administering to the patient a therapeutically effective amount of a compound, pharmaceutically acceptable salt, pharmaceutically acceptable N-oxide, pharmaceutically active metabolite, pharmaceutically acceptable prodrug, or pharmaceutically acceptable solvate of a compound described herein. In one embodiment, the compound used in the method is a selective HDAC8 inhibitor compound described herein.

In one aspect the disease or condition is an autoimmune disease or condition. In one embodiment, the disease or condition is selected from among osteoarthritis, rheumatoid arthritis, septic arthritis, gout, pseudogout, juvenile arthritis, Still's disease, Ankylosing spondylitis, systemic lupus erythematosus (SLE), Henoch-Schönlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, and myalgia.

In some embodiments, the methods provided herein include administering to the subject a second therapeutic agent other than a selective inhibitor of histone deacetylase 8. In one aspect, the second therapeutic agent is selected from among immunosuppressants, glucocorticoids, non-steroidal anti-inflammatory drugs, Cox-2-specific inhibitors, leflunomide, gold thioglucose, gold thiomalate, aurofin, sulfasalazine, hydroxychloroquinine, minocycline, anti-TNF-α agents, abatacept, anakinra, interferon-β, interferon-γ, interleukin-2, allergy vaccines, antihistamines, antileukotrienes, beta-agonists, theophylline, and anticholinergics. In another embodiment, the second therapeutic agent is selected from among tacrolimus, cyclosporin, rapamicin, methotrexate, cyclophosphamide, azathioprine, mercaptopurine, mycophenolate, or FTY720, prednisone, cortisone acetate, prednisolone, methylprednisolone, dexamethasone, betamethasone, triamcinolone, beclometasone, fludrocortisone acetate, deoxycorticosterone acetate, aldosterone, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502, JTE-522, L-745,337 and NS398, leflunomide, gold thioglucose, gold thiomalate, aurofin, sulfasalazine, hydroxychloroquinine, minocycline, infliximab, etanercept, adalimumab, abatacept, anakinra, interferon-β, interferon-γ, interleukin-2, allergy vaccines, antihistamines, antileukotrienes, beta-agonists, theophylline, and anticholinergics.

In one embodiment, provided herein is a use of a compound described herein for the formulation of a medicament for the selective inhibition of histone deacetylase 8 activity or for the treatment of a disease or condition that would benefit from the selective inhibition of the activity of histone deacetylase 8. In one embodiment, the compound is a selective HDAC8 inhibitor.

Articles of manufacture, which include packaging material, a selective HDAC8 inhibitor compound described herein, which is effective for selectively inhibiting histone deacetylase 8 activity, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of histone deacetylase 8, or for the treatment, prevention or amelioration of one or more symptoms of a disease or condition that would benefit from inhibition of histone deacetylase 8 activity, are provided.

One aspect described herein relates to treating T-cell lymphoma by administering (e.g., orally, bucally, transdermally, intranasally, intravenously, or rectally) to a subject in need (e.g., a human) a pharmaceutical composition containing a therapeutically effective amount of a selective HDAC8 inhibitor. The T-cell lymphoma can be, e.g., peripheral T cell lymphoma, lymphoblastic lymphoma, cutaneous T cell lymphoma, NK/T-cell lymphoma, or adult T cell leukemia/lymphoma. In one embodiment, the subject to be treated also suffers from an insulin deficiency. In some embodiments, the subject is administered, in addition to the selective HDAC8 inhibitor composition, one or more anticancer agents, e.g., topical steroids, BCNU (Carmustine), nitrogen mustards, photo therapy, topical imiquimod, EBD, MTX, doxorubicin (Doxil), gemcitibine, etoposide, pentostatin, cytokines, interferon, 5-aza-2′-deoxycytidine, all trans retinoic acid, doxorubicin, vincristine, etoposide, gemcitabine, imatinib (Gleevec®), 17-N-Allylamino-17-Demethoxygeldanamycin (17-AAG), flavopiridol, LY294002, bortezomib, trastuzumab, BAY 11-7082, PKC412, or PD184352 in any combination.

Another aspect described herein relates to treating T-cell lymphoma by administering to a subject in need a plurality of autologous T-cells that have been exposed to a pharmaceutical composition containing a therapeutically effective amount of a selective HDAC8 inhibitor ex vivo.

A further aspect described herein relates to decreasing somatostatin expression in a subject in need by administering to the subject a pharmaceutical composition containing a therapeutically effective amount of a selective HDAC8 inhibitor. In some embodiments, the subject in need of decreased somatostatin expression suffers from a growth hormone deficiency. In one embodiment, the subject suffering from a growth hormone deficiency is administered, in addition to the selective HDAC8 inhibitor composition, a therapeutically effective amount of a growth hormone secretagogue, or growth hormone.

Yet another aspect described herein relates to increasing growth hormone expression in a subject in need by administering to the subject a pharmaceutical composition containing a therapeutically effective amount of a selective HDAC8 inhibitor. In some embodiments, the subject in need of increased growth hormone expression is administered, in addition to the selective HDAC8 inhibitor composition, a therapeutically effective amount of a growth hormone secretagogue (e.g., arginine, L-3,4-dihydroxyphenylalanine, glucagon, vasopressin, pituitary adenylyl cyclase activating peptide, a muscarinic receptor agonist, or growth hormone releasing peptide), or growth hormone.

In some embodiments of any of the methods described herein, the selective inhibitor of HDAC8 binds to HDAC8.

In one aspect of any of the methods described herein, the selective HDAC8 inhibitor has an IC₅₀ for HDAC8 that is at least 10 fold lower than the IC₅₀ for HDAC1, HDAC2, HDAC3, HDAC6, HDAC10, or HDAC11.

In some embodiments, selective HDAC8 inhibitors described herein have an IC₅₀ for HDAC8 that is at least 15 fold lower than the IC₅₀ for HDAC1, HDAC2, HDAC3, HDAC6, and HDAC10. In some embodiments, selective HDAC8 inhibitors described herein have an IC₅₀ for HDAC8 that is at least 20 fold lower than the IC₅₀ for HDAC1, HDAC2, HDAC3, HDAC6, and HDAC10. In some embodiments, selective HDAC8 inhibitors described herein have an IC₅₀ for HDAC8 that is at least 100 fold lower than the IC₅₀ for HDAC1, HDAC2, HDAC3, HDAC6, and HDAC10.

In some embodiments, selective HDAC8 inhibitors described herein have an IC₅₀ for HDAC8 that is at least 10 fold lower than the IC₅₀ for HDAC1. In some embodiments, selective HDAC8 inhibitors described herein have an IC₅₀ for HDAC8 that is at least 20 fold lower than the IC₅₀ for HDAC1. In some embodiments, selective HDAC8 inhibitors described herein have an IC₅₀ for HDAC8 that is at least 40 fold lower than the IC₅₀ for HDAC1. In some embodiments, selective HDAC8 inhibitors described herein have an IC₅₀ for HDAC8 that is at least 100 fold lower than the IC₅₀ for HDAC1. In some embodiments, selective HDAC8 inhibitors described herein have an IC₅₀ for HDAC8 that is at least 150 fold lower than the IC₅₀ for HDAC1.

In some embodiments, compounds provided herein are administered to a human.

In some embodiments, compounds provided herein are orally administered.

In some embodiments, compounds provided herein are used for inhibiting the activity of HDAC8. In some embodiments, compounds provided herein are used for inhibiting the activity of HDAC8 or for the treatment of a disease or condition that would benefit from inhibition of HDAC8 activity.

In other embodiments, compounds provided herein are used for the formulation of a medicament for the inhibition of HDAC8 activity.

In another aspect are methods of improving the pharmacokinetic properties of a hydroxamic acid HDAC inhibitor compound, wherein said method comprises replacing the hydroxamic acid functional group of the hydroxamic acid HDAC inhibitor compound with a thiohydroxamic functional group to provide a thiohydroxamic acid HDAC inhibitor compound.

In a further embodiment of such a method, the “replacing the hydroxamic acid functional group of the hydroxamic acid HDAC inhibitor compound with a thiohydroxamic functional group to provide a thiohydroxamic acid HDAC inhibitor compound” comprises:
a) synthesizing the carboxylic acid ester compound that is the final precursor compound to the hydroxamic acid HDAC inhibitor compound, wherein treatment of the carboxylic acid ester compound with hydroxylamine would provide the hydroxamic acid HDAC inhibitor compound;
b) treating the carboxylic acid ester compound of a) with P₄S₁,o and hexamethyldisiloxane (HMDO) to obtain a carbothioester compound; and
c) reacting the carbothioester compound of b) with hydroxylamine to provide a thiohydroxamic acid HDAC inhibitor compound.

In a further embodiment of such methods, the pharmacokinetic property is bioavailability. In a further or alternative embodiment of such methods, the pharmacokinetic property is stability.

Other objects, features and advantages of the methods, compounds, and compositions described herein will become apparent from the following detailed description. It should be understood, however, that the detailed description and the specific examples, while indicating specific embodiments, are given by way of illustration only, since various changes and modifications within the spirit and scope of the disclosure will become apparent to those skilled in the art from this detailed description.

### BRIEF DESCRIPTION OF THE FIGURES

FIG. 1 presents the structures of a representative hydroxamate and thiohydroxamate.

FIG. 2 is an illustrative synthesis of a representative thiohydroxamate.

FIG. 3 is an illustrative synthesis of a representative thiohydroxamate and hydroxamate.

FIG. 4 is an illustrative comparison of the activity of a representative hydroxamate and thiohydroxamate in A549 cells.

FIG. 5 is an illustrative comparison of the activity of a representative hydroxamate and thiohydroxamate in K1 Chinese Hamster Ovary cells.

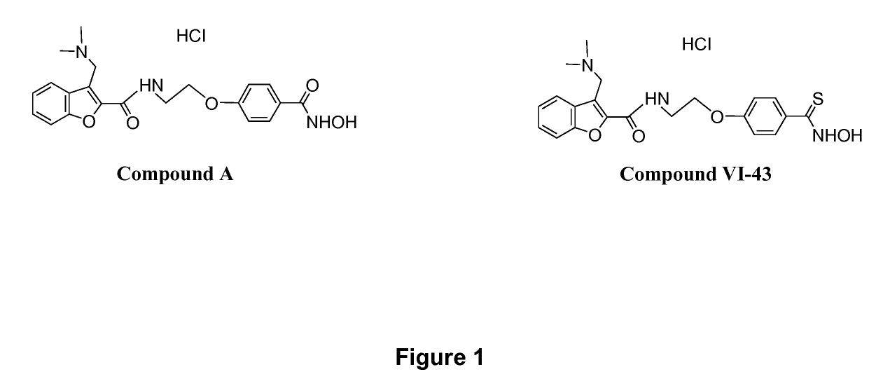

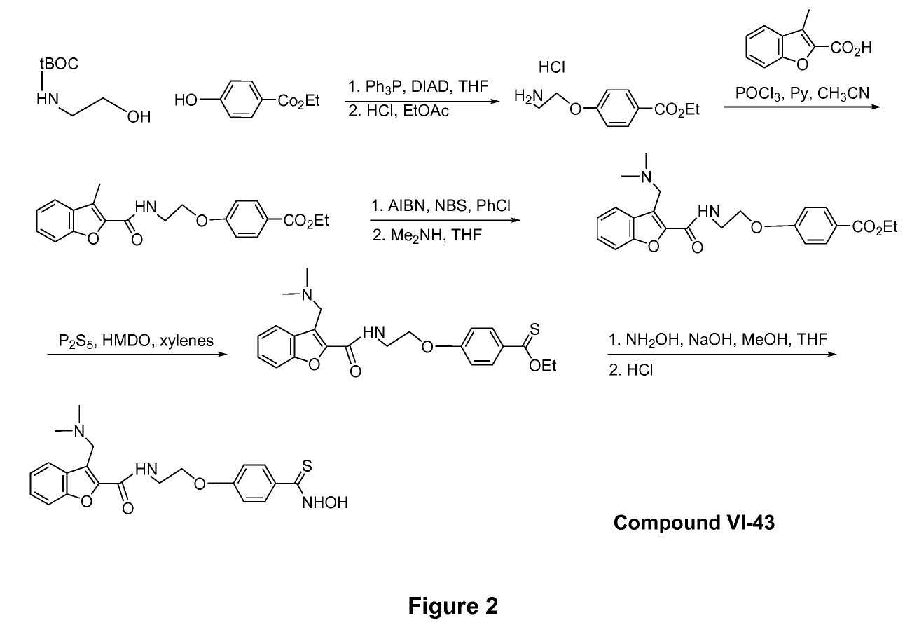

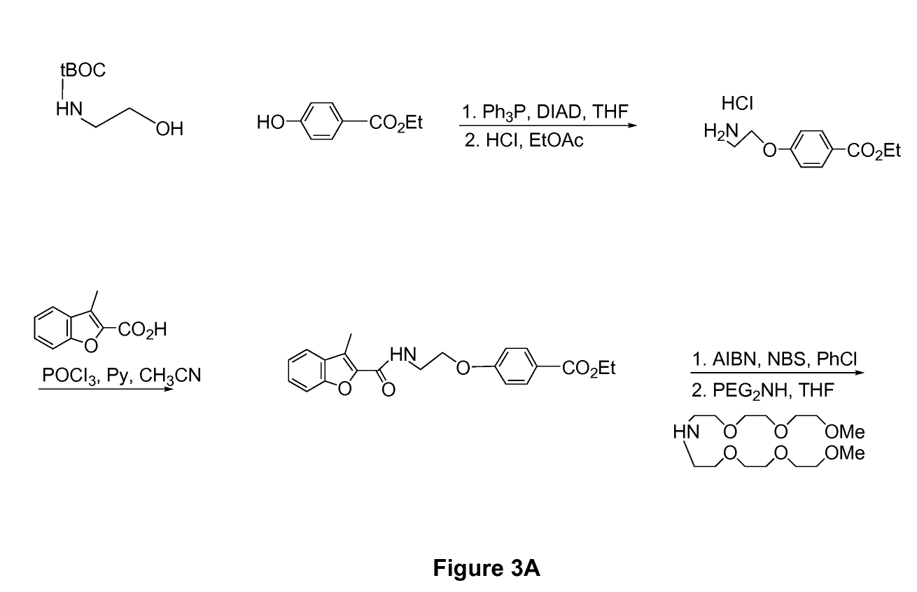

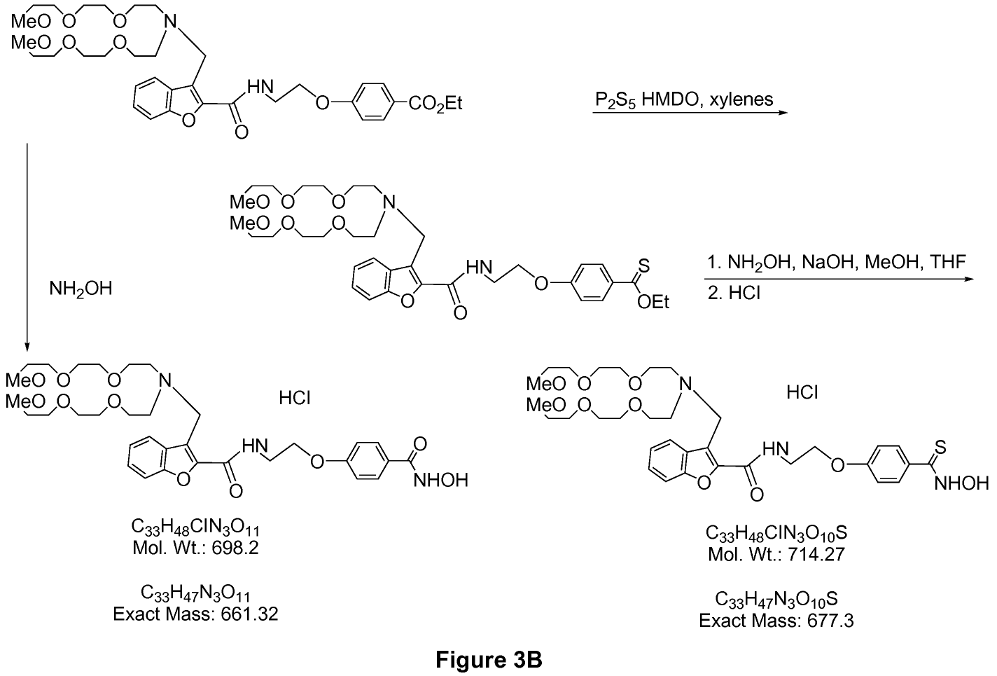

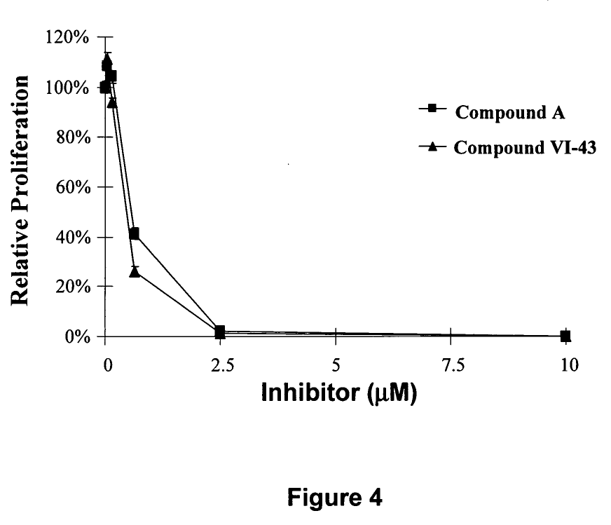

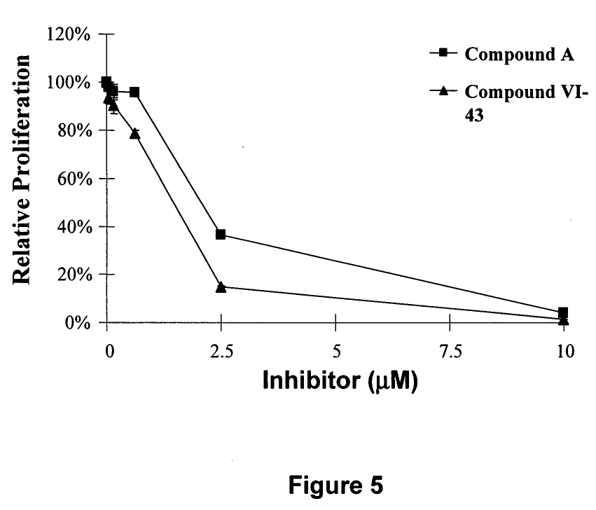

### DETAILED DESCRIPTION

Covalent modification of histone proteins through acetylation and deacetylation is an important determinant of chromatin structure and a regulator of gene expression. Acetylation of histone proteins occurs on lysine residues near the N-termini of these proteins. In conjunction with other modifications of histone proteins and DNA, the acetylation state of histones determines whether the chromatin is in a condensed, transcriptionally silent state or in a form more accessible to the transcription machinery of the cell. In general, hyperacetylation of histone proteins is associated with transcriptional activation of genes. The steady-state histone acetylation level arises from the opposing action of histone acetyltransferase (HAT) and histone deacetylase (HDAC) enzymes.

Histone deacetylases (HDACs) catalyze the removal of acetyl groups from lysine ε-amino groups near the N-termini of histones. This reaction promotes the condensation of chromatin, leading to repression of transcription.

HDAC inhibitors (HDIs) modify gene expression positively or negatively in a cell- and gene-specific manner. HDIs increase the accumulation of acetylated histones, directly influencing chromatin structure and, thereby, the relationship of the nucleosome to gene promoter elements.

Histone deacetylase (HDAC) enzymes modulate gene expression through the deacetylation of acetylated lysine residues on histone proteins. They operate in biological systems as part of multiprotein corepressor complexes.

Histone deacetylases have been grouped into three classes. Class I and class II histone deacetylases (HDACs) are zinc containing hydrolase enzymes. The division of the proteins into classes I and II is based on protein size, sequence similarity, and organization of the protein domains.

Members of class I are related to the yeast RPD3 gene product. Class I HDACs include: HDAC1 (GenBank Accession Number NP_{—}004955; Wolffe, A. P., Science 272, 5260, 371-372, 1996); HDAC2 (GenBank Accession Number NP_{—}001518; Furukawa, et al., Cytogenet. Cell Genet. 73; 1-2, 130-133, 1996); HDAC3 (GenBank Accession Number NP_{—}003874; Yang, et al., J. Biol. Chem. 272,44,28001-28007, 1997); HDAC8 (GenBank Accession Number NP_{—}060956; Buggy, et al., Biochem. J. 350 Pt 1, 199-205, 2000); HDAC11 (GenBank Accession Number NP_{—}079103; Gao, L. et al., J. Biol. Chem. 277, 28, 25748-25755, 2002).

HDAC8 is a 377 residue, 42 kDa protein localized to the nucleus of a wide array of tissues, as well as several human tumor cell lines. The wild-type form of full length HDAC8 is described in GenBank Accession Number NP 060956; Buggy, J. J. et al., Biochem. J., 350 (Pt 1), 199-205 (2000). The HDAC8 structure was solved with four different hydroxamate inhibitors bound (Somoza et al., Structure, 2004, 12, 1325)

Class II are homologues of the yeast HDA1 protein (de Ruijter et. al. Biochem J. 2003, 3 70(Pt 3), 737-49), and include: HDAC4 (GenBank Accession Number NP_{—}006028; Wolffe, A. P., Nature 387, 6628, 16-17, 1997); HDAC5 (GenBank Accession Number NP_{—}631944; Nagase, T., et al., DNA Res. 5 (1), 31-39, 1998); HDAC6 (GenBank Accession Number NP_{—}006035; Wolffe, A. P., Nature 387,6628,16-17, 1997); HDAC7 (GenBank Accession Number NP_{—}057680; Kao, H. Y., et al., Genes Dev. 14 (1), 55-66, 2000); HDAC9 (GenBank Accession Number NP 478056; Sparrow, et al., EMBO J. 18(18), 5085-5098,1999); HDAC10 (GenBank Accession Number NP_{—}114408; Kao, H. Y., et al., J. Biol. Chem. 277 (1), 187-193, 2002).

Class II HDACs have been further subdivided into classes Ia (HDACs 4, 5, 7, and 9) and IIb (HDACs 6 and 10).

The third class of deacetylases consists of the members of the Sir2 family of enzymes. These enzymes have histone deacetylase activity but are structurally and evolutionarily unrelated to the class I and class II proteins. They are (nicotinamide adenine dinucleotide) NAD-dependent and unlike class I HDACs and class II HDACs, they do not contain a catalytic zinc site

In the cell, HDAC proteins are recruited as part of multicomponent repressor complexes. Several HDAC containing complexes have been characterized, including the N-CoR/SMRT, Sin3, NuRD, and CoREST complexes. Within these complexes, HDACs 1 and 2 typically interact with the mSin3, Mi-2, or CoREST proteins. HDAC3 and the class IIa HDACs have been shown to interact with SMRT and the related N-CoR protein. A large number of transcription factors have been shown to bind to one of the corepressor complexes as a means of regulating transcription. The recruitment of HDACs by DNA-binding proteins allows histone deacetylation to be directed toward specific regions of the chromatin in order to promote targeted transcriptional repression

HDAC proteins are promising therapeutic targets on account of their involvement in regulating genes involved in cell cycle progression and control. Inhibition of HDACs has been shown to upregulate genes, including p21WAF/CIP1, p27, p53, and cyclin E, and to downregulate genes such as cyclin A and cyclin D. Growth inhibition in several lines of cancer cells has been observed upon treatment with HDAC inhibitors, and in vivo studies have shown that some of these inhibitors are efficacious in slowing tumor growth. The biological activity of each of the HDAC isozymes is determined by a combination of the intrinsic activity of the enzyme and the effects of cofactor binding on reactivity and substrate recognition (Schultz et al., Biochemistry, 2004, 43, 11083-11091).

Methods for determining HDAC activity in vivo or in vitro are known in the art, as disclosed in, e.g., Kim et al. (2006), Methods Mol Biol., 325:273-283.

Non-selective HDAC inhibitors inhibit the deacetylase activity of most, if not all, of the HDACs with equal potency. The mechanisms of the anticancer effects of SAHA, a non-selective HDAC inhibitor, are not completely understood, and likely result from both altered gene expression and altered function of proteins regulating cell proliferation and cell death pathways. Non-selective HDAC inhibitors, such as SAHA, can induce the accumulation of acetylated histone proteins and non histone proteins. Non-histone proteins that are acetylated include, but are not limited to:

Bcl-6 (Oncoprotein); LEF/TCF (Lymphoid enhancer factor); P53 (Tumor suppressor); Ku70 (Autoantigen with multiple function, including DNA repair); H1F-1α (angiogenesis); GATA-1 (Transcription factor); WRN (Werner helicase); E2F-1 (Transcription factor); Smad7 (Transcription factor); Rb (Tumor suppressor); TFIIF (Transcription machinery); c-Jun (Transcription factor); α-Tubulin (Structural protein); HMGI(Y) (Chromatin structure); ACTR (Nuclear receptor coactivator); Androgen Receptor (Signal transduction); EKLF (Erythroid kruppel-like factor); YY-1 (Transcription factor); NF-κB (RelA) (Transcription factor); MyoD (Transcription factor); Importin a7 (Nuclear pore protein); Hsp90 (Chaperone protein); TFIIE (Transcription machinery); b-Catenin (Signaltransduction); TFJB (Transcription factor).

Genes whose transcription is altered by histone deacetylase inhibitors include:

1) Genes that are induced by HDAC inhibitors: Cell cycle (p1 and cyclin E); Proapoptotic (Bak, BAX, CD95, and its ligand gelsolin, GADD45β, p53, Apaf-1 DFF45a, Bim, BAD, TRAIL, DR5, Fas and its ligand, and Caspase 9, -8 and -3); Redox Components (Thioredoxin-binding protein-1, thioredoxin, glutaredoxin and methallothionein 1L); Chromatin structure (Histone H₂B); Retinoic acid pathway (RARβ).

2) Genes that are repressed by HDAC inhibitors: Cell cycle (Cyclin D1 and A, and thymidylate synthase); Antiapoptotic (Bcl-2, Bcl-XL, c-FLIP, survivin, XIAP); Angiogenic factor (Vascular endothelial growth factor and HIF-Loc); Lipopolysaccharide-induced inflammatory cytokines (TNF-a, IFN-g and IL-1b and -6); Signaltransducer and activator of transcription 5-controlled genes (STAT5).

The pharmaceutical industry seeks novel anti-cancer therapies targeting the underlying molecular defects that cause cancer with less toxicity than traditional chemotherapy (Miller et al., J. Med. Chem., 2003, vol. 46, no. 24, 5097-5116). The inappropriate deacetylation of tumour suppressor genes, a molecular defect, may silence these genes, resulting in the progression of cancer. HDAC inhibitors selectively switch on these tumour suppressor genes, something traditional chemotherapy may not do.

HDAC enzymes or isoforms appear to be involved in many different types of cancer. Inhibition of HDACs with HDAC inhibitors results in multiple and desirable anti-cancer effects such as, but not limited to, (i) the inhibition of cancer cell proliferation, (ii) the induction of apoptosis (cell death) of cancer cells, (iii) cell cycle regulation, (iv) the induction of tumour suppressor genes, and (v) the blocking of tumour angiogenesis (development of new tumour blood vessels). These multiple effects provided by HDAC inhibitors provide a method of treating cancer.

Interest in histone deacetylase enzymes (HDACs) as targets for pharmaceutical development has centered on the role of HDACs in regulating genes associated with cell-cycle progression and the development and progression of cancer (Kramer et. al. Trends Endocrinol. Metab. 12, 294-300, (2001)). Several studies have shown that treatment of various cell lines with HDAC inhibitors leads to hyper acetylation of histone proteins and cell-cycle arrest in late G₁ phase or at the G₂/M transition. Genes involved in the cell cycle that have been shown to be up regulated by HDAC inhibitors include p21, p27, p53 and cyclin E. Cyclin A and cyclin D have been reported to be down regulated by HDAC inhibitors. In tumor cell lines, several studies have shown that treatment with HDAC inhibitors can lead to growth inhibition, growth arrest, terminal differentiation and/or apoptosis. In vivo studies have demonstrated growth inhibition of tumors and a reduction in tumor metastasis as a result of treatment with HDAC inhibitors.

The clearest link between abnormal HDAC activity and cancer occurs in acute promyelocytic leukemia. In this condition, a chromosomal translocation leads to the fusion of the retinoic acid receptor RARα with the promyelocytic leukemia (PML) or promyelocytic leukemia zinc-finger (PLZF) proteins. Both PML-RARα and PLZF-RARα promote the progression of leukemia by repressing retinoic acid-regulated genes through the abnormal recruitment of SMRT-mSin3-HDAC complex (Lin et. al. Nature 391, 811-814 (1998)); Grignani et al. Nature 391, 815-818 (1998)). Whereas the PML-RARα form of the disease is treatable with retinoic acid, the PLZF-RARα form is resistant to this treatment. For a patient with the retinoic acid-resistant form of the disease, the addition of the HDAC inhibitor sodium butyrate to the dosing regimen led to complete clinical and cytogenic remission (Warrell et al. J. Natl. Cancer. Inst. 90, 1621-1625, (1998)). HDACs have also been associated with Huntington's disease (Steffan, et al., Nature 413:739-744, “Histone deacetylase inhibitors arrest polyglutamine-dependent neurodegeneration in Drosophila”).

In general, almost all of the inhibitors targeting HDACs are broad spectrum compounds, inhibiting all of the HDAC isoforms with equal potency. These broad spectrum HDAC inhibitors cause the induction of differentiation, growth arrest and/or apoptosis in a large number of tumor cell lines in vitro.

As described herein, HDAC8 is expressed primarily in delta cells of the islets of Langerhans in the pancreas; in small intestinal epithelial cells; and in neuroendocrine cells. Of note, delta cells express and secrete somatostatin, a peptide hormone that inhibits the secretion of insulin and growth hormone. Without being bound by theory, it is believed that HDAC8 activity drives the expression of somatostatin in delta cells. Thus, inhibiting HDAC8 activity is expected to decrease somatostatin expression and secretion from delta cells, and consequently increase systemic insulin and growth hormone levels.

Described herein are methods for inhibiting somatostatin expression in a subject by administering to the subject a selective HDAC8 inhibitor composition. Further, described herein are methods for treating a subject suffering from an insulin deficiency or a growth hormone deficiency by administering a selective HDAC8 inhibitor to the subject.

### T-Cell Lymphomas or Leukemias

HDAC8 is expressed at unusually high levels in tumor cell lines, e.g., Jurkat, HuT78, K562, PC3, and OVCR-3. In fact, as described herein, inhibiting HDAC8 activity decreases proliferation of T-cell derived tumor cells (e.g., Jurkat cells) by apoptosis. In contrast, HDAC8 inhibition does not affect the proliferation of either non-cancerous cells (e.g., peripheral blood mononuclear cells) or tumor cell lines other than T-cell derived lines. Thus, selective HDAC8 inhibitors are useful for slowing or arresting the progression of T-cell derived cancers with lessened or no toxicity to non-cancerous cells.

Selective HDAC8 inhibitor compounds described herein were screened against a large panel of tumor cell lines in vitro, and were found to induce apoptosis in cell lines derived from T-cell lymphomas or leukemias. Selective HDAC8 inhibitor compounds described herein at low micromolar concentrations inhibits the growth of Jurkat and HuT78 cell lines while doses as high as 20 micromolar have no effect on B cell or myeloid-derived lymphomas or solid tumor lines. Unlike broad spectrum inhibitors, selective HDAC8 inhibitor compounds described herein do not cause detectable histone or tubulin acetylation, but lead to a dose dependent decrease in HDAC8 protein levels in treated cells. Selective HDAC8 inhibitor compounds described herein activated caspases 3, 8 and 9, showing that both intrinsic and extrinsic apoptic pathways were involved; accordingly, apoptosis was blocked completely by pan-caspase inhibitors but only partially by inhibitors of specific caspases. Thus, selective HDAC8 inhibitor compounds described herein may be of benefit in the treatment of T-cell lymphomas and leukemias.

Described herein are methods for treating a subject suffering from a T-cell lymphoma by administering to the subject a selective HDAC8 inhibitor composition. Also described herein are methods for treating a subject suffering from a T-cell lymphoma by administering to the subject a population of autologous T-cells that have been exposed to a selective HDAC8 inhibitor composition ex vivo.

Selective HDAC8 inhibitor compounds and compositions thereof can be used to treat a subject suffering from a T-cell lymphoma, e.g., a peripheral T-cell lymphoma, a lymphoblastic lymphoma, a cutaneous T-cell lymphoma, or an adult T-cell lymphoma.

In some embodiments, the T-cell lymphoma treatment method includes administering to a subject a therapeutically effective amount of a selective HDAC8 inhibitor pharmaceutical composition.

In other embodiments, the T-cell lymphoma treatment can include administering, in addition to a selective HDAC8 inhibitor pharmaceutical composition, one or more additional anti-cancer agents described herein in any combination.

The methods described herein include administering a pharmaceutical composition containing a selective HDAC8 inhibitor in a quantity sufficient to decrease HDAC8 deacetylase activity in vivo by a therapeutically effective amount. In some embodiments, cells derived from a subject to be treated (i.e. autologous cells) are exposed, ex vivo, to a pharmaceutical composition containing a selective HDAC8 inhibitor composition in a quantity sufficient to decrease HDAC8 deacetylase activity in vitro.

In one embodiment, T-cells from a donor subject suffering a T-cell lymphoma are cultured and expanded, ex vivo, in the presence of a selective HDAC8 inhibitor at a concentration that is effective for selectively killing transformed T-cells. Afterwards, the expanded T-cell population, free of transformed T-cells, can be introduced into the donor subject. T-cell culture, in vitro expansion, and in vivo transfer is described in, e.g., Porter et al. (2006), Blood, 107(4):1325-1331; Rapoport et al. (2005), Nat. Med., 1230-1237; Laport et al. (2003), Blood, 102(6):2004-2013.

### Inhibition of IL-1b Secretion

Pan-HDAC inhibitors have been examined for their application in immunological disorders. Cytokine production is regulated through selective patterns of histone acetylation (Avni et al. Nature Immunol., 2002, 3, 643). It is known that broad spectrum HDAC inhibitors (e.g. suberoylanilide hydroxamic acid (SAHA), trichostatin (TSA)) decrease LPS-stimulated cytokine production in vitro and in vivo and have anti-inflammatory properties (Leoni et al. PNAS USA, 2002, 99, 2995; Mascagni et al., International Patent Publication WO 03/013493). Nonselective HDAC inhibitors, such as SAHA and TSA, have also been shown to antagonize systemic lupus erythematosus in a mouse model (International patent publication WO 02/55017; Mishra et al., J. Clin. Invest., 2003, 111, 539).

Histone deacetylase inhibitors prevent exocytosis of interleukin-1b-containing secretory lysosomes. A functional microtubule network is required for IL-1b secretion and disruption of tubulin is the mechanism by which HDAC inhibitors reduce cytokine secretion (Carta et al. Blood. 2006, 1; 108(5):1618-26). Carta et al. examined the effects of pan-HDAC inhibitors (e.g. SAHA, TSA) on IL-1b secretion, and concluded that the effects of the pan-HDAC inhibitors on secretion of IL-1b is achieved by the inhibition cytoplasmic HDAC6. Carta et al. also showed that HDAC inhibitors inhibit the secretion of IL-1b, but do not inhibit the synthesis of its precursor.

As described herein, selective HDAC8 inhibitor compounds described herein reduce the secretion of proinflammatory cytokines including but not limited to interleukin-1 beta (IL-1b). Thus, HDAC8 is the HDAC enzyme involved in cytokine secretion. The use of selective HDAC8 inhibitor compounds provides a method of reducing cytokine secretion with reduced toxicity, due to the selective inhibition of one HDAC isoform (vs. the use of pan-HDAC inhibitors that inhibit all of the HDAC isoforms).

Selective HDAC8 inhibitor compounds described herein inhibit, in a dose dependent fashion, lipopolysaccharide (LPS) and/or ATP stimulated secretion of IL-1b from purified human peripheral blood mononuclear cells (PBMCs) as well as from the monocyte cell line THP-1. In some embodiments, the EC₅₀ for inhibition ranges from 0.5 micromolar to 5 micromolar.

The production and secretion of IL-1b is via a non-classical pathway of protein secretion, involving potassium efflux, the autocatalytic processing of procaspase-1, the cleavage by active caspase-1 of the IL-1b precursor, the influx of calcium ions, and the activation of specific phospholipases including PLA-2. Selective HDAC8 inhibitor compounds described herein inhibit one or more steps in this secretory pathway.

Selective HDAC8 inhibitors may be used to treat diseases or conditions that are mediated or linked to IL-1b secretion and activity. Selective HDAC8 inhibitor compounds may be used to treat autoimmune diseases or conditions in which IL-1b is contributor to the signs and symptoms of the diseases or conditions (Burger et al., Best Practice & Research Clinical Rheumatology, Vol. 20, No. 5, pp. 879-896, 2006; Dayer et al., Current Opinions in Rheum., 2001, 13:170-176; Abramson et al., Rheumatology, 2002; 41; 972-980). Selective HDAC8 inhibitor compounds may be used to inhibit IL-1b secretion and thus find utility in the treatment of diseases or conditions that are linked to IL-1b secretion and activity, which include, but are not limited to, osteoarthritis, rheumatoid arthritis, septic arthritis, gout, pseudogout, juvenile arthritis, Still's disease, Ankylosing spondylitis, systemic lupus erythematosus (SLE), Henoch-Schönlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, myalgia; Adult Still's disease, Systemic-onset juvenile idiopathic arthritis, Lupus arthritis, Ankylosing spondylitis, familial Mediterranean fever (FMF), TNF receptor-associated periodic syndrome (TRAPS), hyperimmunoglobulinemia D with periodic fever syndrome (HIDS), Blau syndrome, FCAS, MWS, neonatal-onset multisystem inflammatory disease (NOMID) and cryopyrin-associated periodic syndrome (CAPS), familial cold autoinflammatory syndrome (FCAS); Muckle-Wells syndrome (MWS); neonatal-onset multisystem inflammatory disease (NOMID); chronic infantile neurologic, cutaneous, articular syndrome (CINCA); cryopyrin-associated periodic syndrome (CAPS); pyogenic sterile arthritis, pyoderma gangrenosum, and acne syndrome (PAPA).

Chronic inflammation in patients has been linked to cancer development (Coussens et al., Nature, 420, 860-867, 2002). Cancers associated with chronic inflammation include, but are not limited to, lung, esophageal, gastric, pancreatic, cervical, bladder, prostate and colorectal cancers. The role of the inflammatory microenvironment as a causative factor in the etiology of cancer is also supported by findings that regular use of non-steroidal anti-inflammatory drugs (NSAIDs) is associated with a reduced incidence of colorectal, breast and gastric cancer. Pro-inflammatory cytokines are mediators of chronic inflammatory responses, and have effects on malignant processes.

Pro-inflammatory cytokines are involved in carcinogenesis and malignant transformation, tumor growth, invasion and metastasis. Persistent expression of proinflammatory cytokines, in or near tumors, exerts a range of effects, including but not limited to, increasing growth and invasiveness of the malignant cells, metastasis, tumorigenesis, to activation of immune-mediated mechanisms, leading to the destruction of tumor cells and inhibition of tumor growth. IL-1b-transfected tumor cells have been reported to fail to induce effective antitumor immune responses. In several human cancers, local IL-1b expression by the malignant cells or the microenvironment has been associated with aggressive tumor growth and poor prognosis.

In IL-1b-transfected fibrosarcoma cells, an up-regulation of MMP-2 and MMP-9 and TGFβ, genes that are involved in invasiveness, was observed, as opposed to the shut-off of these genes in IL-1α-transfected fibrosarcomas cells. IL-1b may also enhance the invasiveness of already existing tumor cells by switching on angiogenesis and by the induction of inflammatory molecules, such as MMPs, heparanase, chemokines or integrins on the malignant cells or endothelial cells, leading to tumor dissemination and metastasis. Il-1b induces secretion of growth and invasiveness-promoting factors, e.g. matrix metalloproteinases and angiogenic factors (i.e. VEGF and bFGF and ELR-positive CXC chemokines, i.e. IL-8 and MCP-1). (Apte et al., seminars in Cancer Biology, vol. 12, 2002, 277-290).

Secreted IL-1b seems to be involved in tumor growth and invasion. Inhibition of IL-1b secretion, e.g. by using selective HDAC8 compounds, in malignant cells, or in the tumor's microenvironment may provide a method for cancer therapy.

Thus in one embodiment, selective HDAC8 compounds described herein, may be used in cancer therapy. In one embodiment, selective HDAC8 compounds described herein, may be used in the treatment of sarcomas. In another embodiment, selective HDAC8 compounds described herein, may be used in the treatment of sarcomas selected from among alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, malignant fibrous histiocytoma, neurofibrosarcoma, rhabdomyosarcoma, synovial sarcoma, askin's tumor, ewing's, malignant hemangioendothelioma, malignant schwannoma, osteosarcoma, chondrosarcoma.

### Hepatitis C:

Chronic hepatitis C is a slowly progressive disease having a significant impact on morbidity and mortality. While many patients who contract hepatitis C will have sub clinical or mild disease, at least 80% of the individuals who contract HCV develop chronic infection and hepatitis. Twenty to fifty percent of these eventually progress to cirrhosis and 1-2% develop liver cancer (Hoofnagle, J. H.; 1997; Hepatology 26: 15S-20S). There are an estimated 170 million HCV carriers world-wide, and HCV-associated end-stage liver disease is now one of the leading cause of liver transplantation. In the United States alone, hepatitis C is responsible for 8,000 to 10,000 deaths annually.

At the present time, interferon-α2b/ribavirin combination therapy is the only available treatment. Sustained virologic response to IFN-α2b -ribavirin combination therapy occurs in about 40-45% of those treated. For those patients who fail interferon-α2b/ribavirin combination therapy, there is currently no alternative to prevent the progression of liver disease. Thus, a need exists for alternative therapies for the treatment of chronic HCV infection. The compounds of Formula (I) and (II) in the present disclosure fulfill this need.

### Uses of HDAC Inhibitor Compounds

The compounds of Formula (I) and (II) of this disclosure are inhibitors of histone deacetylase enzymes and are therefore useful in the treatment of proliferative diseases such as cancer such as lung, colon, AML, MML, skin, breast, ovarian, prostate, liver, brain and skin, psoriasis, fibroproliferative disorder such as liver fibrosis, smooth muscle proliferative disorder such as atherosclerosis and restenosis, inflammatory diseases such as arthritis, diseases involving angiogenesis such as cancer, diabetic retinopathy, haematopoietic disorder such as anaemia, fungal, parasitic and bacterial infections, viral infection, autoimmune diseases such as arthritis, multiple sclerosis, lupus, allergies, asthma, allergic rhinitis, and organ transplant, and bipolar disorders. Additionally, the compounds of the present disclosure are useful in the treatment of hepatitis C infection.

### Compounds

### Compounds of Formula (I)

In one embodiment, described herein are compounds of Formula (I):

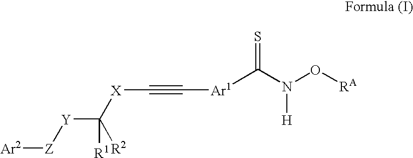

wherein:
R^{A} is hydrogen, alkyl, or alkylcarbonyl;
Ar¹ is an arylene or heteroarylene optionally substituted with one or two substituents independently selected from among alkyl, halo, alkoxy, haloalkoxy, and haloalkyl;
X and Y are independently a bond or an alkylene optionally substituted with halo, haloalkyl, hydroxy, alkoxy, haloalkoxy, amino, alkylamino, or dialkylamino;
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form a cycloalkylene or heterocycloalkylene;
Z is —C(═O)NR³—, —NR³C(═O)—, C(═S)NR³—, —NR³C(═S)—, SO₂NR³—, —NR³SO₂—, —NR³C(═O)NR⁴—, —NR³C(═S)NR⁴—, —NR³SO₂NR⁴—, —OC(═O)NR³—, —OC(═S)NR³—, NR³C(═O)O— or NR³C(═S)O—;
R³ and R⁴ are independently selected from hydrogen, alkyl, hydroxyalkyl, haloalkyl, haloalkoxy, alkoxyalkyl, aralkyl, or heteroaralkyl; and
Ar² is aryl, aralkyl, aralkenyl, heteroaryl, heteroaralkyl, heteroaralkenyl, heterocycloalkyl, or heterocycloalkylalkyl; and individual stereoisomers, individual geometric isomers, or mixtures thereof;
provided that Ar²-Z-Y—(CR¹R²)—X-≡- and C(═S)NHOR^{A} do not occupy adjacent positions on Ar¹; or
a pharmaceutically acceptable salt thereof.

For any and all of the embodiments of Formula (I), substituents can be selected from among from a subset of the listed alternatives. For example, in some embodiments, Z is —C(═O)NR³—, —NR³C(═O)—, SO₂NR³—, —NR³SO₂—, —NR³C(═O)NR⁴—, —NR³SO₂NR⁴—, —OC(═O)NR³—, or NR³C(═O)O—. In other embodiments, Z is —CONR³—, —NR³CO—, SO₂NR³—, —NR³SO₂—, —NR³CONR⁴—, or NR³SO₂NR⁴, where R³ and R⁴ are independently selected from hydrogen, alkyl, hydroxyalkyl, alkoxyalkyl, aralkyl, or heteroaralkyl. In another embodiment, Z is —CONR³—, —NR³CO—, SO₂NR³—, or —NR³SO₂—. In yet another embodiment, Z is —CONR³—. In some embodiments, R³ and R⁴ are hydrogen. In other embodiments, Z is —CONH—, SO₂NH—, or —NHCONH—. In yet other embodiments, Z is —CONH—.

In one embodiment, R^{A} is hydrogen; Ar¹ is arylene optionally substituted with one or two substituents independently selected from alkyl, halo, or haloalkyl. In another embodiment, Ar¹ is phenylene optionally substituted with one or two substituents independently selected from among methyl, fluoro, and trifluoromethyl. In another embodiment, Ar¹ is phenylene and Ar²-Z-Y—(CR¹R²)—X-≡- is attached to Ar¹ in the para position relative to the C(═S)NHOR^{A} group.

In one embodiment, R^{A} is hydrogen; Ar¹ is heteroarylene optionally substituted with one or two substituents independently selected from among alkyl, halo, and haloalkyl. In one aspect, Ar¹ is thienyl.

In another aspect, X and Y are a bond; and R¹ and R² are independently hydrogen or alkyl. In another aspect, R¹ and R² are independently hydrogen or methyl. In another aspect, R¹ and R² are hydrogen.

In another aspect, X and Y are independently a bond or alkylene provided that both X and Y are not a bond.

In another aspect, X and Y are independently a bond, methylene, ethylene, or propylene. In another aspect, X and Y are independently a bond or methylene; and R¹ and R² are independently hydrogen or alkyl. In another aspect, R¹ and R² are independently hydrogen or methyl. In another aspect, R¹ and R² are hydrogen.

In another aspect, X and Y are a bond; and R¹ and R² together with the carbon atom to which they are attached form a cycloalkylene. In one aspect, R¹ and R² together with the carbon atom to which they are attached form a cyclopropylene, cyclopentylene, cyclohexylene, or cycloheptylene.

In another aspect, X and Y are a bond; and R¹ and R² together with the carbon atom to which they are attached form a heterocycloalkylene. In one aspect, R¹ and R² together with the carbon atom to which they are attached form a piperidin-4-yl optionally substituted with alkyl or haloalkyl. In one aspect, R¹ and R² together with the carbon atom to which they are attached form a piperidin-4-yl optionally substituted with methyl, ethyl, or 2,2,2-trifluoroethyl.

In another aspect, X and Y are independently a bond or alkylene provided that both are not a bond; and R¹ and R² together with the carbon atom to which they are attached form cycloalkylene. In another aspect, X and Y are independently a bond, methylene, ethylene, or propylene, provided that both are not a bond. In another aspect, X and Y are independently a bond, or methylene, provided that both are not a bond.

In another aspect, R¹ and R² together with the carbon atom to which they are attached form a cyclopropylene, cyclopentylene, cyclohexylene, or cycloheptylene.

In another aspect, X and Y are independently a bond or alkylene provided that both are not bond; and R¹ and R² together with the carbon atom to which they are attached form heterocycloalkylene. In another aspect, X and Y are independently a bond, methylene, ethylene, or propylene, provided that both are not a bond. In another aspect, X and Y are independently a bond, or methylene, provided that both are not a bond.

In another aspect, R¹ and R² together with the carbon atom to which they are attached form a piperidin-4,4-diyl optionally substituted with alkyl or haloalkyl. In another aspect, R¹ and R² together with the carbon atom to which they are attached form a piperidin-4,4-diyl optionally substituted with methyl, ethyl, or 2,2,2-trifluoroethyl.

In another aspect, Ar² is aryl. In another aspect, Ar² is phenyl optionally substituted with one, two, or three substituents independently selected from among alkyl, alkoxy, alkoxyalkyl, halo, haloalkyl, haloalkoxy, alkylamino, dialkylamino, hydroxy, hydroxyalkyl, hydroxyalkyloxy, aminoalkyl, aminoalkoxy, alkoxyalkyloxy, optionally substituted phenyloxyalkyl, optionally substituted heteroaralkyloxy, optionally substituted heteroaryloxyalkyl, optionally substituted heterocycloalkylalkyloxy, and optionally substituted heterocycloalkylalkyl. In another aspect, Ar² is phenyl optionally substituted with one, two, or three substituents independently selected from among methyl, ethyl, methoxy, chloro, trifluoromethyl, fluoro, 2-methoxyethoxy, 2-(morpholin-4-yl)ethoxy, pyridin-3-ylmethoxy, 2-hydroxyethoxy, 2-(N,N-dimethylamino)ethoxy, methoxymethyl, 2-morpholino-4-ylethyl, morpholino-4-ylmethyl, N,N-dimethylaminomethyl, iso-propoxymethyl, and phenoxymethyl.

In another aspect, Ar² is heteroaryl. In another aspect, Ar² is thienyl, pyridinyl, quinolinyl, thiazolyl, benzthiazolyl, benzoxazolyl, furanyl, benzimidazolyl, benzothienyl, pyrrolyl, indolyl, isoindolyl, or isoquinolinyl optionally substituted with one or two substituents independently selected from among alkyl, alkoxy, alkoxyalkyl, halo, haloalkyl, haloalkoxy, alkylamino, dialkylamino, hydroxy, hydroxyalkyl, hydroxyalkyloxy, aminoalkyl, aminoalkoxy, alkoxyalkyloxy, optionally substituted phenyl, optionally substituted heteroaryl, optionally substituted phenyloxyalkyl, optionally substituted heteroaralkyloxy, optionally substituted heteroaryloxyalkyl, optionally substituted heterocycloalkylalkyloxy, or optionally substituted heterocycloalkylalkyl. In one aspect, Ar² is optionally substituted with one or two substituents independently selected from methyl, ethyl, methoxy, chloro, trifluoromethyl, fluoro, 2-methoxyethoxy, 2-(morpholin-4-yl)ethoxy, pyridin-3-ylmethoxy, 2-hydroxyethoxy, 2-(N,N-dimethylamino)ethoxy, methoxymethyl, 2-morpholino-4-ylethyl, morpholino-4-ylmethyl, N,N-dimethylamino-methyl, 2-N,N-dimethylaminoethyl, aminomethyl, iso-propoxymethyl, phenyl, or phenoxymethyl.

In yet another aspect, Ar² is 4-phenylthiazol-2-yl, 2-(4-H₂NCH₂-phenyl)oxazol-5-yl, benzothien-2-yl, 5-Cl-benzofuran-2-yl, 5-Cl-indol-2-yl, benzofuran-2-yl, indol-2-yl, 3-(CF₃CH₂OCH₂)benzofuran-2-yl, benzothiazol-2-yl, 4-CF₃-benzothien-2-yl, benzimidazol-2-yl, 5-F-benzothien-2-yl, 3-(CH₃)₂N(CH₂)₂-benzofuran-2-yl, 1-(CH₃)₂N(CH₂)₂-benzoimidzol-2-yl, 4-(CH₃)₂N(CH₂)₂O-quinolin-2-yl, or 4-CH₃O-quinolin-2-yl.

In one aspect, compounds of Formula (I) have a structure represented by Formula (Ia):

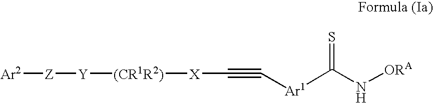

where:
R^{A} is hydrogen, alkyl, or alkylcarbonyl;
Ar¹ is an arylene or heteroarylene optionally substituted with one or two substituents independently selected from among alkyl, halo, alkoxy, haloalkoxy, and haloalkyl;
X and Y are independently a bond or an alkylene optionally substituted with halo, haloalkyl, hydroxy, alkoxy, haloalkoxy, amino, alkylamino, or dialkylamino;
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form a cycloalkylene or heterocycloalkylene;
Z is —C(═O)NR³—, —NR³C(═O)—, C(═S)NR³—, —NR³C(═S)—, SO₂NR³—, —NR³SO₂—, —NR³C(═O)NR⁴—, —NR³C(═S)NR⁴—, —NR³SO₂NR⁴—, —OC(═O)NR³—, —OC(═S)NR³—, NR³C(═S)O— or NR³C(═O)O—;
R³, and R⁴, are independently selected from hydrogen, alkyl, hydroxyalkyl, haloalkyl, haloalkoxy, alkoxyalkyl, aralkyl, or heteroaralkyl; and
Ar² is aryl, or heteroaryl; and
individual stereoisomers, individual geometric isomers, or mixtures thereof;
provided that Ar²-Z-Y—(CR¹R²)—X-≡- and C(═S)NHOR^{A} do not occupy adjacent positions on Ar¹; or
a pharmaceutically acceptable salt thereof.

For any and all of the embodiments, substituents can be selected from among from a subset of the listed alternatives. For example, in some embodiments, Z is —C(═O)NR³—, —NR³C(═O)—, SO₂NR³—, —NR³SO₂—, —NR³C(═O)NR⁴—, —NR³SO₂NR⁴—, —OC(═O)NR³—, or NR³C(═O)O—.

In one aspect, R^{A} is hydrogen; Z is —CONH—; and Ar¹ is arylene optionally substituted with one or two substituents independently selected from alkyl, halo, or haloalkyl. In another aspect, Ar¹ is phenylene optionally substituted with one or two substituents independently selected from methyl, fluoro or trifluoromethyl. In yet another aspect, Ar¹ is phenylene and Ar²-Z-Y—(CR¹R²)—X-≡- is attached to Ar¹ in the para position relative to the C(═S)NHOR^{A} group.

In another aspect, R^{A} is hydrogen; Z is —CONH—; and Ar¹ is heteroarylene optionally substituted with one or two substituents independently selected from among alkyl halo, and haloalkyl. In another aspect, Ar² is heteroarylene optionally substituted with one or two substituents independently selected from among methyl, fluoro, and trifluoromethyl.

In another aspect, X and Y are a bond; and R¹ and R² are independently hydrogen or alkyl. In another aspect, R¹ and R² are independently hydrogen or methyl. In another aspect, R¹ and R² are hydrogen.

In another aspect, X and Y are independently a bond or alkylene provided that both X and Y are not a bond.

In another aspect, X and Y are independently a bond, methylene, ethylene, or propylene. In another aspect, X and Y are independently a bond or methylene. In another aspect, R¹ and R² are independently hydrogen or alkyl. In another aspect, R¹ and R² are independently hydrogen or methyl. In another aspect, R¹ and R² are hydrogen.

In one aspect, X and Y are bond; and R¹ and R² together with the carbon atom to which they are attached form a cycloalkylene. In one aspect, R¹ and R² together with the carbon atom to which they are attached form a cyclopropylene, cyclopentylene, cyclohexylene, or cycloheptylene.

In one aspect, X and Y are bond; and R¹ and R² together with the carbon atom to which they are attached form heterocycloalkylene.

In one aspect, X and Y are independently a bond or alkylene provided that both X and Y are not a bond. In another aspect, X and Y are independently a bond, methylene, ethylene, or propylene. In yet another aspect, X and Y are independently a bond or methylene. In another aspect, R¹ and R² together with the carbon atom to which they are attached form cycloalkylene, preferably cyclopropylene, cyclopentylene, cyclohexylene, or cycloheptylene.

In one aspect, X and Y are independently bond or alkylene provided that both are not a bond. In one aspect, X and Y are independently a bond, methylene, ethylene, or propylene. In another embodiment, X and Y are independently a bond or methylene.

In one aspect, R¹ and R² together with the carbon atom to which they are attached form a heterocycloalkylene. In another embodiment, R¹ and R² together with the carbon atom to which they are attached form a piperidin-4,4-diyl optionally substituted with alkyl or haloalkyl. In another embodiment, R¹ and R² together with the carbon atom to which they are attached form a piperidin-4,4-diyl optionally substituted with methyl, ethyl, or 2,2,2-trifluoroethyl.

In one aspect, compounds of Formula (I) have a formula represented by Formula (Ib):

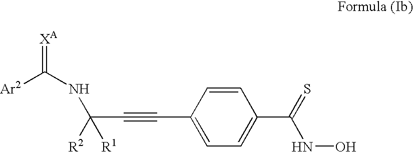

where:
X^{A} is O or S;
R¹ and R² are independently hydrogen or alkyl; and
Ar² is heteroaryl.

In one aspect, compounds of Formula (I) have a formula represented by Formula (Ib):

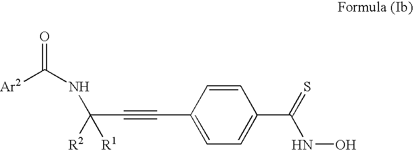

where:
R¹ and R² are independently hydrogen or alkyl; and
Ar² is heteroaryl.

In one aspect, R¹ and R² are independently hydrogen or methyl. In another aspect, R¹ and R² are hydrogen.

In one aspect, Ar² is a substituted or unsubstituted heteroaryl selected from among thiazolyl, quinolinyl, oxazolyl, benzothienyl, indolyl, benzofuranyl, benzthiazolyl, and benzimidazolyl, wherein if Ar² is substituted, then the substitutents on Ar² are selected from among hydrogen, halo, haloalkyl, alkoxy, haloalkoxyalkyl, aminoalkoxy, aminoalkyl, alkoxyalkyloxy, alkoxyalkyloxyalkyl, optionally substituted heterocycloalkyloxy, optionally substituted heterocycloalkylalkyloxy, or phenyl optionally substituted with -(alkylene)NRR′ where R and R′ are independently hydrogen or alkyl.

In another aspect, if Ar² is substituted then each substitutent is selected from among hydrogen, aminoalkoxy, aminoalkyl, alkoxyalkyloxy, alkoxyalkyloxyalkyl, optionally substituted heterocycloalkyloxy, and optionally substituted heterocycloalkylalkyloxy.

In one aspect, Ar² is 4-phenylthiazol-2-yl, 4-MeO-quinolin-2-yl, 2-(4-H₂NCH₂-phenyl)-oxazol-5-yl, benzothien-2-yl, 5-Cl-benzofuran-2-yl, 5-Cl-1H-indol-2-yl, benzofuran-2-yl, 1H-indol-2-yl, 3-(CF₃CH₂OCH₂)-benzofuran-2-yl, benzthiazol-2-yl, 4-CF₃-benzothien-2-yl, benzimidazol-2-yl, 5-F-benzothien-2-yl, 3-(CH₃)₂NCH₂-benzofuran-2-yl, 1-(CH₃)₂N(CH₂)₂-benzimidzol-2-yl, 4-MeO-benzofuran-2-yl, 4-(CH₃)₂N(CH₂)₂O-benzofuran-2-yl, 4-MeO-1H-indol-2-yl, 4-(CH₃)₂N(CH₂)₂O-1H-indol-2-yl, 5-MeO-1H-indol-2-yl, 5-(CH₃)₂N(CH₂)₂O-1H-indol-2-yl, 3-CH₃O(CH₂)₂OCH₂-benzofuran-2-yl, 5-CH₃O(CH₂)₂OCH₂-1H-indol-2-yl, 5-(tetrahydropyran-4-yloxy)-benzofuran-2-yl, 5-(2-pyrrolidin-1-ylethyloxy)benzofuran-2-yl, 5-CH₃O(CH₂)₂O-benzofuran-2-yl, 5-(1-CF₃CH₂-piperidin-4-yloxy)benzofuran-2-yl, 5-(1-cyclopropylpiperidin-4-yloxy)-benzofuran-2-yl, 5-tetrahydropyran-4-ylmethoxy-1H-indol-2-yl, or 5-(2-morpholin-4-ylethoxy)-benzofuran-2-yl.

In yet another aspect, compounds of Formula (I) have a structure of Formula (Ic):

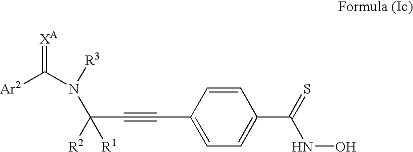

wherein:
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form a cycloalkylene or heterocycloalkylene;
R³ is hydrogen, alkyl, haloalkyl, alkoxyalkyl, aralkyl, or heteroaralkyl; and
X^{A} is O or S;
Ar² is aryl, aralkyl, aralkenyl, heteroaryl, heteroaralkyl, heteroaralkenyl, heterocycloalkyl, or heterocycloalkylalkyl; and
individual stereoisomers, individual geometric isomers, or mixtures thereof; or
a pharmaceutically acceptable salt thereof.

In yet another aspect, compounds of Formula (I) have a structure of Formula (Ic):

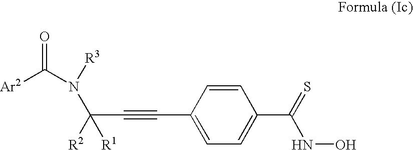

wherein:
R¹ is hydrogen or alkyl;
R² is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R¹ and R² together with the carbon to which they are attached form a cycloalkylene or heterocycloalkylene;
R³ is hydrogen, alkyl, haloalkyl, alkoxyalkyl, aralkyl, or heteroaralkyl; and
Ar² is aryl, aralkyl, aralkenyl, heteroaryl, heteroaralkyl, heteroaralkenyl, heterocycloalkyl, or heterocycloalkylalkyl; and
individual stereoisomers, individual geometric isomers, or mixtures thereof; or
a pharmaceutically acceptable salt thereof.

In one aspect, R³ is hydrogen or alkyl. In another aspect, R³ is hydrogen.

In one aspect, R¹ is hydrogen or alkyl; R² is hydrogen or alkyl; or R¹ and R² together with the carbon to which they are attached form a cycloalkylene or heterocycloalkylene.

In one aspect, Ar² is aryl, aralkyl, aralkenyl, heteroaryl, heteroaralkyl, or heteroaralkenyl. In another aspect, Ar² is aryl, aralkyl, or aralkenyl. In another aspect, Ar² is heteroaryl, heteroaralkyl, or heteroaralkenyl.

In one aspect, Ar² is selected from among phenyl, phenyl-CH═CH—, oxazolyl, thiazolyl, benzthiazolyl, benzimidazolyl, benzothienyl, quinolinyl, indolyl, and benzofuranyl, and if Ar² is substituted, then each substituent is independently selected from among hydrogen, phenyl; 4-H₂NCH₂-phenyl; F; Cl; CF₃—; CF₃O—; CF₃CH₂OCH₂; MeO; (CH₃)₂NCH₂—; (CH₃)₂N(CH₂)₂—;(CH₃)₂N(CH₂)₂O—; CH₃O(CH₂)₂OCH₂—; tetrahydropyran-4-yl-O—; 2-pyrrolidin-1-ylethyloxy; CH₃O(CH₂)₂O—; 4-(CH₃)₂N(CH₂)₂O—; (1-CF₃CH₂-piperidin-4-yloxy); (1-cyclopropylpiperidin-4-yloxy); tetrahydropyran-4-ylmethoxy; and (2-morpholin-4-ylethoxy).

Any combination of the groups described above for the various variables is contemplated herein.

Non-limiting examples of compounds of Formula (I) are disclosed in Table I, Table II, and Table III.

Table I describes representative compounds of Formula (I), where R^{A} and R¹ are hydrogen; Ar¹ is 1,4-phenylene; X and Y are a bond; Z is —CONR³— where R³ is as defined in Table I below; and R² and Ar² are as defined in Table I below:

TABLE I
Representative Compounds of Formula (I)

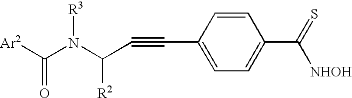

| Compound # | Ar²— | R² | R³ |
|---|---|---|---|
| I-1 | trans phenyl-CH═CH— | H | H |
| I-2 | 4-phenylthiazol-2-yl | H | H |
| I-3 | trans phenyl-CH═CH | (S)—CH₃ | H |
| I-4 | 4-methoxy-quinolin-2-yl | H | H |
| I-5 | 2-(4-H₂NCH₂-phenyl)-oxazol-5-yl | H | H |
| I-6 | 4-phenylthiazol-2-yl | (S)—CH₃ | H |
| I-7 | Phenyl | H | H |
| I-8 | trans phenyl-CH═CH | H | CH₃ |
| I-9 | 4-phenylthiazol-2-yl | H | CH₃ |
| I-10 | benzothien-2-yl | H | H |
| I-11 | 5-Cl-benzofuran-2-yl | H | H |
| I-12 | 5-Cl-1H-indol-2-yl | H | H |
| I-13 | benzofuran-2-yl | H | H |
| I-14 | benzothien-2-yl | (S)—CH₃ | H |
| I-15 | 1H-indol-2-yl | H | H |
| I-16 | 3-(CF₃CH₂OCH₂)-benzofuran-2-yl | H | H |
| I-17 | benzthiazol-2-yl | H | H |
| I-18 | 4-CF₃-benzothien-2-yl | H | H |
| I-19 | benzimidazol-2-yl | H | H |
| I-20 | benzothien-2-yl | H | CH₃ |
| I-21 | 5-F-benzothien-2-yl | H | H |
| I-22 | 3-(CH₃)₂NCH₂-benzofuran-2-yl | H | H |
| I-23 | 1-(CH₃)₂N(CH₂)₂-benzimidazol-2-yl | H | H |
| I-24 | 4-MeO-benzofuran-2-yl | H | H |
| I-25 | 4-(CH₃)₂N(CH₂)₂O-benzofuran-2-yl | H | H |
| I-26 | 4-MeO-1H-indol-2-yl | H | H |
| I-27 | 4-(CH₃)₂N(CH₂)₂O-1H-indol-2-yl | H | H |
| I-28 | 5-MeO-1H-indol-2-yl | H | H |
| I-29 | 5-(CH₃)₂N(CH₂)₂O-1H-indol-2-yl | H | H |
| I-30 | 3-CH₃O(CH₂)₂OCH₂-benzofuran-2-yl | H | H |
| I-31 | 5-CH₃O(CH₂)₂O-1H-indol-2-yl | H | H |
| I-32 | 5-(tetrahydropyran-4-yloxy)- | H | H |
| | benzofuran-2-yl | | |
| I-33 | 5-(2-pyrrolidin-1-ylethyloxy) | H | H |
| | benzofuran-2-yl | | |
| I-34 | 5-CH₃O(CH₂)₂O-benzofuran-2-yl | H | H |
| I-35 | 4-(CH₃)₂N(CH₂)₂O-quinolin-2-yl | H | H |
| I-36 | 5-(1-CF₃CH₂-piperidin-4- | H | H |
| | yloxy)benzofuran-2-yl | | |
| I-37 | 5-(1-cyclopropylpiperidin-4-yloxy)- | H | H |
| | benzofuran-2-yl | | |
| I-38 | 5-tetrahydropyran-4-ylmethoxy-1H- | H | H |
| | indol-2-yl | | |
| I-39 | 5-(2-morpholin-4-ylethoxy)- | H | H |
| | benzofuran-2-yl | | |

Compounds in Table I are named:
4-[3-(3-phenylacryloylamino)prop-1-ynyl]-phenylthiohydroxamic acid (Compound I-1);
4-[3-(4-phenylthiazol-2-ylcarbonylamino)prop-1-ynyl]-phenylthiohydroxamic acid (Compound I-2);
4-[3S-(3-phenylacryloylamino)-but-1-ynyl]-phenylthiohydroxamic acid (Compound I-3);
4-[3-(4-methoxyquinolin-2-ylcarbonylamino)prop-1-ynyl]-phenylthiohydroxamic acid (Compound I-4);
4-{3-[2-(4-aminomethylphenyl)oxazol-5-ylcarbonylamino)prop-1-ynyl]-phenylthiohydroxamic acid (Compound I-5);
4-[3S-(4-phenylthiazol-2-ylcarbonylamino)but-1-ynyl]-phenylthiohydroxamic acid (Compound I-6);
4-[3-(phenylcarbonylamino)prop-1-ynyl]-phenylthiohydroxamic acid (Compound I-7);
4-{3-[methyl-(3-phenylacryloyl)amino]prop-1-ynyl}phenylthiohydroxamic acid (Compound I-8);
4-{3-[methyl-(4-phenylthiazol-2-ylcarbonyl)amino]prop-1-ynyl}-phenylthiohydroxamic acid (Compound I-9);
4-[3-(benzothien-2-ylcarbonylamino)prop-1-ynyl]-phenylthiohydroxamic acid (Compound I-10);
4-[3-(5-chlorobenzofuran-2-ylcarbonylamino)prop-1-ynyl]-phenylthiohydroxamic acid (Compound I-11);
4-[3-(5-indol-2-ylcarbonylamino)prop-1-ynyl]-phenylthiohydroxamic acid (Compound I-12);
4-[3-(benzofuran-2-ylcarbonylamino)prop-1-ynyl]-phenylthiohydroxamic acid (Compound I-13);
4-[3S-(benzothien-2-ylcarbonylamino)but-1-ynyl]-phenylthiohydroxamic acid (Compound I-14);
4-[3-(indol-2-ylcarbonylamino)prop-1-ynyl]-phenylthiohydroxamic acid (Compound I-15);
4-{3-[3-(2,2,2-trifluoroethyloxymethyl)benzofuran-2-yl-carbonyl-amino]prop-1-ynyl}-phenylthiohydroxamic acid (Compound I-16);
4-[3-(benzthiazol-2-ylcarbonylamino)prop-1-ynyl]-phenylthiohydroxamic acid (Compound I-17);
4-[3-(4-trifluoromethylbenzothien-2-ylcarbonyl-amino)prop-1-ynyl]-phenylthiohydroxamic acid (Compound I-18);
4-[3-(benzimidazol-2-ylcarbonylamino)prop-1-ynyl]-phenylthiohydroxamic acid (Compound I-19);
4-{3-[methyl-(benzothien-2-ylcarbonyl)amino]prop-1-ynyl}-phenylthiohydroxamic acid (Compound I-20);
4-[3-(5-fluorobenzothien-2-ylcarbonylaminoprop-1-ynyl]-phenylthiohydroxamic acid (Compound I-21);
4-[3-(3-N,N-dimethylaminomethylbenofuran-2-ylcarbonylamino)prop-1-ynyl]-phenylthiohydroxamic acid (Compound I-22);
4-{3-[1-(2-N,N-dimethylaminoethyl)benzimidazol-2-ylcarbonyl-amino]prop-1-ynyl}phenylthiohydroxamic acid (Compound I-23);
4-[3-(4-methoxybenzofuran-2-ylcarbonylamino)prop-1-ynyl]-phenylthiohydroxamic acid (Compound I-24);
4-[3-(4-N,N-dimethylaminoethoxybenzofuran-2-ylcarbonylamino)prop-1-ynyl]-phenylthiohydroxamic acid (Compound I-25);
4-[3-(4-methoxyindol-2-ylcarbonylamino)prop-1-ynyl]-phenylthiohydroxamic acid (Compound I-26);
4-[3-(4-N,N-dimethylaminoethoxyindol-2-yl-carbonylamino)prop-1-ynyl]-phenylthiohydroxamic acid (Compound I-27);
4-[3-(5-methoxyindol-2-ylcarbonylamino)prop-1-ynyl]-phenylthiohydroxamic acid (Compound I-28);
4-[3-(5-N,N-dimethylaminoethoxyindol-2-ylcarbonyl-amino)prop-1-ynyl]-phenylthiohydroxamic acid (Compound I-29);
4-{3-[3-(2-methoxyethyloxymethyl)benzofuran-2-yl-carbonylamino]prop-1-ynyl}phenylthiohydroxamic acid (Compound I-30);
4-{3-[3-(2-methoxyethyloxy)indol-2-yl-carbonylamino]prop-1-ynyl}-phenylthiohydroxamic acid (Compound I-31);
4-[3-(5-tetrahydropyran-4-yloxybenzofuran-2-yl-carbonylamino)prop-1-ynyl]phenylthiohydroxamic acid (Compound I-32);
4-{3-[5-(2-pyrrolidin-1-ylethoxy)benzofuran-2-yl-carbonylamino]prop-1-ynyl}phenylthiohydroxamic acid (Compound I-33);
4-{3-[5-(2-methoxyethyloxy)benzofuran-2-yl-carbonylamino]prop-1-ynyl}-phenylthiohydroxamic acid (Compound I-34);
4-{3-[4-(N,N-dimethylaminoethyloxy)quinolin-2-yl-carbonylamino]prop-1-ynyl}phenylthiohydroxamic acid (Compound I-35);
4-{3-[5-(1-(2,2,2-trifluoroethyl)piperidin-4-yloxy)benzofuran-2-ylcarbonyl-amino]prop-1-ynyl}phenylthiohydroxamic acid (Compound I-36);
4-{3-[5-(1-cyclopropylpiperidin-4-yloxy)benzofuran-2-yl-carbonylamino]-prop-1-ynyl}phenylthiohydroxamic acid (Compound I-37);
4-{3-[5-(tetrahydropyran-4-ylmethyloxy)benzofuran-2-ylcarbonylamino]-prop-1-ynyl}phenylthiohydroxamic acid (Compound I-38); and
4-{3-[5-(2-morpholin-4-ylethyloxy)benzofuran-2-yl-carbonylamino]prop-1-ynyl}phenylthiohydroxamic acid (Compound I-39).

Also described herein are pharmaceutically acceptable salts of compounds of Table I. Table II describes representative compounds of Formula (I), where Ar¹ is phenylene; X and Y are bond; R^{A}, R¹ and R² are hydrogen; and Z and Ar² are as defined in Table II below:

TABLE II
Representative Compounds of Formula (I)

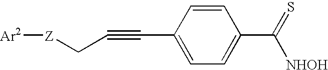

| Compound # | Ar²— | Z |
|---|---|---|
| II-1 | 4-Cl-phenyl | —NHCONH— |
| II-2 | 4-CF₃-phenyl | —NHCONH— |
| II-3 | phenyl | —NHCONH— |
| II-4 | 2-CF₃O-phenyl | —NHCONH— |
| II-5 | phenyl | —SO₂NH— |

Compounds in Table II are named:
4-{3-[3-(4-chlorophenyl)ureido]prop-1-ynyl}phenylthiohydroxamic acid (Compound II-1);
4-{3-[3-(4-trifluoromethylphenyl)ureido]prop-1-ynyl}phenylthiohydroxamic acid (Compound II-2);
4-{3-[3-(phenyl)ureido]prop-1-ynyl}phenylthiohydroxamic acid (Compound II-3);
4-{3-[3-(2-trifluoromethoxyphenyl)ureido]prop-1-ynyl}phenylthiohydroxamic acid (Compound II-4); and
4-[3-(phenylsulfonylamino)prop-1-ynyl]-phenylthiohydroxamic acid (Compound II-5).

Also described herein are pharmaceutically acceptable salts of compounds of Table II.

Table III describes representative compounds of Formula (I) where Ar¹ is 1,4-phenylene; X and Y are a bond; R^{A} is hydrogen; Z is —CONH—; and R¹, R², and Ar² are as defined in Table III below:

TABLE III
Representative Compounds of Formula (I)

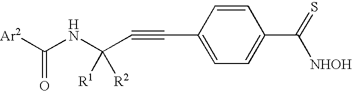

| Compound # | Ar²— | >CR¹R² |
|---|---|---|
| III-1 | trans-phenyl-CH═CH— | >C(CH₃)₂ |
| III-2 | 4-phenylthiazol-2-yl | >C(CH₃)₂ |
| III-3 | benzothien-2-yl | >C(CH₃)₂ |
| III-4 | benzofuran-2-yl | >C(CH₃)₂ |
| III-5 | benzofuran-2-yl | 1,1-cyclopropylene |
| III-6 | 1H-indol-2-yl | 1,1-cyclopropylene |
| III-7 | benzofuran-2-yl | 1,1-cyclobutylene |
| III-8 | benzofuran-2-yl | 1,1-cyclopentylene |
| III-9 | benzofuran-2-yl | 1,1-cyclohexylene |
| III-10 | 1H-indol-2-yl | 1,1-cyclohexylene |
| III-11 | benzofuran-2-yl | 1,1-cycloheptylene |
| III-12 | 1H-indol-2-yl | 1,1-cycloheptylene |
| III-13 | 1H-indol-2-yl | 1,1-cyclopentylene |
| III-14 | 1H-indol-2-yl | 1,1-cyclobutylene |
| III-15 | benzothien-2-yl | 1,1-cyclopropylene |
| III-16 | 1H-indol-2-yl | 1,1-tetrahydropyran-4-yl |
| III-17 | 4-CH₃O-1H-indol-2-yl | 1,1-cyclopropylene |
| III-18 | 5-CH₃O-1H-indol-2-yl | 1,1-cyclopropylene |
| III-19 | 1H-indol-2-yl | piperidin-4,4-diyl |
| III-20 | benzofuran-2-yl | piperidin-4,4-diyl |
| III-21 | 1H-indol-2-yl | 1-CF₃CH₂-piperidin-4,4-diyl |

Compounds in Table III are named:
4-[3-methyl-3-(3-phenylacryloylamino)but-1-ynyl]-phenylthiohydroxamic acid (Compound III-1);
4-[3-methyl-3-(4-phenylthiazol-2-ylcarbonylamino)but-1-ynyl]-phenylthiohydroxamic acid (Compound III-2);
4-[3-methyl-3-(benzthiazol-2-ylcarbonylamino)but-1-ynyl]-phenylthiohydroxamic acid (Compound III-3);
4-[3-methyl-3-(benzofuran-2-ylcarbonylamino)but-1-ynyl]-phenylthiohydroxamic acid (Compound III-4);
4-[1-(benzofuran-2-yl-carbonylamino)-cycloprop-1-ylethynyl]-phenylthiohydroxamic acid (Compound III-5);
4-[1-(1H-indol-2-yl-carbonylamino)-cycloprop-1-yl-ethynyl]-phenylthiohydroxamic acid (Compound III-6);
4-[1-(benzofuran-2-ylcarbonylamino)-cyclobut-1-yl-ethynyl]-phenylthiohydroxamic acid (Compound III-7);
4-[1-(benzofuran-2-yl-carbonylamino)-cyclohept-1-yl-ethynyl]-phenylthiohydroxamic acid (Compound III-8);
4-[1-(benzofuran-2-yl-carbonylamino)-cyclohex-1-yl-ethynyl]-phenylthiohydroxamic acid (Compound III-9);
4-[1-(1H-indol-2-yl-carbonylamino)-cyclohex-1-yl-ethynyl]-phenylthiohydroxamic acid (Compound III-10);
4-[1-(benzofuran-2-yl-carbonylamino)-cyclohept-1-yl-ethynyl]-phenylthiohydroxamic acid (Compound II-11);
4-[1-(1H-indol-2-yl-carbonylamino)-cyclohept-1-yl-ethynyl]-phenylthiohydroxamic acid (Compound III-12);
4-[1-(1H-indol-2-yl-carbonylamino)-cyclopent-1-yl-ethynyl]-phenylthiohydroxamic acid (Compound III-13);
4-[1-(1H-indol-2-yl-carbonylamino)-cyclobut-1-yl-ethynyl]-phenylthiohydroxamic acid (Compound III-14);
4-[1-(benzothien-2-yl-carbonylamino)-cycloprop-1-yl-ethynyl]-phenylthiohydroxamic acid (Compound III-15);
4-[4-(1H-indol-2-yl-carbonylamino)tetrahydrofuran-4-yl-ethynyl]-phenylthiohydroxamic acid (Compound III-16);
4-[1-(4-methoxyindol-2-yl-carbonylamino)-cycloprop-1-yl-ethynyl]-phenylthiohydroxamic acid (Compound III-17);
4-[1-(5-methoxyindol-2-yl-carbonylamino)-cycloprop-1-yl-ethynyl]-phenylthiohydroxamic acid (Compound III-18);
4-[4-(1H-indol-2-yl-carbonylamino)piperidin-4-yl-ethynyl]-phenylthiohydroxamic acid (Compound III-19);
4-[4-(benzofuran-2-yl-carbonylamino)piperidin-4-yl-ethynyl]-phenylthiohydroxamic acid (Compound III-20); and
4-[4-(1H-indol-2-yl-carbonylamino)-1-(2,2,2-trifluoroethyl)piperidin-4-yl-ethynyl]-phenylthiohydroxamic acid (Compound III-21).

Also described herein are pharmaceutically acceptable salts of compounds of Table III.

### Compounds of Formula (II)

In one aspect, described herein are compounds of Formula (II):

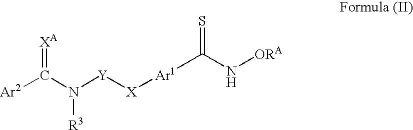

wherein:
R^{A} is hydrogen or alkyl;
X is a bond, O—, —NR¹—, or S(O)ₙ;
n is 0, 1, or 2;
X^{A} is O or S;
R¹ is hydrogen or alkyl;
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl, halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

In some embodiments, X^{A} is S. In other embodiments, X^{A} is O.

In another aspect, described herein are compounds of Formula (II):

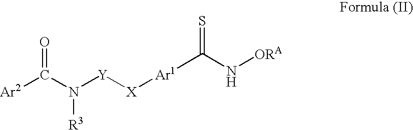

wherein:
R^{A} is hydrogen or alkyl
X is a bond, —O—, —NR¹—, or S(O)ₙ;
n is 0, 1, or 2;
R¹ is hydrogen or alkyl
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl, halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

For any and all of the embodiments of Formula (II), substituents can be selected from among from a subset of the listed alternatives. For example, in some embodiments, R^{A} is hydrogen or alkyl; X is —O—, —NR¹—, or S(O)ₙ; n is 0-2; Y is alkylene optionally substituted with cycloalkyl, optionally substituted phenyl, alkylthio, alkysulfonyl, optionally substituted phenylalkylthio, optionally substituted phenylalkylsulfonyl, or hydroxy; Ar¹ is phenylene or heteroarylene wherein said Ar¹ is optionally substituted with one or two groups independently selected from alkyl, halo, hydroxy, alkoxy, haloalkoxy, or haloalkyl; R³ is hydrogen, alkyl, hydroxyalkyl, or optionally substituted phenyl; and Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl.

In another aspect, R^{A} and R³ are hydrogen, X is —O—; and Y is ethylene or n-propylene. In another aspect, R^{A} and R³ are hydrogen, X is —O—; and Y is ethylene.

In another aspect, R^{A} and R³ are hydrogen; X is —O—; and Y is —CH(C₂H₅)CH₂—, CH(i-C₃H₇)CH₂—, or —CH(CH₃)CH₂—. In another aspect, Y is —CH(C₂H₅)CH₂—. In yet another aspect, the stereochemistry at the chiral carbon in the Y substituent is (S).

In another aspect, R^{A} and R³ are hydrogen; X is —O—; and Y is —CH₂CH(CH₃)—. In yet another aspect, the stereochemistry at the chiral carbon in the Y substitutent is (R).

In another aspect, Ar¹ is phenylene in which C(═S)NHOR^{A} and Ar²—C(═O)NR³—Y—X— are para to each other; and Ar² is phenyl.

In another aspect, Ar² is phenyl and is optionally substituted with one or two substituents independently selected from methoxy, ethoxy, phenyl, methyl, tert-butyl, pyrrol-1-yl, cyclohexene-3-oxy, pyridin-3-yl, pyridin-2-yl, benzoylamino, fluoro, chloro, or thiophen-2-ylmethoxy.

In yet another aspect, Ar² is phenyl, 4-biphenyl, 3-biphenyl, 4-tert-butylphenyl, 4-pyrrol-1-ylphenyl, 4-(cyclohexene-3-oxy)phenyl, 4-(pyridin-2-yl)phenyl, 4-(pyridin-3-yl)-phenyl, 2,4-difluorophenyl, 3,4-dimethoxyphenyl, 3,5-dimethoxyphenyl, 3,4-difluorophenyl, 2,5-dimethylphenyl, 2,3-dichlorophenyl, 2,3-dimethylphenyl, 4-chloro-2-methoxyphenyl, 3-ethoxyphenyl, 4-methoxy-2-methylphenyl, 3-fluoro-4-methoxyphenyl, 2-thiophen-2-ylmethoxyphenyl, 3-thiophen-2-ylmethoxyphenyl, 2-biphenyl, or 2-pyrrol-1-ylphenyl.

In another aspect, Ar¹ is phenylene in which C(═S)NHOR^{A} and Ar²—C(═O)NR³—Y—X— are para to each other; and Ar² is trans-aryl-CH═CH—.

In another aspect, Ar² is trans-phenyl-CH═CH— and is optionally substituted with alkoxy. In another aspect, Ar² is trans-phenyl-CH═CH— and is optionally substituted with methoxy. In another aspect, Ar² is trans phenyl-CH═CH—.

In another aspect, Ar¹ is phenylene in which C(═S)NHOR^{A} and Ar²—C(═O)NR³—Y—X— are para to each other and Ar² is heteroaryl-CH═CH—.

In another aspect, Ar² is pyridinyl-CH═CH—.

In another aspect, Ar² is trans-(5-bromothien-2-yl)-CH═CH— or trans-(indol-3-yl)-CH═CH—.

In another aspect, Ar¹ is phenylene in which C(═S)NHOR¹ and Ar²—C(═O)NR³—Y—X— are para to each other; and Ar² is heteroaryl.

In another aspect, Ar² is pyridin-3-yl, thien-2-yl, quinolin-6-yl, thiazol-2-yl, benzthiazol-2-yl, benzoxazol-2-yl, furanyl, pyrrol-2-yl, indol-5-yl, indol-3-yl, indazol-3-yl, quinolin-3-yl, quinolin-1-yl, quinolin-8-yl, benzotriazol-4-yl, benzofuran-5-yl, isoquinolin-1-yl, isoquinolin-3-yl, quinoxalin-2-yl, quinolin-2-yl, or benzimidazol-5-yl wherein Ar² is optionally substituted with phenyl, pyridin-4-yl, methyl, methoxy, or dimethylaminomethyl.

In another aspect, Ar¹ is phenylene in which C(═S)NHOR^{A} and Ar²—C(═O)NR³—Y—X— are para to each other; and Ar² is indol-2-yl, benzofuran-2-yl or benzothien-2-yl, where Ar² is optionally substituted with alkyl, alkoxy, halo, haloalkyl, alkoxyalkyloxy, -alkyl-N(heteroalkyl)₂, optionally substituted heterocycloalkylalkyloxy, optionally substituted heteroaralkyloxy, hydroxyalkoxy, aminoalkyl, aminoalkyloxy, alkoxyalkyloxy, alkoxyalkyl, optionally substituted phenyloxyalkyl, or optionally substituted heterocycloalkylalkyl.

In another aspect, Ar² is benzofuran-2-yl or benzothien-2-yl wherein benzofuran-2-yl or benzothien-2-yl is optionally substituted with methoxy, methyl, chloro, trifluoromethyl, fluoro, 2-methoxyethoxy, 2-morpholin-4-ylethoxy, pyridin-3-ylmethoxy, 2-hydroxyethoxy, 2-N,N-dimethylaminoethoxy, ethyl, methoxymethyl, 2-propyloxymethyl, phenoxymethyl, morpholin-4-ylmethyl, or N,N-dimethylaminomethyl.

In another aspect, Ar² is benzofuran-2-yl or benzothien-2-yl wherein benzofuran-2-yl or benzothien-2-yl is optionally substituted with methoxy, methyl, chloro, trifluoromethyl, fluoro, 2-methoxyethoxy, 2-morpholin-4-ylethoxy, pyridin-3-ylmethoxy, 2-hydroxyethoxy, 2-N,N-dimethylaminoethoxy, ethyl, methoxymethyl, 2-propyloxymethyl, phenoxymethyl, morpholin-4-ylmethyl, N,N-dimethylaminomethyl, N,N-di(methoxyethoxyethoxyethoxymethyl)aminomethyl, or N,N-di(methoxyethoxyethoxymethyl)aminomethyl, N,N-di(methoxyethoxymethyl)aminomethyl.

In another aspect, Ar² is benzofuran-2-yl or benzothien-2-yl wherein benzofuran-2-yl or benzothien-2-yl is optionally substituted at the 3-position or 5-position with methoxy, methyl, chloro, trifluoromethyl, fluoro, 2-methoxyethoxy, 2-morpholin-4-ylethoxy, pyridin-3-ylmethoxy, 2-hydroxyethoxy, 2-N,N-dimethylaminoethoxy, ethyl, methoxymethyl, 2-propyloxymethyl, phenoxymethyl, morpholin-4-ylmethyl, or N,N-dimethylaminomethyl.

In another aspect, Ar² is benzofuran-2-yl or benzothien-2-yl, wherein benzofuran-2-yl or benzothien-2-yl is optionally substituted at the 3-position with methoxy, methyl, chloro, trifluoromethyl, fluoro, 2-methoxyethoxy, 2-morpholin-4-ylethoxy, pyridin-3-ylmethoxy, 2-hydroxyethoxy, 2-N,N-dimethylaminoethoxy, ethyl, methoxymethyl, 2-propyloxymethyl, phenoxymethyl, morpholin-4-ylmethyl, or N,N-dimethylaminomethyl.

In another aspect, Ar² is benzofuran-2-yl, 3-(N,N-dimethylaminomethyl)benzofuran-2-yl, or 3-(phenoxymethyl)benzofuran-2-yl.

In another aspect, Ar¹ is phenylene in which C(═S)NHOR^{A} and Ar²—C(═O)NR³—Y—X— are para to each other; and Ar² is indol-2-yl, benzofuran-2-yl or benzothien-2-yl, where Ar² is substituted with phenyloxyalkyl, substituted heteroaryloxyalkyl, substituted heterocycloalkyloxyalkyl, or haloalkoxyalkyl which are located at the 3-position of the benzothien-2-yl and benzofuran-2-yl rings.

In another aspect, Ar² is 3-(2,2,2-trifluoroethyloxymethyl)benzofuran-2-yl.

In another aspect, Ar¹ is heteroarylene; and Ar² is aryl.

In another aspect, Ar¹ is five membered heteroarylene ring containing one, two, or three heteroatoms independently selected from N, O or S.

In another aspect, Ar¹ is isoxazolyl where Ar²—C(═O)NR³—Y—X— and C(═S)NHOR^{A} and Ar²—C(═O)NR³—Y—X— are located at the 5- and 3-position of the isoxazolyl ring, the oxygen atom in the ring being position 1; and Ar² is aryl.

In another aspect, Ar² is phenyl that is optionally substituted with one or two substituents independently selected from methoxy, ethoxy, (phenyl optionally substituted with ethoxy or methyl), methyl, tert-butyl, pyrrol-1-yl, cyclohexene-3-oxy, pyridin-3-yl, pyridin-2-yl, benzoylamino, fluoro, chloro, or thiophen-2-ylmethoxy.

In another aspect, Ar² is phenyl, 4-biphenyl, 3-biphenyl, 2-(2-ethoxyphenyl)phenyl, 3-methylbiphen-4-yl, 4-tert-butylphenyl, 4-pyrrol-1-ylphenyl, 4-(cyclohexene-3-oxy)phenyl, 4-(pyridin-2-yl)phenyl, 4-(pyridin-3-yl)-phenyl, 2,4-difluorophenyl, 3,4-dimethoxyphenyl, 3,5-dimethoxyphenyl, 3,4-difluorophenyl, 2,5-dimethylphenyl, 2,3-dichlorophenyl, 2,3-dimethylphenyl, 4-chloro-2-methoxyphenyl, 3-ethoxyphenyl, 4-methoxy-2-methylphenyl, 3-fluoro-4-methoxyphenyl, 2-thien-2-ylmethoxyphenyl, 3-thiophen-2-ylmethoxyphenyl, 2-biphenyl, or 2-pyrrol-1-ylphenyl.

In another aspect, Ar¹ is heteroarylene and Ar² is aryl-CH═CH—.

In another aspect, Ar¹ is a five membered heteroarylene ring containing one, two, or three heteroatoms independently selected from N, O or S.

In another embodiment, Ar¹ is isoxazolyl where C(═S)NHOR^{A} and Ar²—C(═O)NR³—Y—X— are located at the 5- and 3-position of the isoxazolyl ring, the oxygen atom in the isoxazolyl ring being position 1; and Ar² is phenyl-CH═CH— and is optionally substituted with alkoxy.

In another embodiment, Ar¹ is heteroarylene and Ar² is heteroaryl-CH═CH—.

In another embodiment, Ar¹ is a five membered heteroarylene ring containing one, two, or three heteroatoms independently selected from N, O or S.

In another embodiment, Ar¹ is isoxazolyl where C(═S)NHOR^{A} and Ar²—C(═O)NR³—Y—X— are located at the 5- and 3-position of the isoxazolyl ring, the oxygen atom in the isoxazolyl ring being position 1; and Ar² is pyridinyl-CH═CH—.

In another embodiment, Ar¹ is heteroarylene; and Ar² is heteroaryl. In another embodiment, Ar¹ is a five membered heteroarylene ring containing one, two, or three heteroatoms independently selected from N, O or S. In another embodiment, Ar¹ is isoxazolyl where C(═S)NHOR^{A} and Ar²—C(═O)NR³—Y—X— are located at the 5- and 3-position of the isoxazolyl ring, the oxygen atom in the isoxazolyl ring being position 1; and Ar² is pyridin-3-yl, thien-2-yl, quinolin-6-yl, thiazol-2-yl, benzthiazol-2-yl, benzoxazol-2-yl, furanyl, pyrrol-2-yl, indol-5-yl, indol-3-yl, indazol-3-yl, quinolin-3-yl, quinolin-8-yl, benzotriazol-4-yl, isoquinolin-1-yl, isoquinolin-3-yl, quinoxalin-2-yl, quinolin-2-yl, or benzimidazol-5-yl wherein Ar² is optionally substituted with phenyl, pyridin-4-yl, methyl, methoxy, or dimethylaminomethyl.

In another embodiment, Ar¹ is heteroarylene; and Ar² is indol-2-yl, benzofuran-2-yl or benzothien-2-yl, where Ar² is optionally substituted with alkyl, alkoxy, halo, haloalkyl, alkoxyalkyloxy, optionally substituted heterocycloalkylalkyloxy, optionally substituted heteroaralkyloxy, hydroxyalkoxy, aminoalkyloxy, alkoxyalkyloxy, alkoxyalkyl, optionally substituted phenyloxyalkyl, or optionally substituted heterocycloalkylalkyl. In another embodiment, Ar¹ is a five membered heteroarylene ring containing one, two, or three heteroatoms independently selected from N, O or S. In another embodiment, Ar¹ is isoxazolyl where C(═S)NHOR^{A} and Ar²—C(═O)NR³—Y—X— are located at the 5- and 3-position of the isoxazolyl ring, the oxygen atom in the isoxazolyl ring being position 1; and Ar² is benzofuran-2-yl or benzothien-2-yl, where benzofuran-2-yl or benzothien-2-yl which are optionally substituted at the 3-position with methoxy, methyl, chloro, trifluoromethyl, fluoro, 2-methoxyethoxy, 2-morpholin-4-ylethoxy, pyridin-3-ylmethoxy, 2-hydroxyethoxy, 2-N,N-dimethylaminoethoxy, ethyl, methoxymethyl, phenoxymethyl, morpholin-4-ylmethyl, or dimethylaminomethyl. In another embodiment, Ar² is benzofuran-2-yl or 3-phenoxymethylbenzofuran-2-yl.

In another embodiment, Ar² is substituted with alkoxyalkyloxy, optionally substituted heterocycloalkylalkyloxy, hydroxyalkoxy, aminoalkyloxy, alkoxyalkyloxy, alkoxyalkyl, optionally substituted phenyloxyalkyl, optionally substituted heteroaryloxyalkyl, or optionally substituted heterocycloalkylalkyl.

In another embodiment, X is O—; and R^{A} and R³ are hydrogen.

In another embodiment, X is S(O)ₙ and R^{A} and R³ are hydrogen.

In another embodiment, Y is alkylene.

In yet another embodiment, Y is alkylene substituted with cycloalkyl, optionally substituted phenyl, alkylthio, alkylsulfinyl, alkysulfonyl, optionally substituted phenylalkylthio, optionally substituted phenylalkylsulfonyl, hydroxyl, or optionally substituted phenoxy.

In another embodiment, Ar¹ is phenylene.

In another embodiment, Ar¹ is heteroarylene.

In another embodiment, Ar¹ is phenylene, a particularly preferred group of compounds is that wherein C(═S)NHOR^{A} and Ar²—C(═O)NR³—Y—X— are at the 1 and 4 position of the phenylene ring.

In another embodiment, Ar¹ is phenylene; X is O—; R^{A} and R³ are hydrogen; and C(═S)NHOR^{A} and Ar²—C(═O)NR³—Y—X— are at the 1 and 4 position of the phenylene ring.

In another embodiment, Y is alkylene.

In another embodiment, Y is alkylene substituted with cycloalkyl, optionally substituted phenyl, alkylthio, alkylsulfinyl, alkysulfonyl, optionally substituted phenylalkylthio, optionally substituted phenylalkylsulfonyl, hydroxyl, or optionally substituted phenoxy.

In another embodiment, Ar² is aralkenyl. In another embodiment, Ar² is aryl(C₂-C₃)alkenyl. In another embodiment, Ar² is represented by the formulae:

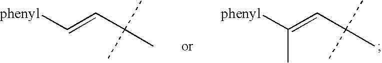

where phenyl is optionally substituted with one or two substituents independently selected from alkyl, alkoxy, methylenedioxy, dialkylamino, or hydroxy. In another embodiment, phenyl is optionally substituted with alkyl, alkoxy, methylenedioxy, or hydroxy.

In another embodiment, Ar² is trans-phenyl-CH═CH—, trans-(4-methoxy-phenyl)-CH═CH—, trans-(3,4-methylenedioxyphenyl)-CH═CH—, trans-(3-hydroxyphenyl)-CH═CH—, trans-(4-hydroxyphenyl)-CH═CH—, trans-(2-methoxyphenyl)-CH═CH—, trans-(3-methoxyphenyl)-CH═CH—, trans-(3-tolyl)-CH═CH—, trans-(4-tolyl)-CH═CH—, trans-(4-dimethylaminophenyl)-CH═CH—, trans-(2-tolyl)-CH═CH—, or trans-(2-hydroxyphenyl)-CH═CH—.

In another embodiment, Ar² is heteroaryl(C₂-C₃)alkenyl. In another embodiment, Ar² is trans-heteroaryl-CH═CH— or trans-heteroaryl-C(CH₃)═CH—. In another embodiment, Ar² is trans-heteroaryl-CH═CH— or trans-heteroaryl-C(CH₃)═CH—, where the heteroaryl of Ar² is pyridinyl, benzofuranyl, thienyl, furanyl, or indolyl optionally substituted with one or two substituents selected from hydroxyl, alkoxy, halo, or optionally substituted heterocycloalkoxy.

In another embodiment, Ar² is trans pyridin-3-yl-CH═CH—, trans-(5-hydroxybenzofuran-2-yl)-C(CH₃)═CH—, trans-(5-(1-cyclopropylpiperidin-4-yloxy)benzofuran-2-yl)-C(CH₃)═CH—, trans-(5-methoxybenzofuran-2-yl)-C(CH₃)═CH—, trans-(benzofuran-2-yl)-CH═CH—, trans-(5-bromothien-2-yl)-CH═CH—, trans-(furan-3-yl)-CH═CH—, trans-(thien-3-yl)-CH═CH—, trans-(thien-2-yl)-CH═CH—, trans-(benzofuran-2-yl)-C(CH₃)═CH—, cis-(benzofuran-2-yl)-C(CH₃)═CH—, trans-(indol-3-yl)-CH═CH—, trans-(7-methoxybenzofuran-2-yl)-CH═CH—, trans-(5-methoxybenzofuran-2-yl)-C(CH₃)═CH—, or trans-(furan-2-yl)-CH═CH.

In another embodiment, Ar² is aryl. In another embodiment, Ar² is aryl and the substitutents on the aryl ring are independently selected from among hydrogen, optionally substituted phenyl, alkyl, alkoxy, halo, optionally substituted heteroaryl, optionally substituted cycloalkenyloxy, optionally substituted heteroaralkyloxy, optionally substituted heterocycloalkyl, optionally substituted phenylcarbonylamino, and methylenedioxy. In another embodiment, Ar² is phenyl, 4-biphenyl, 3-biphenyl, 4-tert-butylphenyl, 4-pyrrol-1-ylphenyl, 4-(pyridin-3-yl)phenyl, 4-(pyridin-2-yl)phenyl, 4-(benzoylamino)phenyl, 2,4-difluorophenyl, 3,4-methylenedioxyphenyl, 3,4-dimethoxyphenyl, 3,5-dimethoxyphenyl, 3,4-difluorophenyl, 2,5-dimethylphenyl, 2,3-dichlorophenyl, 2,3-dimethylphenyl, 4-chloro-2-methoxyphenyl, 3-ethoxyphenyl, 4-methoxy-2-methylphenyl, 3-fluoro-4-methoxyphenyl, 2-(thien-2-ylmethoxy)phenyl, 3-(thien-2-ylmethoxy)-phenyl, 2-biphenyl, naphth-1-yl, 2-pyrrol-1-yl-phenyl, 4-fluoronaphth-1-yl, 3-methoxy-naphth-2-yl, 2-methoxy-naphth-1-yl, naphth-2-yl, 4-(2-pyridin-4-ylthiazol-5-yl)phenyl, 4-[2-(4-methylpiperazin-1-yl)thiazol-5-yl]-phenyl, 4-(2-pyridin-4-ylaminothiazol-5-yl)phenyl, 4-(4-methylpiperazin-1-yl)phenyl, 4-(4-hydroxypiperidin-1-yl)phenyl, 4-(4-morpholin-4-ylmethylthiazol-2-yl)phenyl, 4-[2-(4-methylpiperazin-1-ylmethyl)thiazol-5-yl]phenyl, 1-methoxynaphth-2-yl, 3′-(2-hydroxyethyl)biphen-4-yl, 3′-(2-hydroxyethyl)biphen-3-yl, 2′-(2-hydroxyethyl)biphen-4-yl, 2′-(2-hydroxyethyl)biphen-3-yl or 4-[4-(2-morpholin-4-yl-ethyl)thiazol-2-yl]phenyl.

In another embodiment, Ar² is heteroaryl. In another embodiment, Ar² is heteroaryl optionally substituted with one or two substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR¹-alkyleneCONR^{c}R^{d} (where R^{c} is hydroxyl and R^{d} and R^{e} are independently hydrogen or alkyl), -alkylene-N(heteroalkyl)₂, or carboxyalkylaminoalkyl.

In another embodiment, Ar² is thien-2-yl, pyridin-3-yl, quinolin-6-yl, benzothiazol-2-yl, benzoxazol-2-yl, benzofuran-2-yl, benzofuran-5-yl, benzothien-2-yl, furan-2-yl, 1H-benzimidazol-2-yl, 1H-pyrrol-2-yl, thiazol-2-yl, 1H-indol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, quinolin-1-yl, pyridin-2-yl, quinolin-2-yl, furan-3-yl, or thiophen-3-yl. In another embodiment, Ar² is benzofuran-2-yl, or benzothien-2-yl, wherein Ar² is optionally substituted with one or two substituents selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR¹-alkyleneCONR^{C}R^{d} (where R^{c} is hydroxyl and R^{d} and R^{e} are independently hydrogen or alkyl), -alkylene-N(heteroalkyl)₂, and carboxyalkylaminoalkyl.

In another embodiment, Ar² is benzofuran-2-yl and is mono-substituted at the 3-, 4- or 5-position or disubstituted at the 4- and 7-positions, with a substituent selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR¹-alkyleneCONR^{C}R^{d} (where R^{c} is hydroxyl and R^{d} and R^{e} are independently hydrogen or alkyl), -alkylene-N(heteroalkyl)₂, and carboxyalkylaminoalkyl.

In another embodiment, Ar² is benzofuran-2-yl and is monosubstituted at the 3- or 5-position with a substituent selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR-alkyleneCONR^{c}R^{d} (where R^{c} is hydroxyl and R^{d} and R^{e} are independently hydrogen or alkyl), -alkylene-N(heteroalkyl)₂, and carboxyalkylaminoalkyl.

In another embodiment, Ar² is benzofuran-2-yl and is mono-substituted at the 3-, 4- or 5-position or disubstituted at the 4- and 7-positions with a substitutent selected from among chloro, fluoro, trifluoromethyl, methyl, ethyl, methoxy, 1-cyclopropylpiperidin-4-yloxy, 1-(2,2,2-trifluoroethyl)piperidin-4-yloxy, N,N-dimethylaminomethyl, N,N-diethylaminomethyl, 2-methoxyethoxymethyl, phenoxymethyl, 2-methoxyethoxy, 2-morpholin-4-ylethoxy, pyridin-3-ylmethoxy, 2-hydroxyethoxy, 2-N,N-dimethylaminoethoxy, methoxymethyl, 3-i-propoxymethyl, morpholin-4-ylmethyl, 3-hydroxypropyloxymethyl, 2-fluorophenoxymethyl, 3-fluorophenoxymethyl, 4-fluorophenoxy-methyl, 3-methoxypropyloxymethyl, pyridin-4-yloxymethyl, 2,4,6-trifluorophenoxymethyl, 2-oxopyridin-1-ylmethyl, 2,2,2-trifluoroethoxymethyl, 4-imidazol-1-ylphenoxymethyl, 4-[1.2.4-triazin-1-yl-phenoxymethyl, 2-phenylethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, pyridin-3-ylmethyloxymethyl, tetrahydropyran-4-yloxy, 2,2,2-trifluoroethyloxy, 2-pyrrolidin-1-ylethyloxy, piperidin-4-yloxy, N-methyl-N-benzylaminomethyl, N-methyl-N-2-phenylethylaminomethyl, 3-hydroxypropylthiomethyl, 3-hydroxypropylsulfinylmethyl, 3-hydroxypropylsulfonyl-methyl, N-methyl-N-2-indol-3-ylethylaminomethyl, 2-(4-trifluoromethylphenyl)ethyl, 2-(3-trifluoromethoxyphenyl)ethyl, N-hydroxyaminocarbonyl-methylaminomethyl, and 2-carboxyethylamino-methyl.

In another embodiment, Ar² is benzofuran-2-yl that is substituted at the 3-position with N,N-dimethylaminomethyl, N,N-diethylaminomethyl, N,N-di(methoxyethoxyethoxyethoxymethyl)aminomethyl, or N,N-di(methoxyethoxyethoxymethyl)aminomethyl, N,N-di(methoxyethoxymethyl)aminomethyl, 2-fluorophenoxymethyl, 3-fluorophenoxymethyl, 4-fluorophenoxymethyl, pyridin-4-yloxymethyl, 2,4,6-trifluorophenoxy-methyl, 2-oxopyridin-1-ylmethyl, 2,2,2-trifluoroethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethyl-piperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenylethyl)-, N-methyl-N-benzyl-aminomethyl, N-methyl-N-2-phenylethylaminomethyl, 3-hydroxypropylthiomethyl, 3-hydroxypropylsulfinyl-methyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylamino-methyl.

In another embodiment, Ar² is benzofuran-2-yl that is substituted at the 3-position with N,N-dimethylaminomethyl, N,N-diethylaminomethyl, 2-fluorophenoxymethyl, 3-fluorophenoxymethyl, 4-fluorophenoxymethyl, pyridin-4-yloxymethyl, 2,4,6-trifluorophenoxy-methyl, 2-oxopyridin-1-ylmethyl, 2,2,2-trifluoroethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethyl-piperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenylethyl)-, N-methyl-N-benzyl-aminomethyl, N-methyl-N-2-phenylethylaminomethyl, 3-hydroxypropylthiomethyl, 3-hydroxypropylsulfinyl-methyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylamino-methyl.

In another embodiment, Ar² is benzofuran-2-yl that is substituted at the 5-position with 1-cyclopropylpiperidin-4-yloxy, piperidin-4-yloxy, tetrahydropyran-4-yloxy, 2,2,2-trifluoroethoxy, 2-pyrrolidin-1-ylethyloxy, or 1-(2,2,2-trifluoroethyl)piperidin-4-yloxy.

In another embodiment, Ar² is 7-chloro-4-methylbenzofuran-2-yl, 4-methyl-benzofuran-2-yl, 7-fluoro-4-methylbenzofuran-2-yl, or 7-fluoro-4-phenoxymethylbenzofuran-2-yl.

In another embodiment, Ar² is thien-2-yl, pyridin-3-yl, 5-phenylthien-2-yl, quinolin-6-yl, 4-phenylthiazol-2-yl, benzothiazol-2-yl, benzoxazol-2-yl, furan-2-yl, 1H-benzimidazol-2-yl, 1H-pyrrol-2-yl, 4-(pyridin-4-yl)-thiazol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, 1-methyl-indol-3-yl, 4-methoxy-quinolin-2-yl, quinolin-4-yl, 4-hydroxyquinolin-2-yl, pyridin-2-yl, 3-hydroxypyridin-2-yl, 6-hydroxypyridin-2-yl, 6-(4-nitrophenoxy)pyridin-2-yl, 4-(2-methoxyethoxy)quinolin-2-yl, 4-(2-dimethylaminoethoxy)quinolin-2-yl, 6-bromopyridin-2-yl, 5-bromopyridin-3-yl, 4-methoxyquinolin-2-yl, 5-phenylpyridin-3-yl, 6-benzyloxypyridin-2-yl, 6-(2-methylpropyloxy)-pyridin-2-yl, 6-(2-phenylethyloxy)pyridin-2-yl, 4-(3,3,3-trifluoropropyloxy)quinolin-2-yl, 5-thiophen-3-ylpyridin-3-yl, 6-(4-acetylaminophenoxy)-pyridin-2-yl, 6-(4-aminophenoxy)-pyridin-2-yl, or 5-(4-dimethylaminophenyl)pyridin-3-yl.

In another embodiment, Ar² is heteroaryl. In another embodiment, Ar² is heteroaryl optionally substituted with one or two substituents independently selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0 to 2 and R¹ is hydroxyalkyl or optionally substituted phenyl), -alkylene-NR^{c}-alkyleneCONR^{C}R^{d} (where R^{c} is hydroxyl and R^{d} and R^{e} are independently hydrogen or alkyl), -alkylene-N(heteroalkyl)₂, and carboxyalkylaminoalkyl.

In another embodiment, Ar² is thien-2-yl, pyridin-3-yl, quinolin-6-yl, benzothiazol-2-yl, benzoxazol-2-yl, benzofuran-2-yl, benzofuran-5-yl, benzothien-2-yl, furan-2-yl, 1H-benzimidazol-2-yl, 1H-pyrrol-2-yl, thiazol-2-yl, 1H-indol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, quinolin-1-yl, pyridin-2-yl, quinolin-2-yl, furan-3-yl, or thien-3-yl. In another embodiment, Ar² is benzofuran-2-yl, or benzothien-2-yl, where the benzofuran-2-yl, or the benzothien-2-yl, is optionally substituted with one or two substituents independently selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0 to 2 and R¹ is hydroxyalkyl or optionally substituted phenyl), -alkylene-NR¹-alkyleneCONR^{C}R^{d} (where R^{c} is hydroxyl and R^{d} and R^{e} are independently hydrogen or alkyl), -alkylene-N(heteroalkyl)₂, and carboxyalkylaminoalkyl.

In another embodiment, Ar² is benzofuran-2-yl and is mono-substituted at the 3-, 4- or 5-position or disubstituted at the 4 and 7 positions. In another embodiment, the benzofuran-2-yl is monosubstituted at the 3 or 5 position with a substituent described in the paragraph immediately above. In another embodiment, the substituents on the benzofuran-2-yl are independently selected from chloro, fluoro, trifluoromethyl, methyl, ethyl, methoxy, 1-cyclopropylpiperidin-4-yloxy, 1-(2,2,2-trifluoroethyl)piperidin-4-yloxy, N,N-dimethylaminomethyl, N,N-diethylaminomethyl, 2-methoxyethoxymethyl, phenoxymethyl, 2-methoxyethoxy, 2-morpholin-4-ylethoxy, pyridin-3-ylmethoxy, 2-hydroxyethoxy, 2-N,N-dimethylaminoethoxy, methoxymethyl, 3-i-propoxymethyl, morpholin-4-ylmethyl, 3-hydroxypropyloxymethyl, 2-fluorophenoxymethyl, 3-fluorophenoxymethyl, 4-fluorophenoxy-methyl, 3-methoxypropyloxymethyl, pyridin-4-yloxymethyl, 2,4,6-trifluorophenoxymethyl, 2-oxopyridin-1-ylmethyl, 2,2,2-trifluoroethoxymethyl, 4-imidazol-1-ylphenoxymethyl, 4-[1.2.4-triazin-1-yl-phenoxymethyl, 2-phenylethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, pyridin-3-ylmethyloxymethyl, tetrahydropyran-4-yloxy, 2,2,2-trifluoroethyloxy, 2-pyrrolidin-1-ylethyloxy, piperidin-4-yloxy, N-methyl-N-benzylaminomethyl, N-methyl-N-2-phenylethylaminomethyl, 3-hydroxypropylthiomethyl, 3-hydroxypropylsulfinylmethyl, 3-hydroxypropylsulfonyl-methyl, N-methyl-N-2-indol-3-ylethylaminomethyl, 2-(4-trifluoromethylphenyl)ethyl, 2-(3-trifluoromethoxyphenyl)ethyl, N-hydroxyaminocarbonyl-methylaminomethyl, or 3-(2-carboxyethylamino-methyl).

In another embodiment, Ar² is benzofuran-2-yl that is substituted at the 3-position with N,N-dimethylaminomethyl, N,N-diethylaminomethyl, 2-fluorophenoxymethyl, 3-fluorophenoxymethyl, 4-fluorophenoxymethyl, pyridin-4-yloxymethyl, 2,4,6-trifluorophenoxy-methyl, 2-oxopyridin-1-ylmethyl, 2,2,2-trifluoroethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethyl-piperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenylethyl)-, N-methyl-N-benzyl-aminomethyl, N-methyl-N-2-phenylethylaminomethyl, 3-hydroxypropylthiomethyl, 3-hydroxypropylsulfinyl-methyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylamino-methyl.

In another embodiment, Ar² is benzofuran-2-yl that is substituted at the 5-position with 1-cyclopropylpiperidin-4-yloxy, piperidin-4-yloxy, tetrahydropyran-4-yloxy, 2,2,2-trifluoroethoxy, 2-pyrrolidin-1-ylethyloxy, or 1-(2,2,2-trifluoroethyl)piperidin-4-yloxy.

In another embodiment, Ar² is 7-chloro-4-methylbenzofuran-2-yl, 4-methyl-benzofuran-2-yl, 7-fluoro-4-methylbenzofuran-2-yl, or 7-fluoro-4-phenoxymethylbenzofuran-2-yl.

In another embodiment, Ar² is thien-2-yl, pyridin-3-yl, 5-phenylthien-2-yl, quinolin-6-yl, 4-phenylthiazol-2-yl, benzothiazol-2-yl, benzoxazol-2-yl, furan-2-yl, 1H-benzimidazol-2-yl, 1H-pyrrol-2-yl, 4-(pyridin-4-yl)-thiazol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, 1-methyl-indol-3-yl, 4-methoxy-quinolin-2-yl, quinolin-4-yl, 4-hydroxyquinolin-2-yl, pyridin-2-yl, 3-hydroxypyridin-2-yl, 6-hydroxypyridin-2-yl, 6-(4-nitrophenoxy)pyridin-2-yl, 4-(2-methoxyethoxy)quinolin-2-yl, 4-(2-dimethylaminoethoxy)quinolin-2-yl, 6-bromopyridin-2-yl, 5-bromopyridin-3-yl, 4-methoxyquinolin-2-yl, 5-phenylpyridin-3-yl, 6-benzyloxypyridin-2-yl, 6-(2-methylpropyloxy)-pyridin-2-yl, 6-(2-phenylethyloxy)pyridin-2-yl, 4-(3,3,3-trifluoropropyloxy)quinolin-2-yl, 5-thiophen-3-ylpyridin-3-yl, 6-(4-acetylaminophenoxy)-pyridin-2-yl, 6-(4-aminophenoxy)-pyridin-2-yl, or 5-(4-dimethylaminophenyl)pyridin-3-yl.

In another embodiment, Y is a straight chain alkylene. In another embodiment, Y is ethylene or n-propylene. In another embodiment, Y is ethylene.

In another embodiment, Y is a branched alkylene. In another embodiment, Y is —CH(C₂H₅)CH₂—, CH(i-C₃H₇)CH₂—, or CH(CH₃)CH₂—. In another embodiment, Y is —CH(C₂H₅)CH₂—, CH(i-C₃H₇)CH₂—, or CH(CH₃)CH₂—, and the stereochemistry at the chiral carbon of Y is (S). In another embodiment, Y is —CH(C₂H₅)CH₂—. In another embodiment, Y is —CH(C₂H₅)CH₂—, and the stereochemistry at the chiral carbon of Y is (S).

In another embodiment, Y is —CH₂CH(CH₃)— and the stereochemistry at the chiral carbon of Y is (R).

In another embodiment, Y is —CH(CH₂R′)CH₂— or CH(CH₂CH₂R′)CH₂—, where R′ is alkylthio, alkysulfonyl, optionally substituted phenylalkylthio, optionally substituted phenylalkylsulfonyl, hydroxy, or optionally substituted phenoxy, preferably, phenyl, phenoxy, 4-chlorophenyl, cyclohexyl, benzylthio, benzylsulfonyl, methylthio, methylsulfonyl, or hydroxy.

In another embodiment, X is O—; R^{A} and R³ are hydrogen; Ar¹ is phenylene; Ar² is aralkenyl; Y is branched alkylene; and C(═S)NHOR^{A} and Ar²—C(═O)NR³—Y—X— are at the 1 and 4 position of the phenylene ring. In another embodiment, Ar² is trans-phenyl-CH═CH— wherein the phenyl is optionally substituted with one or two substituents independently selected from alkyl, alkoxy, methylenedioxy, or hydroxyl.

In one aspect, described herein are compounds of Formula (IIa):

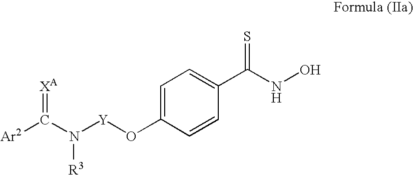

wherein:
X^{A} is O or S;
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

In some embodiments, X^{A} is S. In other embodiments, X^{A} is O.

In one aspect, described herein are compounds of Formula (IIa):

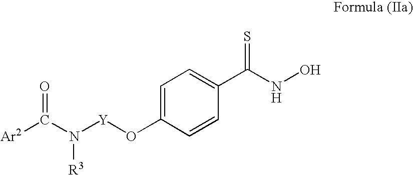

wherein:
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

In one aspect, R³ is hydrogen; methyl; ethyl; propyl; butyl; i-propyl; 2-hydroxy-ethyl; phenyl; or benzyl. In one aspect, R³ is hydrogen.

In one aspect, Y is —CH₂—CH₂-; —CH₂—CH(CH₃)—; —CH(CH₃)—CH₂—; —CH(CH₂CH₃)—CH₂—; —CH(i-propyl)-CH₂—; —CH(i-butyl)-CH₂—; —CH(n-butyl)-CH₂—; —CH(cyclohexyl-methyl)-CH₂—; —CH(benzyl)-CH₂—; —CH(2-phenylethyl)-CH₂—; —CH(benzylS-methyl)-CH₂—; —CH(benzylSO₂-methyl)-CH₂—; —CH(2-MeS-ethyl)-CH₂—; —CH(hydroxy-methyl)-CH₂—; —CH(2-MeSO₂-ethyl)-CH₂—; —CH(4-Cl-benzyl)-CH₂—; —CH(benzyl-5-methyl)-CH₂—; —CH(phenyl)-CH₂—; —CH(benzylSO₂-methyl)-CH₂—; —CH₂—CH₂—CH₂—; —CH(CH₃ Smethyl)-CH₂—; —CH(CH₃ SO₂-methyl)-CH₂—; or —CH₂CH-(phenoxymethyl)-.

Any combination of the groups described above for the various variables is contemplated herein.

Representative compounds of Formula (II) include, but are not limited to those compounds described in Table IV, Table V, Table VI, and Table VII.

In one aspect, representative compounds of Formula (II) where R¹ and R³ are hydrogen, Ar¹ is phenyl, are disclosed in Table IV:

TABLE IV
Representative Compounds of Formula II.

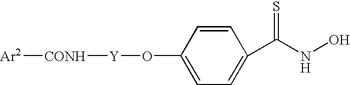

| Compound # | Ar² | Y |
|---|---|---|
| IV-1 | phenyl | —CH₂—CH₂— |
| IV-2 | trans phenyl-CH═CH— | —CH₂—CH₂— |
| IV-3 | trans phenylcyclopropyl | —CH₂—CH₂— |
| IV-4 | trans 4-MeO-phenyl-CH═CH— | —CH₂—CH₂— |
| IV-5 | 2-phenylethyl | —CH₂—CH₂— |
| IV-6 | 1H-indol-3-ylmethyl | —CH₂—CH₂— |
| IV-7 | thiophen-2-yl | —CH₂—CH₂— |
| IV-8 | pyridin-3-yl | —CH₂—CH₂— |
| IV-9 | 4-biphenyl | —CH₂—CH₂— |
| IV-10 | 3-biphenyl | —CH₂—CH₂— |
| IV-11 | 5-phenylthiophen-2-yl | —CH₂—CH₂— |
| IV-12 | thiophen-2-ylmethyl | —CH₂—CH₂— |
| IV-13 | naphth-2-yl | —CH₂—CH₂— |
| IV-14 | quinolin-6-yl | —CH₂—CH₂— |
| IV-15 | 4-phenylthiazol-2-yl | —CH₂—CH₂— |
| IV-16 | 4-tert-butylphenyl | —CH₂—CH₂— |
| IV-17 | trans pyridin-3-yl-CH═CH— | —CH₂—CH₂— |
| IV-18 | 4-pyrrol-1-ylphenyl | —CH₂—CH₂— |
| IV-19 | 4-(cyclohexene-3-oxy)-phenyl | —CH₂—CH₂— |
| IV-20 | benzothiazol-2-yl | —CH₂—CH₂— |
| IV-21 | benzoxazol-2-yl | —CH₂—CH₂— |
| IV-22 | octahydroisoquinolin-2-ylmethyl | —CH₂—CH₂— |
| IV-23 | 4-pyridin-4-yl-piperazin-1-ylmethyl | —CH₂—CH₂— |
| IV-24 | furan-2-yl | —CH₂—CH₂— |
| IV-25 | 4-(pyridin-3-yl)-phenyl | —CH₂—CH₂— |
| IV-26 | 4-(pyridin-2-yl)-phenyl | —CH₂—CH₂— |
| IV-27 | 1H-benzimidazol-2-yl | —CH₂—CH₂— |
| IV-28 | 1H-pyrrol-2-yl | —CH₂—CH₂— |
| IV-29 | 4-(benzoylamino)-phenyl | —CH₂—CH₂— |
| IV-30 | 4-(pyridin-4-yl)-thiazol-2-yl | —CH₂—CH₂— |
| IV-31 | adamantan-1-yl | —CH₂—CH₂— |
| IV-32 | 2,4-difluorophenyl | —CH₂—CH₂— |
| IV-33 | trans 3,4-methylenedioxyphenylCH═CH— | —CH₂—CH₂— |
| IV-34 | 3,4-methylenedioxyphenyl | —CH₂—CH₂— |
| IV-35 | 3,4-dimethoxyphenyl | —CH₂—CH₂— |
| IV-36 | 3,5-dimethoxyphenyl | —CH₂—CH₂— |
| IV-37 | 3,4-difluorophenyl | —CH₂—CH₂— |
| IV-38 | 2,5-dimethylphenyl | —CH₂—CH₂— |
| IV-39 | 2,3-dichlorophenyl | —CH₂—CH₂— |
| IV-40 | 2,3-dimethylphenyl | —CH₂—CH₂— |
| IV-41 | 4-chloro-2-methoxyphenyl | —CH₂—CH₂— |
| IV-42 | 3-ethoxyphenyl | —CH₂—CH₂— |
| IV-43 | 4-methoxy-2-methylphenyl | —CH₂—CH₂— |
| IV-44 | 3-fluoro-4-methoxyphenyl | —CH₂—CH₂— |
| IV-45 | 2-(thiophen-2-ylmethoxy)phenyl | —CH₂—CH₂— |
| IV-46 | 3-(thiophen-2-ylmethoxy)-phenyl | —CH₂—CH₂— |
| IV-47 | 2-phenylphenyl | —CH₂—CH₂— |
| IV-48 | 1H-indol-5-yl | —CH₂—CH₂— |
| IV-49 | 1H-indol-3-yl | —CH₂—CH₂— |
| IV-50 | quinolin-3-yl | —CH₂—CH₂— |
| IV-51 | quinolin-8-yl | —CH₂—CH₂— |
| IV-52 | 1H-indazol-3-yl | —CH₂—CH₂— |
| IV-53 | 1H-benzotriazol-5-yl | —CH₂—CH₂— |
| IV-54 | isoquinolin-1-yl | —CH₂—CH₂— |
| IV-55 | isoquinolin-3-yl | —CH₂—CH₂— |
| IV-56 | quinoxalin-2-yl | —CH₂—CH₂— |
| IV-57 | naphth-1-yl | —CH₂—CH₂— |
| IV-58 | quinolin-2-yl | —CH₂—CH₂— |
| IV-59 | 2-pyrrol-1-yl-phenyl | —CH₂—CH₂— |
| IV-60 | 4-fluoronaphth-1-yl | —CH₂—CH₂— |
| IV-61 | 1H-benzimidazol-5-yl | —CH₂—CH₂— |
| IV-62 | 1-methyl-indol-3-yl | —CH₂—CH₂— |
| IV-63 | 4-MeO-quinolin-2-yl | —CH₂—CH₂— |
| IV-64 | 3-MeO-naphth-2-yl | —CH₂—CH₂— |
| IV-65 | 2-MeO-naphth-1-yl | —CH₂—CH₂— |
| IV-66 | quinolin-4-yl | —CH₂—CH₂— |
| IV-67 | trans phenyl-CH═C(CH₃)— | —CH₂—CH₂— |
| IV-68 | 2-N,N-dimethylaminomethylbenzofuran-5-yl | —CH₂—CH₂— |
| IV-69 | indolin-1-yl | —CH₂—CH₂— |
| IV-70 | 1,2,3,4-tetrahydroquinolin-1-yl | —CH₂—CH₂— |
| IV-71 | trans 5-hydroxybenzofuran-2-yl-C(CH₃)═CH— | —(S)—CH(CH₂CH₃)—CH₂— |
| IV-72 | trans 5-(1-cyclopropylpiperidin-4-yloxy)benzofuran-2-yl- | —(S)—CH(CH₂CH₃)—CH₂— |
| | C(CH₃)═CH— | |
| IV-73 | benzofuran-2-yl | —(S)—CH(2-phenylethyl)-CH₂— |
| IV-74 | 5-(1-cyclopropylpiperidin-4-yloxy)benzofuran-2-yl | —CH₂—CH₂— |
| IV-75 | 5-(1-cyclopropylpiperidin-4-yloxy)benzofuran-2-yl | —(S)—CH(CH₂CH₃)—CH₂— |
| IV-76 | 5-(1-cyclopropylpiperidin-4-yloxy)benzofuran-2-yl | —(R)—CH₂—CH(CH₃)— |
| IV-77 | 5-[1-(2,2,2-trifluoroethyl)piperidin-4-yloxy]benzofuran-2-yl | —CH₂—CH₂— |
| IV-78 | benzofuran-2-yl | —(R)—CH(benzylSO₂-methyl)-CH₂— |
| IV-79 | benzofuran-2-yl | —(R)—CH(benzylS-methyl)-CH₂— |
| IV-80 | trans 5-methoxybenzofuran-2-yl-C(CH₃)═CH— | —CH₂—CH₂— |
| IV-81 | 1,2,3,4-tetrahydroisoquinolin-2-yl | —CH₂—CH₂— |
| IV-82 | isoindolin-2-yl | —CH₂—CH₂— |
| IV-83 | morpholin-4-yl | —CH₂—CH₂— |
| IV-84 | 4-benzyl-piperazin-1-yl | —CH₂—CH₂— |
| IV-85 | (R)-3-HO-pyrrolidin-1-yl | —CH₂—CH₂— |
| IV-86 | piperidin-1-yl | —CH₂—CH₂— |
| IV-87 | 6-CH₃-1,2,3,4-tetrahydroquinolin-1-yl | —CH₂—CH₂— |
| IV-88 | 2-CH₃-indolin-1-yl | —CH₂—CH₂— |
| IV-89 | 6-F-2-CH₃-1,2,3,4-tetrahydroquinolin-1-yl | —CH₂—CH₂— |
| IV-90 | isoindolin-1-yl | —(S)—CH(CH₂CH₃)—CH₂— |
| IV-91 | trans phenyl-CH═CH— | —CH₂—CH₂—CH₂— |
| IV-92 | trans 4-CH₃O-phenyl-CH═CH— | —CH₂—CH₂—CH₂— |
| IV-93 | 4-phenylthiazol-2-yl | —CH₂—CH₂—CH₂— |
| IV-94 | trans phenyl-CH═CH— | —(S)—CH(methyl)-CH₂— |
| IV-95 | trans phenyl-CH═CH— | —(R)—CH(methyl)-CH₂— |
| IV-96 | trans phenyl-CH═CH— | —(S)—CH(i-butyl)-CH₂— |
| IV-97 | trans phenyl-CH═CH— | —(S)—CH(cyclohexyl-methyl)-CH₂— |
| IV-98 | trans phenyl-CH═CH— | —(S)—CH(i-propyl)-CH₂— |
| IV-99 | trans phenyl-CH═CH— | —(S)—CH(benzyl)-CH₂— |
| IV-100 | trans phenyl-CH═CH— | —(R)—CH(benzyl)-CH₂— |
| IV-101 | trans phenyl-CH═CH— | —(R)—CH(i-butyl)-CH₂— |
| IV-102 | trans phenyl-CH═CH— | —(R)—CH(i-propyl)-CH₂— |
| IV-103 | trans phenyl-CH═CH— | —(RS)—CH(n-butyl)-CH₂— |
| IV-104 | trans phenyl-CH═CH— | —(RS)—CH(4-Cl-benzyl)-CH₂— |
| IV-105 | trans phenyl-CH═CH— | —(S)—CH(CH₂CH₃)—CH₂— |
| IV-106 | trans phenyl-CH═CH— | —(R)—CH(CH₂CH₃)—CH₂— |
| IV-107 | trans phenyl-CH═CH— | —(S)—CH(2-MeS-ethyl)-CH₂— |
| IV-108 | trans phenyl-CH═CH— | —(R)—CH(2-MeS-ethyl)-CH₂— |
| IV-109 | trans phenyl-CH═CH— | —(S)—CH(phenyl)-CH₂— |
| IV-110 | trans phenyl-CH═CH— | —(R)—CH(phenyl)-CH₂— |
| IV-111 | trans phenyl-CH═CH— | —(S)—CH(2-MeSO₂-ethyl)-CH₂— |
| IV-112 | trans phenyl-CH═CH— | —(R)—CH(2-MeSO₂-ethyl)-CH₂— |
| IV-113 | trans phenyl-CH═CH— | —(R)—CH(benzylSO₂-methyl)-CH₂— |
| IV-114 | thiophen-2-yl | —(S)—CH(CH₂CH₃)—CH₂— |
| IV-115 | 4-biphenyl | —(S)—CH(CH₂CH₃)—CH₂— |
| IV-116 | naphth-2-yl | —(S)—CH(CH₂CH₃)—CH₂— |
| IV-117 | trans phenyl-CH═CH— | —(R)—CH(benzyl-S-methyl)-CH₂— |
| IV-118 | phenyl | —(S)—CH(CH₂CH₃)—CH₂— |
| IV-119 | benzyl | —(S)—CH(CH₂CH₃)—CH₂— |
| IV-120 | 2-phenylethyl | —(S)—CH(CH₂CH₃)—CH₂— |
| IV-121 | trans phenyl-CH═CH— | —(S)—CH(hydroxy-methyl)-CH₂— |
| IV-122 | 4-phenylthiazol-2-yl | —(S)—CH(CH₂CH₃)—CH₂— |
| IV-123 | trans 4-CH₃O-phenyl-CH═CH— | —(S)—CH(CH₂CH₃)—CH₂— |
| IV-124 | 2-N,N-dimethylaminomethyl-benzofuran-5-yl- | —(S)—CH(CH₂CH₃)—CH₂— |
| IV-125 | trans phenyl-CH═CH— | —(R)—CH₂—CH(CH₃)— |
| IV-126 | trans phenyl-CH═CH— | —(S)—CH₂—CH(CH₃)— |
| IV-127 | 4-phenyl-thiazol-2-yl | —(R)—CH₂—CH(CH₃)— |
| IV-128 | 4-phenyl-thiazol-2-yl | —(S)—CH₂—CH(CH₃)— |
| IV-129 | 4-biphenyl | —(R)—CH₂—CH(CH₃)— |
| IV-130 | trans 4-CH₃O-phenyl-CH═CH— | —(R)—CH₂—CH(CH₃)— |
| IV-131 | 4-(2-pyridin-4-ylthiazol-5-yl)phenyl | —CH₂—CH₂— |
| IV-132 | 7-chloro-4-methylbenzofuran-2-yl | —CH₂—CH₂— |
| IV-133 | 4-[2-(4-methylpiperazin-1-yl)thiazol-5-yl]-phenyl | —CH₂—CH₂— |
| IV-134 | 4-(2-pyridin-4-ylaminothiazol-5-yl)phenyl | —CH₂—CH₂— |
| IV-135 | 4-(4-methylpiperazin-1-yl)phenyl | —CH₂—CH₂— |
| IV-136 | 4-(4-hydroxypiperidin-1-yl)phenyl | —CH₂—CH₂— |
| IV-137 | 4-(4-morpholin-4-ylmethylthiazol-2-yl)phenyl | —CH₂—CH₂— |
| IV-138 | 7-fluoro-4-methylbenzofuran-2-yl | —CH₂—CH₂— |
| IV-139 | 7-fluoro-4-(2-methoxyethoxymethyl)-benzofuran-2-yl | —CH₂—CH₂— |
| IV-140 | 4-hydroxyquinolin-2-yl | —CH₂—CH₂— |
| IV-141 | 7-fluoro-4-phenoxymethylbenzothran-2-yl | —CH₂—CH₂— |
| IV-142 | 4-[2-(4-methylpiperazin-1-ylmethyl)thiazol-5-yl]phenyl | —CH₂—CH₂— |
| IV-143 | pyridin-2-yl | —CH₂—CH₂— |
| IV-144 | 3-hydroxypyridin-2-yl | —CH₂—CH₂— |
| IV-145 | 6-hydroxypyridin-2-yl | —CH₂—CH₂— |
| IV-146 | 6-(4-nitrophenoxy)pyridin-2-yl | —CH₂—CH₂— |
| IV-147 | 4-(2-methoxyethoxy)quinolin-2-yl | —CH₂—CH₂— |
| IV-148 | 4-(2-dimethylaminoethoxy)quinolin-2-yl | —CH₂—CH₂— |
| IV-149 | 6-bromopyridin-2-yl | —CH₂—CH₂— |
| IV-150 | 5-bromopyridin-3-yl | —CH₂—CH₂— |
| IV-151 | 4-methoxyquinolin-2-yl | —(S)—CH(CH₂CH₃)—CH₂— |
| IV-152 | 1-methoxynaphth-2-yl | —CH₂—CH₂— |
| IV-153 | 4-methoxyquinolin-2-yl | —(R)—CH₂—CH(CH₃ )— |
| IV-154 | 5-phenylpyridin-3-yl | —CH₂—CH₂— |
| IV-155 | 6-benzyloxypyridin-2-yl | —CH₂—CH₂— |
| IV-156 | 6-(2-methylpropyloxy)pyridin-2-yl | —CH₂—CH₂— |
| IV-157 | 6-(2-phenylethyloxy)pyridin-2-yl | —CH₂—CH₂— |
| IV-158 | 4-(3,3,3-trifluoropropyloxy)quinolin-2-yl | —CH₂—CH₂— |
| IV-159 | 4-(3,3,3-trifluoropropyloxy)quinolin-2-yl | —(S)—CH(CH₂CH₃)—CH₂— |
| IV-160 | 4-(3,3,3-trifluoropropyloxy)quinolin-2-yl | —(R)—CH₂—CH(CH₃)— |
| IV-161 | trans 3-hydroxyphenyl-CH═CH— | —CH₂—CH₂— |
| IV-162 | trans 4-hydroxyphenyl-CH═CH— | —CH₂—CH₂— |
| IV-163 | 3′-(2-hydroxyethyl)biphen-4-yl | —CH₂—CH₂— |
| IV-164 | 3′-(2-hydroxyethyl)biphen-3-yl | —CH₂—CH₂— |
| IV-165 | 2′-(2-hydroxyethyl)biphen-4-yl | —CH₂—CH₂— |
| IV-166 | trans benzofuran-2-yl-CH═CH— | —CH₂—CH₂— |
| IV-167 | 2′-(2-hydroxyethyl)biphen-3-yl | —CH₂—CH₂— |
| IV-168 | 5-thiophen-3-ylpyridin-3-yl | —CH₂—CH₂— |
| IV-169 | 6-(4-acetylaminophenoxy)pyridin-2-yl | —CH₂—CH₂— |
| IV-170 | 6-(4-aminophenoxy)pyridin-2-yl | —CH₂—CH₂— |
| IV-171 | trans 2-methoxyphenyl-CH═CH— | —CH₂—CH₂— |
| IV-172 | trans 3-methoxyphenyl-CH═CH— | —CH₂—CH₂— |
| IV-173 | 5-(4-dimethylaminophenyl)pyridin-3-yl | —CH₂—CH₂— |
| IV-174 | trans 5-bromothiophen-2-yl-CH═CH— | —CH₂—CH₂— |
| IV-175 | trans furan-3-yl-CH═CH— | —CH₂—CH₂— |
| IV-176 | trans thiophen-3-yl-CH═CH— | —CH₂—CH₂— |
| IV-177 | trans thiophen-2-yl-CH═CH— | —CH₂—CH₂— |
| IV-178 | trans 3-tolyl-CH═CH— | —CH₂—CH₂— |
| IV-179 | trans 4-tolyl-CH═CH— | —CH₂—CH₂— |
| IV-180 | trans benzofuran-2-yl-C(CH₃)═CH— | —CH₂—CH₂— |
| IV-181 | cis benzofuran-2-yl-C(CH₃)═CH— | —CH₂—CH₂— |
| IV-182 | trans 4-dimethylaminophenyl-CH═CH— | —CH₂—CH₂— |
| IV-183 | trans indol-3-yl-CH═CH— | —CH₂—CH₂— |
| IV-184 | trans 2-tolyl-CH═CH— | —CH₂—CH₂— |
| IV-185 | trans 2-hydroxyphenyl-CH═CH— | —CH₂—CH₂— |
| IV-186 | trans 7-methoxybenzofuran-2-yl-CH═CH— | —CH₂—CH₂— |
| IV-187 | trans 7-methoxybenzofuran-2-yl-CH═CH— | —(R)—CH(CH₂CH₃)—CH₂— |
| IV-188 | trans 5-methoxybenzofuran-2-yl-C(CH₃)═CH— | —(S)—CH(CH₂CH₃)—CH₂— |
| IV-189 | trans furan-2-yl-CH═CH— | —CH₂—CH₂— |
| IV-190 | 4-[4-(2-morpholin-4-ylethyl)thiazol-2-yl]phenyl | —CH₂—CH₂— |

Compounds in Table IV are named:
4-(2-benzenecarbonylamino-ethoxy)phenylthiohydroxamic acid (Compound IV-1);
4-(2-trans-cinnamoylaminoethoxy)phenylthiohydroxamic acid (Compound IV-2);
4-(2-trans-2-phenylcyclopropylcarbonylaminoethoxy)phenylthiohydroxamic acid (Compound IV-3);
4-(2-trans-4-methoxycinnamoylaminoethoxy)phenylthiohydroxamic acid (Compound IV-4);
4-[2-(2-phenylethylcarbonylamino)ethoxy]phenylthiohydroxamic acid (Compound IV-5);
4-[2-(1H-indol-3-ylmethylcarbonylamino)ethoxy]phenylthiohydroxamic acid (Compound IV-6);
4-(2-thiophen-2-ylcarbonylaminoethoxy)phenylthiohydroxamic acid (Compound IV-7);
4-(2-pyridin-3-ylcarbonylaminoethoxy)phenylthiohydroxamic acid (Compound IV-8);
4-(2-biphen-4-ylcarbonylaminoethoxy)phenylthiohydroxamic acid (Compound IV-9);
4-(2-biphen-3-ylcarbonylaminoethoxy)phenylthiohydroxamic acid (Compound IV-10);
4-[2-(5-phenylthiophen-2-ylcarbonylamino)ethoxy]phenylthiohydroxamic acid (Compound IV-11);
4-[2-(thiophen-2-ylmethylcarbonylamino)ethoxy]phenylthiohydroxamic acid (Compound IV-12);
4-[2-(napth-2-ylcarbonylamino)ethoxy]phenylthiohydroxamic acid (Compound IV-13);
4-[2-(quinolin-6-ylcarbonylamino)ethoxy]phenylthiohydroxamic acid (Compound IV-14);
4-[2-(4-phenylthiazol-2-ylcarbonylamino)ethoxy]phenylthiohydroxamic acid (Compound IV-15);
4-[2-(4-tert-butylphenylcarbonylamino)ethoxy]phenylthiohydroxamic acid (Compound IV-16);
4-[2-(trans-3-pyridin-3-ylacryloylamino)ethoxy]phenylthiohydroxamic acid (Compound IV-17);
4-[2-(4-pyrrol-1-ylphenylcarbonylamino)ethoxy]phenylthiohydroxamic acid (Compound IV-18);
4-[2-(4-cyclohexene-3-oxyphenylcarbonylamino)ethoxy]phenylthiohydroxamic acid (Compound IV-19);
4-[2-(benzthiazol-2-ylcarbonylamino)ethoxy]phenylthiohydroxamic acid (Compound IV-20);
4-[2-(benzoxazol-2-ylcarbonylamino)ethoxy]phenylthiohydroxamic acid (Compound IV-21);
4-[2-(octahydroisoquinolin-2-ylmethylcarbonylamino)ethoxy]phenylthiohydroxamic acid (Compound IV22);
4-[2-(4-pyridin-4-ylpiperazin-1-ylmethylcarbonylamino)ethoxy]phenylthiohydroxamic acid (Compound IV-23);
4-[2-(furan-2-ylcarbonylamino)ethoxy]phenylthiohydroxamic acid (Compound IV-24);
4-[2-(4-pyridin-3-ylphenylcarbonylamino)ethoxy]phenylthiohydroxamic acid (Compound IV-25);
4-[2-(4-pyridin-2-ylphenylcarbonylamino)ethoxy]phenylthiohydroxamic acid (Compound IV-26);
4-[2-(benzimidazol-2-ylcarbonylamino)ethoxy]phenylthiohydroxamic acid (Compound IV-27);
4-[2-(1H-pyrrol-2-ylcarbonylamino)ethoxy]phenylthiohydroxamic acid (Compound IV-28);
4-[2-(4-benzoylaminophenylcarbonylamino)ethoxy]phenylthiohydroxamic acid (Compound IV-29);
4-[2-(4-pyridin-4-ylthiazol-2-ylcarbonylamino)ethoxy]phenylthiohydroxamic acid (Compound IV-30);
4-[2-(admantan-1-ylcarbonylamino)ethoxy]phenylthiohydroxamic acid (Compound IV-31);
4-[2-(2,4-difluorophenylcarbonylamino)ethoxy]phenylthiohydroxamic acid (Compound IV-32);
4-[2-(3-trans-3,4-methylenedioxyphenylacryloylamino)ethoxy]-phenylthiohydroxamic acid (Compound IV-33);
4-[2-(3,4-methylenedioxyphenylcarbonylamino)ethoxy]phenylthiohydroxamic acid (Compound IV-34);
4-[2-(3,4-dimethoxyphenylcarbonylamino)ethoxy]phenylthiohydroxamic acid (Compound IV-35);
4-[2-(3,5-dimethoxyphenylcarbonylamino)ethoxy]phenylthiohydroxamic acid (Compound IV-36);
4-[2-(3,4-difluorophenylcarbonylamino)ethoxy]phenylthiohydroxamic acid (Compound IV-37);
4-[2-(2,5-dimethylphenylcarbonylamino)ethoxy]phenylthiohydroxamic acid (Compound IV-38);
4-[2-(2,3-dichlorophenylcarbonylamino)ethoxy]phenylthiohydroxamic acid (Compound IV-39);
4-[2-(2,3-dimethylphenylcarbonylamino)ethoxy]phenylthiohydroxamic acid (Compound IV-40);
4-[2-(4-chloro-2-methoxyphenylcarbonylamino)ethoxy]phenylthiohydroxamic acid (Compound IV-41);
4-[2-(3-ethoxyphenylcarbonylamino)ethoxy]phenylthiohydroxamic acid (Compound IV-42);
4-[2-(4-methoxy-2-methylphenylcarbonylamino)ethoxy]phenylthiohydroxamic acid (Compound IV-43);
4-[2-(3-fluoro-4-methoxyphenylcarbonylamino)ethoxy]phenylthiohydroxamic acid (Compound IV-44);
4-[2-(2-thiophen-2-ylmethoxyphenylcarbonylamino)ethoxy]phenylthiohydroxamic acid (Compound IV-45);
4-[2-(3-thiophen-2-ylmethoxyphenylcarbonylamino)ethoxy]phenylthiohydroxamic acid (Compound IV-46);
4-[2-(biphen-2-ylcarbonylamino)ethoxy]phenylthiohydroxamic acid (Compound IV-47);
4-[2-(1H-indol-5-ylcarbonylamino)ethoxy]phenylthiohydroxamic acid (Compound IV-48);
4-[2-(1H-indol-3-ylcarbonylamino)ethoxy]phenylthiohydroxamic acid (Compound IV-49);
4-[2-(quinolin-3-ylcarbonylamino)ethoxy]phenylthiohydroxamic acid (Compound IV-50);
4-[2-(quinolin-8-ylcarbonylamino)ethoxy]phenylthiohydroxamic acid (Compound IV-51);
4-[2-(1H-indazol-3-ylcarbonylamino)ethoxy]phenylthiohydroxamic acid (Compound IV-52);
4-[2-(1H-benzotriazol-5-ylcarbonylamino)ethoxy]phenylthiohydroxamic acid (Compound IV-53);
4-[2-(isoquinolin-1-ylcarbonylamino)ethoxy]phenylthiohydroxamic acid (Compound IV-54);
4-[2-(isoquinolin-3-ylcarbonylamino)ethoxy]phenylthiohydroxamic acid (Compound IV-55);
4-[2-(quinoxalin-2-ylcarbonylamino)ethoxy]phenylthiohydroxamic acid (Compound IV-56);
4-[2-(naphth-1-ylcarbonylamino)ethoxy]phenylthiohydroxamic acid (Compound IV-57);
4-[2-(quinolin-2-ylcarbonylamino)ethoxy]phenylthiohydroxamic acid (Compound IV-58);
4-[2-(2-pyrrol-1-ylphenylcarbonylamino)ethoxy]phenylthiohydroxamic acid (Compound IV-59);
4-[2-(4-fluoronapth-1-ylcarbonylamino)ethoxy]phenylthiohydroxamic acid (Compound IV-60);
4-[2-(1H-benzimidazol-5-ylcarbonylamino)ethoxy]phenylthiohydroxamic acid (Compound IV-61);
4-[2-(1-methylindol-3-ylcarbonylamino)ethoxy]phenylthiohydroxamic acid (Compound IV-62);
4-[2-(4-methoxyquinolin-2-ylcarbonylamino)ethoxy]phenylthiohydroxamic acid (Compound IV-63);
4-[2-(3-methoxynapth-2-ylcarbonylamino)ethoxy]phenylthiohydroxamic acid (Compound IV-64);
4-[2-(2-methoxynapth-1-ylcarbonylamino)ethoxy]phenylthiohydroxamic acid (Compound IV-65);
4-[2-(quinolin-4-ylcarbonylamino)ethoxy]phenylthiohydroxamic acid (Compound IV-66);
4-[2-(trans-2-methylcinnamoylamino)ethoxy]phenylthiohydroxamic acid (Compound IV-67);
4-[2-(2-N,N-dimethylaminomethylbenzofuran-5-ylcarbonylamino)ethoxy]-phenylthiohydroxamic acid (Compound IV-68);
4-(2-indolin-1-ylcarbonylaminoethoxy)phenylthiohydroxamic acid (Compound IV-69);
4-[2-(1,2,3,4-tetrahydroquinolin-1-ylcarbonylamino)ethoxy]phenylthiohydroxamic acid (Compound IV-70);
4-{2S-[trans-3-(5-hydroxybenzofuran-2-yl)but-2-enoylamino]butoxy}phenylthiohydroxamic acid (Compound IV-71);
4-{2S-[trans-3-(5-(1-cyclopropylpiperidin-4-yloxy)benzofuran-2-yl)but-2-enoyl-amino]butoxy}phenylthiohydroxamic acid (Compound IV-72);
4-[2S-(benzofuran-2-ylcarbonylamino)-4-phenylbutoxy)phenylthiohydroxamic acid (Compound IV-73);
4-{2-[5-(1-cyclopropylpiperidin-4-yloxy)benzofuran-2-ylcarbonylamino]-ethoxy}phenylthiohydroxamic acid (Compound IV-74);
4-{2S-[5-(1-cyclopropylpiperidin-4-yloxy)benzofuran-2-ylcarbonylamino]-butoxy}phenylthiohydroxamic acid (Compound IV-75);
4-{2-[5-(1-cyclopropylpiperidin-4-yloxy)benzofuran-2-ylcarbonylamino]-1R-methylethoxy}phenylthiohydroxamic acid (Compound IV-76);
4-{2-[5-(1-(2,2,2-trifluoroethyl)piperidin-4-yloxy)benzofuran-2-ylcarbonyl-amino]-ethoxy}phenylthiohydroxamic acid (Compound IV-77);
4-[2R-(benzofuran-2-ylcarbonylamino)-3-benzylsulfonylpropoxy]phenylthiohydroxamic acid (Compound IV-78);
4-[2R-(benzofuran-2-ylcarbonylamino)-3-benzylthiopropoxy]phenylthiohydroxamic acid (Compound IV-79);
4-[2-(trans-3-(5-methoxybenzofuran-2-yl)but-2-enoylcarbonylamino)-ethoxy]phenylthiohydroxamic acid (Compound IV-80);
4-[2-(1,2,3,4-tetrahydroisoquinolin-2-ylcarbonylamino)ethoxy]phenylthiohydroxamic acid (Compound IV-81);
4-[2-(isoindolin-2-ylcarbonylamino)ethoxy]phenylthiohydroxamic acid (Compound IV-82);
4-[2-(morpholin-4-ylcarbonylamino)ethoxy]phenylthiohydroxamic acid (Compound IV-83);
4-[2-(4-benzylpiperazin-1-ylcarbonylamino)ethoxy]phenylthiohydroxamic acid (Compound IV-84);
4-[2-(3(R)-hydroxypyrrolidin-1-ylcarbonylamino)ethoxy]phenylthiohydroxamic acid (Compound IV-85);
4-[2-(piperidin-1-ylcarbonylamino)ethoxy]phenylthiohydroxamic acid (Compound IV-86);
4-[2-(6-methyl-1,2,3,4-tetrahydroquinolin-1-ylcarbonylamino)ethoxy]-phenylthiohydroxamic acid (Compound IV-87);
4-[2-(2-methylindolin-1-ylcarbonylamino)ethoxy]phenylthiohydroxamic acid (Compound IV-88);
4-[2-(6-fluoro-2-methyl-1,2,3,4-tetrahydroquinolin-1-ylcarbonylamino)-ethoxy]-phenylthiohydroxamic acid (Compound IV-89);
4-[2S-(isoindolin-1-ylcarbonylamino)butoxy]phenylthiohydroxamic acid (Compound IV-90);
4-[3-(trans-cinnamoylamino)propoxy]phenylthiohydroxamic acid (Compound IV-91);
4-[3-(trans-4-methoxycinnamoylamino)propoxy]phenylthiohydroxamic acid (Compound IV-92);
4-[3-(4-phenylthiazol-2-ylcarbonylamino)propoxy]phenylthiohydroxamic acid (Compound IV-93);
4-[2S (trans-cinnamoylamino)propoxy)phenylthiohydroxamic acid (Compound IV-94);
4-[2R-(trans-cinnamoylamino)propoxy)phenylthiohydroxamic acid (Compound IV-95);
4-[2S-(trans-cinnamoylamino)-4-methylpentoxy]phenylthiohydroxamic acid (Compound IV-96);
4-[2S-(trans-cinnamoylamino)-3-cyclohexylpropoxy]phenylthiohydroxamic acid (Compound IV-97);
4-[2S-(trans-cinnamoylamino)-3-methylbutoxy]phenylthiohydroxamic acid (Compound IV-98);
4-[2S-(trans-cinnamoylamino)-3-phenylpropoxy]phenylthiohydroxamic acid (Compound IV-99);
4-[2R-(trans-cinnamoylamino)-3-phenylpropoxy]phenylthiohydroxamic acid (Compound IV-100);
4-[2R-(trans-cinnamoylamino)-4-methylpentoxy]phenylthiohydroxamic acid (Compound IV-101);
4-[2R-(trans-cinnamoylamino)-3-methylbutoxy]phenylthiohydroxamic acid (Compound IV-102);
4-[2RS-(trans-cinnamoylamino)hexyloxy]phenylthiohydroxamic acid (Compound IV-103);
4-[2RS-(trans-cinnamoylamino)-3-(4-chlorophenyl)propoxy)phenylthiohydroxamic acid (Compound IV-104);
4-[2S-(trans-cinnamoylamino)butoxy]phenylthiohydroxamic acid (Compound IV-105);
4-[2R-(trans-cinnamoylamino)butoxy]phenylthiohydroxamic acid (Compound IV-106);
4-[2S-(trans-cinnamoylamino)-4-methylthiobutoxy)phenylthiohydroxamic acid (Compound IV-107);
4-[2R-(trans-cinnamoylamino)-4-methylthiobutoxy]phenylthiohydroxamic acid (Compound IV-108);
4-[2S-(trans-cinnamoylamino)-2-phenylethoxy]phenylthiohydroxamic acid (Compound IV-109);
4-[2R-(trans-cinnamoylamino)-2-phenylethoxy]phenylthiohydroxamic acid (Compound IV-110);
4-[2S-(trans-cinnamoylamino)-4-methylsulfonylbutoxy]phenylthiohydroxamic acid (Compound IV-111);
4-[2R-(trans-cinnamoylamino)-4-methylsulfonylbutoxy]phenylthiohydroxamic acid (Compound IV-112);
4-[2R-(trans-cinnamoylamino)-3-benzylsulfonylpropoxy]phenylthiohydroxamic acid (Compound IV-113);
4-[2S-(thiophen-2-ylcarbonylamino)butoxy]phenylthiohydroxamic acid (Compound IV-114);
4-[2S-(biphen-4-ylcarbonylamino)butoxy]phenylthiohydroxamic acid (Compound IV-115);
4-[2S-(naphth-2-ylcarbonylamino)butoxy]phenylthiohydroxamic acid (Compound IVI-116);
4-[2R-(trans-cinnamoylamino)-3-benzylthiopropoxy]phenylthiohydroxamic acid (Compound IV-117);
4-[2S-(benzenecarbonylamino)butoxy]phenylthiohydroxamic acid (Compound IV-118);
4-[2S-(benzylcarbonylamino)butoxy]phenylthiohydroxamic acid (Compound IV-119);
4-[2S-(2-phenylethylcarbonylamino)butoxy]phenylthiohydroxamic acid (Compound IV-120);
4-[2S-(trans-cinnamoylamino)-3-hydroxypropoxy]phenylthiohydroxamic acid (Compound IV-121);
4-[2S-(4-phenylthiazol-2-ylcarbonylamino)butoxy]phenylthiohydroxamic acid (Compound IV-122);
4-[2S-(trans-4-methoxycinnamoylamino)butoxy]phenylthiohydroxamic acid (Compound IV-123);
4-[2S-(2-N,N-dimethylaminomethylbenzofuran-5-ylcarbonylamino)butoxy]-phenylthiohydroxamic acid (Compound IV-124);
4-[2-(trans-cinnamoylamino)-1R-methylethoxy]phenylthiohydroxamic acid (Compound IV-125);
4-[2-(trans-cinnamoylamino)-1S-methylethoxy)phenylthiohydroxamic acid (Compound IV-126);
4-[2-(4-phenylthiazol-2-ylcarbonylamino)-1R-methylethoxy]phenylthiohydroxamic acid (Compound IV-127);
4-[2-(4-phenylthiazol-2-ylcarbonylamino)-1S-methylethoxy]phenylthiohydroxamic acid (Compound IV-128);
4-[2-(biphen-4-ylcarbonylamino)-1R-methylethoxy]phenylthiohydroxamic acid (Compound IV-129);
4-[2-(trans-4-methoxycinnamoylamino)-1R-methylethoxy]phenylthiohydroxamic acid (Compound IV-130);
4-{2-[4-pyridin-2-ylthiazol-5-yl)phenylcarbonylamino]ethoxy}phenylthiohydroxamic acid (Compound IV-131);
4-[2-(7-chloro-4-methylbenzofuran-2-ylcarbonylamino)ethoxy]phenylthiohydroxamic acid (Compound IV-132);
4-{2-[4-(2-(4-methylpiperazin-1-yl)thiazol-5-yl)phenylcarbonylamino]-ethoxy}-phenylthiohydroxamic acid (Compound IV-133);
4-{2-[4-(2-pyridin-4-ylaminothiazol-5-yl)phenylcarbonylamino]ethoxy}-phenylthiohydroxamic acid (Compound IV-134);
4-{2-[4-(4-methylpiperazin-1-yl)phenylcarbonylamino]ethoxy}phenylthiohydroxamic acid (Compound IV-135);
4-{2-[4-(4-hydroxypiperidin-1-yl)phenylcarbonylamino]ethoxy}phenylthiohydroxamic acid (Compound IV-136);
4-{2-[4-(4-morpholin-4-ylmethylthiazol-5-yl)phenylcarbonylamino]ethoxy}phenylthiohydroxamic acid (Compound IV-137);
4-[2-(7-fluoro-4-methylbenzofuran-2-ylcarbonylamino)ethoxy]phenylthiohydroxamic acid (Compound IV-138);
4-{2-[7-fluoro-(2-methoxyethoxymethyl)benzofuran-2-ylcarbonylamino)-ethoxy}phenylthiohydroxamic acid (Compound IV-139);
4-[2-(4-hydroxyquinolin-2-ylcarbonylamino)ethoxy]phenylthiohydroxamic acid (Compound IV-140);
4-[2-(7-fluoro-4-phenoxymethylbenzofuran-2-ylcarbonylamino)ethoxy]-phenylthiohydroxamic acid (Compound IV-141);
4-{2-[4-(2-(4-methylpiperazin-1-ylmethyl)thiazol-5-yl)phenylcarbonyl-amino]-ethoxy}phenylthiohydroxamic acid (Compound IV-142);
4-[2-(pyridin-2-ylcarbonylamino)ethoxy]phenylthiohydroxamic acid (Compound IV-143);
4-[2-(3-hydroxypyridin-2-ylcarbonylamino)ethoxy]phenylthiohydroxamic acid (Compound IV-144);
4-[2-(6-hydroxypyridin-2-ylcarbonylamino)ethoxy]phenylthiohydroxamic acid (Compound IV-145);
4-{2-[6-(4-nitrophenoxy)pyridin-2-ylcarbonylamino]ethoxy}phenylthiohydroxamic acid (Compound IV-146);
4-{2-[4-(2-methoxyethoxy)quinolin-2-ylcarbonylamino]ethoxy}phenylthiohydroxamic acid (Compound IV-147);
4-{2-[4-(2-N,N-dimethylaminoethoxy)quinolin-2-ylcarbonylamino]ethoxy}-phenylthiohydroxamic acid (Compound IV-148);
4-[2-(6-bromopyridin-2-ylcarbonylamino)ethoxy]phenylthiohydroxamic acid (Compound IV-149);
4-[2-(5-bromopyridin-3-ylcarbonylamino)ethoxy]phenylthiohydroxamic acid (Compound IV-150);
4-[2S-(4-methoxyquinolin-2-ylcarbonylamino)butoxy]phenylthiohydroxamic acid (Compound IV-151);
4-[2-(1-methoxynaphth-2-ylcarbonylamino)ethoxy]phenylthiohydroxamic acid (Compound IV-152);
4-[2-(4-methoxyquinolin-2-ylcarbonylamino)-1R-methylethoxy]phenylthiohydroxamic acid (Compound IV-153);
4-[2-(5-phenylpyridin-3-ylcarbonylamino)ethoxy]phenylthiohydroxamic acid (Compound IV-154);
4-[2-(6-benzyloxypyridin-2-ylcarbonylamino)ethoxy]phenylthiohydroxamic acid (Compound IV-155);
4-{2-[6-(2-methylpropyloxy)pyridin-2-ylcarbonylamino]ethoxy}phenylthiohydroxamic acid (Compound IV-156);
4-{2-[6-(2-phenylethyloxy)pyridin-2-ylcarbonylamino]ethoxy}phenylthiohydroxamic acid (Compound IV-157);
4-{2-[4-(3,3,3-trifluoropropyloxy)quinolin-2-ylcarbonylamino]ethoxy}-phenylthiohydroxamic acid (Compound IV-158);
4-{2S-[4-(3,3,3-trifluoropropyloxy)quinolin-2-ylcarbonylamino]butoxy}-phenylthiohydroxamic acid (Compound IV-159);
4-{2-[4-(3,3,3-trifluoropropyloxy)quinolin-2-ylcarbonylamino]-1R-methyl-ethoxy}phenylthiohydroxamic acid (Compound IV-160);
4-[2-(trans-3-hydroxycinnamoylamino)ethoxy]phenylthiohydroxamic acid (Compound IV-161);
4-[2-(trans-4-hydroxycinnamoylamino)ethoxy]phenylthiohydroxamic acid (Compound IV-162);
4-{2-[3′-(2-hydroxyethyl)biphen-4-ylcarbonylamino]ethoxy}phenylthiohydroxamic acid (Compound IV-163);
4-{2-[3′-(2-hydroxyethyl)biphen-3-ylcarbonylamino]ethoxy}phenylthiohydroxamic acid (Compound IV-164);
4-{2-[2′-(2-hydroxyethyl)biphen-4-ylcarbonylamino]ethoxy}phenylthiohydroxamic acid (Compound IV-165);
4-[2-(trans-2-benzofuran-2-ylacryloylamino)ethoxy]phenylthiohydroxamic acid (Compound IV-166);
4-{2-[2′-(2-hydroxyethyl)biphen-3-ylcarbonylamino]ethoxy}phenylthiohydroxamic acid (Compound IV-167);
4-{2-[5-(thiophen-3-yl)pyridin-3-ylcarbonylamino]ethoxy}phenylthiohydroxamic acid (Compound IV-168);
4-{2-[6-(4-acetylaminophenoxy)pyridin-2-ylcarbonylamino]ethoxy}-phenylthiohydroxamic acid (Compound IV-169);
4-{2-[6-(4-aminophenoxy)pyridin-2-ylcarbonylamino]ethoxy}phenylthiohydroxamic acid (Compound IV-170);
4-[2-(trans-2-methoxycinnamoylamino)ethoxy]phenylthiohydroxamic acid (Compound IV-171);
4-[2-(trans-3-methoxycinnamoylamino)ethoxy]phenylthiohydroxamic acid (Compound IV-172);
4-{2-[5-(4-dimethylaminophenyl)pyridin-3-ylcarbonylamino]ethoxy}-phenylthiohydroxamic acid (Compound IV-173);
4-{2-[trans-3-(5-bromothiophen-2-yl)acryloylamino]ethoxy}phenylthiohydroxamic acid (Compound IV-174);
4-[2-(trans-3-furan-3-ylacryloylamino)ethoxy]phenylthiohydroxamic acid (Compound IV-175);
4-[2-(trans-3-thiophen-3-ylacryloylamino)ethoxy]phenylthiohydroxamic acid (Compound IV-176);
4-[2-(trans-thiophen-2-ylacryloylamino)ethoxy]phenylthiohydroxamic acid (Compound IV-177);
4-{2-[trans-3-methylcinnamoylamino]ethoxy}phenylthiohydroxamic acid (Compound IV-178);
4-{2-[trans-4-methylcinnamoylamino]ethoxy}phenylthiohydroxamic acid (Compound IV-179);
4-{2-[trans-3-(benzofuran-2-yl)but-2-enoylamino]ethoxy}phenylthiohydroxamic acid (Compound IV-180);
4-{2-[cis-3-(benzofuran-2-yl)but-2-enoylamino]ethoxy}phenylthiohydroxamic acid (Compound IV-181);
4-[2-(trans-4-dimethylaminocinnamoylamino)ethoxy]phenylthiohydroxamic acid (Compound IV-182);
4-[2-(trans-3-indol-3-ylacryloylamino)ethoxy]phenylthiohydroxamic acid (Compound IV-183);
4-{2-[trans-2-methylcinnamoyl amino]ethoxy}phenylthiohydroxamic acid (Compound IV-184);
4-[2-(trans-2-hydroxycinnamoylamino)ethoxy]phenylthiohydroxamic acid (Compound IV-185);
4-{2-[trans-3-(7-methoxybenzofuran-2-yl)acryloylamino]ethoxy}phenylthiohydroxamic acid (Compound IV-186);
4-{2R-[trans-3-(7-methoxybenzofuran-2-yl)acryloylamino]butoxy}phenylthiohydroxamic acid (Compound IV-187);
4-{2S-[trans-3-(5-methoxybenzofuran-2-yl)but-2-enoylamino]butoxy}phenylthiohydroxamic acid (Compound IV-188);
4-[2-(trans-3-furan-2-ylacryloylamino)ethoxy]phenylthiohydroxamic acid (Compound IV-189); and
4-{2-[4-(4-(2-morpholin-4-ylethyl)thiazol-2-yl)phenylcarbonylamino]ethoxy}-phenylthiohydroxamic acid (Compound IV-190).

Also described herein are pharmaceutically acceptable salts of compounds of Table IV.

In one aspect, representative compounds of Formula (II) where R¹ is hydrogen, Ar¹ is phenyl are disclosed in Table V:

TABLE V
Representative compounds of Formula (II).

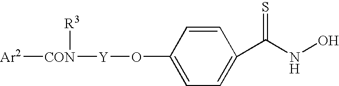

| Com- | | | |
|---|---|---|---|
| pound # | R³ | Ar² | Y |
| V-1 | 2-hydroxy- | trans phenyl-CH═CH— | —CH₂—CH₂— |
| | ethyl | | |
| V-2 | phenyl | trans phenyl-CH═CH— | —CH₂—CH₂— |
| V-3 | CH₃ | trans phenyl-CH═CH— | —CH₂—CH₂— |
| V-4 | i-propyl | benzothien-2-yl | —CH₂—CH₂— |
| V-5 | i-propyl | trans phenyl-CH═CH— | —CH₂—CH₂— |
| V-6 | CH₃ | trans phenyl-CH═CH— | —CH₂—CH₂—CH₂— |

Compounds in Table V are named:
4-(2-N-trans-cinnamoyl-N-hydroxyethylaminoethoxy)phenylthiohydroxamic acid (Compound V-1);
4-(2-N-trans-cinnamoyl-N-phenylaminoethoxy)phenylthiohydroxamic acid (Compound V-2);
4-(2-N-trans-cinnamoyl-N-methylaminoethoxy)phenylthiohydroxamic acid (Compound V-3);
4-(2-N-benzothien-2-yl-N-isopropylaminoethoxy)phenylthiohydroxamic acid (Compound V-4);
4-(2-N-trans-cinnamoyl-N-isopropylaminoethoxy)phenylthiohydroxamic acid (Compound V-5); and
4-(3-N-trans-cinnamoyl-N-methylaminopropoxy)phenylthiohydroxamic acid (Compound V-6).

Also described herein are pharmaceutically acceptable salts of compounds of Table V.

In one aspect, representative compounds of Formula (II) where R¹ and R³ are hydrogen, Ar¹ is phenyl, are disclosed in Table VI:

TABLE VI
Representative Compounds of Formula (II).

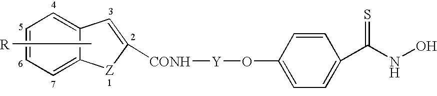

| Compound # | Z | R | Y |
|---|---|---|---|
| VI-1 | S | H | —CH₂—CH₂— |
| VI-2 | O | H | —CH₂—CH₂— |
| VI-3 | NH | H | —CH₂—CH₂— |
| VI-4 | NMe | H | —CH₂—CH₂— |
| VI-5 | S | H | —CH₂—CH₂—CH₂— |
| VI-6 | O | H | —CH₂—CH₂—CH₂— |
| VI-7 | S | H | —(S)—CH(i-propyl)-CH₂— |
| VI-8 | S | H | —(S)—CH(ethyl)-CH₂— |
| VI-9 | S | H | —(S)—CH(methyl)-CH₂— |
| VI-10 | S | H | —(R)—CH(methyl)-CH₂— |
| VI-11 | O | H | —(S)—CH(ethyl)-CH₂— |
| VI-12 | S | H | —(R)—CH₂—CH(CH₃)— |
| VI-13 | S | H | —(S)—CH₂—CH(CH₃)— |
| VI-14 | O | H | —(R)—CH₂—CH(CH₃)— |
| VI-15 | S | 6-methoxy | —CH₂—CH₂— |
| VI-16 | S | 5-methyl | —CH₂—CH₂— |
| VI-17 | S | 3-chloro | —CH₂—CH₂— |
| VI-18 | O | 5-methyl | —CH₂—CH₂— |
| VI-19 | O | 6-methyl | —CH₂—CH₂— |
| VI-20 | S | 4-CF₃ | —CH₂—CH₂— |
| VI-21 | S | 5-fluoro | —CH₂—CH₂— |
| VI-22 | S | 5-methoxy | —CH₂—CH₂— |
| VI-23 | O | 5-chloro | —CH₂—CH₂— |
| VI-24 | O | 7-methoxy | —CH₂—CH₂— |
| VI-25 | O | 5-methoxy | —CH₂—CH₂— |
| VI-26 | O | 5-(2-methoxyethoxy)- | —CH₂—CH₂— |
| VI-27 | O | 5-(2-morpholin-4-ylethoxy)- | —CH₂—CH₂— |
| VI-28 | O | 5-pyridin-3-ylmethoxy | —CH₂—CH₂— |
| VI-29 | O | 3-methyl | —CH₂—CH₂— |
| VI-30 | S | 3 methyl | —CH₂—CH₂— |
| VI-31 | O | 5-(2-hydroxyethoxy)- | —CH₂—CH₂— |
| VI-32 | O | 5-(2-N,N-dimethylaminoethoxy)- | —CH₂—CH₂— |
| VI-33 | O | 6-CH₃OCH₂CH₂O | —CH₂—CH₂— |
| VI-34 | O | 6-(2-morpholin-4-ylethoxy)- | —CH₂—CH₂— |
| VI-35 | O | 6-pyridin-3ylmethoxy- | —CH₂—CH₂— |
| VI-36 | O | 3-ethyl | —CH₂—CH₂— |
| VI-37 | NH | 5-fluoro | —CH₂—CH₂— |
| VI-38 | NH | 5-methoxy | —CH₂—CH₂— |
| VI-39 | O | 3-CH₃OCH₂ | —CH₂—CH₂— |
| VI-40 | O | 3-phenoxymethyl | —CH₂—CH₂— |
| VI-41 | NH | 5,6-dimethoxy | —CH₂—CH₂— |
| VI-42 | O | 3-morpholino-4-ylmethyl | —CH₂—CH₂— |
| VI-43 | O | 3-N,N-dimethylaminomethyl | —CH₂—CH₂— |
| VI-44 | O | 3-i-propoxymethyl | —CH₂—CH₂— |
| VI-45 | O | 7-phenoxymethyl | —CH₂—CH₂— |
| VI-46 | O | 7-CH₃OCH₂ | —CH₂—CH₂— |
| VI-47 | O | 7-morpholino-4-ylmethyl | —CH₂—CH₂— |
| VI-48 | O | 7-N,N-dimethylaminomethyl | —CH₂—CH₂— |
| VI-49 | S | 5-methyl | —CH₂—CH₂—CH₂— |
| VI-50 | S | 6-methoxy | —CH₂—CH₂—CH₂— |
| VI-51 | O | 7-CH₃OCH₂ | —CH₂—CH₂—CH₂— |
| VI-52 | O | 7-phenoxymethyl | —CH₂—CH₂—CH₂— |
| VI-53 | O | 5-CH₃OCH₂CH₂O | —(R)—CH₂—CH(CH₃)— |
| VI-54 | O | H | (R)—CH(CH₃Smethyl)-CH₂— |
| VI-55 | O | H | (R)—CH(CH₃SO₂-methyl)-CH₂— |
| VI-56 | O | 3-(2-phenylethyl)- | —CH₂—CH₂— |
| VI-57 | O | 3-(N-methyl-N-benzylaminomethyl)- | —CH₂—CH₂— |
| VI-58 | O | 3-(N-methyl-N-2-phenylethyl-aminomethyl)- | —CH₂—CH₂— |
| VI-59 | O | 3-(3-hydroxypropylthiomethyl)- | —CH₂—CH₂— |
| VI-60 | O | 3-(3-hydroxypropylsulfinylmethyl)- | —CH₂—CH₂— |
| VI-61 | O | 3-(3-hydroxypropylsulfonylmethyl)- | —CH₂—CH₂— |
| VI-62 | O | 3-(N-methyl-N-2-indol-3-yl- | —CH₂—CH₂— |
| | | ethylaminomethyl)- | |
| VI-63 | O | 3-[2-(3-trifluoromethylphenyl)-ethyl]- | —CH₂—CH₂— |
| VI-64 | O | 3-[2-(3-trifluoromethoxyphenyl)-ethyl]- | —CH₂—CH₂— |
| VI-65 | O | 3-(N-hydroxyaminocarbonyl- | —CH₂—CH₂— |
| | | methylaminomethyl)- | |
| VI-66 | O | 3-(2-carboxyethylamino-methyl)- | —CH₂—CH₂— |
| VI-67 | O | H | (RS)—CH₂CH-(phenoxymethyl)- |
| VI-68 | O | 3-(3-hydroxypropyloxymethyl)- | —CH₂—CH₂— |
| VI-69 | O | 3-(2-fluorophenoxymethyl)- | —CH₂—CH₂— |
| VI-70 | O | 3-(3-fluorophenoxymethyl)- | —CH₂—CH₂— |
| VI-71 | O | 3-(4-fluorophenoxymethyl)- | —CH₂—CH₂— |
| VI-72 | O | 3-(2-methoxyethyloxymethyl)- | —CH₂—CH₂— |
| VI-73 | O | 3-(pyridin-4-yloxymethyl)- | —CH₂—CH₂— |
| VI-74 | O | 3-(2,4,6-trifluorophenoxymethyl)- | —CH₂—CH₂— |
| VI-75 | O | 3-(2-oxopyridin-1-ylmethyl)- | —CH₂—CH₂— |
| VI-76 | O | 3-(2,2,2-trifluoroethoxymethyl)- | —CH₂—CH₂— |
| VI-77 | O | 3-(4-imidazol-1-ylphenoxymethyl)- | —CH₂—CH₂— |
| VI-78 | O | 3-(4-[1.2.4]-triazin-1-ylphenoxy-methyl)- | —CH₂—CH₂— |
| VI-79 | O | 3-(pyrrolidin-1-ylmethyl)- | —CH₂—CH₂— |
| VI-80 | O | 3-(piperidin-1-ylmethyl)- | —CH₂—CH₂— |
| VI-81 | O | 3-(4-trifluoromethylpiperidin-ylmethyl)- | —CH₂—CH₂— |
| VI-82 | O | 3-(4-methylpiperazin-1-yl-methyl)- | —CH₂—CH₂— |
| VI-83 | O | 3-(3,3,3-trifluoropropyloxy-methyl)- | —CH₂—CH₂— |
| VI-84 | O | 4-methyl | —CH₂—CH₂— |
| VI-85 | O | 3-(4-fluorophenylthiomethyl)- | —CH₂—CH₂— |
| VI-86 | O | 3-(4-fluorophenylsulfinyl-methyl)- | —CH₂—CH₂— |
| VI-87 | O | 3-(4-fluorophenylsulfonyl-methyl)- | —CH₂—CH₂— |
| VI-88 | O | 3-(2,2,2-trifluoroethoxy-methyl)- | (S)—CH(ethyl)-CH₂— |
| VI-89 | O | 4-hydroxy | —CH₂—CH₂— |
| VI-90 | O | 5-chloro | (S)—CH(ethyl)-CH₂— |
| VI-91 | O | 5-chloro | (R)—CH₂—CH(methyl)- |
| VI-92 | O | 4-pyridin-3-ylmethyloxy-methyl | —CH₂—CH₂— |
| VI-93 | O | 4-methoxy | —CH₂—CH₂— |
| VI-94 | O | 4-(2-methoxyethyloxy)- | —CH₂—CH₂— |
| VI-95 | O | 4-pyridin-3-ylmethyloxy | —CH₂—CH₂— |
| VI-96 | NH | 4-methoxy | —CH₂—CH₂— |
| VI-97 | O | 3-(2-methoxyethyloxymethyl)- | (S)—CH(ethyl)-CH₂— |
| VI-98 | O | 3-(2-methoxyethyloxymethyl)- | (R)—CH₂—CH(methyl)- |
| VI-99 | O | 3-N,N-diethylaminomethyl | —CH₂—CH₂— |
| VI-100 | O | 5-(2-methoxyethyloxy)- | (S)—CH(ethyl)-CH₂— |
| VI-101 | O | 5-tetrahydropyran-4-yloxy | —CH₂—CH₂— |
| VI-102 | O | 5-tetrahydropyran-4-yloxy | (S)—CH(ethyl)-CH₂— |
| VI-103 | O | 5-tetrahydropyran-4-yloxy | (R)—CH₂—CH(methyl)- |
| VI-104 | O | 5-(2,2,2-trifluoroethyloxy)- | —CH₂—CH₂— |
| VI-105 | O | 5-(2-pyrrolidin-1-ylethyloxy)- | —CH₂—CH₂— |
| VI-106 | O | 5-(2-pyrrolidin-1-ylethyloxy)- | (S)—CH(ethyl)-CH₂— |
| VI-107 | O | 5-(2-pyrrolidin-1-ylethyloxy)- | (R)—CH₂—CH(methyl)- |
| VI-108 | O | 5-(piperidin-4-yloxy)- | —CH₂—CH₂— |
| VI-109 | O | H | (S)—CH(2-CH₃Sethyl)-CH₂— |
| VI-110 | O | H | (S)—CH(2-CH₃SO₂ethyl)-CH₂— |
| VI-111 | O | —CH₂—N(CH₂CH₂OCH₃)₂ | —CH₂—CH₂— |
| VI-112 | O | —CH₂—N(CH₂CH₂OCH₂CH₂OCH₃)₂ | —CH₂—CH₂— |
| VI-113 | O | —CH₂—N(CH₂CH₂OCH₂CH₂OCH₂CH₂OCH₃)₂ | —CH₂—CH₂— |

Compounds in Table VI are named:
4-[2-(benzothien-2-ylcarbonylamino)ethoxy]-phenylthiohydroxamic acid (Compound VI-1);
4-[2-(benzofuran-2-ylcarbonylamino)ethoxy]-phenylthiohydroxamic acid (Compound VI-2);
4-[2-(1H-indol-2-ylcarbonylamino)ethoxy]-phenylthiohydroxamic acid (Compound VI-3);
4-[2-(1-methylindol-2-ylcarbonylamino)ethoxy]-phenylthiohydroxamic acid (Compound VI-4);
4-[3-(benzothien-2-ylcarbonylamino)propoxy]-phenylthiohydroxamic acid (Compound VI-5);
4-[3-(benzofuran-2-ylcarbonylamino)propoxy]-phenylthiohydroxamic acid (Compound VI-6);
4-[2S-(benzothien-2-ylcarbonylamino)-3-methylbutoxy]-phenylthiohydroxamic acid (Compound VI-7);
4-[2S-(benzothien-2-ylcarbonylamino)butoxy]-phenylthiohydroxamic acid (Compound VI-8);
4-[2S-(benzothien-2-ylcarbonylamino)-propoxy]-phenylthiohydroxamic acid (Compound VI-9);
4-[2R-(benzothien-2-ylcarbonylamino)-propoxy]-phenylthiohydroxamic acid (Compound VI-10);
4-[2S-(benzofuran-2-ylcarbonylamino)butoxy]-phenylthiohydroxamic acid (Compound VI-11);
4-[2-(benzothien-2-ylcarbonylamino)-1R-methylethoxy]-phenylthiohydroxamic acid (Compound VI-12);
4-[2-(benzothien-2-ylcarbonylamino)-1S-methylethoxy]-phenylthiohydroxamic acid (Compound VI-13);
4-[2-(benzofuran-2-ylcarbonylamino)-1R-methylethoxy]-phenylthiohydroxamic acid (Compound VI-14);
4-[2-(6-methoxybenzothien-2-ylcarbonylamino)ethoxy]-phenylthiohydroxamic acid (Compound VI-15);
4-[2-(5-methylbenzothien-2-ylcarbonylamino)ethoxy]-phenylthiohydroxamic acid (Compound VI-16);
4-[2-(3-chlorobenzothien-2-ylcarbonylamino)ethoxy]-phenylthiohydroxamic acid (Compound VI-17);
4-[2-(5-methylbenzofuran-2-ylcarbonylamino)ethoxy]-phenylthiohydroxamic acid (Compound VI-18);
4-[2-(6-methylbenzofuran-2-ylcarbonylamino)ethoxy]-phenylthiohydroxamic acid (Compound VI-19);
4-[2-(4-trifluoromethylbenzothien-2-ylcarbonylamino)ethoxy]-phenylthiohydroxamic acid (Compound VI-20);
4-[2-(5-fluorobenzothien-2-ylcarbonylamino)ethoxy]-phenylthiohydroxamic acid (Compound VI-21);
4-[2-(5-methoxybenzothien-2-ylcarbonylamino)ethoxy]-phenylthiohydroxamic acid (Compound VI-22);
4-[2-(5-chlorobenzofuran-2-ylcarbonylamino)ethoxy]-phenylthiohydroxamic acid (Compound VI-23);
4-[2-(7-methoxybenzofuran-2-ylcarbonylamino)ethoxy]-phenylthiohydroxamic acid (Compound VI-24);
4-[2-(5-methoxybenzofuran-2-ylcarbonylamino)ethoxy]-phenylthiohydroxamic acid (Compound VI-25);
4-{2-[5-(2-methoxyethoxy)benzofuran-2-ylcarbonylamino]ethoxy}-phenylthiohydroxamic acid (Compound VI-26);
4-{2-[5-(2-morpholin-4-ylethoxy)benzofuran-2-ylcarbonylamino]ethoxy}-phenylthiohydroxamic acid (Compound VI-27);
4-{2-[5-(pyridin-3-ylmethoxy)benzofuran-2-ylcarbonylamino]ethoxy}-phenylthiohydroxamic acid (Compound VI-28);
4-[2-(3-methylbenzofuran-2-ylcarbonylamino)ethoxy]-phenylthiohydroxamic acid (Compound VI-29);
4-[2-(3-methylbenzothien-2-ylcarbonylamino)ethoxy]-phenylthiohydroxamic acid (Compound VI-30);
4-{2-[5-(2-hydroxyethoxy)benzofuran-2-ylcarbonylamino]ethoxy}phenylthiohydroxamic acid (Compound VI-31);
4-{2-[5-(2-N,N-dimethylaminoethoxy)benzofuran-2-ylcarbonylamino]-ethoxy}-phenylthiohydroxamic acid (Compound VI-32);
4-{2-[6-(2-methoxyethoxy)benzofuran-2-ylcarbonylamino]ethoxy}-phenylthiohydroxamic acid (Compound VI-33);
4-{2-[6-(2-morpholin-4-ylethoxy)benzofuran-2-ylcarbonylamino]ethoxy}-phenylthiohydroxamic acid (Compound VI-34);
4-{2-[6-(pyridin-3-ylmethoxy)benzofuran-2-ylcarbonylamino]ethoxy}-phenylthiohydroxamic acid (Compound VI-35);
4-[2-(3-ethylbenzofuran-2-ylcarbonylamino)ethoxy]-phenylthiohydroxamic acid (Compound VI-36);
4-[2-(5-fluoroindol-2-ylcarbonylamino)ethoxy]-phenylthiohydroxamic acid (Compound VI-37);
4-[2-(5-methoxyindol-2-ylcarbonylamino)ethoxy]-phenylthiohydroxamic acid (Compound VI-38);
4-{2-[3-(methoxymethyl)benzofuran-2-ylcarbonylamino]ethoxy}phenylthiohydroxamic acid (Compound VI-39);
4-{2-[3-(phenoxymethyl)benzofuran-2-ylcarbonylamino]ethoxy}phenylthiohydroxamic acid (Compound VI-40);
4-[2-(5,6-dimethoxyindol-2-ylcarbonylamino)ethoxy]-phenylthiohydroxamic acid (Compound VI-41);
4-{2-[3-(morpholin-4-ylmethyl)benzofuran-2-ylcarbonylamino]ethoxy}-phenylthiohydroxamic acid (Compound VI-42);
4-{2-[3-(N,N-dimethylaminomethyl)benzofuran-2-ylcarbonylamino]ethoxy}-phenylthiohydroxamic acid (Compound VI-43);
4-{2-[3-(i-propoxymethyl)benzofuran-2-ylcarbonylamino]ethoxy}phenylthiohydroxamic acid (Compound VI-44);
4-{2-[7-(phenoxymethyl)benzofuran-2-ylcarbonylamino]ethoxy}phenylthiohydroxamic acid (Compound VI-45);
4-{2-[7-(methoxymethyl)benzofuran-2-ylcarbonylamino]ethoxy}phenylthiohydroxamic acid (Compound VI-46);
4-{2-[7-(morpholin-4-ylmethyl)benzofuran-2-ylcarbonylamino]ethoxy}-phenylthiohydroxamic acid (Compound VI-47);
4-{2-[7-(N,N-dimethylaminomethyl)benzofuran-2-ylcarbonylamino]ethoxy}-phenylthiohydroxamic acid (Compound VI-48);
4-{3-[5-(methyl)benzothien-2-ylcarbonylamino]propoxy}-phenylthiohydroxamic acid (Compound VI-49);
4-{3-[6-(methoxy)benzothien-2-ylcarbonylamino]propoxy}-phenylthiohydroxamic acid (Compound VI-50);
4-{3-[7-(methoxymethyl)benzofuran-2-ylcarbonylamino]propoxy}-phenylthiohydroxamic acid (Compound VI-51);
4-{3-[7-(phenoxymethyl)benzofuran-2-ylcarbonylamino]propoxy}-phenylthiohydroxamic acid (Compound VI-52);
4-{2-[5-(2-methoxyethoxy)benzofuran-2-ylcarbonylamino]-1R-methyl ethoxy}phenylthiohydroxamic acid (Compound VI-53).
4-(2R-benzofuran-2-ylcarbonylamino-3-methylthiopropoxy)phenylthiohydroxamic acid (Compound VI-54);
4-(2R-benzofuran-2-ylcarbonylamino-3-methylsulfonylpropoxy)phenylthiohydroxamic acid (Compound VI-55);
4-{2-[3-(2-phenylethyl)benzofuran-2-ylcarbonylamino]ethoxy}phenylthiohydroxamic acid (Compound VI-56);
4-{2-[3-(N-methyl-N-benzylaminomethyl)benzofuran-2-ylcarbonylamino]-ethoxy}phenylthiohydroxamic acid (Compound VI-57);
4-{2-[3-(N-methyl-N-2-phenylethylaminomethyl)benzofuran-2-ylcarbonylamino]-ethoxy}phenylthiohydroxamic acid (Compound VI-58);
4-{2-[3-(3-hydroxypropylthiomethyl)benzofuran-2-ylcarbonylamino]-ethoxy}phenylthiohydroxamic acid (Compound VI-59);
4-{2-[3-(3-hydroxypropylsulfinylmethyl)benzofuran-2-ylcarbonylamino]-ethoxy}phenylthiohydroxamic acid (Compound VI-60);
4-{2-[3-(3-hydroxypropylsulfonylmethyl)benzofuran-2-ylcarbonylamino]-ethoxy}phenylthiohydroxamic acid (Compound VI-61);
4-{2-[3-(N-methyl-N-2-indol-3-ylethylaminomethyl)benzofuran-2-ylcarbonylamino]-ethoxy}phenylthiohydroxamic acid (Compound VI-62);
4-{2-[3-(2-(3-trifluoromethylphenyl)ethyl)benzofuran-2-ylcarbonylamino]-ethoxy}phenylthiohydroxamic acid (Compound VI-63);
4-{2-[3-(2-(3-trifluoromethoxyphenyl)ethyl)benzofuran-2-ylcarbonylamino]-ethoxy}phenylthiohydroxamic acid (Compound VI-64);
4-{2-[3-(N-hydroxyaminocarbonylmethylaminomethyl)benzofuran-2-ylcarbonylamino]ethoxy}phenylthiohydroxamic acid (Compound VI-65);
4-{2-[3-(2-carboxyethylaminomethy)benzofuran-2-ylcarbonylamino]-ethoxy}phenylthiohydroxamic acid (Compound VI-66);
4-[2-(benzofuran-2-ylcarbonylamino)-1RS-phenoxymethylethoxy}-phenylthiohydroxamic acid (Compound VI-67);
4-{2-[3-(3-hydroxypropoxymethyl)benzofuran-2-ylcarbonylamino]ethoxy}-phenylthiohydroxamic acid (Compound VI-68);
4-{2-[3-(2-fluorophenoxymethyl)benzofuran-2-ylcarbonylamino]ethoxy}-phenylthiohydroxamic acid (Compound VI-69);
4-{2-[3-(3-fluorophenoxymethyl)benzofuran-2-ylcarbonylamino]ethoxy}-phenylthiohydroxamic acid (Compound VI-70);
4-{2-[3-(4-fluorophenoxymethyl)benzofuran-2-ylcarbonylamino]ethoxy}-phenylthiohydroxamic acid (Compound VI-71);
4-{2-[3-(2-methoxyethyloxymethyl)benzofuran-2-ylcarbonylamino]ethoxy}-phenylthiohydroxamic acid (Compound VI-72);
4-{2-[3-(pyridin-4-yloxymethyl)benzofuran-2-ylcarbonylamino]ethoxy}-phenylthiohydroxamic acid (Compound VI-73);
4-{2-[3-(2,4,6-trifluorophenoxymethyl)benzofuran-2-ylcarbonylamino]-ethoxy}phenylthiohydroxamic acid (Compound VI-74);
4-{2-[3-(2-oxopyridin-1-ylmethyl)benzofuran-2-ylcarbonylamino]ethoxy}-phenylthiohydroxamic acid (Compound VI-75);
4-{2-[3-(2,2,2-trifluoroethoxymethyl)benzofuran-2-ylcarbonylamino]-ethoxy}phenylthiohydroxamic acid (Compound VI-76);
4-{2-[3-(4-imidazol-1-ylphenoxymethyl)benzofuran-2-ylcarbonylamino]-ethoxy}phenylthiohydroxamic acid (Compound VI-77);
4-{2-[3-(4-[1.2.4]-triazin-1-ylphenoxymethyl)benzofuran-2-ylcarbonyl-amino]ethoxy}phenylthiohydroxamic acid (Compound VI-78);
4-{2-[3-(pyrrolidin-1-methyl)benzofuran-2-ylcarbonylamino]ethoxy}-phenylthiohydroxamic acid (Compound VI-79);
N-hydroxy-4-{2-[3-(piperidin-1-methyl)benzofuran-2-ylcarbonylamino]ethoxy}-phenylthiohydroxamic acid (Compound VI-80);
4-{2-[3-(4-trifluoromethylpiperidin-1-methyl)benzofuran-2-ylcarbonylamino]-ethoxy}phenylthiohydroxamic acid (Compound VI-81);
4-{2-[3-(4-methylpiperazin-1-methyl)benzofuran-2-ylcarbonylamino]-ethoxy}phenylthiohydroxamic acid (Compound VI-82);
4-{2-[3-(3,3,3-trifluoropropyloxymethyl)benzofuran-2-ylcarbonylamino]-ethoxy}phenylthiohydroxamic acid (Compound VI-83);
4-[2-(4-methylbenzofuran-2-ylcarbonylamino)-ethoxy]phenylthiohydroxamic acid (Compound VI-84);
4-{2-[3-(4-fluorophenylthiomethyl)benzofuran-2-ylcarbonylamino]-ethoxy}-phenylthiohydroxamic acid (Compound VI-85);
4-{2-[3-(4-fluorophenylsulfinylmethyl)benzofuran-2-ylcarbonylamino]-ethoxy}-phenylthiohydroxamic acid (Compound VI-86);
4-{2-[3-(4-fluorophenylsulfonylmethyl)benzofuran-2-ylcarbonylamino]-ethoxy}-phenylthiohydroxamic acid (Compound VI-87);
4-{2S-[3-(2,2,2-trifluoroethoxymethyl)benzofuran-2-ylcarbonylamino]-butoxy}-phenylthiohydroxamic acid (Compound VI-88);
4-[2-(4-hydroxybenzofuran-2-ylcarbonylamino)ethoxy]phenylthiohydroxamic acid (Compound VI-89);
4-[2S-(5-chlorobenzofuran-2-ylcarbonylamino)butoxy]phenylthiohydroxamic acid (Compound VI-90);
4-[2-(5-chlorobenzofuran-2-ylcarbonylamino]-1R-methylethoxy]phenylthiohydroxamic acid (Compound VI-91);
4-[2-(4-pyridin-3-ylmethyloxymethylbenzofuran-2-ylcarbonylamino)-ethoxy]phenylthiohydroxamic acid (Compound VI-92);
4-[2-(4-methoxybenzofuran-2-ylcarbonylamino)ethoxy]phenylthiohydroxamic acid (Compound VI-93);
4-{2-[4-(2-methoxyethyloxy)benzofuran-2-ylcarbonylamino)ethoxy}-phenylthiohydroxamic acid (Compound VI-94);
4-[2-(4-pyridin-3-ylmethyloxybenzofuran-2-ylcarbonylamino)-ethoxy]phenylthiohydroxamic acid (Compound VI-95);
4-[2-(4-methoxyindol-2-ylcarbonylamino)ethoxy]phenylthiohydroxamic acid (Compound VI-96);
4-{2S-[3-(2-methoxyethyloxymethyl)benzofuran-2-ylcarbonylamino]-butoxy}phenylthiohydroxamic acid (Compound VI-97);
4-{2-[3-(2-methoxyethyloxymethyl)benzofuran-2-ylcarbonylamino]-1R-methyl-ethoxy}phenylthiohydroxamic acid (Compound VI-98);
4-{2-[3-(N,N-diethylaminomethyl)benzofuran-2-ylcarbonylamino]ethoxy}-phenylthiohydroxamic acid (Compound VI-99);
4-{2S-[5-(2-methoxyethyloxy)benzofuran-2-ylcarbonylamino]butoxy}-phenylthiohydroxamic acid (Compound VI-100);
4-{2-[5-(tetrahydropyran-4-yloxy)benzofuran-2-ylcarbonylamino]ethoxy}-phenylthiohydroxamic acid (Compound VI-101);
4-{2S-[5-(tetrahydropyran-4-yloxy)benzofuran-2-ylcarbonylamino]butoxy}-phenylthiohydroxamic acid (Compound VI-102);
4-{2-[5-(tetrahydropyran-4-yloxy)benzofuran-2-ylcarbonylamino]-1R-methyl-ethoxy}phenylthiohydroxamic acid (Compound VI-103);
4-{2-[5-(2,2,2-trifluoroethyloxy)benzofuran-2-ylcarbonylamino]ethoxy}-phenylthiohydroxamic acid (Compound VI-104);
4-{2-[5-(2-pyrrolidin-1-ylethyloxy)benzofuran-2-ylcarbonylamino]ethoxy}-phenylthiohydroxamic acid (Compound VI-105);
4-{2S-[5-(2-pyrrolidin-1-ylethyloxy)benzofuran-2-ylcarbonylamino]butoxy}-phenylthiohydroxamic acid (Compound VI-106);
4-{2-[5-(2-pyrrolidin-1-ylethyloxy)benzofuran-2-ylcarbonylamino]-1R-methyl-ethoxy}phenylthiohydroxamic acid (Compound VI-107);
4-{2-[5-(piperidin-4-yloxy)benzofuran-2-ylcarbonylamino]ethoxy}-phenylthiohydroxamic acid (Compound VI-108);
4-[2S-(benzofuran-2-ylcarbonylamino)-4-methylthiobutoxy]phenylthiohydroxamic acid (Compound VI-109); and
4-[2S-(benzofuran-2-ylcarbonylamino)-4-methylsulfonylbutoxy]phenylthiohydroxamic acid (Compound VI-110).

Also described herein are pharmaceutically acceptable salts of compounds of Table VI.

In one aspect, representative compounds of Formula (II) where R¹ and R³ are hydrogen, Ar¹ is isoxazol-5-yl are disclosed in Table VII:

TABLE VII
Representative Compounds of Formula (II)

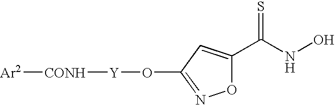

| Compound # | Ar² | Y |
|---|---|---|
| VII-1 | 4-biphenyl | (R)—CH₂—CH(CH₃)— |
| VII-2 | 4-biphenyl | (S)—CH(ethyl)-CH₂— |
| VII-3 | benzofuran-2-yl | —CH₂—CH₂— |
| VII-4 | trans phenyl-CH═CH— | —CH₂—CH₂— |
| VII-5 | 4-(2-ethoxyphenyl)phenyl | —CH₂—CH₂—CH₂— |
| VII-6 | 3-biphenyl | —CH₂—CH₂—CH₂— |
| VII-7 | 4-biphenyl | —CH₂—CH₂—CH₂— |
| VII-8 | naphth-2-yl | —CH₂—CH₂— |
| VII-9 | 3-methylbiphen-4-yl | —CH₂—CH₂— |
| VII-10 | 2′-ethoxybiphen-4-yl | —CH₂—CH₂— |
| VII-11 | 3-methylbiphen-4-yl | —CH₂—CH₂—CH₂— |
| VII-12 | 4-phenylthiazol-2-yl | —CH₂—CH₂—CH₂— |
| VII-13 | naphth-2-yl | —CH₂—CH₂—CH₂— |
| VII-14 | naphth-1-yl | —CH₂—CH₂—CH₂— |
| VII-15 | 2-(2-phenylethyl)phenyl | —CH₂—CH₂—CH₂— |
| VII-16 | naphth-1-yl | —CH₂—CH₂— |
| VII-17 | benzofuran-2-yl | (S)—CH(ethyl)-CH₂— |
| VII-18 | 3-biphenyl | (S)—CH(ethyl)-CH₂— |
| VII-19 | benzofuran-2-yl | (R)—CH₂—CH(methyl)- |
| VII-20 | 3-biphenyl | (R)—CH₂—CH(methyl)- |
| VII-21 | 3-biphenyl | —CH₂—CH₂— |
| VII-22 | 4-biphenyl | —CH₂—CH₂— |
| VII-23 | 4-phenylthiazol-2-yl | —CH₂—CH₂— |
| VII-24 | 2-(2-phenylethyl)phenyl | —CH₂—CH₂— |
| VII-25 | 2-biphenyl | —CH₂—CH₂— |
| VII-26 | 2-biphenyl | —CH₂—CH₂—CH₂— |
| VII-27 | naphth-2-yl | (S)—CH(ethyl)-CH₂— |
| VII-28 | naphth-1-yl | (S)—CH(ethyl)-CH₂— |
| VII-29 | naphth-2-yl | (R)—CH₂—CH(methyl)- |
| VII-30 | naphth-1-yl | (R)—CH₂—CH(methyl)- |
| VII-31 | benzofuran-2-yl | —CH₂—CH₂—CH₂— |
| VII-32 | trans phenylCH═CH— | —CH₂—CH₂—CH₂— |
| VII-33 | 3-(phenoxymethyl)benzofuran- | —CH₂—CH₂— |
| | 2-yl | |

Compounds in Table VII are named:
3-[2-(biphen-4-ylcarbonylamino)-1R-methylethoxy]isoxazol-5-ylthiohydroxamic acid (Compound VII-1);
3-[2S-(biphen-4-ylcarbonylamino)butoxy]isoxazol-5-ylthiohydroxamic acid (Compound VII-2);
3-[2-(benzofuran-2-ylcarbonylamino)ethoxy]isoxazol-5-ylthiohydroxamic acid (Compound VII-3);
3-[2-(trans-cinnamoylamino)ethoxy]isoxazol-5-ylthiohydroxamic acid (Compound VII-4);
3-[3-(4-(2-ethoxyphenyl)phenylcarbonylamino)propoxy]isoxazol-5-ylthiohydroxamic acid (Compound VII-5);
3-[3-(biphen-3-ylcarbonylamino)propoxy]isoxazol-5-ylthiohydroxamic acid (Compound VII-6);
3-[3-(biphen-4-ylcarbonylamino)propoxy]isoxazol-5-ylthiohydroxamic acid (Compound VII-7);
3-[2-(naphth-2-ylcarbonylamino)ethoxy]isoxazol-5-ylthiohydroxamic acid (Compound VII-8);
3-[2-(3-methylbiphen-4-ylcarbonylamino)ethoxy]isoxazol-5-ylthiohydroxamic acid (Compound VII-9);
3-[2-(2′-ethoxylbiphen-4-ylcarbonylamino)ethoxy]isoxazol-5-ylthiohydroxamic acid (Compound VII-10);
3-[3-(3-methylbiphen-4-ylcarbonylamino)propoxy]isoxazol-5-ylthiohydroxamic acid (Compound VII-11);
3-[3-(4-phenylthiazol-2-ylcarbonylamino)propoxy]isoxazol-5-ylthiohydroxamic acid (Compound VII-12);
3-[3-(naphth-2-ylcarbonylamino)propoxy]isoxazol-5-ylthiohydroxamic acid (Compound VII-13);
3-[3-(naphth-1-ylcarbonylamino)propoxy]isoxazol-5-ylthiohydroxamic acid (Compound VII-14);
3-{3-[2-(2-phenylethyl)phenylcarbonylamino]propoxy} isoxazol-5-ylthiohydroxamic acid (Compound VII-15);
3-[2-(naphth-1-ylcarbonylamino)ethoxy]isoxazol-5-ylthiohydroxamic acid (Compound VII-16);
3-[2S-(benzofuran-2-ylcarbonylamino)butoxy]isoxazol-5-ylthiohydroxamic acid (Compound VII-17);
3-[2S-(biphen-3-ylcarbonylamino)butoxy]isoxazol-5-ylthiohydroxamic acid (Compound VII-18);
3-[2-(benzofuran-2-ylcarbonylamino)-1R-methylethoxy]isoxazol-5-ylthiohydroxamic acid (Compound VII-19);
3-[2-(biphen-3-ylcarbonylamino)-1R-methylethoxy]isoxazol-5-ylthiohydroxamic acid (Compound VII-20);
3-[2-(biphen-3-ylcarbonylamino)ethoxy]isoxazol-5-ylthiohydroxamic acid (Compound VII-21);
3-[2-(biphen-4-ylcarbonylamino)ethoxy]isoxazol-5-ylthiohydroxamic acid (Compound VII-22);
3-[2-(4-phenylthiazol-2-ylcarbonylamino)ethoxy]isoxazol-5-ylthiohydroxamic acid (Compound VII-23);
3-{2-[2-(2-phenylethyl)phenylcarbonylamino]ethoxy} isoxazol-5-ylthiohydroxamic acid (Compound VII-24);
3-[2-(biphen-2-ylcarbonylamino)ethoxy]isoxazol-5-ylthiohydroxamic acid (Compound VII-25);
3-[3-(biphen-2-ylcarbonylamino)propoxy]isoxazol-5-ylthiohydroxamic acid (Compound VII-26);
3-[2S-(naphth-2-ylcarbonylamino)butoxy]isoxazol-5-ylthiohydroxamic acid (Compound VII-27);
3-[2S-(naphth-1-ylcarbonylamino)butoxy]isoxazol-5-ylthiohydroxamic acid (Compound VII-28);
3-[2-(naphth-2-ylcarbonylamino)-1R-methylethoxy]isoxazol-5-ylthiohydroxamic acid (Compound VII-29);
3-[2-(naphth-1-ylcarbonylamino)-1R-methylethoxy]isoxazol-5-ylthiohydroxamic acid (Compound VII-30);
3-[3-(benzofuran-2-ylcarbonylamino)propoxy]isoxazol-5-ylthiohydroxamic acid (Compound VII-31);
3-[3-(trans-cinnamoylamino)propoxy]isoxazol-5-ylthiohydroxamic acid (Compound VII-32); and
3-[2-(3-phenoxymethylbenzofuran-2-ylcarbonylamino)ethoxy]isoxazol-5-yl-thiohydroxamic acid (Compound VII-33).

Also described herein are pharmaceutically acceptable salts of compounds of Table VII.

### Selective HDAC8 Inhibitor Thiohydroxamic Acid Compounds

In one embodiment, provided herein are 1,3-disubstituted-1H-indole-6-thiohydroxamic acid compounds and 1,3-disubstituted-1H-indole-5-thiohydroxamic acid compounds, pharmaceutically acceptable salts, pharmaceutically acceptable N-oxides, pharmaceutically active metabolites, pharmaceutically acceptable prodrugs, or pharmaceutically acceptable solvates thereof, which inhibit HDAC8 activity, and may be used to treat patients where inhibition of HDAC8 activity provides benefit. 1,3-Disubstituted-1H-indole-6-thiohydroxamic acid compounds and 1,3-disubstituted-1H-indole-5-thiohydroxamic acid compounds described herein are selective HDAC8 inhibitor compounds.

In some embodiments of any of the methods described herein, the selective HDAC8 inhibitor has an IC₅₀ for HDAC8 that is at least 10 fold lower than the IC₅₀ for HDAC1, HDAC2, HDAC3, HDAC6, HDAC10, or HDAC11. In some embodiments of any of the methods described herein, the selective HDAC8 inhibitor has an IC₅₀ for HDAC8 that is less than 100 nM and that is at least 10 fold lower than the IC₅₀ for HDAC1, HDAC2, HDAC3, HDAC6, HDAC10, or HDAC11. In some embodiments of any of the methods described herein, the selective HDAC8 inhibitor has an IC₅₀ for HDAC8 that is less than 50 nM and that is at least 10 fold lower than the IC₅₀ of the selective inhibitor for HDAC1, HDAC2, HDAC3, HDAC6, HDAC10, or HDAC11.

In some embodiments, selective HDAC8 inhibitors described herein have an IC₅₀ for HDAC8 that is at least 15 fold lower than the IC₅₀ for HDAC1, HDAC2, HDAC3, HDAC6, and HDAC10. In some embodiments, selective HDAC8 inhibitors described herein have an IC₅₀ for HDAC8 that is at least 20 fold lower than the IC₅₀ for HDAC1, HDAC2, HDAC3, HDAC6, and HDAC10. In some embodiments, selective HDAC8 inhibitors described herein have an IC₅₀ for HDAC8 that is at least 100 fold lower than the IC₅₀ for HDAC1, HDAC2, HDAC3, HDAC6, and HDAC10. In addition, selective HDAC8 inhibitors described herein have an IC₅₀ for HDAC8 that is less than 100 nM while the IC₅₀ for HDAC1, HDAC2, HDAC3, HDAC6, and HDAC10 is greater than 100 nM.

In some embodiments, selective HDAC8 inhibitors described herein have an IC₅₀ for HDAC8 that is at least 10 fold lower than the IC₅₀ for HDAC1. In some embodiments, selective HDAC8 inhibitors described herein have an IC₅₀ for HDAC8 that is at least 20 fold lower than the IC₅₀ for HDAC1. In some embodiments, selective HDAC8 inhibitors described herein have an IC₅₀ for HDAC8 that is at least 40 fold lower than the IC₅₀ for HDAC1. In some embodiments, selective HDAC8 inhibitors described herein have an IC₅₀ for HDAC8 that is at least 100 fold lower than the IC₅₀ for HDAC1. In some embodiments, selective HDAC8 inhibitors described herein have an IC₅₀ for HDAC8 that is at least 150 fold lower than the IC₅₀ for HDAC1. In yet other embodiments, selective HDAC8 inhibitors described herein have an IC₅₀ for HDAC8 that is at least 200 fold lower than the IC₅₀ for HDAC1.

In some embodiments, selective HDAC8 inhibitors described herein have IC₅₀ for HDAC8 that is less than 100 nM and that is at least 20 fold lower than the IC₅₀ for other HDAC isoforms (HDAC1, HDAC2, HDAC3, HDAC6, HDAC10), wherein the IC₅₀ for the other HDAC isoforms is greater than 100 nM.

In one embodiment, described herein are substituted indole-6-thiohydroxamic acid compounds and substituted indole-5-thiohydroxamic acid compounds that are selective HDAC8 inhibitors. Compounds described herein are selective histone deacetylase 8 (HDAC8) inhibitors. In one embodiment, the selective HDAC8 inhibitor has an IC₅₀ for histone deacetylase 8 activity that is at least 10 fold lower than the IC₅₀ of the selective HDAC8 inhibitor for activity of histone deacetylase 1, histone deacetylase 2, histone deacetylase 3, histone deacetylase 6, histone deacetylase 10, or histone deacetylase 11.

In one embodiment, described herein is a 1,3-disubstituted-1H-indole-6-thiohydroxamic acid compound, wherein the substituent at the 1-position is X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; C(═O)—, and C(═O)—C₁-C₆alkylene;
R² is a substituted or unsubstituted group selected from among aryl, heteroaryl, cycloalkyl, and heterocycloalkyl;
   where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
      R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
      R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
   X⁶ is a C₁-C₆alkylene, C₁-C₆fluoroalkylene, C₂-C₆alkenylene, C₂-C₆heteroalkylene;
   R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷
   X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
   R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
      R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
      R^{a} and R⁷ together with the N atom to which they are attached form a 5-, 6-, or 7-membered heterocycloalkyl;
         or an active metabolite, pharmaceutically acceptable solvate, pharmaceutically acceptable salt, pharmaceutically acceptable N-oxide, or pharmaceutically acceptable prodrug thereof.

For any and all of the embodiments, substituents can be selected from among from a subset of the listed alternatives. For example, in some embodiments, X² is a substituted or unsubstituted group selected from among C₁-C₆alkylene, C₂-C₆alkenylene, C₁-C₆fluoroalkylene, C₂-C₆fluoroalkenylene, and C₁-C₆heteroalkylene. In other embodiments, X² is a substituted or unsubstituted group selected from among C₁-C₆alkylene, and C₂-C₆alkenylene. In some embodiments, X² is —CH₂—, —CH₂CH₂—, —CH(CH₃)—, —(CH₂)₃—, or —CH₂CH═CH—. In some embodiments, X² is —CH₂—.

In some embodiments, R² is an optionally substituted group selected from among phenyl, naphthyl, monocyclic heteroaryl, bicyclic heteroaryl, C₃-C₈ cycloalkyl, monocyclic heterocycloalkyl, and bicyclic heterocycloalkyl. In other embodiments, R² is an optionally substituted group selected from among phenyl, naphthyl, (monocyclic heteroaryl containing 0-2 N atoms, 0-10 atoms, and 0-1 S atoms), (bicyclic heteroaryl containing 0-2 N atoms, 0-10 atoms, and 0-1 S atoms), C₃-C₈ cyclolalkyl, monocyclic heterocycloalkyl containing 0-2 N atoms, and bicyclic heterocycloalkyl 0-2 N atoms; where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, phenyl, and heteroaryl; R¹¹ is a substituted or unsubstituted group selected from among C₁-C₆alkyl, C₁-C₆fluoroalkyl, C₃-C₈cycloalkyl, C₂-C₈heterocycloalkyl, phenyl, and heteroaryl.

In some embodiments, R² is an optionally substituted group selected from among phenyl, naphthyl, (monocyclic heteroaryl containing 0-2 N atoms, 0-10 atoms, and 0-1 S atoms), (bicyclic heteroaryl containing 0-2 N atoms, 0-10 atoms, and 0-1 S atoms), C₃-C₈ cyclolalkyl; where if R² is substituted, then each substituent on R² is selected from among hydrogen, halogen, —CN, —NO₂, —S(═O)₂NH₂, —CO₂H, —CO₂R¹⁰, —C(═O)R¹, —S—R¹¹, —S(═O)—R¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, and phenyl; R¹¹ is a substituted or unsubstituted group selected from among C₁-C₆alkyl, C₁-C₆fluoroalkyl, and phenyl.

In some embodiments, R² is selected from among phenyl, 2-methylphenyl, 3-methylphenyl, 4-methylphenyl, 3,4-dimethylphenyl, 2-fluorophenyl, 3-fluorophenyl, 4-fluorophenyl, 3,4-difluorophenyl, 2-chlorophenyl, 3-chlorophenyl, 4-chlorophenyl, 3,4-dichlorophenyl, 3-methoxyphenyl, 3-methoxyphenyl, 4-methoxyphenyl, 3,5-dimethoxyphenyl, 3,4,5-trimethoxyphenyl, naphth-2-yl, cyclopentyl, cyclohexyl, cycloheptyl, 2-(trifluoromethyl)-phenyl, 3-(trifluoromethyl)-phenyl, 4-(trifluoromethyl)-phenyl, 2-(trifluoromethoxy)-phenyl, 3-(trifluoromethoxy)-phenyl, 4-(trifluoromethoxy)-phenyl, benzo[2,1,3]oxadiazol-5-yl, 3-fluoro-4-methoxy-phenyl, 2-(difluoromethoxy)-phenyl, 3-(difluoromethoxy)-phenyl, 4-(difluoromethoxy)-phenyl, N-(t-butoxycarbonyl)piperidin-4-yl, piperidin-4-yl, N-methylsulfonyl-2-aminophenyl, N-methylsulfonyl-3-aminophenyl, N-methylsulfonyl-4-aminophenyl, N-phenylsulfonyl-2-aminophenyl, N-phenylsulfonyl-3-aminophenyl, N-phenylsulfonyl-4-aminophenyl, 2-nitrophenyl, 3-nitrophenyl, 4-nitrophenyl, 2-aminophenyl, 3-aminophenyl, 4-aminophenyl, 2-dimethylaminophenyl, 3-dimethylaminophenyl, 4-dimethylaminophenyl, N-acetyl-2-aminophenyl, N-acetyl-3-aminophenyl, N-acetyl-4-aminophenyl, N-benzoyl-2-aminophenyl, N-benzoyl-3-aminophenyl, and N-benzoyl-4-aminophenyl.

In other embodiments, R² is selected from among phenyl, 2-methylphenyl, 2-fluorophenyl, 3-fluorophenyl, 4-fluorophenyl, 2-chlorophenyl, 3-chlorophenyl, 4-chlorophenyl, 3,4-dichlorophenyl, 3-methoxyphenyl, 4-methoxyphenyl, 3,5-dimethoxyphenyl, 3,4,5-trimethoxyphenyl, naphth-2-yl, cyclohexyl, 4-(trifluoromethoxy)-phenyl, benzo[2,1,3]oxadiazol-5-yl, 3-methylphenyl, 4-methylphenyl, 3-fluoro-4-methoxy-phenyl, 4-(difluoromethoxy)-phenyl, N-(t-butoxycarbonyl)piperidin-4-yl, piperidin-4-yl, and N-methylsulfonyl-3-aminophenyl.

In some embodiments, R³ is hydrogen, halogen, substituted or unsubstituted C₁-C₆alkyl, substituted or unsubstituted C₂-C₆alkenyl, substituted or unsubstituted C₁-C₆heteroalkyl, substituted or unsubstituted phenyl, or —X⁶—R⁶; X⁶ is a C₁-C₆alkylene, C₁-C₆fluoroalkylene, C₂-C₆alkenylene, or C₂-C₆heteroalkylene; R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷; X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—; R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or R^{a} and R⁷ together with the N atom to which they are attached form a 5-, 6-, or 7-membered heterocycloalkyl.

In some embodiments, R³ is hydrogen, halogen, substituted or unsubstituted C₁-C₆alkyl, or —X⁶—R⁶.

In some embodiments, X⁶ is C₁-C₆alkylene; R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl containing 0-2 N atoms, phenyl, heteroaryl containing 0-2 N atoms, or X⁷—R⁷; X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—; R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or R^{a} and R⁷ together with the N atom to which they are attached form a 5-, 6-, or 7-membered heterocycloalkyl.

In some embodiments, R⁶ is X⁷—R⁷.

In some embodiments, X⁷ is a bond, —O—, —S—, —S(═O)—, —S(═O)₂—, —NR^{a}—, or —C(═O)—.

In some embodiments, R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, phenyl, phenylC₁-C₄alkyl, heteroaryl, heteroarylC₁-C₄alkyl; R^{a} is selected from among hydrogen, C₁-C₆alkyl, hydroxy, C₁-C₆alkoxy, C₁-C₆fluoroalkoxy, C₁-C₆heteroalkyl; or R^{a} and R⁷ together with the N atom to which they are attached form a 5-, or 6-membered heterocycloalkyl.

In some embodiments, X⁷ is a bond, —O—, or —NR^{a}—. In some embodiments, X⁷ is a bond, or —NR^{a}—.

In some embodiments, R^{a} is selected from among hydrogen, C₁-C₆alkyl, hydroxy, C₁-C₆alkoxy, C₁-C₆heteroalkyl. In other embodiments, R^{a} is selected from among hydrogen, C₁-C₆alkyl, and C₁-C₆heteroalkyl.

In some embodiments, R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, phenyl, phenylC₁-C₄alkyl, heteroaryl, heteroarylC₁-C₄alkyl; R^{a} is selected from among hydrogen, C₁-C₆alkyl, hydroxy, C₁-C₆alkoxy, C₁-C₆heteroalkyl; or R^{a} and R⁷ together with the N atom to which they are attached form a 5-, or 6-membered heterocycloalkyl.

In some embodiments, R³ is selected from among hydrogen, methyl, ethyl, propyl, benzyl, dimethylaminomethyl, N-morpholinomethyl, N-pyrrolidinomethyl, N-piperidinomethyl, and N-benzylaminomethyl. In some embodiments, R³ is selected from among hydrogen, methyl, ethyl, propyl, benzyl, dimethylaminomethyl, N-morpholinomethyl, N-pyrrolidinomethyl, and N-benzylaminomethyl.

Any combination of the groups described above for the various variables is contemplated herein.

In one embodiment, provided herein is a 1,3-disubstituted-1H-indole-5-thiohydroxamic acid compound, wherein the substituent at the 1-position is R⁴ and the substituent at the 3-position is X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or X⁸—R⁸;
   X⁸ is a C₂-C₆alkylene, C₂-C₆fluoroalkylene, C₂-C₆alkenylene, or C₂-C₆heteroalkylene;
   R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹;
   X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
   R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
      R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
      R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, C(═O)—, and C(═O)—C₁-C₆alkylene;
R⁵ is a substituted or unsubstituted group selected from among aryl, heteroaryl, C₃-C₈cycloalkyl, and heterocycloalkyl;
   where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
      R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
      R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
         or an active metabolite, pharmaceutically acceptable solvate, pharmaceutically acceptable salt, pharmaceutically acceptable N-oxide, or pharmaceutically acceptable prodrug thereof.

For any and all of the embodiments, substituents can be selected from among from a subset of the listed alternatives. For example, in some embodiments, R⁴ is hydrogen, substituted or unsubstituted C₁-C₆alkyl, substituted or unsubstituted C₂-C₆alkenyl, substituted or unsubstituted C₁-C₆alkoxy, substituted or unsubstituted C₁-C₆heteroalkyl, substituted or unsubstituted phenyl, or X⁸—R⁸; X⁸ is a C₂-C₆alkylene, C₂-C₆fluoroalkylene, C₂-C₆alkenylene, or C₂-C₆heteroalkylene; R¹ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹; X⁹ is a bond, —O—, —S—, —NR^{a}—, —C(═O)—; R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, and C₁-C₆heteroalkyl; or R¹ and R⁹ together with the N atom to which they are attached form a 5-, or 6-membered heterocycloalkyl.

In other embodiments, R⁴ is hydrogen, substituted or unsubstituted C₁-C₆alkyl, substituted or unsubstituted C₁-C₆heteroalkyl, substituted or unsubstituted phenyl, or X¹—R⁸; X⁸ is a C₂-C₆alkylene; R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁹—R⁹; X⁹ is a bond, —O—, —S—, —NR^{a}—, —C(═O)—; R⁹ is hydrogen, C₁-C₆alkyl, C₁-C₆heteroalkyl, C₁-C₆haloalkyl, C₂-C₈heterocycloalkyl, heterocycloalkylalkyl, phenyl, phenylalkyl, heteroaryl, heteroarylalkyl; R^{a} is selected from among hydrogen, C₁-C₆alkyl, hydroxy, C₁-C₆alkoxy; or R¹ and R⁹ together with the N atom to which they are attached form a 5-, or 6-membered heterocycloalkyl.

In some embodiments, R⁴ is selected from among hydrogen, methyl, ethyl, propyl, isopropyl, phenyl, and benzyl.

In some embodiments, X⁵ is a bond, or a substituted or unsubstituted group selected from among C₁-C₆alkylene, C₂-C₆alkenylene, C₁-C₆fluoroalkylene, and C₁-C₆heteroalkylene. In other embodiments, X⁵ is a bond, or a substituted or unsubstituted C₁-C₆alkylene. In some embodiments, X⁵ is —CH₂—, —CH₂CH₂—, —CH(CH₃)—, —(CH₂)₃—, or —CH₂CH═CH—. In some embodiments, X⁵ is —CH₂—.

In some embodiments, R⁵ is a substituted or unsubstituted group selected from among phenyl, naphthyl, (heteroaryl containing 0-2 N atoms, 0-10 atoms, 0-1 S atoms), C₃-C₈cycloalkyl, and heterocycloalkyl containing 0-2 N atoms.

In some embodiments, if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹, —S(═O)—R¹, —S(═O)₂—R¹, —NR¹⁰C(═O)—R¹, —C(═O)N(R¹⁰)₂, —S(═O)₂N(R¹⁰)₂, —NR¹⁰S(═O)₂—R¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted heterocycloalkyl, substituted or unsubstituted phenyl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, phenyl, and heteroaryl; R¹¹ is a substituted or unsubstituted group selected from among C₁-C₆alkyl, C₁-C₆fluoroalkyl, phenyl, and heteroaryl.

In some embodiments, R⁵ is a substituted or unsubstituted group selected from among phenyl, naphthyl, (monocyclic heteroaryl containing 0-2 N atoms, 0-10 atoms, 0-1 S atoms), and C₂-C₈heterocycloalkyl containing 0-2 N atoms.

In some embodiments, R⁵ is selected from among phenyl, 2-methylphenyl, 3-methylphenyl, 4-methylphenyl, 3,4-dimethylphenyl, 2-fluorophenyl, 3-fluorophenyl, 4-fluorophenyl, 3,4-difluorophenyl, 2-chlorophenyl, 3-chlorophenyl, 4-chlorophenyl, 3,4-dichlorophenyl, 3-methoxyphenyl, 3-methoxyphenyl, 4-methoxyphenyl, 3,5-dimethoxyphenyl, 3,4,5-trimethoxyphenyl, naphth-2-yl, cyclopentyl, cyclohexyl, cycloheptyl, 2-(trifluoromethyl)-phenyl, 3-(trifluoromethyl)-phenyl, 4-(trifluoromethyl)-phenyl, 2-(trifluoromethoxy)-phenyl, 3-(trifluoromethoxy)-phenyl, 4-(trifluoromethoxy)-phenyl, benzo[2,1,3]oxadiazol-5-yl, 3-fluoro-4-methoxy-phenyl, 2-(difluoromethoxy)-phenyl, 3-(difluoromethoxy)-phenyl, 4-(difluoromethoxy)-phenyl, N-(t-butoxycarbonyl)piperidin-4-yl, piperidin-4-yl, N-methylsulfonyl-2-aminophenyl, N-methylsulfonyl-3-aminophenyl, N-methylsulfonyl-4-aminophenyl, N-phenylsulfonyl-2-aminophenyl, N-phenylsulfonyl-3-aminophenyl, N-phenylsulfonyl-4-aminophenyl, 2-nitrophenyl, 3-nitrophenyl, 4-nitrophenyl, 2-aminophenyl, 3-aminophenyl, 4-aminophenyl, 2-dimethylaminophenyl, 3-dimethylaminophenyl, 4-dimethylaminophenyl, N-acetyl-2-aminophenyl, N-acetyl-3-aminophenyl, N-acetyl-4-aminophenyl, 2-(phenylcarbonylamino)-phenyl, 3-(phenylcarbonylamino)-phenyl, and 4-(phenylcarbonylamino)-phenyl. In some embodiments, R⁵ is selected from among phenyl, 4-nitrophenyl, 4-aminophenyl, 4-(phenylcarbonylamino)-phenyl, 4-fluorophenyl, and 4-(t-butoxycarbonyl)piperazin-1-yl.

Any combination of the groups described above for the various variables is contemplated herein.

In another aspect, provided herein is a compound having a structure selected from among Formula (IIIa) and (IVa):

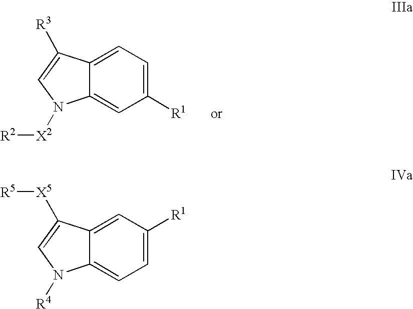

wherein:
R¹ is —C(═S)NHOH;
X² is a bond, alkylene, or alkenylene, where the alkylene or alkenylene is optionally substituted with one, two, three, four, or five halo;
R² is aryl, cycloalkyl, heteroaryl, or heterocycloalkyl where the aryl, cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro;
R³ is hydrogen, alkyl, substituted alkyl, alkenyl, substituted alkenyl, hydroxy, alkoxy, or haloalkoxy;
R⁴ is hydrogen, alkyl, substituted alkyl, alkenyl, substituted alkenyl, hydroxy, alkoxy, haloalkoxy, or optionally substituted phenyl;
X⁵ is a bond, alkylene, or alkenylene where the alkylene or alkenylene is optionally substituted with halo; and
R⁵ is aryl, cycloalkyl, heteroaryl, or heterocycloalkyl where the aryl, cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro; or
   an active metabolite, pharmaceutically acceptable solvate, pharmaceutically acceptable salt, pharmaceutically acceptable N-oxide, or pharmaceutically acceptable prodrug thereof.

In another embodiment, provided herein is a compound having a structure selected from among Formula IIIb or IVb:

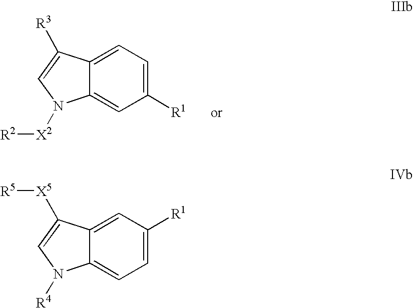

wherein:
R¹ is —C(═S)NHOH;
X² is a bond, alkylene, or alkenylene where the alkylene or alkenylene is optionally substituted with one, two, three, four, or five halo;
R² is aryl, cycloalkyl, heteroaryl, or heterocycloalkyl where the aryl is substituted with one, two, or three acyloxy, alkyl, substituted alkyl, alkenyl, substituted alkenyl, alkylamino, dialkylamino, or haloalkoxy; where the cycloalkyl is optionally substituted with one, two, or three acyl, acylamino, acyloxy, alkyl, substituted alkyl, alkenyl, substituted alkenyl, alkoxy, alkoxycarbonyl, amino, alkylamino, dialkylamino, carboxy, cyano, halo, haloalkoxy, or nitro; and where the heteroaryl and the heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, or nitro;
R³ is hydrogen, alkenyl, substituted alkenyl, hydroxy, alkoxy, haloalkoxy, or —X⁶—R⁶ where X⁶ is alkylene or alkenylene and X⁶ is additionally optionally substituted with one, two, three, four, of five halo; and R⁶ is alkylcarbonyl, alkenylcarbonyl, optionally substituted cycloalkylcarbonyl, alkylcarbonyloxy, alkenylcarbonyloxy, amino, alkylamino, dialkylamino, cyano, cyanoalkylaminocarbonyl, alkoxy, alkenyloxy, hydroxyalkoxy, halo, alkylcarbonylamino, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, substituted alkyl, alkenyl, substituted alkynyl, hydroxy, alkoxy, or alkenyloxy), alkylaminocarbonyloxy, dialkylaminocarbonyloxy, alkylaminoalkyloxy, dialkylaminoalkyloxy, alkoxycarbonylamino, alkylaminocarbonylamino, dialkylaminocarbonylamino, alkoxyalkyloxy, or —C(O)NR^{a}R^{b} (where R^{a} and R^{b} are independently hydrogen, alkyl, substituted alkyl, alkenyl, alkynyl, substituted alkynyl, hydroxy, alkoxy, or alkenyloxy);
R⁴ is hydrogen, alkyl, substituted alkyl, alkenyl, substituted alkenyl, hydroxy, alkoxy, haloalkoxy, or optionally substituted phenyl; and
X⁵ is a bond; and R⁵ is phenyl, 3- to 8-membered monocyclic cycloalkyl, 5- or 6-membered monocyclic heteroaryl, or 3- to 8-membered monocyclic heterocycloalkyl where the 3- to 8-membered monocyclic cycloalkyl, 5- or 6-membered monocyclic heteroaryl, and 3- to 8-membered monocyclic heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro; and the phenyl is substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro; provided that R⁵ is not optionally substituted pyrrole or optionally substituted 2,5-dioxo-pyrrole; or
X⁵ is alkylene or alkenylene where the alkylene or alkenylene is optionally substituted with halo; and R⁵ is aryl, cycloalkyl, heteroaryl, or heterocycloalkyl where the cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro; and the aryl is substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halo, haloalkoxy, or nitro; or
   an active metabolite, pharmaceutically acceptable solvate, pharmaceutically acceptable salt, pharmaceutically acceptable N-oxide, or pharmaceutically acceptable prodrug thereof.

In one embodiment, provided herein is a compound of Formula (IIIa).

In another embodiment, provided herein is a compound of Formula (IIIb).

In yet another embodiment, provided herein is a compound of Formula (IVa).

In a further embodiment, provided herein is a compound of Formula (IVb).

For any and all of the embodiments, substituents can be selected from among from a subset of the listed alternatives. For example, in some embodiments, X² is a bond, alkylene, or alkenylene where the alkylene or alkenylene is optionally substituted with one, two, three, four, or five halogens. In another embodiment, X² is alkylene or alkenylene. In other embodiments, X² is —CH₂—, —CH₂CH₂—, —CH(CH₃)—, —(CH₂)₃—, or —CH₂CH═CH—. In some embodiments, X² is —CH₂—.

In some embodiments, R² is aryl, cycloalkyl, heteroaryl, or heterocycloalkyl where the aryl, cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro. In other embodiments, R² is aryl, cycloalkyl, heteroaryl, or heterocycloalkyl, where the aryl, cycloalkyl, heteroaryl, and heterocycloalkyl are optionally substituted with one, two, or three substituents selected from among alkyl, alkoxy, alkoxycarbonyl, halogen, and haloalkoxy. In some other embodiments, R² is aryl, cycloalkyl, heteroaryl, or heterocycloalkyl, where the aryl is optionally substituted with one, two, or three substituents selected from among alkyl, alkoxy, halo, and haloalkoxy, and the heterocycloalkyl is optionally substituted with alkoxycarbonyl. In further embodiments, R² is cyclohexyl, benzooxadiazolyl, naphth-2-yl, phenyl, or piperidinyl, where the phenyl is optionally substituted with one, two, or three substituents selected from among methyl, methoxy, chloro, fluoro, trifluoromethoxy, and difluoromethoxy, and the piperidinyl is optionally substituted with t-butoxycarbonyl. In yet other embodiments, R² is cyclohexyl, benzo[2,1,3]oxadiazol-5-yl, phenyl, naphth-2-yl, 2-methylphenyl, 3-methylphenyl, 4-methylphenyl, 3-methoxyphenyl, 4-methoxyphenyl, 3,5-dimethoxyphenyl, 3,4,5-trimethoxyphenyl, 2-chlorophenyl, 3-chlorophenyl, 4-chlorophenyl, 3,4-dichlorophenyl, 2-fluorophenyl, 3-fluorophenyl, 4-fluorophenyl, 4-(difluoromethoxy)-phenyl, 4-(trifluoromethoxy)-phenyl, 3-fluoro-4-methoxy-phenyl, piperidin-4-yl, or N-(t-butoxycarbonyl)piperidin-4-yl.

In some embodiments, R² is benzo[2,1,3]oxadiazol-5-yl, 4-methoxyphenyl, 4-chlorophenyl, 4-(difluoromethoxy)-phenyl, or 3-fluoro-4-methoxy-phenyl.

In some embodiments, R³ is hydrogen, alkyl, substituted alkyl, alkenyl, substituted alkenyl, hydroxy, alkoxy, or haloalkoxy. In other embodiments, R³ is hydrogen.

In some embodiments, R⁴ is hydrogen, alkyl, substituted alkyl, alkenyl, substituted alkenyl, hydroxy, alkoxy, haloalkoxy, or optionally substituted phenyl. In yet other embodiments, R⁴ is alkyl or optionally substituted phenyl. In some other embodiments, R⁴ is methyl, ethyl, isopropyl, or phenyl. In some embodiments, R⁴ is methyl, ethyl, or isopropyl.

In some embodiments, X⁵ is a bond, alkylene, or alkenylene where the alkylene or alkenylene is optionally substituted with halo. In other embodiments, X⁵ is alkylene. In yet other embodiments, X⁵ is —CH₂—.

In some embodiments, R⁵ is aryl, cycloalkyl, heteroaryl, or heterocycloalkyl where the aryl, cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In yet other embodiments, R⁵ is heterocycloalkyl optionally substituted with alkoxycarbonyl or R⁵ is aryl optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In some embodiments, R⁵ is piperazinyl optionally substituted with t-butoxycarbonyl, or R⁵ is phenyl optionally substituted with one, two, or three substituents selected from among acylamino, amino, halogen, and nitro. In some other embodiments, R⁵ is 4-(t-butoxycarbonyl)piperazin-1-yl, phenyl, 4-aminophenyl, 4-(phenylcarbonylamino)-phenyl, 4-fluorophenyl, or 4-nitrophenyl. In yet other embodiments, R⁵ is phenyl, 4-aminophenyl, 4-(phenylcarbonylamino)-phenyl, 4-fluorophenyl, or 4-nitrophenyl.

In some embodiments, R² is aryl, cycloalkyl, heteroaryl, or heterocycloalkyl, where the aryl is substituted with one, two, or three substituents selected from among acyloxy, alkyl, substituted alkyl, alkenyl, substituted alkenyl, alkylamino, dialkylamino, and haloalkoxy; where the cycloalkyl is optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro; and where the heteroaryl and the heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, and nitro. In other embodiments, R² is aryl, cycloalkyl, heteroaryl, or heterocycloalkyl where the aryl is substituted with one, two, or three substituents selected from among alkyl and haloalkoxy, and the heterocycloalkyl is optionally substituted with alkoxycarbonyl. In yet other embodiments, R² is cyclohexyl; benzooxadiazolyl; phenyl substituted with one, two, or three substituents selected from among methyl, trifluoromethoxy, or difluoromethoxy; or piperidinyl optionally substituted with t-butoxycarbonyl.

In some embodiments, R² is cyclohexyl, benzo[2,1,3]oxadiazol-5-yl, 2-methylphenyl, 3-methylphenyl, 4-methylphenyl, 4-(difluoromethoxy)-phenyl, 4-(trifluoromethoxy)-phenyl, N-(t-butoxycarbonyl)piperidin-4-yl, or piperidin-4-yl. In yet other embodiments, R² is benzo[2, 1,3]oxadiazol-5-yl or 4-(difluoromethoxy)-phenyl.

In some embodiments, R³ is hydrogen, alkenyl, substituted alkenyl, hydroxy, alkoxy, haloalkoxy, or —X⁶—R⁶, where X⁶ is alkylene or alkenylene and X⁶ is additionally optionally substituted with one, two, three, four, or five halogens; and R⁶ is alkylcarbonyl, alkenylcarbonyl, optionally substituted cycloalkylcarbonyl, alkylcarbonyloxy, alkenylcarbonyloxy, amino, alkylamino, dialkylamino, cyano, cyanoalkylaminocarbonyl, alkoxy, alkenyloxy, hydroxyalkoxy, halo, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, substituted alkyl, alkenyl, substituted alkynyl, hydroxy, alkoxy, or alkenyloxy), alkylaminocarbonyloxy, dialkylaminocarbonyloxy, alkylaminoalkyloxy, dialkylaminoalkyloxy, alkoxycarbonylamino, alkylaminocarbonylamino, dialkylaminocarbonylamino, alkoxyalkyloxy, or —C(O)NR^{a}R^{b} (where R^{a} and R^{b} are independently hydrogen, alkyl, substituted alkyl, alkenyl, alkynyl, substituted alkynyl, hydroxy, alkoxy, or alkenyloxy). In some embodiments, R³ is hydrogen.

In some embodiments, X⁵ is a bond; and R⁵ is phenyl, 3- to 8-membered monocyclic cycloalkyl, 5- or 6-membered monocyclic heteroaryl, or 3- to 8-membered monocyclic heterocycloalkyl where the 3- to 8-membered monocyclic cycloalkyl, 5- or 6-membered monocyclic heteroaryl, and 3- to 8-membered monocyclic heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; and the phenyl is substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; provided that R⁵ is not optionally substituted pyrrole or optionally substituted 2,5-dioxo-pyrrole; or X⁵ is alkylene or alkenylene where the alkylene or alkenylene is optionally substituted with halogen; and R⁵ is aryl, cycloalkyl, heteroaryl, or heterocycloalkyl where the cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; and the aryl is substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In some embodiments, X⁵ is alkylene or alkenylene; and R⁵ is aryl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In other embodiments, R⁵ is phenyl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In some other embodiments, R⁵ is phenyl substituted with one, two, or three substituents selected from among optionally substituted arylcarbonylamino, amino, halo, and nitro. In yet other embodiments, R⁵ is 4-(phenylcarbonylamino)-phenyl, 4-aminophenyl, 4-fluorophenyl, or 4-nitrophenyl.

In some embodiments, R³ is hydrogen; X² is alkylene or alkenylene; and R² is aryl, cycloalkyl, or heteroaryl, where the aryl, cycloalkyl, and heteroaryl are optionally substituted with one, two, or three substituents selected from among alkyl, alkoxy, alkoxycarbonyl, halogen, and haloalkoxy. In other embodiments, R³ is hydrogen; X² is alkylene or alkenylene; and R² is naphthyl, phenyl, cycloalkyl, heteroaryl, or heterocycloalkyl optionally substituted with methyl, methoxy, t-butoxycarbonyl, chloro, fluoro, trifluoromethoxy, or difluoromethoxy. In some other embodiments, R³ is hydrogen; X² is alkylene or alkenylene; and R² is phenyl where the phenyl is optionally substituted with one, two, or three substituents selected from among methyl, methoxy, chloro, fluoro, trifluoromethoxy, and difluoromethoxy; or R² is benzooxadiazolyl.

In some embodiments, R⁴ is hydrogen or alkyl; X⁵ is alkylene or alkenylene; and R⁵ is aryl optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; or R⁵ is heterocycloalkyl optionally substituted with alkoxycarbonyl. In other embodiments, R⁴ is alkyl; X⁵ is alkylene; and R⁵ is phenyl optionally substituted with one, two, or three substituents selected from among acylamino, amino, halogen, and nitro.

In some embodiments, R³ is hydrogen; X² is alkylene or alkenylene; and R² is cycloalkyl, aryl, heteroaryl, or heterocycloalkyl, where the cycloalkyl is optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro; where the aryl is substituted with one, two, or three substituents selected from among acyloxy, alkyl, substituted alkyl, alkenyl, substituted alkenyl, alkylamino, dialkylamino, and haloalkoxy; where the heteroaryl and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, and nitro.

In some embodiments, R³ is hydrogen; X² is alkylene or alkenylene; and R² is cycloalkyl; phenyl substituted with one, two, or three alkyl or haloalkoxy; benzooxadiazolyl; or piperidinyl optionally substituted with alkoxycarbonyl. In some other embodiments, R³ is hydrogen; X² is alkylene or alkenylene; and R² is benzooxadiazolyl or phenyl where the phenyl is substituted with one, two, or three substituents selected from among methyl, chloro, fluoro, trifluoromethoxy, or difluoromethoxy.

In some embodiments, R⁴ is hydrogen or alkyl; X⁵ is a bond and R⁵ is heterocycloalkyl optionally substituted with alkoxycarbonyl; or X⁵ is alkylene or alkenylene and R⁵ is aryl substituted with one, two, or three substituents selected from among acyl, acylamino, acyloxy, alkyl substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In yet other embodiments, R⁴ is hydrogen or alkyl; X⁵ is alkylene; and R⁵ is phenyl substituted with one, two, or three substituents selected from among acyl, acylamino, acyloxy, alkyl substituted alkyl, alkenyl, substituted alkenyl, alkoxy, alkoxycarbonyl, amino, alkylamino, dialkylamino, carboxy, cyano, halogen, haloalkoxy, and nitro. In yet other embodiments, R⁴ is hydrogen or alkyl; X⁵ is alkylene; and R⁵ is phenyl substituted with one, two, or three substituents selected from among optionally substituted arylcarbonylamino, amino, halogen, and nitro.

Any combination of the groups described above for the various variables is contemplated herein.

Further embodiments of compounds described herein (e.g. 1,3-disubstituted-1H-indole-6-thiohydroxamic acid compounds; 1,3-disubstituted-1H-indole-5-thiohydroxamic acid compounds; Formula (IIIa); Formula (IIIb); Formula (IVa); Formula (IVb)) include, but are not limited to, compounds in Table VIII and Table IX.

TABLE VIII
1,3-substituted-1H-indole-6-carboxylic acid hydroxyamides.

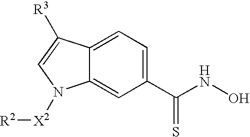

| Compound No. | R² | R³ | X² |
|---|---|---|---|
| VIII-1 | 3,4-dichlorophenyl | H | —CH₂— |
| VIII-2 | 2-methylphenyl | H | —CH₂— |
| VIII-3 | 3,4,5-trimethoxyphenyl | H | —CH₂— |
| VIII-4 | 3-fluorophenyl | H | —CH₂— |
| VIII-5 | 3-methylphenyl | H | —CH₂— |
| VIII-6 | phenyl | H | —CH₂— |
| VIII-7 | 3,5-dimethoxyphenyl | H | —CH₂— |
| VIII-8 | phenyl | H | —CH(CH₃)— |
| VIII-9 | 4-fluorophenyl | H | —CH₂— |
| VIII-10 | 2-fluorophenyl | H | —CH₂— |
| VIII-11 | 2-chlorophenyl | H | —CH₂— |
| VIII-12 | 3-methoxyphenyl | H | —CH₂— |
| VIII-13 | naphth-2-yl | H | —CH₂— |
| VIII-14. | phenyl | H | —(CH₂)₃— |
| VIII-15 | cyclohexyl | H | —CH₂— |
| VIII-16 | phenyl | H | —CH═CHCH₂— |
| VIII-17 | 4-(trifluoromethoxy)-phenyl | H | —CH₂— |
| VIII-18 | 4-chlorophenyl | H | —CH₂— |
| VIII-19 | benzo[2,1,3]oxadiazol-5-yl | H | —CH₂— |
| VIII-20 | 4-methylphenyl | H | —CH₂— |
| VIII-21 | 3-fluoro-4-methoxy-phenyl | H | —CH₂— |
| VIII-22 | 4-(difluoromethoxy)-phenyl | H | —CH₂— |
| VIII-23 | 4-methoxyphenyl | H | —CH₂— |
| VIII-24 | phenyl | H | —CH₂CH₂— |
| VIII-25 | 3-chlorophenyl | H | —CH₂— |
| VIII-26 | N-(t-butoxycarbonyl)piperidin-4-yl | H | —CH₂— |
| VIII-27 | piperidin-4-yl | H | —CH₂— |
| VIII-28 | N-methylsulfonyl-3-aminophenyl | H | —CH₂— |
| VIII-29 | 4-methoxyphenyl | dimethylaminomethyl | —CH₂— |
| VIII-30 | 4-methoxyphenyl | N-morpholinomethyl | —CH₂— |
| VIII-31 | 4-methoxyphenyl | N-pyrrolidinomethyl | —CH₂— |
| VIII-32 | 4-methoxyphenyl | N-benzylaminomethyl | —CH₂— |
| VIII-33 | 4-methoxyphenyl | ethyl | —CH₂— |

Compounds in Table VIII are named:
1-(3,4-dichloro-phenylmethyl)-1H-indole-6-thiohydroxamic acid (Compound VIII-1);
1-(2-methyl-phenylmethyl)-1H-indole-6-thiohydroxamic acid (Compound VIII-2);
1-(3,4,5-trimethoxy-phenylmethyl)-1H-indole-6-thiohydroxamic acid (Compound VIII-3);
1-(3-fluoro-phenylmethyl)-1H-indole-6-thiohydroxamic acid (Compound VIII-4);
1-(3-methyl-phenylmethyl)-1H-indole-6-thiohydroxamic acid (Compound VIII-5);
1-(benzyl)-1H-indole-6-thiohydroxamic acid (Compound VIII-6);
1-(3,5-dimethoxy-phenylmethyl)-1H-indole-6-thiohydroxamic acid (Compound VIII-7);
1-(1-methyl-1-phenylmethyl)-1H-indole-6-thiohydroxamic acid (Compound VIII-8);
1-(4-fluoro-phenylmethyl)-1H-indole-6-thiohydroxamic acid (Compound VIII-9);
1-(2-fluoro-phenylmethyl)-1H-indole-6-thiohydroxamic acid (Compound VIII-10);
1-(2-chloro-phenylmethyl)-1H-indole-6-thiohydroxamic acid (Compound VIII-11);
1-(3-methoxy-phenylmethyl)-1H-indole-6-thiohydroxamic acid (Compound VIII-12);
1-(naphth-2-ylmethyl)-1H-indole-6-thiohydroxamic acid (Compound VIII-13);
1-(3-phenylpropyl)-1H-indole-6-thiohydroxamic acid (Compound VIII-14);
1-(cyclohexylmethyl)-1H-indole-6-thiohydroxamic acid (Compound VIII-15);
1-[1-(phenyl)-propen-3-yl]-1H-indole-6-thiohydroxamic acid (Compound VIII-16);
1-[4-(trifluoromethoxy)-phenylmethyl]-1H-indole-6-thiohydroxamic acid (Compound VIII-17);
1-(4-chloro-phenylmethyl)-1H-indole-6-thiohydroxamic acid (Compound VIII-18);
1-(benzo[2,1,3]oxadiazol-5-ylmethyl)-1H-indole-6-thiohydroxamic acid (Compound VIII-19;
1-(4-methyl-phenylmethyl)-1H-indole-6-thiohydroxamic acid (Compound VIII-20);
1-(3-fluoro-4-methoxy-phenylmethyl)-1H-indole-6-thiohydroxamic acid (Compound VIII-21);
1-[4-(difluoromethoxy)-phenylmethyl]-1H-indole-6-thiohydroxamic acid (Compound VIII-22);
1-(4-methoxy-phenylmethyl)-1H-indole-6-thiohydroxamic acid (Compound VIII-23);
1-(phenethyl)-1H-indole-6-thiohydroxamic acid (Compound VIII-24);
1-(3-chloro-phenylmethyl)-1H-indole-6-thiohydroxamic acid (Compound VIII-25);
1-[N-(t-butoxycarbonyl)piperidin-4-ylmethyl]-1H-indole-6-thiohydroxamic acid (Compound VIII-26);
1-(piperidin-4-ylmethyl)-1H-indole-6-thiohydroxamic acid (Compound VIII-27);
1-(N-methylsulfonyl-3-aminobenzyl)-1H-indole-6-thiohydroxamic acid (Compound VIII-28);
3-(Dimethylaminomethyl)-1-(4-methoxybenzyl)-1H-indole-6-thiohydroxamic acid (Compound VIII-29);
3-(N-Morpholinomethyl)-1-(4-methoxybenzyl)-1H-indole-6-thiohydroxamic acid (Compound VIII-3 0);
3-(N-Pyrrolidinomethyl)-1-(4-methoxybenzyl)-1H-indole-6-thiohydroxamic acid (Compound VIII-31);
3-(N-Benzylaminomethyl)-1-(4-methoxybenzyl)-1H-indole-6-thiohydroxamic acid (Compound VIII-32); and
3-(Ethyl)-1-(4-methoxybenzyl)-1H-indole-6-thiohydroxamic acid (Compound VIII-33).

Also described herein are pharmaceutically acceptable salts of compounds of Table VIII.

TABLE IX
1,3-substituted-1H-indole-5-carboxylic acid hydroxyamides

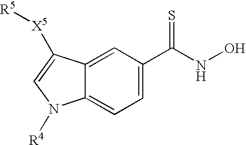

| Compound No. | R⁴ | X⁵ | R⁵ |
|---|---|---|---|
| IX-1 | methyl | —CH₂— | 4-nitrophenyl |
| IX-2 | ethyl | —CH₂— | phenyl |
| IX-3 | methyl | —CH₂— | 4-(phenylcarbonylamino)-phenyl |
| IX-4 | isopropyl | —CH₂— | phenyl |
| IX-5 | methyl | —CH₂— | 4-aminophenyl |
| IX-6 | methyl | —CH₂— | 4-fluorophenyl |
| IX-7 | phenyl | —CH₂— | phenyl |
| IX-8 | methyl | —CH₂— | 4-(t-butoxycarbonyl)piperazin- |
| | | | 1-yl |

Compounds in Table IX. are named:
1-methyl-3-(4-nitro-phenylmethyl)-1H-indole-5-thiohydroxamic acid (Compound IX-1);
1-ethyl-3-(phenylmethyl)-1H-indole-5-thiohydroxamic acid (Compound IX-2);
1-methyl-3-[4-(phenylcarbonylamino)-phenylmethyl]-1H-indole-5-thiohydroxamic acid (Compound IX-3);
1-isopropyl-3-(phenylmethyl)-1H-indole-5-thiohydroxamic acid (Compound IX-4);
1-methyl-3-(4-amino-phenylmethyl)-1H-indole-5-thiohydroxamic acid (Compound IX-5);
1-methyl-3-(4-fluoro-phenylmethyl)-1H-indole-5-thiohydroxamic acid (Compound IX-6);
1-phenyl-3-(phenylmethyl)-1H-indole-5-thiohydroxamic acid (Compound IX-7); and
1-methyl-3-[4-(t-butoxycarbonyl)piperazin-1-ylmethyl]-1H-indole-5-thiohydroxamic acid (Compound IX-8).

Also described herein are pharmaceutically acceptable salts of compounds of Table IX.

Throughout the specification, groups and substituents thereof can be chosen by one skilled in the field to provide stable moieties and compounds.

### Further Forms of Compounds

Compounds described herein may possess one or more stereocenters and each center may exist in the R or S configuration. The compounds presented herein include all diastereomeric, enantiomeric, and epimeric forms as well as the appropriate mixtures thereof. Separation of steroisomers may be performed by chromatography. Alternatively, individual stereoisomers may be obtained by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are also possible (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or by separation/resolution techniques based upon differences in solubility. The optically pure enantiomer(s) is/are then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981, herein incorporated by reference for this disclosure. Stereoisomers may also be obtained by stereoselective synthesis.

In some situations, compounds may exist as tautomers. All tautomers are included within the formulas described herein.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds described herein in unoxidized form can be prepared from the corresponding N-oxides compounds by treating with a reducing agent, such as, but not limited to, sulfur, sulfur dioxide, triphenyl phosphine, lithium borohydride, sodium borohydride, phosphorus trichloride, phosphorus tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prod rug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prod rugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure.

Sites on the aromatic ring portion of compounds described herein can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶C, respectively. Certain isotopically-labeled compounds described herein, for example those into which radioactive isotopes such as H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion. In some cases, compounds described herein may form coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

### Synthesis of Compounds

The synthesis of compounds described herein may be accomplished using means described in the chemical literature, using the methods described herein, or by a combination thereof. In addition, solvents, temperatures and other reaction conditions presented herein may vary according to those of skill in the art.

The starting materials and reagents used for the synthesis of the compounds described herein may be synthesized or can be obtained from commercial sources, such as, but not limited to, Aldrich Chemical Co. (Milwaukee, Wis.), Sigma Chemical Co. (St. Louis, Mo.), or Bachem (Torrance, Calif.).

The compounds described herein, and other related compounds having different substituents can be synthesized using techniques and materials described herein as well as those described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999), US patent publication 20050187261, US patent publication 20050131018 (all of which are incorporated by reference for such disclosure). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

The compounds prepared by the methods disclosed herein are purified by conventional means known in the art, such as, for example, filtration, recrystallization, chromatography, distillation, and combinations thereof.

Formation of Covalent Linkages by Reaction of an Electrophile with a Nucleophile

The compounds described herein can be modified using various electrophiles and/or nucleophiles to form new functional groups or substituents. Table 3 entitled “Examples of Covalent Linkages and Precursors Thereof” lists selected non-limiting examples of covalent linkages and precursor functional groups which yield the covalent linkages. Table 3 may be used as guidance toward the variety of electrophiles and nucleophiles combinations available that provide covalent linkages. Precursor functional groups are shown as electrophilic groups and nucleophilic groups.

TABLE 3
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In the reactions described, it may be necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. It is preferred that each protective group be removable by a different means. Protective groups that are cleaved under totally disparate reaction conditions fulfill the requirement of differential removal.

Protective groups can be removed by acid, base, reducing conditions (such as, for example, hydrogenolysis), and/or oxidative conditions. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, which include conversion to alkyl esters, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

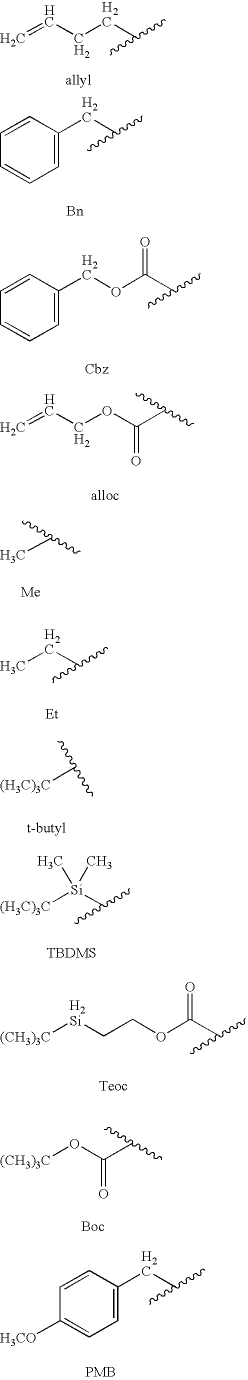

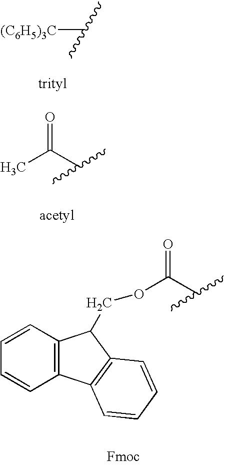

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### General Synthesis

Non-limiting methods for the preparation of thiohydroxamic acid functional groups include the following:

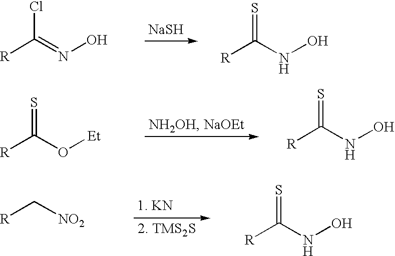

Non-limiting examples of reactions conditions for the synthesis of thiohydroxamic functional groups are described in the following: Hwu, Jih Ru, Tsay, Shwu Chen., “Counterattack reagents: thiosilanes in the conversion of nitro compounds to thiohydroxamic acids and thiohydroximates.” Tetrahedron (1990), 46(21), 7413-28; Purohit, Bharat Kumar, et al., “Metal(II) complexes of thioformohydroxamic acid and their antifungal activity.” Anales de Quimica, Serie B: Quimica Inorganica y Quimica Analitica (1989), 85(2), 190-1; Mathur, S. P.; Sharma, Bharat Kurnar; Dubey, S. P.; Sharma, R. C. “Preparation and insecticidal activity of thioanalogs of hydroxylamine derivatives.” Acta Ciencia Indica, Chemistry (1985), 11(2), 124-5; Mitchell Annabel J. Murray, Keith S Newman Peter J. Clark, Paul E “Thiohydroxamato complexes of iron(III). Synthesis, magnetism and Moessbauer spectra.” Australian Journal of Chemistry (1977), 30(11), 2439-53; Zagrebina, R. F. “Thioglycohydroxamic acid, a reagent for determination and separation of a series of elements.” USSR. Tr. Molodykh. Uch. Saratov. Univ., Vyp. Khim. (1971), No. 2 138-42; Sato, Tomohiro; et al., “Metal complexes of thiohydroxamic acid. II. Crystal structure of bis(thioacetohydroxamato)nickel(II). Journal of the Chemical Society [Section] B: Physical Organic (1969), 2 125-30; Mizukami, Satoshi; Nagata, Koichi. “Thiohydroxamic acids and their metal chelates. I. Synthesis of thiohydroxamic acids and O-methylthiohydroxamic acids.” Chemical & Pharmaceutical Bulletin (1966), 14(11), 1249-55; Bacchetti, Tullio, Alemagna, Andreina. “Thiohydroxamic acids. III. New synthesis from chlorohydroxamic acids.” Atti accad. nazl. Lincei. Rend., Classe sci. fis., mat. e nat. (1958), 24 161-3.

In one embodiment, compounds described herein may be synthesized with a carboxylic acid moiety or an ester of a carboxylic ester in place, which is then converted to the corresponding thiohydroxamic acid moiety using methods known in the art. For example, esters of carboxylic acids may be converted to thiohydroxamic acids using the two step sequence shown below. Treatment of the carboxylic ester with P₄S₁₀ is followed by treatment with a base and hydroxylamine. In the first step of the two step sequence, other ester groups (—C(═O)O— or OC(═O)—), ketone groups (—C(═O)—) or amide groups (—NHC(═O)— or C(═O)NH—) that are present on R may react with P₄S₁₀ to provide the analogous sulfur containing functional groups (e.g. —C(═S)O— or OC(═S)— or —C(═S)— or —NHC(═S)— or —C(═S)NH—). Thus, a single —C(═S)— may exist in the final product, or multiple —C(═S)— may exist in the final product.

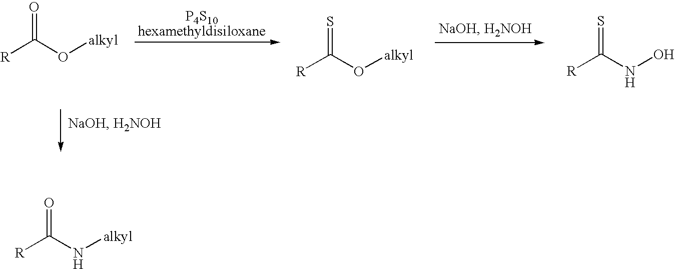

For comparison, esters of carboxylic acids may be treated with base and hydroxylamine to furnish hydroxamic acid compounds.

### Synthesis of Compounds of Formula (I)

Compounds of Formula (I) where Z is —CONR³—, —SO₂NR³—, —NR³CONR⁴—, —NR³SO₂NR⁴—, or OCONR³— can by following the sequence shown in Scheme I-A below.

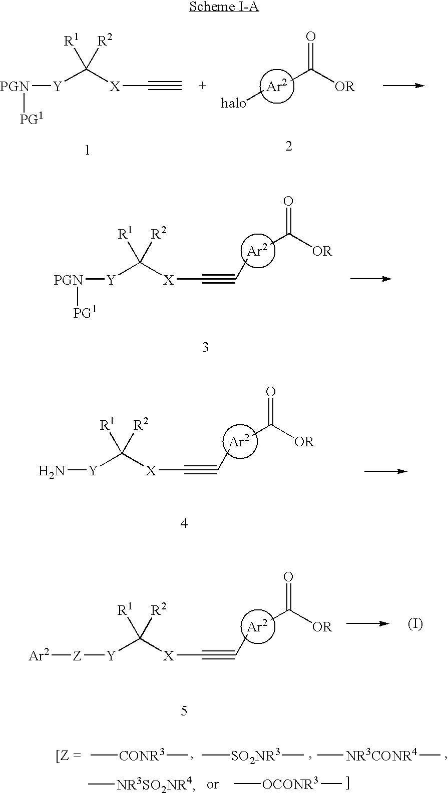

Alkynes of structure 1 (where PG is a suitable amino protecting group and PG¹ is hydrogen or a suitable amino protecting group such as, but not limited to, tert-butoxycarbonyl, benzyloxycarbonyl), with a compound of formula 2 (where R is alkyl, e.g methyl or ethyl), provides a compound of formula 3. The reaction is carried out in a suitable organic solvent such as dimethylformamide and in the presence of an organic base such as triethylamine, and the like and a suitable catalyst such as PdCl₂(Ph₃P)₂, and the like.

Compounds of formula 1 can be prepared from commercially available starting materials by known methods. For example, N-tert-butoxycarbonyl-propargylamine can be prepared by reacting commercially available propargylamine with tert-butoxycarbonyl anhydride in the presence of a base such as triethylamine, ethylisopropylamine, and the like. Compounds of formula 1 can also be prepared from N-protected amino acids by first reducing the acid group to give the corresponding aldehyde by known methods and then reacting the aldehyde with Ohira reagent (see Ohira, S. Synth. Comm n., 19, 561-564, (1989)) to give a compound of formula 1. Amino acids suitable for preparing compounds of formula 1 are commercially available. For example, 1-amino-cyclobutanecarboxylic acid, homoalanine, aspartic acid, gamma-n-butyric acid, 3-amino-3-phenylpropionic acid, 4-amino-2,2-dimethyl butyric acid, piperidine-3-carboxylic acid, 4-aminopiperidine-4-carboxylic acid, 4-amino-4-carboxytetrahydropyran, 2-aminocyclopentaneacetic acid, and 2-amino-1-cyclopentanecarboxylic acid are commercially available. Alpha and beta amino acids can also be prepared by methods described in Duthaler, R. O. Tetrahedron, 50, 1539-1650 (1994) and Cole, D.C. Tetrahedron, 50, 9517-9582, (1994), which are incorporated herein by reference for such disclosure.

Compounds of formula 1 where PG¹ is an amino protecting group can also be prepared from commercially available alcohols under Mitsunobu reaction conditions.

Removal of the amino-protecting group provides a compound of formula 4. The reaction conditions employed depend on the nature of the protecting group. For example, if the amino-protecting group is tert-butoxycarbonyl, it is removed by treating a compound of formula 3 with an acid such as trifluoroacetic acid, hydrochloric acid, and the like, in a suitable organic solvent such as dioxane, tetrahydrofuran, methanol, dichloromethane, and the like.

Compounds of formula 2 are either commercially available or they can be prepared by known methods. For example, methyl 4-iodobenzoate is commercially available. 4-Iodo-2-thiophenecarboxylic methyl ester can be prepared from commercially available 4-iodo-2-thiophenecarboxylic acid under standard esterification reaction conditions.

Compound 4 is then converted to a compound of formula 5 where Z is —CONR³—, —SO₂NR³—, —NR³CONR⁴—, —NR³SO₂NR⁴—, or OCONR³— by known methods.

A compound of formula 5 where Z is —CONH— or —SO₂NH— is prepared by reacting a compound of formula 4 with an acylating or sulfoylating agent of formula Ar²COL or Ar²SO₂L respectively, where L is a leaving group under acylating or sulfonylating reaction conditions such as halo (e.g. chloro or bromo). Suitable for solvents for the reaction include organic solvents such as dichloromethane, tetrahydrofuran, dioxane, dimethylformamide, and the like. The reaction is carried out in the presence of an organic base such as triethyamine, pyridine, and the like. Acylating or sulfonylating agent of formula Ar²COL or Ar²SO₂L are either commercially available or they can be readily prepared. For example, Ar²COL can be prepared by reacting the corresponding acids with a halogenating agent such as oxalyl chloride, thionyl chloride, and the like.

Alternatively, a compound of Formula (I) where Z is —CONH— can be prepared by heating 4 with an acid anhydride. Suitable solvents for the reaction are tetrahydrofuran, dioxane, and the like.

Alternatively, a compound of Formula (I) when Z is —CONH— can be prepared by reacting an acid of formula Ar²—COOH in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC.HCl), or 1,3-dicyclohexylcarbodiimide (DCC), optionally in the presence of 1-hydroxybenzotriazole hydrate (HOBt-H₂O), and a base such as N,N-diisopropylethylamine, triethylamine, N-methylmorpholine, and the like. The reaction is typically carried out at 20 to 30° C., and requires 2 to 24 h to complete. Suitable reaction solvents are inert organic solvents such as halogenated organic solvents (e.g., methylene chloride, chloroform, and the like), acetonitrile, dimethylformamide, ethereal solvents such as tetrahydrofuran, dioxane, and the like. Acids of formula Ar²—COOH such as benzoic acid, cinnamic acid, phenylacetic acid, nicotinic acid, isonicotinic acid, and benzofuran-2-carboxylic acid are commercially available. Others such as 3-phenoxymethylbenzofuran-2-carboxylic acid can be readily prepared from commercially available 3-methylbenzofuran-2-carboxylic acid by first converting it to 2-bromomethylbenzofuran-2-carboxylic acid (brominating it with N-bromosuccinimide under known conditions) followed by reacting with phenol.

A compound of formula 5 where Z is NR³CONH— is prepared by reacting a compound of formula 4 with an activating agent such as carbonyl diimidazole, followed by displacement of the imidazole group with a primary or secondary amine of formula Ar²NHR⁷. Suitable reaction solvents include tetrahydrofuran, dioxane, and the like.

Alternatively, a compound of formula 5 where Z is —NR⁷CONH— is prepared by reacting compound 4 with a carbamoyl halide of formula Ar²NR⁷COL or an isocyanate of formula Ar²N═C═O under known conditions.

A compound of formula 5 where Z is NR⁹SO₂NH— is prepared by reacting a compound of formula 4 with a sulfamoyl halide of formula Ar²NR⁹SO₂L under reaction conditions described above. Sulfamoyl halides are either commercially available or may be prepared by methods such as those described in Graf, R., German Patent 931225 and Catt, J. D. and Matler, W. L., J. Org. Chem., 1974, 39, 577-568.

A compound of formula 5 where Z is OC(O)NH— is prepared by reacting a compound of formula 4 with acylating agent of formula Ar²OC(O)L under reaction conditions described above.

Conversion of compound of formula 5 to compounds of Formula (I) uses reactions known in the art. Esters of structure 5 are treated with P₄S₁o and hexamethyldisiloxane (HMDO) in xylenes and refluxed to provide the corresponding thioesters. The thioester compounds are treated with a base, such as sodium hydroxide, and hydroxylamine in an alcohol solvent to furnish the thiohydroxamic acid compounds.

### Synthesis of Compounds of Formula (II)

Compounds of Formula (II) can be prepared by the procedure outlined in Scheme II-A.

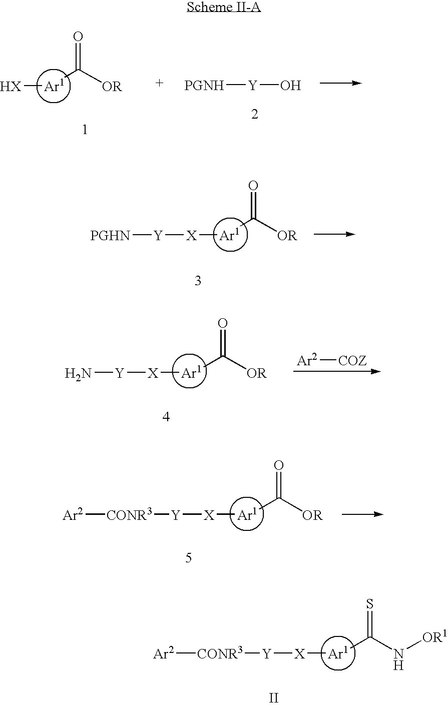

Reaction of a compound of structure 1, where R is alkyl, X is O— or S—; and Ar¹ is as defined herein with an aminoalcohol of structure 2, where PG is a suitable amino protecting group provides a compound of structure 3. The reaction is carried out in the presence of triphenylphosphine and diisopropyl azodicarboxylate in a suitable organic solvent such as, but not limited to, tetrahydrofuran.

Compounds of structure 1, such as, but not limited to methyl 4-hydroxybenzoate, methyl 4-mercaptobenzoate, and methyl 3-hydroxyisoxazole-5-carboxylate, are commercially available. Compounds of structure 2 can be prepared from commercially available aminoalcohols by reacting the free amine with a suitable amino protecting group such as, but not limited to benzyloxycarbonyl and tert-butoxycarbonyl, under known conditions. A description of suitable amino protecting groups and reaction conditions for their preparation can be found in T. W. Greene, Protecting Groups in Organic Synthesis, John Wiley & Sons, Inc. 1981 the teaching of which is incorporated herein by reference for this purpose. Aminoalcohols such as 2-ethanolamine, 2-amino-1-propanol, 2-methylaminoethanol, 2-amino-2-methyl-1-propanol, 2-amino-1-propanol, 4-amino-2-butanol, and 1-amino-2-butanol are commercially available and may be used to prepare compounds of structure 2. Alternatively, compounds of structure 2 can be prepared from commercially available aminoacids by protecting the amino group with a suitable protecting group followed by reduction of the acid group to the hydroxy group with a suitable reducing agent under known conditions. If compounds of Formula (II) where X is —SO₂— are desired, the corresponding compound of structure 3 where X is S— can be treated with an oxidizing agent such as OXONE®, m-chloroperbenzoic acid, and the like.

Removal of the amino protecting group in compounds of structure 3 provides a compound of structure 4. The reaction conditions employed for removal of the amino protecting group depend on the nature of the protecting group. For example, if the amino protecting group is tert-butoxycarbonyl, it is removed under acid reaction conditions. Suitable acids for the removal of a tert-butoxycarbonyl group from an amino include, but are not limited to, trifluoroacetic acid, hydrochloric acid. If the amino protecting group is benzyl or benzyloxycarbonyl, it can be removed under catalytic hydrogenation reaction conditions. Suitable catalyst for the removal of benzyl or benzyloxycarbonyl groups from amino moieties include, but are not limited to, palladium based catalysts. Other suitable reaction conditions for their removal can be found in T. W. Greene, Protecting Groups in Organic Synthesis, John Wiley & Sons, Inc. 1981. The reaction can be carried out in an inert organic solvent such as, but not limited to, methylene chloride, tetrahydrofuran, dioxane.

Reaction of compounds of structure 4 with an acid or acid derivative (e.g., Ar²—COZ where Z is hydroxy or halo) provides a compound of structure 5. The reaction conditions employed depend on the nature of the Z group. If Z is hydroxy, then the reaction is typically carried out in the presence of a suitable coupling agent e.g., benzotriazole-1-yloxytrispyrrolidino-phosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC. HCl), or 1,3-dicyclohexylcarbodiimide (DCC), optionally in the presence of 1-hydroxybenzotriazole hydrate (HOBT. H₂O), and a base such as N,N-diisopropylethylamine, triethylamine, N-methylmorpholine, and the like. The reaction is typically carried out at 20 to 30° C., and requires 2 to 24 hours to complete. Suitable reaction solvents include inert organic solvents such as halogenated organic solvents (e.g., methylene chloride, chloroform, and the like), acetonitrile, N,N-dimethylformamide, ethereal solvents such as tetrahydrofuran, dioxane, and the like. Preferably, the reaction is carried out with HOBt. H₂O, EDC.HCl in dichloromethane or N,N-dimethylformamide.

When Ar²COZ is an acid halide, the reaction is carried out in the presence of a suitable base (e.g. triethylamine, diisopropylethylamine, pyridine, and the like). Suitable reaction solvents include polar organic solvents such as tetrahydrofuran, acetonitrile, N,N-dimethylformamide (DMF), dichloromethane, or any suitable mixtures thereof. The acid halide can be prepared by reacting the corresponding acids with a halogenating agent such as oxalyl chloride, thionyl chloride, phosphorus oxychloride, and the like. Acids of structure Ar²COZ are either commercially available or they can be prepared from commercially available starting materials by methods known in the art. For example, benzoic acid, cinnamic acid, phenylacetic acid, nicotinic acid, isonicotinic acid, 3-methylbenzofuran-2-carboxylic acid, and benzofuran-2-carboxylic acid are commercially available. Others such as 3-phenoxymethylbenzofuran-2-carboxylic acid can be readily prepared from commercially available 3-methylbenzofuran-2-carboxylic acid by first converting it to 2-bromomethylbenzofuran-2-carboxylic acid (brominating it with N-bromosuccinimide under known conditions) followed by reacting with phenol. Compound of structure 5 where R³ is hydrogen can optionally be converted to a corresponding compound of structure 5 where R³ is other than hydrogen by reacting it with an alkylating agent under known conditions.

Compounds of structure 5 can then be converted into compounds of Formula (II) by converting the ester to a thioester, using for example, P₄S₁₀ and hexamethyldisiloxane. The thioester can then be reacted with aqueous hydroxylamine in the presence of a base such as sodium hydroxide and a mixture of organic solvents such as tetrahydrofuran and methanol.

### Compounds of Formula IIIa, IIIb, IVa, IVb

Indole compounds described herein may be prepared from commercially available materials or they may be prepared by methods known in the art.

Indole compounds described herein may be synthesized starting from indole compounds that are available from commercial sources or they can be prepared using procedures known in the art or outlined herein.

Using the reaction conditions described herein, 1,3-substituted-1H-indole-5-thiohydroxamic acids and 1,3-substituted-1H-indole-6-thiohydroxamic acids as disclosed herein are obtained in good yields and purity. The compounds prepared by the methods disclosed herein are purified by conventional means known in the art, such as, for example, filtration, recrystallization, chromatography, distillation, and combinations thereof.

In one embodiment, compounds of structure 1 and structure 2 are used as starting materials for the synthesis of compounds described herein.

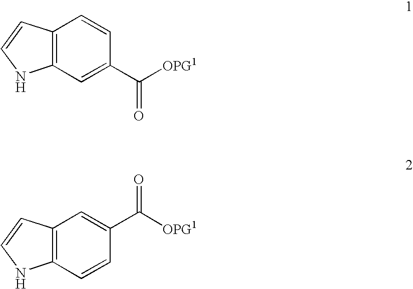

PG¹ represents carboxylic acid protecting groups. In one embodiment, PG¹ represents a substituted or unsubstituted alkyl group, such as, but not limited to, methyl, ethyl, propyl, benzyl, and p-methoxybenzyl.

Indoles of general structure 1 and structure 2 may also be prepared by methods known in the art. Indole containing compounds described herein can be prepared using standard literature procedures such as those found in Katritzky, “Handbook of Heterocyclic Chemistry” Pergamon Press, Oxford, 1986; Pindur et al, J. Heterocyclic Chem., vol 25, 1, 1987, and Robinson “The Fisher Indole Synthesis”, John Wiley & Sons, Chichester, New York, 1982, each of which is herein incorporated by reference for such disclosure.

Additional non-limiting examples of synthetic strategies toward the synthesis of indole compounds described herein, include modifications to various syntheses of indoles, including, but not limited to: Batcho-Leimgruber Indole Synthesis, Reissert Indole Synthesis, Hegedus Indole Synthesis, Fukuyama Indole Synthesis, Sugasawa Indole Synthesis, Bischler Indole Synthesis, Gassman Indole Synthesis, Fischer Indole Synthesis, Japp-Klingemann Indole Synthesis, Buchwald Indole Synthesis, Larock Indole Synthesis, Bartoli Indole Synthesis, Castro Indole Synthesis, Hemetsberger Indole Synthesis, Mori-Ban Indole Synthesis, Madelung Indole Synthesis, Nenitzescu Indole Synthesis, and other unnamed reactions.

In one embodiment, the functionalization of the 1-position and/or 3-position of indoles of structure 1 and structure 2 may be achieved by using any of the indole forming reactions mentioned above with appropriate starting materials.

In another embodiment, the 1-position of indoles described herein may be functionalized as outlined in Scheme 1.

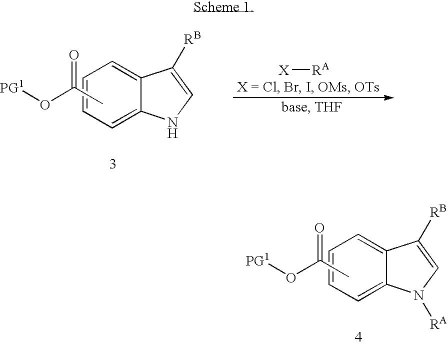

Indoles of general structure 4 (where R^{B} is H, R³, or —X⁵—R⁵; R^{A} is R⁴ or —X²—R²) are obtained from the N-alkylation of indoles of structure 3 with, for example, an alkyl halide (or benzyl halide, or tosylate (OTs) or mesylate (OMs), or carboxylic acid halide) in a solvent such as tetrahydrofuran (THF) or dimethylformamide (DMF) in the presence of a base, such as, for example, NaH or potassium carbonate or sodium carbonate. In other embodiments, N-arylation of indoles can be achieved using a metal mediated cross coupling of N—H indoles of general structure 3 with aryl halides or triflates (R^{A} is aryl, heteroaryl; Old et al. Org. Lett., 2 (10), 1403-1406, 2000.).

In addition, when R^{B} is a bromo or iodine, standard cross coupling reactions allow the introduction of a variety of functional groups using known procedures. Indoles of structure 3, where R^{B} is a halide can be prepared using standard bromination conditions or iodination conditions. Metal mediated coupling reactions include, but are not limited to Suzuki reactions, Sonogashira couplings, Heck reactions, Stille cross couplings, Negishi couplings, Kumada couplings, Ullmann reactions, Buchwald-Hartwig reactions, and variants thereof (Metal-Catalyzed Cross-Coupling Reactions, Armin de Meijere (Editor), François Diederich (Editor), John Wiley & Sons; 2nd edition, 2004).

Other non-limiting approaches to the functionalization of indoles at the 1-position and/or 3-position are shown in scheme 2.

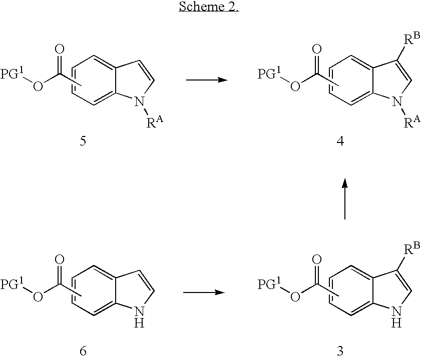

Functionalzation at the 3-position of 3-H-indoles of structure 5 (R^{A} is R⁴ or —X²—R²) can be achieved using a variety of reactions and procedures to allow the introduction of a wide range of substituents. By way of example only, acylation using an acid chloride (or anhydride) in the presence of a Lewis acid such as AlCl₃, allows for the introduction of acyl groups at the 3-position of indoles (Murakami et al. Heterocycles, v14, 1939-1941, 1980 and references cited therein.). Selective reduction of the carbonyl at the 3-position of the indole provides compounds of structure 4 (where R^{B} is R³, or —X⁵—R⁵, which is a substituted or unsubstituted alkyl; R^{A} is R⁴ or —X²—R²).

The reaction of electron deficient olefins with 3-H indoles of structure 5 (R^{A} is R⁴ or —X²—R²) or structure 6 in the presence of a Lewis acid (such as, for example, Yb(OTf)₃.3H₂O) allows the installation of alkyl substituents at the 3-position of the indole compounds to provide indoles of the general structure 4 or 3 (where R^{B} is R³, or —X⁵—R⁵, which is a substituted alkyl group; see Harrington and Kerr, Synlett, 1047-1048, 1996). Alternatively, indoles of structure 6 can be reacted with benzyl derivatives in warm DMF to yield indoles of structure 3 where R^{B} is R³, or —X⁵—R⁵, which is a substituted benzyl group (Jacobs et al, J. Med. Chem., v36, 394-409, 1993).

In other embodiments, indoles of general structure 5 or 6 can be reacted with methyl ketones in the presence of a base and copper catalyst in order to provide indoles of general structure 3 or 4, where R^{B} is a substituted alkyl.

In other embodiments, compounds of general structure 5 can be reacted with alkyl halides in the presence of a lewis acid, such as, silver oxide, to provide compounds of general structure 4.

As shown in Scheme 3, 3-formyl indoles of general structure 7 can be condensed with a variety of amines in the presence of a hydride source to provide substituted 3-aminoalkyls of general structure 8.

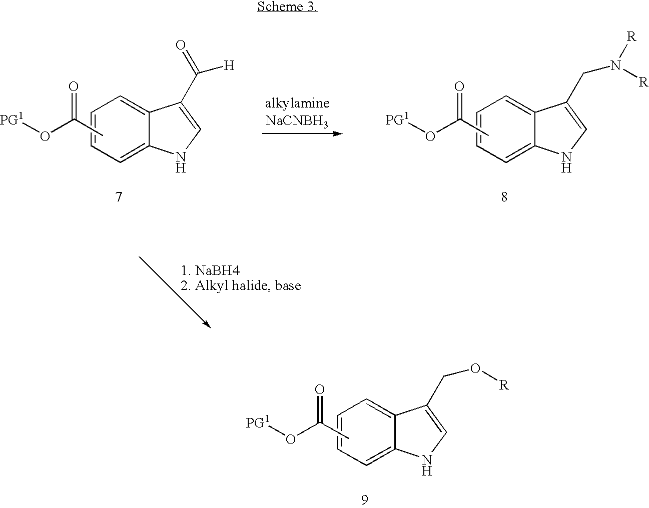

In other embodiments, 3-formyl indoles of general structure 7 can be reduced to the alcohol by treatment with a mild hydride source, such as, but not limited to, sodium borohydride. The alcohol can be coupled with a variety of electrophiles, such as, but not limited to, alkyl halides, carboxylic acid halides, to provide compounds of structure 9. 3-Formyl indoles of structure 7 may be prepared using the Vilsmeir reaction or are commercially available.

Conversion of the indoles of general structure 4 (where R^{B} is R³ or —X⁵—R⁵; R^{A} is R⁴ or —X²—R²) to the corresponding 1,3-substituted-1H-indole-5-thiohydroxamic acid or 1,3-substituted-1H-indole-6-thiohydroxamic acid is shown in Scheme 4.

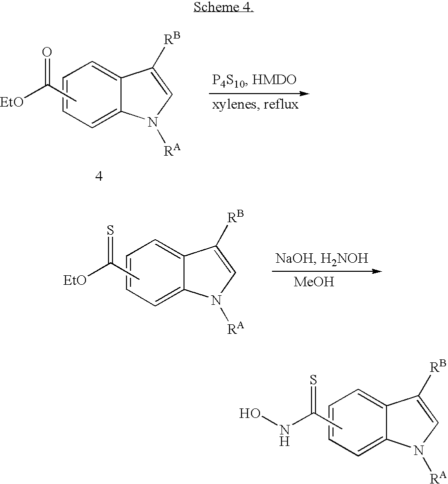

Indoles of structure 4 are treated with P₄S₁o and hexamethyldisiloxane (HMDO) in xylenes and refluxed to provide the corresponding thioesters. The thioester compounds are treated with a base, such as sodium hydroxide, and hydroxylamine in an alcohol solvent to furnish the thiohydroxamic acid compounds.

Throughout the specification, groups and substituents thereof can be chosen by one skilled in the field to provide stable moieties and compounds.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry terms may be found in books, such as Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. In addition, nucleic acid and amino acid sequences for HDAC8 are known in the art as disclosed in, e.g., U.S. Pat. No. 6,875,598. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ, C₁-Cₓ refers to the number of carbon atoms that make up the moiety to which it designates (excluding optional substitutents).

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl, iso-pentyl, neo-pentyl, and hexyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Alkyl groups can be substituted or unsubstituted. Depending on the structure, an alkyl group can be a monoradical or a diradical (i.e., an alkylene group).

An “alkoxy” group refers to a (alkyl)O— group, where alkyl is as defined herein. Examples of alkoxy groups include, but are not limited to, methoxy, ethoxy, propoxy, isopropoxy, butyloxy, cyclopropyloxy, cyclopentyloxy, cyclohexyloxy, and the like.

“Hydroxyalkyl” refers to an alkyl group substituted with hydroxy group(s).

“Hydroxyalkoxy” refers to an alkoxy substituted with hydroxy group(s).

“Alkoxyalkyl” refers to alkyl group substituted with alkoxy group(s).

“Alkoxyalkyloxy” refers to an alkoxy group as defined herein substituted with alkoxy group as defined herein.

“Alkoxycarbonyl” refers to a —C(═O)O-(alkyl) group, where alkyl as defined herein. Non-limiting examples of alkoxycarbonyl groups include, e.g., methoxycarbonyl, ethoxycarbonyl, and the like.

“Alkoxycarbonylamino” refers to a NR(C═O)—O-(alkyl), where alkyl is as defined herein and R is H, alkyl, heteroalkyl, haloalkyl, and the like.

The term “alkenyl” refers to a type of alkyl group in which the first two atoms of the alkyl group form a double bond that is not part of an aromatic group. That is, an alkenyl group begins with the atoms —C(R)=CR₂, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group). Alkenyl groups may have 2 to 6 carbons. Alkenyl groups can be substituted or unsubstituted. Depending on the structure, an alkenyl group can be a monoradical or a diradical (i.e., an alkenylene group).

“Alkenylcarbonyl” refers to a —C(O)-(alkenyl) group, where alkenyl is as defined herein.

“Alkenylcarbonyloxy” refers to a —OC(O)-(alkenyl) group, where alkenyl is as defined herein.

“Alkenyloxy” refers to a O-(alkenyl) group, where alkenyl is as defined herein.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. An alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group, or an N-oxide derivative.

The term “alkylamine” or “alkylamino” refers to the N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a N(alkyl)₂ group, where alkyl is as defined herein.

“Aminoalkyl” refers to an alkyl group as is defined herein that is substituted with an amino group.

“Aminoalkoxy” refers to an alkoxy group substituted with an amino group.

“Aminocarbonyl” refers to a —CONH₂ group.

“Aminosulfonyl” means an —S(O)₂NH₂ radical.

The term “alkylaminoalkyl” refers to an alkyl group, as is defined herein, substituted with an alkylamine as is defined herein. “Dialkylaminoalkyl” refers to an alkyl group that is substituted with a dialkylamino group.

“Alkylaminoalkyloxy” refers to a alkoxy substituted with an alkylamine.

“Alkylaminocarbonyl” means a —C(O)R radical where R is alkylamino as defined herein.

“Alkylaminocarbonylamino” refers to —NHC(═O)-(alkylamino).

“Alkylaminocarbonyloxy” refers to —OC(═O)-(alkylamino).

“Alkylaminosulfonyl” refers to —S(═O)₂NHR radical where R is alkyl, as defined herein.

“Alkylcarbonyl” means a —C(═O)R radical where R is alkyl as defined herein.

“Alkylcarbonylamino” means a —NR′C(═O)-(alkyl), where R′ is hydrogen, alkyl haloalkyl, heteroalkyl.

“Alkylcarbonyloxy” means a —OC(═O)R radical where R is alkyl as defined herein.

“Dialkylaminoalkyloxy” refers to a alkoxy substituted with a dialkylamino.

“Dialkylaminocarbonyl” refers to —C(═O)R, where R is dialkylamino.

“Dialkylaminocarbonylamino” refers to —NR′—C(═O)-(dialkylamino), where R′ is hydrogen, alkyl, heteroalkyl, haloalkyl, and dialkylaminocarbonyl as defined herein.

“Dialkylaminocarbonyloxy” means an O(C═O)-(dialkylamino), dialkylaminocarbonyl as defined herein.

“Dialkylaminosulfonyl” refers to —S(O)₂NR₂, where R is alkyl as defined herein.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (A), thereby forming a prodrug. Any amine, or carboxyl side chain on the compounds described herein can be amidified. The procedures and specific groups to make such amides are found in sources such as Greene and Wuts, Protective Groups in Organic Synthesis, 3^{rd} Ed., John Wiley & Sons, New York, N.Y., 1999, which is incorporated herein by reference for such disclosure.

The term “ester” refers to a chemical moiety with formula C(═O)OR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters can be found in sources such as Greene and Wuts, Protective Groups in Organic Synthesis, 3^{rd} Ed., John Wiley & Sons, New York, N.Y., 1999, which is incorporated herein by reference for such disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, phenyl, pyridine, piperidine, morpholine, piperazine, pyridazine, pyrimidine, pyrazine, pyran and thiopyran are 6-membered rings; and cyclopentyl, pyrrolidine, imidazole, oxazole, thiazole, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles can be optionally substituted.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

“Aralkyl” or “arylalkyl” refers to an alkyl group as is defined herein substituted with an aryl group as is defined herein.

“Phenylalkyl” refers to an alkyl substituted with a phenyl.

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring. Cycloalkyl groups include groups having from 3 to 10 ring atoms. Illustrative examples of cycloalkyl groups include, but are not limited to, the following moieties:

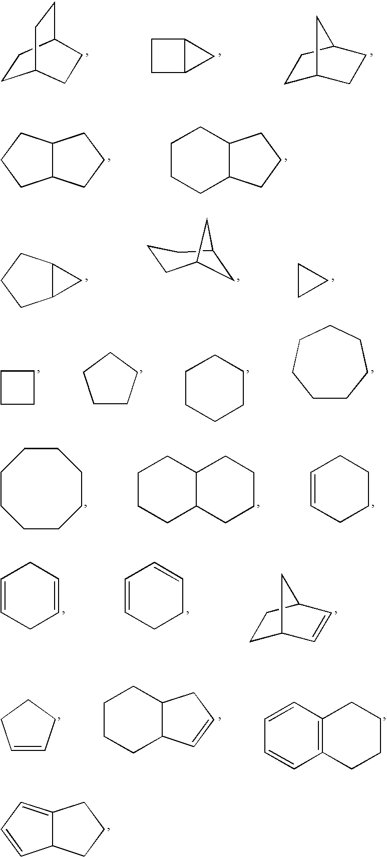

and the like. Monocyclic cycloalkyls include, but are not limited to, cyclopropyl, cyclobutyl, cyclopentyl, cyclohexyl, cycloheptyl, and cyclooctyl.

“Cycloalkylalkyl” refers to an alkyl, as is defined herein, substituted with a cycloalkyl, as is defined herein.

“Cycloalkylcarbonyl” refers to —C(═O)-cycloalkyl.

The term “heterocycle” refers to heteroaromatic and heteroalicyclic groups containing one to four ring heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, and furopyridinyl. The foregoing groups may be C-attached or N-attached where such is possible. For example, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Polycyclic heteroaryl groups may be fused or non-fused. Illustrative examples of heteroaryl groups include the following moieties:

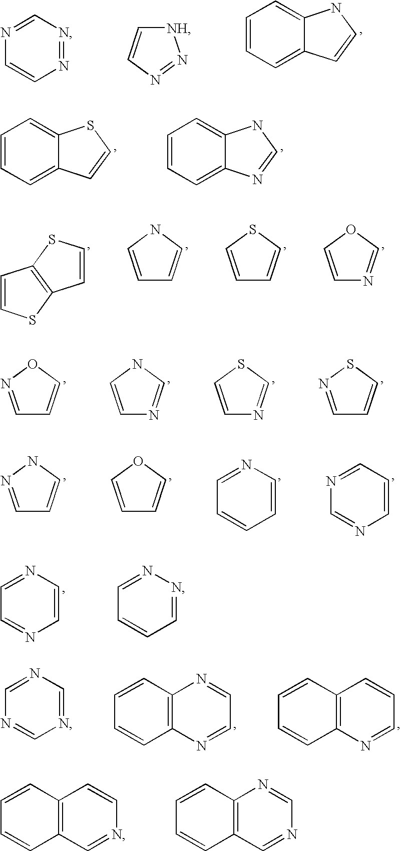

and the like.

In some embodiments, substituted or unsubstituted heteroaryl groups may be selected from among pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In other embodiments, substituted or unsubstituted heteroaryl groups may be selected from among pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In yet other embodiments, substituted or unsubstituted heteroaryl groups may be selected from among pyridinyl, pyrimidinyl, pyrazinyl, quinolinyl, isoquinolinyl, pyridazinyl, quinazolinyl, quinoxalinyl. In still other embodiments, substituted or unsubstituted heteroaryl groups may be selected from among pyridinyl, and quinolinyl.

“Heteroaralkyl” or “heteroarylalkyl” refers to an alkyl, as is defined herein, substituted with a heteroaryl as is defined herein.

A “heteroalicyclic” group or “heterocycloalkyl” group refers to a cycloalkyl group, wherein at least one skeletal ring atom is a heteroatom selected from nitrogen, oxygen and sulfur. The radicals may be fused with an aryl or heteroaryl. Illustrative examples of heterocycloalkyl groups, also referred to as non-aromatic heterocycles, include:

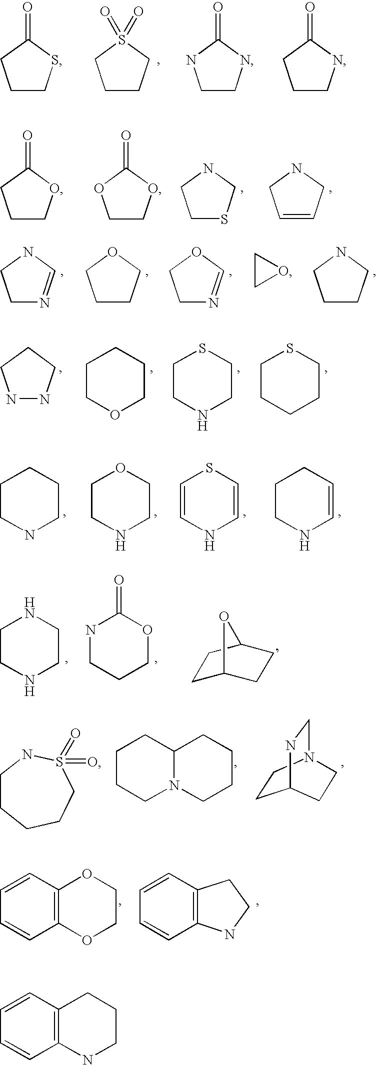

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e skeletal atoms of the heterocycloalkyl ring).

In some embodiments, substituted or unsubstituted heterocycloalkyl groups may be selected from among quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, indanyl, tetrahydronaphthalenyl, tetrahydroquinolinyl, tetrahydroisoquinolinyl, and tetrahydrothienyl. In other embodiments, substituted or unsubstituted heterocycloalkyl groups may be selected from among piperidinyl, morpholinyl, piperazinyl, dihydropyrrolyl, dihydroimidazolyl, tetrahydrofuranyl, dihydrooxazolyl, pyrrolidinyl, pyrazolidinyl, dihydrothienyl, imidazolidinonyl, pyrrolidinonyl, piperidinonyl, indolinyl, indanyl, tetrahydronaphthalenyl, tetrahydroquinolinyl, tetrahydroisoquinolinyl, and tetrahydrothienyl. In yet other embodiments, substituted or unsubstituted heterocycloalkyl groups may be selected from among piperidinyl, morpholinyl, piperazinyl, tetrahydrofuranyl, pyrrolidinyl, pyrrolidinonyl, piperidinonyl, indolinyl, indanyl, tetrahydronaphthalenyl, tetrahydroquinolinyl, and tetrahydrothienyl.

“Heterocycloalkylalkyl” refers to an alkyl, as defined herein, substituted with a heterocycloalkyl, as defined herein.

As used herein, “1,3-substituted-1H-indole-6-thiohydroxamic acid” refers to:

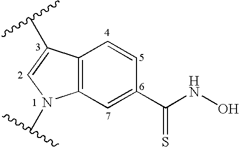

As used herein, “1,3-substituted-1H-indole-5-thiohydroxamic acid” refers to:

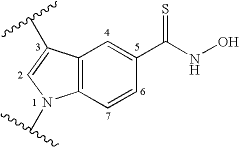

The term “thiohydroxamate”, “thiohydroxamic acid”, refers to:

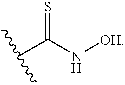

The term “hydroxamate”, “hydroxamic acid”, refers to:

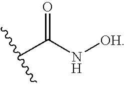

As used herein, hydroxamic acid HDAC inhibitor compound refers to a compound that includes at least one hydroxamic acid functional group (—C(═O)NHOH) and inhibits the activity of at least one HDAC.

As used herein, thiohydroxamic acid HDAC inhibitor compound refers to a compound that includes at least one thiohydroxamic acid functional group (—C(═S)NHOH) and inhibits the activity of at least one HDAC.

As used herein, carbothioester compound refers to a compound that includes at least one carbothioester functional group (—C(═S)OR).

The term “halo” or, alternatively, “halogen” means fluoro, chloro, bromo and iodo.

The terms “haloalkyl,” “haloalkenyl,” “haloalkynyl” and “haloalkoxy” include alkyl, alkenyl, alkynyl and alkoxy structures that are substituted with one or more halogens. The halogens may the same or they may be different. The terms “fluoroalkyl” and “fluoroalkoxy” include haloalkyl and haloalkoxy groups, respectively, in which the halo is fluorine. Non-limiting examples of haloalkyls include —CH₂Cl, —CF₃, —CHF₂, —CH₂CF₃, —CF₂CF₃, —CF(CH₃)₃, and the like. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of haloalkoxy groups include —OCF₃, —OCHF₂, —OCH₂F, —OCH₂CF₃, —OCF₂CF₃, —OCF₂CF₂CF₃, —OCF(CH₃)₃, and the like.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂OCH₃, —CH₂CH₂OCH₃, —CH₂CH₂OCH₂CH₂OCH₃, —CH₂CH₂OCH₂CH₂OCH₂CH₂OCH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and CH═CH—N(CH₃)—CH₃. In addition, up to two heteroatoms may be consecutive, such as, by way of example, —CH₂—NH—OCH₃ and CH₂—O—Si(CH₃)₃. Excluding the number of heteroatoms, a “heteroalkyl” may have from 1 to 8 carbon atoms, a “heteroalkenyl” may have from 2 to 8 carbons atoms, and a “heteroalkynyl” may have from 2 to 8 carbon atoms. Examples of heteroalkyls include but are not limited to, CH₂—O—CH₃, —CH₂CH₂OCH₃, —CH₂CH₂OCH₂CH₂OCH₃, —CH₂CH₂OCH₂CH₂OCH₂CH₂OCH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₃, —CH₂—S—CH₂—CH₃, —CH₂—CH₂, —S(O)—CH₃, —CH₂—CH₂—S(O)₂—CH₃, —CH₂—CH═N—OCH₃, —CH₂—NH—OCH₃ and CH₂—O—Si(CH₃)₃.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “cyano” group refers to a —CN group.

“Cyanoalkyl” refers to an alkyl, as is defined herein, substituted with a cyano.

“Cyanoalkylaminocarbonyl” refers to a —C(═O)NR′ (cyanoalkyl) group, where R′ is hydrogen, alkyl, heteroalkyl, haloalkyl, as is defined herein, cyanoalkyl is as defined herein.

An “isocyanato” group refers to a —NCO group.

A “thiocyanato” group refers to a —CNS group.

An “isothiocyanato” group refers to a —NCS group.

“Mercaptyl” or “sulfanyl” refers to a —S— moiety.

“Alkylthio” means an —SR radical where R is alkyl as defined herein.

“Acyloxy” refers to a RC(═O)O— group.

“Acyl” refers to a RC(═O)— group.

“Acylamino” refers to a RC(═O)N(R′)— group, where R′ is hydrogen, hydroxy, alkyl, or alkoxy. In some embodiments, R′ is H or R.

“Acyloxy” refers to RC(═O)O— group.

“Sulfinyl” refers to a —S(═O)— moiety.

“Alkylsulfinyl” means an —S(O)R radical where R is alkyl as defined herein.

“Sulfonyl” refers to a —S(═O)₂— moiety.

“Alkylsulfonyl” means a —SO₂R radical where R is alkyl as defined herein.

“Alkylsulfonylamino” means a —N(R′)SO₂R group, where R′ is hydrogen, alkyl, heteroalkyl, haloalkyl, as is defined herein, and R is alkyl as is defined herein.

“Phenylsulfonyl” refers to means a —S(═O)₂-phenyl moiety.

“Phenylsulfonylamino” refers to a —NR′SO₂— (phenyl) where R′ is hydrogen, alkyl, heteroalkyl, haloalkyl, as is defined herein.

“Heteroarylaminocarbonyl” refers to a —C(═O)NR′ (heteroaryl) group, where R′ is hydrogen, alkyl, heteroalkyl, haloalkyl, as is defined herein, and heteroaryl is as defined herein.

“Arylaminocarbonyl” refers to a —C(═O)NR′ (aryl) group, where R′ is hydrogen, alkyl, heteroalkyl, haloalkyl, as is defined herein, and aryl is as defined herein.

“Arylcarbonylamino” refers to —NR′C(═O)-(aryl) group, where R′ is hydrogen, alkyl, heteroalkyl, haloalkyl, as is defined herein, and aryl is as defined herein.

“Carboxy” refers to a —C(═O)OH group.

As used herein, the substituent “R” appearing by itself and without a number designation refers to a substituent selected from among from alkyl, haloalkyl, heteroalkyl, alkenyl, cycloalkyl, cycloalkylalkyl, aryl, arylalkyl, heteroaryl (bonded through a ring carbon), heteroarylalkyl, heterocycloalkyl, and heterocycloalkylalkyl.

The term “optionally substituted” or “substituted” means that the referenced group may be substituted with one or more additional group(s) individually and independently selected from alkyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, cyano, halo, acyl, acyloxy, isocyanato, thiocyanato, isothiocyanato, nitro, haloalkyl, fluoroalkyl, alkoxyalkyloxy, heterocycloalkylalkyloxy, heteroaralkyloxy, hydroxyalkoxy, aminoalkyl, aminoalkyloxy, alkoxyalkyloxy, alkoxyalkyl, phenyloxyalkyl, heterocycloalkylalkyl, amino, including mono- and di-substituted amino groups (e.g. NH₂, —NHR, —N(R)₂), and the protected derivatives thereof, and -alkylNR₂. By way of example, an optional substituents may be L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heterocycloalkyl, and C₁-C₈heteroalkyl. The protecting groups that may form the protective derivatives of the above substituents are found in sources such as Greene and Wuts, above.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (A), as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

A “selective HDAC8 inhibitor,” as used herein, refers to a compound that has an IC₅₀ for inhibition of HDAC8 deacetylase activity that is at least 5 fold to more than 500 fold lower than the IC₅₀ for inhibition of deacetylase activity of another HDAC. In some embodiments, the selective HDAC8 inhibitor has an IC₅₀ for inhibition of HDAC8 deacetylase activity that is about 5, 10, 50, 100, 150, 200, 250, 300, 350, 400, 450 or more than 500 fold lower than the IC₅₀ for inhibition of deacetylase activity of another HDAC. In one embodiment, the selective HDAC8 inhibitor has an IC₅₀ for inhibition of HDAC8 deacetylase activity that is at least 10 fold lower than the IC₅₀ for inhibition of deacetylase activity of at least one of HDAC1, HDAC2, HDAC3, HDAC6, HDAC10, and HDAC11; in another embodiment at least two of HDAC1, HDAC2, HDAC3, HDAC6, HDAC10, and HDAC11; in another embodiment all of HDAC1, HDAC2, HDAC3, HDAC6, HDAC10, and HDAC11. In another embodiment, the selective HDAC8 inhibitor has an IC₅₀ for HDAC8 deacetylase activity that is at least 20 fold lower than the IC₅₀ for inhibition of deacetylase activity of at least one of HDAC1, HDAC2, HDAC3, HDAC6, HDAC10, and HDAC11; in another embodiment at least two of HDAC1, HDAC2, HDAC3, HDAC6, HDAC10, and HDAC11; in another embodiment all of HDAC1, HDAC2, HDAC3, HDAC6, HDAC10, and HDAC11.

As used herein, the term “target protein” refers to a protein or a portion of a protein capable of being bound by a selective binding compound. In certain embodiments, a target protein is HDAC8.

As used herein, the term “selective binding compound” refers to a compound that selectively binds to any portion of one or more target proteins.

As used herein, the term “selectively binds” refers to the ability of a selective binding compound to bind to a target protein, such as, for example, HDAC8, with greater affinity than it binds to a non-target protein. In certain embodiments, specific binding refers to binding to a target with an affinity that is at least 10, 50, 100, 250, 500, 1000 or more times greater than the affinity for a non-target.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target. In certain embodiments, a modulator is an activator, which increases the magnitude of at least one activity of a target. In certain embodiments the presence of a modulator results in an activity that does not occur in the absence of the modulator.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “inhibits”, “inhibiting”, or “inhibitor” of HDAC, as used herein, refer to inhibition of histone deacetylase activity.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that the active ingredients, e.g. an idole compound described herein, and a co-agent, are both administered to a patient simultaneously in the form of a single entity or dosage. The term “non-fixed combination” means that the active ingredients, e.g. an indole compound described herein, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of an indole compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising an indole compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The term “enzymatically cleavable linker,” as used herein refers to unstable or degradable linkages which may be degraded by one or more enzymes.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds.

### Examples of Pharmaceutical Compositions and Methods of Administration

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, and a pharmaceutically acceptable diluent(s), excipient(s), or carrier(s). In addition, the compounds described herein can be administered as pharmaceutical compositions in which compounds described herein are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

A pharmaceutical composition, as used herein, refers to a mixture of an indole compound described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Alternately, one may administer the compounds and/or compositions in a local rather than systemic manner, for example, via injection of the compound directly into an organ, often in a depot preparation or sustained release formulation. Such long acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one indole compound described herein, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone®CL, Kollidon® CL, Polyplasdone® XL-10), larch arabogalactan, Veegum®, polyethylene glycol, waxes, sodium alginate, and the like.

“Bioavailability” refers to the percentage of the weight of at least one compound of any of Formula (I), Formula (Ia), Formula (Ib), Formula (Ic), Formula (II), Formula (Ia), Formula (IIa), Formula (IIIb), Formula (IVa), or Formula (IVb), described herein, that is delivered into the general circulation of the animal or human being studied. The total exposure (AUC(0-∞)) of a drug when administered intravenously is usually defined as 100% bioavailable (F %). “Oral bioavailability” refers to the extent to which at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, are absorbed into the general circulation when the pharmaceutical composition is taken orally as compared to intravenous injection.

“Blood plasma concentration” refers to the concentration of compounds disclosed herein, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound may vary from subject to subject.

“Carrier materials” include any commonly used excipients in pharmaceutics and should be selected on the basis of compatibility with compounds disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108, which are block copolymers of ethylene oxide and propylene oxide); and poloxamines (e.g., Tetronic 908, also known as Poloxamine 908, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel PH101, Avicel PH102, Avicel PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or l of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include at least one compound of any of Formula (I), Formula (Ia), Formula (Ib), Formula (Ic), Formula (II), Formula (Ia), Formula (IIa), Formula (IIIb), Formula (IVa), or Formula (IVb), described herein, can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the compounds described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, with one or more pharmaceutical excipients to form a bulk blend composition. When referring to these bulk blend compositions as homogeneous, it is meant that the particles of the compound described herein, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. In one embodiment, a binder usage level of up to 70% in tablet formulations is used.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax®, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of the at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds described herein, which sufficiently isolate the compound from other non-compatible excipients. Materials compatible with compound of any of Formula (I), Formula (Ia), Formula (Ib), Formula (Ic), Formula (II), Formula (Ia), Formula (IIIa), Formula (IIIb), Formula (IVa), or Formula (IVb), described herein, are those that delay the release of the compounds in vivo.

Exemplary microencapsulation materials useful for delaying the release of the formulations including at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compound of any of Formula (I), Formula (Ia), Formula (Ib), Formula (Ic), Formula (II), Formula (IIa), Formula (IIa), Formula (IIIb), Formula (IVa), or Formula (IVb), described herein, may be formulated by known methods. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compound of any of Formula (I), Formula (Ia), Formula (Ib), Formula (Ic), Formula (II), Formula (IIa), Formula (IIa), Formula (IIIb), Formula (IVa), or Formula (IVb), described herein, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compound of any of Formula (I), Formula (Ia), Formula (Ib), Formula (Ic), Formula (II), Formula (IIa), Formula (IIa), Formula (IIb), Formula (IVa), or Formula (IVb), described herein, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with at least one compound of any of Formula (I), Formula (Ia), Formula (Ib), Formula (Ic), Formula (II), Formula (Ia), Formula (IIa), Formula (IIIb), Formula (IVa), or Formula (IVb), described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When such salts are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, the formulations described herein, which include at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, are solid dispersions. Methods of producing such solid dispersions include, but are not limited to, for example, U.S. Pat. Nos. 4,343,789, 5,340,591, 5,456,923, 5,700,485, 5,723,269, and U.S. Pub. Appl 2004/0013734. In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions include, but are not limited to, for example, U.S. Pat. Nos. 4,151,273, 5,281,420, and 6,083,518.

The pharmaceutical solid oral dosage forms including formulations described herein, which include an at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, can be further formulated to provide a controlled release of the compound of any of Formula (I), Formula (Ia), Formula (Ib), Formula (Ic), Formula (II), Formula (IIa), Formula (IIIa), Formula (IIIb), Formula (IVa), or Formula (IVb). Controlled release refers to the release of the indole compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating for the methods and compositions described herein to achieve delivery to the lower gastrointestinal tract. In some embodiments such polymers are anionic carboxylic polymers. In other embodiments, the polymers and compatible mixtures thereof, and some of their properties, include, but are not limited to:

Shellac, also called purified lac. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman);
methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, are delivered using a pulsatile dosage form. A pulsatile dosage form is capable of providing one or more immediate release pulses at predetermined time points after a controlled lag time or at specific sites. Pulsatile dosage forms including the formulations described herein, may be administered using a variety of pulsatile formulations known in the art. For example, such formulations include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing the formulation described herein. The first group of particles provides a substantially immediate dose of the at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, upon ingestion by a mammal. The first group of particles can be either uncoated or include a coating and/or sealant. The second group of particles includes coated particles, which includes from about 2% to about 75%, preferably from about 2.5% to about 70%, and more preferably from about 40% to about 70%, by weight of the total dose of the at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes at least one compound of any of Formula (I), Formula (Ia), Formula (Ib), Formula (Ic), Formula (II), Formula (IIa), Formula (IIa), Formula (IIIb), Formula (IVa), or Formula (IVb), described herein.

Many other types of controlled release systems are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of the compound of any of Formula (I), Formula (Ia), Formula (Ib), Formula (Ic), Formula (II), Formula (IIa), Formula (IIIa), Formula (IIIb), Formula (IVa), or Formula (IVb), described herein, the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel PH102, Avicel PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68, F88t, and F108, which are block copolymers of ethylene oxide and propylene oxide); or poloxamines (e.g., Tetronic 908®, also known as Poloxamine 908).

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350 and 1450, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms include, but are not limited to, for example, U.S. Pat. Nos. 5,858,401, 6,667,048, and 6,960,563.

The above-listed additives should be taken as merely exemplary, and not limiting, of the types of additives that can be included in formulations described herein.

Intranasal formulations include, for example, U.S. Pat. Nos. 4,476,116, 5,116,817 and 6,391,452. Formulations that include at least one compound of any of Formula (I), Formula (Ia), Formula (Ib), Formula (Ic), Formula (II), Formula (IIa), Formula (IIa), Formula (IIIb), Formula (IVa), or Formula (IVb), described herein, which are prepared according to these and other techniques are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal formulations that include at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, may be administered using a variety of formulations, such as formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound of any of Formula (I), Formula (Ia), Formula (Ib), Formula (Ic), Formula (II), Formula (IIa), Formula (IIIa), Formula (IIIb), Formula (IVa), or Formula (IVb), described herein, described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

Transdermal formulations described herein may be administered using a variety of devices including, but not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate pharmaceutically acceptable excipients. In one embodiments, the transdermal formulations described herein include at least three components: (1) a formulation of at least one compound of any of Formula (I), Formula (Ia), Formula (Ib), Formula (Ic), Formula (II), Formula (Ia), Formula (IIIa), Formula (IIIb), Formula (IVa), or Formula (IVb), described herein, (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compound of any of Formula (I), Formula (Ia), Formula (Ib), Formula (Ic), Formula (II), Formula (Ia), Formula (IIIa), Formula (IIIb), Formula (IVa), or Formula (IVb), described herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Formulations that include at least one compound of any of Formula (I), Formula (Ia), Formula (Ib), Formula (Ic), Formula (II), Formula (IIa), Formula (IIIa), Formula (IIIb), Formula (IVa), or Formula (IVb), described herein, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations may include aqueous or nonaqueous solutions, preferably with physiologically compatible buffers or excipients.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The compounds described herein can be used in the preparation of medicaments for the inhibition of HDAC, or for the treatment of diseases or conditions that would benefit, at least in part, from inhibition of HDAC. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for the compounds described herein described herein are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Combination Treatments

The compounds and compositions described herein can also be used in combination with other known therapeutic agents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one compound described herein in combination with another therapeutic agent. By way of example only, if one of the side effects experienced by a patient upon receiving one of the compounds herein, such as compound of any of Formula (I), Formula (Ia), Formula (Ib), Formula (Ic), Formula (II), Formula (IIa), Formula (IIIa), Formula (IIIb), Formula (IVa), or Formula (IVb), described herein, is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a HDAC8 selective compound described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder or condition from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 5 years, and more preferably from about 1 month to about 3 years.

### Anti-Cancer Agents

Combinations of HDAC inhibitors described herein with other anti-cancer or chemotherapeutic agents are described herein. Examples of such agents can be found in Cancer Principles and Practice of Oncology by V. T. Devita and S. Hellman (editors), 6^{th} edition (Feb. 15, 2001), Lippincott Williams & Wilkins Publishers.

HDAC inhibitors in combination with other anti-cancer agents have been explored. HDAC inhibitors have been reported to be additive or synergistic with a number of anticancer agents, including, but not limited to anthrocyclins, fludarabine, flavopiridol, imatinib, bortezomib, anti-angiogenesis agents and nuclear receptor ligands, such as, all-trans retinoic acid and tumor necrosis factor-related apoptosis-inducing ligand (Fuino L, et al. (2003). Mol Cancer Ther 2: 971-984; Johnstone R W, Licht J D. (2003). Cancer Cell 4: 13-18; Bhalla K N. (2005). J Clin Oncol 23: 3971-3993; Dokmanovic M, Marks P A. (2005). J Cell Biochem 96: 293 304; Minucci S, Pelicci P G. (2006). Nat Rev Cancer 6: 38 51; Yoo C B, Jones P A. (2006). Nat Rev Drug Discov 5: 37-50).

Anti-cancer agents and/or agents used in chemotherapy include, but are not limited to, the following: estrogen receptor modulators, androgen receptor modulators, retinoid receptor modulators, cytotoxic/cytostatic agents, antiproliferative agents, prenyl-protein transferase inhibitors, nitrogen mustards, nitroso ureas, angiogenesis inhibitors, inhibitors of cell proliferation and survival signaling pathway, apoptosis inducing agents, agents that interfere with cell cycle checkpoints, agents that interfere with receptor tyrosine kinases (RTKs), integrin blockers, NSAIDs, inhibitors of inherent multi drug resistance (MDR), anti-emetic agents, agents useful in the treatment of anemia, agents useful in the treatment of neutropenia, immunologic-enhancing drugs, biphosphonates, aromatase inhibitors, agents inducing terminal differencation of neoplastic cells, γ-secretase inhibitors, cancer vaccines, and any combination thereof.

Where the subject is suffering from a cancer (e.g., a T-cell lymphoma), a selective HDAC8 inhibitor can be used in any combination with one or more other anti-cancer agents. Examples of anti-cancer agents include, but are not limited to, any of the following: 5-aza-2′-deoxycytidine, all trans retinoic acid, doxorubicin, vincristine, etoposide, gemcitabine, imatinib (Gleevec®), 17-N-allylamino-17-demethoxygeldanamycin (17-AAG), flavopiridol, LY294002, bortezomib, trastuzumab, BAY 11-7082, PKC412, or PD184352.

Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Other anti-cancer agents that can be employed in combination with a HDAC inhibitor include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine hydrochloride; hydroxyurea; idarubicin hydrochloride; ifosfamide; iimofosine; interleukin Il (including recombinant interleukin II, or r1L2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a HDAC inhibitor include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; ammubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9- dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a selective HDAC8 inhibitor include alkylating agents, antimetabolites, natural products, or hormones, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a HDAC inhibitor include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination with a HDAC inhibitor include, but are not limited to, nitrogen mustards (e.g., mechloroethamine, cyclophosphamide, chlorambucil, meiphalan, etc.), ethylenimine and methylmelamines (e.g., hexamethylmelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with a HDAC inhibitor include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide, SPD-424).

In another embodiment, Dynepo gene activated erythropoietin (Anti-anemic; human erythropoietin) may be administered in combination with HDAC inhibitor compounds.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpr-opanoate, 4,4′-dihydroxybenzophenone-2,4-dinitrophenyl-hydrazone, and SH646. In some embodiments, estrogen receptor modulators are tamoxifen and raloxifene.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylomithine, ILX23-7553, trans-N-(4′-hydroxyphenyl)retinamide, and N-4-carboxyphenyl retinamide.

Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with a selective HDAC8 inhibitor include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine-(chloro)platinum(II)]-tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, ammubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)-benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H)propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)colchic(2,3-d)-1,3-dioxol-6-one, 2,3-(methylenedioxy)-5-methyl-7-hydroxy-8-methoxybenzo[c]-phenanthridinium, 6,9-bis[(2-aminoethyl)-amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2-, 1-c]quinolin-7-one, and dimesna.

“Antiproliferative agents” include antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxy- cytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]-glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chloropheny-1)(1-methyl-1H-imidazol-5-yl)methyl]-4-(3-chlorophenyl)-1-methyl-2(1H)-quinolinone, (±)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethyl-phenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[11-(4-cyanobenzyl)-2-methyl-5-imidazolylmethyl]-2-piperazinone, 1-(2,2-diphenylethyl)-3-[N-(1-(4-cyanobenzyl)-1H-imidazol-5-yl- ethyl)carbamoyl]-piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol-4-ylmethyl}benzonitrile, 4-{3-[4-(2-oxo-2H-[1,2′]bipyridin-5′-ylmethyl]-3H-imidazol-4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6, 10:12,16-dimetheno-1H-imidazo[4,3-c][1,11,4]dioxa-azacyclononadecine-9-carbonitrile, (±)-19,20-dihydro-19-oxo-5H-18,21-ethano-12,14-etheno-6,10-metheno-22H-benzo[d]imidazo[4,3-k][1,6,9,12]-oxatriaza-cyclooctadecine-9-carbonitrile, 19,20-dihydro-19-oxo-5H,17H-18,21-ethano-6,10: 12,16-dimetheno-22H-imidazo[3,4-h][1,8,11,14]oxatriazacyclo-eicosine-9-carbonitrile, and (O)-19,20-dihydro-3-methyl-19-oxo-5H-18,21-ethano-12,14-etheno-6,10-metheno-22H-benzo[d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see J. Of Cancer, Vol. 35, No. 9, pp. 1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232, 632. Examples of reverse transcriptase inhibitors include delaviridine, efavirenz, GS-840, HB Y097, lamivudine, nevirapine, AZT, 3TC, ddC, and ddI. It has been reported (Nat. Med.; 8(3):225-32, 2002) that HIV protease inhibitors, such as indinavir or saquinavir, have potent anti-angiogenic activities and promote regression of Kaposi sarcoma.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valecoxib, and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin., Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105: 141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]-methyl]-1H-1,2,3-triazole-4-carboxamide, CM101, squalamine, combretastatin, RP14610, NX31838, sulfated mannopentose phosphate, 7,7-(carbonyl-bis[imino-N-methyl-4,2-pyrrolocarbonyl-imino[N-methyl-4,2-pyrrole]-carbonylimino]-bis-(1,3-naphthalene disulfonate), and 3-[(2,4-dimethylpyrrol-5-yl)methylene]-2-indolinone (SU5416).

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of CD20 (rituximab), inhibitors of cytokine receptors, inhibitors of MET, inhibitors of PDK (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573). Such agents include small molecule inhibitor compounds and antibody antagonists.

“Apoptosis inducing agents” include, but not limited to, activators of TNF receptor family members (including the TRAIL receptors).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include, but not limited to, tyrosine kinase inhibitors such as inhibitors of c-Kit, Eph, PDGF, Flt3, Lck, Btk, and c-Met. Further agents include inhibitors of RTKs shown as described by Bume-Jensen and Hunter, 2001, Nature 411: 355-365. Examples of “tyrosine kinase inhibitors” include, but not limited to, N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]-quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, ST1571, CEP2563, 4-(3-chlorophenylamino)-5,6-dimethyl-7-H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU11248, ST1571A, N-4-chlorophenyl-4-(4-pyridylmethyl)-1-phthalazinamine, and EMD121974.

HDAC inhibitors are also useful in combination with platelet fibrinogen receptor (GP Iib/IIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, 1999, Platelets 10: 285-292). Therefore, HDAC inhibitors can serve to inhibit metastasis, in combination with GP Iib/IIIa) antagonists. Examples of other fibrinogen receptor antagonists include abciximab, eptifibatide, sibrafiban, lamiflban, lotraflban, cromofiban, and CT50352.

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Other agents used in cancer therapy include those disclosed in U.S. patent publication 2005/0227929, herein incorporated by reference.

Commercially available anti-cancer agents which may be used in combination with an HDAC8 selective agent disclosed herein include, but are not limited to: abarelix (Plenaxis®); aldesleukin (Prokine®); Aldesleukin (Proleukin®); Alemtuzumab (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacizumab (Avastin®); bexarotene (Targretin); bleomycin (Blenoxane®); bortezomib (Velcade®); busulfan (Busulfex®); busulfan (Myleran®); calusterone (Methosarb®); capecitabine Xeloda®); carboplatin Paraplatin®); cannustine (BCNU, BiCNU); cannustine (Gliadel®); celecoxib (Celebrex®); cetuximab (Erbitux®); chlorambucil (Leukeran®); cisplatin (Platinol®); cladribine (Leustatin®); clofarabine (Clolar®); cyclophosphamide (Cytoxan®); cytarabine (Cytosar-U®); cytarabine liposomal (DepoCyt); dacarbazine (DTIC-Dome); dactinomycin(actinomycin D, Cosmegen®); Darbepoetin alfa (Aranesp®); dasatinib (Sprycel®); daunorubicin liposomal (DanuoXome); daunorubicin (daunomycin, Daunorubicin®); daunorubicin(daunomycin, Cerubidine®); decitabine (Dacogen®); denileukin (Ontak®); dexrazoxane (Zinecard®); docetaxel (Taxotere®); doxorubicin (Adriamycin®); doxorubicin liposomal (Doxil); dromostanolone propionate; epirubicin (Ellence®); Epirubicin; Epoetin alfa (EPOGEN®); erlotinib (Tarceva®); estramustine (Emcyt®); etoposide phosphate (Etopophos®); etoposide (VP-16; Vepesid®); exemestane (AROMASIN®); fentanyl citrate (Fentora®); Filgrastim (Neupogen®); floxuridine (FUDR); fludarabine (Fludara®); fluorouracil (5-FU, Adrucil); fulvestrant (Faslodex®); gefitinib (Iressa®); gemcitabine (Gemzar®); gemtuzumab ozogamicin (Mylotarg®); goserelin acetate (Zoladex®); histrelin acetate (Histrelin®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Leucovorin®); Leuprolide Acetate (Eligard®); levamisole (Ergamisol®); lomustine, CCNU CeeBU®); meclorethamine(nitrogen mustard, Mustargen®); megestrol acetate (Megace®); melphalan (Alkeran®); mercaptopurine (6-MP, Purinethol®); mesna (Mesnex®); methotrexate (Rheumatrex®, Trexall®); methoxsalen (Uvadex®); mitomycin C (Mutamycin®); mitomycin C (Mitozytrex®); mitotane (Lysodren®); mitoxantrone (Novantrone®); nandrolone phenpropionate (Durabolin-50); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pegademase (Adagen®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide (VM-26, Vumon®); testolactone (Teslac®); thalidomide (Thalomid®); thioguanine (6-TG, Thioguanine®); thiotepa (Thioplex®); topotecan (Hycamtin®); toremifene (Fareston®); Tositumomab (Bexxar®); Tositumomab/I-131 tositumomab (Bexxar®); trastuzumab (Herceptin®); tretinoin (ATRA, Vesanoid®); Uracil Mustard; valrubicin (Valstar®); vinblastine (Velban®); vincristine (Oncovin®); vinorelbine (Navelbine®); vorinostat (Zolinza®); zoledronate (Zometa®); and zoledronic acid (Zometa®).

The HDAC inhibitor compounds described herein may be used in combination with gene therapy for the treatment of cancer. For an overview of genetic strategies to treating cancer see Hall et al. (Am J Hum Genet 61:785-789, 1997) and Kufe et al. (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), Duc-4, NF-I, NF-2, RB, WT1, BRCA1, BRCA2, a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998, 5(8): 1105-13), and interferon-γ (J. Immunol. 2000; 164:217-222).

The HDAC inhibitor compounds described herein may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The HDAC inhibitor compounds described herein may be employed in conjunction with anti-emetic agents to treat nausea or emesis, including acute, delayed, late-phase, and anticipatory emesis, which may result from the use of a HDAC inhibitor compound described herein, alone or with radiation therapy. For the prevention or treatment of emesis, a HDAC inhibitor compound described herein may be used in conjunction with anti-emetic agents, such as, but not limited to: neurokinin-1 receptor antagonists, 5HT3 receptor antagonists (such as ondansetron, granisetron, tropisetron, Palonosetron, and zatisetron), GABA_{B} receptor agonists (such as baclofen), corticosteroids (such as dexamethasone, prednisone, prednisolone, or others such as disclosed in U.S. Pat. Nos. 2,789,118; 2,990,401; 3,048,581; 3,126,375; 3,929,768; 3,996,359; 3,928,326 and 3,749,712), dopamine antagonists (such as, but not limited to, domperidone, droperidol, haloperidol, chlorpromazine, promethazine, prochlorperazine, metoclopramide), antihistamines (H1 histamine receptor antagonists, such as but not limited to, cyclizine, diphenhydramine, dimenhydrinate, meclizine, promethazine, hydroxyzine), cannabinoids (such as but not limited to, cannabis, marinol, dronabinol), and others (such as, but not limited to, trimethobenzamide; ginger, emetrol, propofol).

In one embodiment, an anti-emesis agent selected from among a neurokinin-1 receptor antagonist, a 5HT3 receptor antagonist and a corticosteroid is administered as an adjuvant for the treatment or prevention of emesis that may result upon administration of the instant compounds.

The HDAC inhibitor compounds described herein may also be administered with an agent useful in the treatment of anemia. Such an anemia treatment agent is, for example, a continuous eythropoiesis receptor activator (such as epoetin-α).

The HDAC inhibitor compounds described herein may also be administered with an agent useful in the treatment of neutropenia. Examples of agents useful in the treatment of neutropenia include, but are not limited to, a hematopoietic growth factor which regulates the production and function of neutrophils such as a human granulocyte colony stimulating factor, (G-CSF). Examples of a G-CSF include filgrastim.

The HDAC inhibitor compounds described herein may also be administered with an immunologic-enhancing drug, such as levamisole, bacillus Calmette-Guerin, octreotide, isoprinosine and Zadaxin.

The HDAC inhibitor compounds described herein may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel®), pamidronate (Aredia®), alendronate (Fosamax®), risedronate(Actonel®), zoledronate (Zometa®), ibandronate (Boniva®), incadronate or cimadronate, clodronate, EB-1053, minodronate, neridronate, piridronate and tiludronate including any and all pharmaceutically acceptable salts, derivatives, hydrates and mixtures thereof.

The HDAC inhibitor compounds described herein may also be useful for treating breast cancer in combination with aromatase inhibitors. Examples of aromatase inhibitors include but are not limited to: anastrozole, letrozole and exemestane.

The HDAC inhibitor compounds described herein may also be useful for treating or preventing cancer in combination with siRNA or RNAi therapeutics.

“DNA methyltransferase inhibitor” refers to compounds which inhibit the methylation of the DNA base cytosine at the C-5 position of that base by the DNA methyltransferase enzyme. Examples of such DNA methyltransferase inhibitor include compounds disclosed in U.S. Pat. Nos. 6,329,412 and 6,268,137. Specific DNA methyltransferase inhibitors include 5-azacytosine and Zebularine®.

### Radiation Therapy

Radiotherapy, also called radiation therapy, is the treatment of cancer and other diseases with ionizing radiation. Ionizing radiation deposits energy that injures or destroys cells in an area being treated (a “target tissue”) by damaging their genetic material, making it impossible for these cells to continue to grow. Although radiation damages both cancer cells and normal cells, the latter are better able to repair themselves and function properly. Radiotherapy can be used to treat localized solid tumors, such as cancers of the skin, tongue, larynx, brain, breast, prostate, colon, uterus and/or cervix. It can also be used to treat leukemia and lymphoma (cancers of the blood-forming cells and lymphatic system, respectively).

A technique for delivering radiation to cancer cells is to place radioactive implants directly in a tumor or body cavity. This is called internal radiotherapy (brachytherapy, interstitial irradiation, and intracavitary irradiation are types of internal radiotherapy.) Using internal radiotherapy, the radiation dose is concentrated in a small area, and the patient stays in the hospital for a few days. Internal radiotherapy is frequently used for cancers of the tongue, uterus, prostate, colon, and cervix.

The term “radiotherapy” or “ionizing radiation” include all forms of radiation, including but not limited to α, β, and γ radiation and ultra violet light. Radiotherapy with or without concurrent or sequential chemotherapy is an effective modality for head and neck, breast, skin, anogenital cancers, and certain nonmalignant diseases such as keloid, desmoid tumor, hemangioma, arteriovenous malformation, and histocytosis X.

Provided are methods of using at least one histone deacetylase inhibitor to reduce side effect caused by at least one other therapeutic treatment, such as radiation-induced normal tissue fibrosis or chemotherapy-induced tissue necrosis, and the methods provided herein also synergistically inhibit tumor cell growth with radiotherapy and other anti-cancer agents.

### Growth Hormone Secretagogues

A inhibitor of HDAC can be used in combination with one or more growth hormone secretagogues including, but not limited to, arginine, L-3,4-dihydroxyphenylalanine (1-Dopa), glucagon, vasopressin, PACAP (pituitary adenylyl cyclase activating peptide), muscarinic receptor agonists and a synthetic hexapeptide, GHRP (growth hormone releasing peptide).

### Agents for Treating Autoimmune Diseases, Inflammatory Diseases, or Allergy Diseases

Where the subject is suffering from or at risk of suffering from an autoimmune disease, an inflammatory disease, or an allergy disease, a HDAC inhibitor compound can be administered in any combination with one or more of the following therapeutic agents: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

In one embodiment, HDAC inhibitor compounds described herein, or compositions and medicaments that include the HDAC inhibitor compounds described herein, may be administered to a patient in combination with an anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398).

Combinations with NSAIDs, which are selective COX-2 inhibitors, are contemplated herein. Such compounds include, but are not limited to those disclosed in U.S. Pat. No. 5,474,995; U.S. Pat. No. 5,861,419; U.S. Pat. No. 6,001,843; U.S. Pat. No. 6,020,343, U.S. Pat. No. 5,409,944; U.S. Pat. No. 5,436,265; U.S. Pat. No. 5,536,752; U.S. Pat. No. 5,550,142; U.S. Pat. No. 5,604,260; U.S. Pat. No. 5,698,584; U.S. Pat. No. 5,710,140; WO 94/15932; U.S. Pat. No. 5,344,991; U.S. Pat. No. 5,134,142; U.S. Pat. No. 5,380,738; U.S. Pat. No. 5,393,790; U.S. Pat. No. 5,466,823; U.S. Pat. No. 5,633,272; and U.S. Pat. No. 5,932,598; all of which are hereby incorporated by reference. Other examples of specific inhibitors of COX-2 include those disclosed in U.S. Pat. No. 6,313,138 the disclosure of which is incorporated herein by reference for such disclosure.

Compounds that have been described as selective COX-2 inhibitors and are therefore useful in the methods or pharmaceutical compositions described herein include, but are not limited to, celecoxib, rofecoxib, lumiracoxib, etoricoxib, valdecoxib, and parecoxib, or a pharmaceutically acceptable salt thereof.

Corticosteroids, include, but are not limited to: betamethasone (Celestone®), prednisone (Deltasone®), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Other agents used as anti-inflammatories include those disclosed in U.S. patent publication 2005/0227929, herein incorporated by reference.

Some commercially available anti-inflammatories include, but are not limited to: Arthrotec® (diclofenac and misoprostol), Asacol®, Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer®, Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Advil®, Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In one embodiment, HDAC inhibitors are administered in combination with leukotriene receptor antagonists including, but are not limited to, BAY u9773, Cuthbert et al EP 00791576 (published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of HDAC, or in which HDAC is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

These examples are provided for illustrative purposes only and not to limit the scope of the claims provided herein. The starting materials and reagents used for the synthesis of the compounds described herein may be synthesized or can be obtained from commercial sources, such as, but not limited to, Aldrich Chemical Co. (Milwaukee, Wis.), Sigma Chemical Co. (St. Louis, Mo.), or Bachem (Torrance, Calif.).

### Synthesis of Compounds

Compounds of Formula (I): Synthesis of compounds of Formula (I) can be achieved using methods described above and/or by using methods analogous to those presented for compounds of Formula (II).

### Compounds of Formula (II)

### Synthesis of 4-{2-[(3-dimethylaminomethylbenzofuran-2-carbonyl)amino]ethoxy}-phenyl thiohydroxamic acid (Compound VI-99)

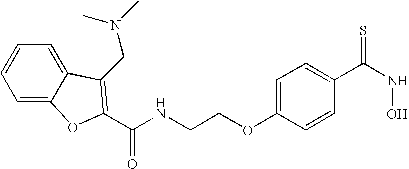

### Step 1: Synthesis of 4-(2-amino-ethoxy)-benzoic acid ethyl ester hydrochloride

Triphenyl phosphine (29.28 g, 112 mmol) and 4-hydroxybenzoic acid, ethyl ester (18.61 g, 112 mmol) were combined in a round bottom flask and dried overnight in vacuo. (2-Hydroxyethylcarbamic acid tert-butyl ester (15.2 g, 94.2 mmol) was weighed into a separate flask and dried in vacuo. The alcohol was transferred by cannula to the dried phosphine and acid using tetrahydrofuran (2×90 mL) and cooled to 0-5° C. A solution of diisopropyl azodicarboxylate (24.6 g, 122 mmol) in tetrahydrofuran (35 mL) was added dropwise over a period of 1 hour. After addition, the reaction was allowed to warm slowly to ambient temperature and stirred overnight. After completion of reaction, solvent was distilled under reduced pressure and the resulting oil was dissolved in absolute ethanol (50 mL) and ethyl acetate (200 mL). Acetyl chloride (20 mL, 283 mmol) was added dropwise over ten minutes and the temperature was raised to 40° C. The resulting suspension was allowed to stir for 2 hours and afterwards cooled to ambient temperature. After completion of reaction, the resulting crystals were filtered on a coarse fritted funnel and washed twice with ethyl acetate (75 mL). The solid was dried in vacuo to provide 16.93 g (61.9%) of 4-(2-amino-ethoxy)-benzoic acid ethyl ester hydrochloride as a white crystalline solid. ESI-MS: m/z 210 [M+H]⁺. ¹H NMR (300 Hz, DMSO): δ 8.39 (s, NH₃, 3H), 7.93 (d, PhH, 2H), 7.07 (d, PhH, 2H), 4.30-4.22 (m, CH₂CH₃, NCH₂CH₂O, 4H), 3.21 (t, NCH₂CH₂O, 2H), 1.29 (t, OCH₂CH₃, 3H) ppm. ¹³C NMR (300 Hz, DMSO): δ 165.1, 161.5, 131.0, 122.6, 114.5, 64.4, 60.2, 38.0, 14.0 ppm.

### Step 2: Synthesis of 4-{2-[(3-methylbenzofuran-2-carbonyl)amino]ethoxy}-benzoic acid ethyl ester

4-(2-Amino-ethoxy)-benzoic acid ethyl ester hydrochloride (6.28 g, 25.6 mmol) and 3-methylbenzofuran-2-carboxylic acid (4.50 g, 25.5 mmol) were combined in a flask and dried in vacuo. Acetonitrile (50 mL) and pyridine (15 mL) were added, and the resulting suspension was cooled to 0-5° C. Phosphorus oxychloride (4.36 g, 28.4 mmol) was added dropwise over ten minutes and the resulting solution was allowed to stir at 0-5° C. for one hour and at ambient temperature for one hour. The solution was added to 1 molar aqueous hydrochloric acid (300 mL), and the product extracted into dichloromethane (300 mL). The dichloromethane solution was washed with water (300 mL), 3% aqueous sodium hydroxide (300 mL), and twice with water (300 mL). Solvent was removed under reduced pressure and the solids dried in vacuo to provide 9.07 g (96.6%) 4-{2-[(3-methylbenzofuran-2-carbonyl)amino]ethoxy}-benzoic acid ethyl ester as a white foam. ESI-MS: m/z 368 [M+H]⁺. ¹H NMR (300 Hz, CDCl₃): δ 8.01 (d, PhH, 2H), 7.62-7.31 (m, ArH, 4H), 6.96 (d, PhH, 2H), 4.34 (q, CH₂CH₃, 2H), 4.21 (t, NCH₂CH₂O, 2H), 3.90 (t, NCH₂CH₂O, 2H), 2.63 (s, ArCH₃, 3H), 1.38 (t, OCH₂CH₃, 3H) ppm. ¹³C NMR (300 Hz, CDCl₃): δ 166.2, 162.2, 160.4, 153.3, 142.5, 131.6, 129.7, 127.1, 123.5, 123.1, 122.7, 120.9, 114.1, 111.5, 67.0, 60.6, 38.4, 14.3, 8.8 ppm.

### Step 3: Synthesis of 4-{2-[(3-dimethylaminomethylbenzofuran-2-carbonyl)amino]ethoxy}-benzoic acid ethyl ester

4-{2-[(3-methylbenzofuran-2-carbonyl)amino]ethoxy}-benzoic acid ethyl ester (2.50 g, 6.80 mmol), 2,2′-azobis-2-methylpropyl-nitrile (56 mg, 0.34 mmol), and N-bromosuccinimide (1.514 g, 8.51 mmol) were combined in a round bottom flask and dried in vacuo. Chlorobenzene (20 mL) was added, and the resulting solution was heated to 80° C. for 1.5 hours. The reaction mixture was allowed to cool to ambient temperature and washed with water (25 mL), 3% aqueous sodium bisulfite (25 mL), and water (25 mL). After washing, solvent was distilled in vacuo at 40° C. The residue was dissolved in dichloromethane (15 mL), and added to a stirred, cooled 2 molar solution of dimethylamine in tetrahydrofuran (10.2 mL). After stirring for one hour at 0.5° C., the solution was allowed to warm to ambient temperature for an additional hour. The reaction mixture was washed with 5% aqueous potassium carbonate (25 mL) and water (25 mL). Solvent was removed under reduced pressure and the resulting crude compound purified by silica gel chromatography using 1-3% methanol in dichloromethane as eluent to provide 1.701 g (60.9%) 4-{2-[(3-dimethylaminomethylbenzofuran-2-carbonyl)amino]ethoxy}-benzoic acid ethyl ester as a white foam. ESI-MS: m/z 411 [M+H]⁺. ¹H NMR (300 Hz, CDCl₃): δ 10.70 (s, NH, 1H), 8.01 (d, PhH, 2H), 7.64-7.30 (m, ArH, 4H), 6.93 (d, PhH, 2H), 4.34 (q, CH₂CH₃, 2H), 4.23 (t, NCH₂CH₂O, 2H), 3.91 (t, NCH₂CH₂O, 2H), 3.69 (s, NCH₂, 2H), 2.28 (s, CH₃, 6H), 1.38 (t, OCH₂CH₃, 3H) ppm. ¹³C NMR (300 Hz, CDCl₃): δ 166.2, 162.4, 160.3, 153.9, 146.9, 131.7, 128.6, 126.7, 123.5, 123.3, 120.2, 119.0, 114.0, 112.3, 67.2, 60.6, 52.0, 44.5, 39.1, 14.3 ppm.

### Step 4: Synthesis of 4-{2-[(3-dimethylaminomethylbenzofuran-2-carbonyl)amino]ethoxy}-thiobenzoic acid ethyl ester

Xylenes (15 mL) were added to 4-{2-[(3-dimethylaminomethylbenzofuran-2-carbonyl)amino]ethoxy}-benzoic acid ethyl ester (1.620 g, 3.95 mmol) and phosphorus pentasulfide (558 mg, 1.255 mmol). Hexamethyldisiloxane (1.048 g, 6.45 mmol) was added, and the resulting suspension was heated to 100° C. for 1 hour. Additional phosphorus pentasulfide (560 mg, 1.26 mmol) was added, and the resulting suspension was heated to 140° C. for 1 hour to form a two-phase solution. The xylenes phase was discarded upon cooling, and remaining solids were dissolved in dichloromethane (25 mL). The dichloromethane solution was washed with 5% aqueous potassium carbonate (30 mL), and dichloromethane removed under reduced pressure. The resulting crude compound was purified by silica gel chromatography using 0-2% methanol in dichloromethane as eluent to provide 0.833 g (49.5%) 4-{2-[(3-dimethylaminomethylbenzofuran-2-carbonyl)amino]ethoxy}-thiobenzoic acid ethyl ester as a yellow foam. ESI-MS: m/z 427 [M+H]⁺. ¹H NMR (300 Hz, CDCl₃): δ 12.87 (s, NH, 1H), 8.00 (d, PhH, 2H), 7.66-7.27 (m, ArH, 4H), 6.93 (d, PhH, 2H), 4.45-4.24 (m, NCH₂CH₂O, CH₃CH₃, 6H), 3.62 (s, NCH₂, 2H), 2.27 (s, CH₃, 6H), 1.38 (t, OCH₂CH₃, 3H) ppm. ¹³C NMR (300 Hz, CDCl₃): δ 185.8, 166.1, 162.2, 154.4, 150.9, 131.7, 128.8, 126.9, 123.6, 123.5, 120.2, 115.4, 113.9, 112.5, 65.7, 60.6, 51.8, 45.4, 44.1, 14.3 ppm.

### Step 5: Synthesis of 4-{2-[(3-dimethylaminomethylbenzofuran-2-carbonyl)amino]ethoxy}-benzoyl thiohydroxamic acid

4-{2-[(3-Dimethylaminomethylbenzofuran-2-carbonyl)amino]ethoxy}-thiobenzoic acid ethyl ester (0.788 g, 1.848 mmol) was dissolved in methanol (20 mL) and tetrahydrofuran (10 mL). To a solution of sodium hydroxide (0.739 g, 18.5 mmol) in water (4 mL), was added a 50% aqueous solution of hydroxylamine (1.83 mL, 27.7 mmol). The resulting hydroxylamine solution was added slowly to the solution of ester at ambient temperature. After stirring for one hour, concentrated aqueous hydrochloric acid (2.5 mL) and tetrahydrofuran (50 mL) were added. The resulting suspension was filtered, and solids washed with tetrahydrofuran (10 mL). Volatile solvents were removed from the combined filtrate under reduced pressure, isopropyl alcohol (10 mL) was added, and the resulting suspension recrystallized by heating and then cooling on ice. The solids were isolated by filtration and washed with water (30 mL), isopropyl alcohol (15 mL), and dried in vacuo to provide 0.380 g (45.7%) of 4-{2-[(3-dimethylaminomethylbenzofuran-2-carbonyl)amino]ethoxy}-benzoyl thiohydroxamic acid hydrochloride salt as yellow crystalline solid. ESI-MS: m/z 414 [M+H]⁺. ¹H NMR (300 Hz, DMSO): δ 13.00 (s, 1H), 11.50 (s, 1H), 9.00 (s, 1H), 7.87-7.03 (m, 8H), 4.38 (t, CH₂CH₂O, 2H), 4.06 (t, CH₂CH₂O, 2H), 3.73 (s, CH₂N, 2H), 2.14 (s, CH₃, 6H) ppm. ¹³C NMR (300 Hz, DMSO): δ 184.1, 166.8, 163.8, 161.7, 160.3, 153.2, 149.9, 131.3, 128.6, 126.9, 125.2, 123.4, 121.2, 116.4, 113.9, 111.5, 65.07, 64.9, 50.8, 45.1 ppm.

### Synthesis of 4-[2-(benzofuran-2-yl-carbonylamino)-ethoxy]-phenylthiohydroxamic acid

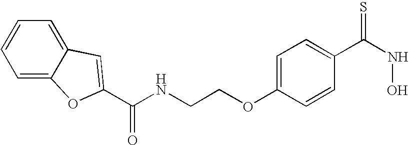

### Step 1

A mixture of benzofuran-2-carboxylic acid (0.162 g, 1 mmol), EDC-HCl (0.268 g, 1.4 mmol) and HOBT-H₂O (0.203 g, 1.5 mmol) in DMF (6 ml) was stirred for 2 h. Ethyl 4-(2-aminoethoxy)benzoate hydrochloride (0.232 g, 1 mmol) was added followed by triethylamine (0.121 g, 1.2 mmol). The reaction mixture was stirred for 2 h and then diluted with ethyl acetate, washed with saturated sodium bicarbonate solution, and brine. The organic layer was concentrated in vacuo to provide ethyl 4-[2-(benzofuran-2-ylcarbonylamino)ethoxy]benzoate.

### Step 2

Ethyl 4-[2-(benzofuran-2-ylcarbonylamino)ethoxy]benzoate (3.0 g), P₄S₁₀ (1.2 g), hexamethyldisiloxane (3.0 mL), were added to a flask and xylenes (25 mL) was added. The mixture is heated to 100 C for 40 minutes. The mixtures was cooled to room temperature, and 4 ml of ethanol were added. The solvent was removed in vacuo. The residue was dissolved in 30 mL of dichloromethane, washed with water (10 mL), and the organic solvent was removed in vacuo. The residue was dissolved in 20 mL of dichloromethane and purified by gravity column chromatography. 2.8 g of ethyl 4-[2-(benzofuran-2-ylcarbonylamino)ethoxy]thiobenzoate was obtained.

### Step 3

Ethyl 4-[2-(benzofuran-2-ylcarbonylamino)ethoxy]thiobenzoate (1 g) was added to methanol (25 mL) and tetrahydrofuran (5 mL). A solution of aqueous hydroxylamine (50% solution, 2.69 mL) was added to a solution of sodium hydroxide (1 g) in water (4 mL) was added to the suspension of ethyl 4-[2-(benzofuran-2-ylcarbonylamino)ethoxy]thiobenzoate. After 1 hour, conc. hydrochloric acid (2.5 mL) was added. Solvents were removed in vacuo. 30 mL of dichloromethane were added and the layers were separated (solid materials did not dissolve). The solids were filtered and washed with dichloromethane and then dried to provide 0.9 g of 4-[2-(benzofuran-2-yl-carbonylamino)-ethoxy]-phenylthiohydroxamic acid (ms (ESI) 356).

### Compounds of Formula IIIa, IIIb, IVa, IVb

### Synthesis of 1-methyl-1H-indole-5-carboxylic acid methyl ester

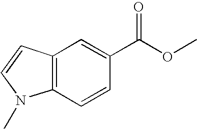

### Step 1

A solution of 1H-indole-5-carboxylic acid (1.6 g, 10 mmol), sodium hydride (1.2 g, 30 mmol), iodomethane (6.2 mL, 100 mmol) and DMF (40 mL) was stirred and room temperature for 24 hr. The reaction mixture was then quenched with water (5 mL) and diluted with ether (150 mL) and ethyl acetate (50 mL). After washing with aq. NH₄Cl (100 mL), water (100 mL) and then brine (100 mL), the organic layer was dried over MgSO₄, filtered and concentrated. The remaining material was subjected to flash chromatography (ethyl acetate/hexane 1:4) to provide 1-methyl-1H-indole-5-carboxylic acid methyl ester as a crystalline solid (1.77 g, 93% yield). ¹H NMR (400 MHz, CDCl₃) δ 8.40 (s, 1H), 7.94 (d, 1H, J=9.0 Hz), 7.34 (d, 1H, J=9.0 Hz), 7.12 (d, 1H, J=2.9 Hz), 6.60 (d, 1H, J=2.9 Hz), 3.94 (s, 3H), 3.83 (s, 3H).

### 1-methyl-1H-indole-6-carboxylic acid ethyl ester

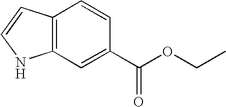

20 mL of ethanol was cooled to 0 C and NaH (3 g) was added. Methyl indole-6-carboxylate (1.3 g) was added and the reaction was stirred for 2 hours at room temperature. The reaction mixture was concentrated under reduced pressure. Dichloromethane was added and filtered. Purification was performed on silica gel using dichloromethane as the eluent. The ethyl ester was obtained (1.36 g).

### 1-(4-Methoxy-phenylmethyl)-1H-indole-6-thiohydroxamic acid (Compound VIII-23)

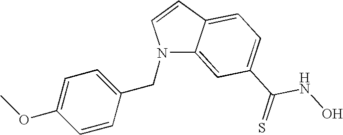

### Step 1

To a solution of 1H-indole-6-carboxylic acid ethyl ester (1.36 g) and 4-methoxybenzyl bromide (2.19 g) in DMF (20 mL) was added potassium carbonate (4.84 g). After stirring at room temperature for approx. 20 hours, the solution was diluted with ethyl acetate (200 mL), and washed with water (200 mL; three times). The organic layer was dried over Na₂SO₄, filtered and concentrated. The remaining residue was subjected to flash chromatography (20% ethyl acetate in hexane) to provide 1-(4-methoxy-phenylmethyl)-1H-indole-6-carboxylic acid ethyl ester (1.65 g).

### Step 2

1-(4-methoxy-phenylmethyl)-1H-indole-6-carboxylic acid ethyl ester (620 mg), P4S10 (222 mg), HMDO (542 mg) and xylenes (3 mL) were placed in a 10 mL round bottom flask and stirred at reflux for 8 hours. The reaction was cooled and purified on silica gel to provide O-ethyl 1-(4-methoxybenzyl)-1H-indole-6-carbothioate.

### Step 3

To a solution of sodium hydroxide (196 mg) and 50% aqueous hydroxylamine (7.308 mmol) was added O-ethyl 1-(4-methoxybenzyl)-1H-indole-6-carbothioate (0.32 g, 1.0 mmol) dissolved in MeOH. After stirring 1 hr at room temperature, the solution was diluted with water (4 mL) and the volatile solvents were removed in vacuo. The solution was then neutralized to pH=7-8 with 1N HC1. The resulting precipitate was isolated by filtration to provide 1-(4-methoxy-phenylmethyl)-1H-indole-6-thiohydroxamic acid (64 mg).

### Biological Examples

### Inhibition of A549 Human Lung Cancer Cell Proliferation by Thiohydroxamic Acids

The proliferation of A549 human lung cells grown in RPMI-1640 medium in the presence of thiohydroxamic acids was used to assess the activity of these compounds under cell culture conditions. A549 cells were allowed to adhere to 96-well microtiter plates (3000 cells per well, Rows B-H) overnight in 180 μL RPMI 1640 medium supplemented with 10% fetal bovine serum. Stock thiohydroxamic acid, as a 20 mM solution in DMSO, was diluted in tissue culture medium to form a secondary stock solution of thiohydroxamic acid (40 μM) and DMSO (0.8% v/v) in medium. This secondary stock solution of thiohydroxamic acid (60 μL) was serially diluted (1:4) in rows B through F (discarding the final 60 μL) on a microtiter plate. Row G was used for no-thiohydroxamic acid control. This process was repeated for each thiohydroxamic acid derivative tested. The plates were incubated at 37° C. under a 5% CO₂/95% air atmosphere. After 72 hours, cells were analyzed for viability using the tetrazolium dye, MTT (Mosmann, T. J. Immunol. Methods 1983, 65, 55-63). In brief, MTT dye (Sigma Chemical, St. Louis, Mo., catalogue no. M 2128, 5 mg/mL solution in phosphate buffered saline) was diluted to 0.5 mg/mL in fresh medium and added to cells to give a final volume of 150 μL. The plates were incubated at 37° C. for ca. 2 hours, whereupon the media was removed and isopropyl alcohol (100 μL/well) was added. Microtiter plates were vortexed for ca. 3 minutes to dissolve MTT formazan, and then analyzed on a microplate reader at 560-650 nm. The average absorption in the absence of cells (Row A) was subtracted from each well as background absorbance. Plate absorbances were normalized to the average of wells containing no thiohydroxamic acid (Row G) to allow plate-to-plate comparison. Data for each concentration of thiohydroxamic acid is the average of 10 wells. A dose-response was observed towards thiohydroxamic acids tested. Other adherent cell lines (e.g., K1 Chinese hamster ovary cells) were tested using a similar protocol.

### Pharmaceutical Compositions

Parenteral Composition

To prepare a parenteral pharmaceutical composition suitable for administration by injection, 100 mg of a water-soluble salt of a HDAC inhibitor compound described herein is dissolved in DMSO and then mixed with 10 mL of 0.9% sterile saline. The mixture is incorporated into a dosage unit form suitable for administration by injection.

In another embodiment, the following ingredients are mixed to form an injectable formulation.

| Ingredient | Amount |
|---|---|
| HDAC inhibitor compound described herein | 1.2 g |
| sodium acetate buffer solution (0.4 M) | 2.0 mL |
| HCl (1 N) or NaOH (1 M) | q.s. to suitable pH |
| water (distilled, sterile) | q.s.to 20 mL |

All of the above ingredients, except water, are combined and heated to 60-70° C. with stirring. A sufficient quantity of water at 60° C. is then added with vigorous stirring to emulsify the ingredients, and water then added q.s. to 100 g.

Oral Composition

To prepare a pharmaceutical composition for oral delivery, 100 mg of a HDAC inhibitor compound described herein is mixed with 750 mg of starch. The mixture is incorporated into an oral dosage unit for, such as a hard gelatin capsule, which is suitable for oral administration.

In another embodiment, the following ingredients are mixed intimately and pressed into single scored tablets.

| Ingredient | Quantity per tablet, mg |
|---|---|
| HDAC inhibitor compound described herein | 400 |
| Cornstarch | 50 |
| croscarmellose sodium | 25 |
| Lactose | 120 |
| magnesium stearate | 5 |

In yet another embodiment, the following ingredients are mixed intimately and loaded into a hard-shell gelatin capsule.

| Ingredient | Quantity per tablet, mg |
|---|---|
| HDAC inhibitor compound described herein | 200 |
| lactose, spray-dried | 148 |
| magnesium stearate | 2 |

In yet another embodiment, the following ingredients are mixed to form a suspension for oral administration.

| Ingredient | Amount | |
|---|---|---|
| HDAC inhibitor compound described herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| Flavoring | 0.035 | mL |
| Colorings | 0.5 | mg |

| | |
|---|---|
| distilled water | q.s. to 100 mL |

Sublingual (Hard Lozenge) Composition

To prepare a pharmaceutical composition for buccal delivery, such as a hard lozenge, mix 100 mg of a HDAC inhibitor compound described herein with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

Inhalation Composition

To prepare a pharmaceutical composition for inhalation delivery, 20 mg of a HDAC inhibitor compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

Rectal Gel Composition

To prepare a pharmaceutical composition for rectal delivery, 100 mg of a HDAC inhibitor compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

Suppository Formulation

A suppository of total weight 2.5 g is prepared by mixing a HDAC inhibitor compound described herein with Witepsol™ H-15 (triglycerides of saturated vegetable fatty acid; Riches-Nelson, Inc., New York), and has the following composition:

| Ingredient | Quantity per tablet, mg |
|---|---|
| HDAC inhibitor compound described herein | 500 |
| Witepsol ® H-15 | balance |

Topical Gel Composition

To prepare a pharmaceutical topical gel composition, 100 mg of a HDAC inhibitor compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

Ophthalmic Solution Composition

To prepare a pharmaceutical opthalmic solution composition, 100 mg of a HDAC inhibitor compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of disclosure and scope of the appended claims.

What is claimed is:

1. A compound of Formula (IIa): 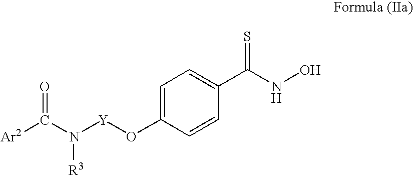
wherein:
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl, where Ar² is substituted or unsubstituted; or
a pharmaceutically acceptable salt thereof.

2. The compound of claim 1, wherein:
R³ is hydrogen; methyl; ethyl; propyl; butyl; i-propyl; 2-hydroxy-ethyl; phenyl; or benzyl.

3. The compound of claim 2, wherein:
Y is —CH₂—CH₂—; —CH₂—CH(CH₃)—; —CH(CH₃)—CH₂—; —CH(CH₂CH₃)—CH₂—; —CH(i-propyl)-CH₂—; —CH(i-butyl)-CH₂—; —CH(n-butyl)-CH₂—; —CH(cyclohexyl-methyl)-CH₂—; —CH(benzyl)-CH₂—; —CH(2-phenylethyl)-CH₂—; —CH(benzylS-methyl)-CH₂—; —CH(benzylSO₂-methyl)-CH₂—; —CH(2-MeS-ethyl)-CH₂—; —CH(hydroxy-methyl)-CH₂—; —CH(2-MeSO₂-ethyl)-CH₂—; —CH(4-Cl-benzyl)-CH₂—; —CH(benzyl-S-methyl)-CH₂—; —CH(phenyl)-CH₂—; —CH(benzylSO₂-methyl)-CH₂—; —CH₂—CH₂—CH₂—; —CH(CH₃Smethyl)-CH₂—; —CH(CH₃SO₂-methyl)-CH₂—; or —CH₂CH-(phenoxymethyl)-.

4. The compound of claim 3, wherein:
Ar² is heteroaryl optionally substituted with one or two substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R^{a} is hydroxyalkyl or optionally substituted phenyl), -alkylene-NR^{c}-alkyleneCONR^{c}R^{d} (where R^{c} is hydroxyl and R^{d} and R^{e} are independently hydrogen or alkyl), -alkylene-N(heteroalkyl)₂, or carboxyalkylaminoalkyl.

5. The compound of claim 4, wherein:
Ar² is thien-2-yl, pyridin-3-yl, quinolin-6-yl, benzothiazol-2-yl, benzoxazol-2-yl, benzofuran-2-yl, benzofuran-5-yl, benzothien-2-yl, furan-2-yl, 1H-benzimidazol-2-yl, 1H-pyrrol-2-yl, thiazol-2-yl, 1H-indol-2-yl, 1H-indol-5-yl, 1H-indol-3-yl, quinolin-3-yl, quinolin-8-yl, 1H-indazol-3-yl, 1H-benzotriazol-5-yl, isoquinolin-1-yl, isoquinolin-3-yl, quinoxalin-2-yl, quinolin-2-yl, 1H-benzimidazol-5-yl, quinolin-1-yl, pyridin-2-yl, quinolin-2-yl, furan-3-yl, or thien-3-yl, wherein Ar² is optionally substituted with one or two substituents selected from among alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)nR^{a} (where n is 0, 1, or 2; and R¹ is hydroxyalkyl or optionally substituted phenyl), -alkylene-NR-alkyleneCONR^{c}R^{d} (where R^{c} is hydroxyl and R^{d} and R^{e} are independently hydrogen or alkyl), -alkylene-N(heteroalkyl)₂, and carboxyalkylaminoalkyl.

6. A pharmaceutical composition comprising a therapeutically effective amount of a compound of claim 1 or pharmaceutically acceptable salts thereof and a pharmaceutically acceptable excipient.

7. A method for treating cancer which method comprises administering to the animal a pharmaceutical composition comprising a therapeutically effective amount of a compound of claim 1 and a pharmaceutically acceptable excipient in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

8. A 1,3-disubstituted-1H-indole-6-thiohydroxamic acid compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆-fluoroalkylene, C₂-C₆-fluoroalkenylene, C₁-C₆haloalkylene, C₂-C₆haloalkenylene, C₁-C₆heteroalkylene; C(═O)—, and C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆-fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl;
or an active metabolite, pharmaceutically acceptable solvate, pharmaceutically acceptable salt, pharmaceutically acceptable N-oxide, or pharmaceutically acceptable prodrug thereof.

9. The compound of claim 8, wherein:
X² is a substituted or unsubstituted group selected from among C₁-C₆alkylene, C₂-C₆alkenylene, C₁-C₆fluoroalkylene, C₂-C₆fluoroalkenylene, and C₁-C₆heteroalkylene.

10. The compound of claim 9, wherein:
R² is an optionally substituted group selected from among phenyl, naphthyl, monocyclic heteroaryl, bicyclic heteroaryl, C₃-C₈ cycloalkyl, monocyclic heterocycloalkyl, and bicyclic heterocycloalkyl.

11. The compound of claim 8, wherein:
R³ is hydrogen, halogen, substituted or unsubstituted C₁-C₆alkyl, substituted or unsubstituted C₂-C₆alkenyl, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, or C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl

12. The compound of claim 8, wherein the compound is a selective histone deacetylase 8 (HDAC8) inhibitor.

13. The compound of claim 12, wherein the selective HDAC8 inhibitor has an IC₅₀ for histone deacetylase 8 activity that is at least 10 fold lower than the IC₅₀ of the selective HDAC8 inhibitor for activity of histone deacetylase 1, histone deacetylase 2, histone deacetylase 3, histone deacetylase 6, histone deacetylase 10, or histone deacetylase 11.

14. A pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of claim 13 and a pharmaceutically acceptable diluent, excipient or binder.

15. A method of treating T-cell lymphoma or leukemia in a subject in need thereof, comprising administering to the subject a pharmaceutical composition containing a therapeutically effective amount of a compound of claim 13.

16. A method of improving the pharmacokinetic properties of a hydroxamic acid HDAC inhibitor compound, wherein said method comprises replacing the hydroxamic acid functional group of the hydroxamic acid HDAC inhibitor compound with a thiohydroxamic functional group to provide a thiohydroxamic acid HDAC inhibitor compound.

17. The method of claim 16, wherein replacing the hydroxamic acid functional group of the hydroxamic acid HDAC inhibitor compound with a thiohydroxamic functional group to provide a thiohydroxamic acid HDAC inhibitor compound comprises:
a) synthesizing the carboxylic acid ester compound that is the final precursor compound to the hydroxamic acid HDAC inhibitor compound, wherein treatment of the carboxylic acid ester compound with hydroxylamine would provide the hydroxamic acid HDAC inhibitor compound;
b) treating the carboxylic acid ester compound of a) with P₄S₁₀, and hexamethyldisiloxane (HMDO) to obtain a carbothioester compound; and
c) reacting the carbothioester compound of b) with hydroxylamine to provide a thiohydroxamic acid HDAC inhibitor compound.
